(19) United States
(12) Patent Application Publication
(10) Pub. No.: US 2023/0279126 A1
(43) Pub. Date: Sep. 7, 2023

(54) IL23 RECEPTOR SYNTHETIC CYTOKINES AND METHODS OF USE

(71) Applicant: Synthekine, Inc., Menlo Park, CA (US)

(72) Inventors: Robert Kastelein, Menlo Park, CA (US); Deepti Rokkam, Menlo Park, CA (US); Patrick J. Lupardus, Menlo Park, CA (US)

(21) Appl. No.: 18/018,444

(22) PCT Filed: Aug. 5, 2021
(86) PCT No.: PCT/US21/44850

§ 371 Date: Jan. 27, 2023

Related U.S. Application Data
(60) Provisional application No. 63/061,562, filed on Aug. 5, 2020.
(60) Provisional application No. 63/078,745, filed on Sep. 15, 2020.
(60) Provisional application No. 63/135,884, filed on Jan. 11, 2021.

(51) Int. Cl. C07K 16/28

(52) U.S. Cl. CPC C07K 16/2866; C07K 2317/569; C07K 2317/22; C07K 2317/31

(57) ABSTRACT

Provided herein are IL23 receptor binding molecules that bind to IL12Rβ1 and IL23R and comprise an anti-IL23R sdAb and an anti-IL23R V_{H}H antibody.

### CROSS-REFERENCES TO RELATED APPLICATIONS

This application is a U.S. National Phase of International Application No. PCT/US2021/044850, filed Aug. 5, 2021, which claims priority to U.S. Provisional Application No. 63/061,562, filed Aug. 5, 2020, U.S. Provisional Application No. 63/078,745, filed Sep. 15, 2020, and U.S. Provisional Application No. 63/135,884, filed Jan. 11, 2021, the disclosures of which are hereby incorporated by reference in their entirety for all purposes.

### SEQUENCE LISTING

10001.11 The instant application contains a Sequence Listing which has been submitted electronically in ASCII format and is hereby incorporated by reference in its entirety. Said ASCII copy, created on Jan. 22, 2023, is named 106249-1361935_SEQ_LST.txt and is 333,770 bytes in size.

### FIELD OF THE INVENTION

The present disclosure relates to biologically active molecules comprising single domain antibodies that specifically bind to the extracellular domain of the IL12 receptor and the IL23 receptor, compositions comprising such single domain antibodies, and methods ofuse thereof.

### BACKGROUND OF THE DISCLOSURE

IL12 is a heterodimeric cytokine comprise of the p35 and p40 subunits produced by dendritic cells, macrophages and neutrophils. The IL12 heterodimer is also referred to as p70. IL12 is typically identified as a T cell stimulating factor which can stimulate the proliferation and activity of T cells. IL12 stimulates the production of IFNgamma and TNFalpha and modulates the cytotoxic activity of NK and CD8+ cytotoxic T cells. IL12 is also involved in the immune cell differentiation in particular the differentiation of naïve T cells into Th1 (CD4+) cells. I112 is also reported to provide anti-antiogenic activity. ILl2 has been proposed for use in the treatment of a variety of neoplastic diseases, viral and bacterial infections.

IL12 binds to the IL12 receptor, a heterodimeric complex of IL12 receptor subunit beta-1 (IL12Rβ1 or IL12RB1) and I112 receptor subunit beta-2 (IL12Rβ2 or IL12RB2). IL12Rβ1 and IL12Rβ2 are members of the class I cytokine receptor family and have homology to gp130. The expression of IL12Rβ1 and IL12Rβ2 are upregulated in response to IL2 with the majority of IL12Rβ2 is found on activated T cells.

IL12Rβ1 (also known as CD212) is a constitutively expressed type I transmembrane protein that belongs to the hemopoietin receptor superfamily. IL12Rβ1 binds with low affinity to IL12. IL2Rβ1 is required for high-affinity binding to the IL12p40 subunit and it is associated with the Janus kinase (Jak) family member Tyk-2. The binding IL12p40 and IL12p35 to IL12Rβ1 and IL12Rβ2, respectively results in the activation of the Tyk-2 and Jak-2 Janus kinases occurs. The phosphorylated intracellular signaling domain of IL12Rβ2 provides a binding site for STAT4, which are phosphorylated and translocate to the nucleus regulating IFNgamma gene transcription.

In addition to forming one of the components of the IL12 receptor, IL12Rβ1 is also a component of the 1123 receptor. The IL23 receptor is a heterodimer of IL23R and I12Rβ1. IL23 binds IL23R with an affinity of 44 nM but binds to IL12Rβ1 with a significantly lower affinity of 2 μM. There is no apparent direct binding of IL23R to IL12Rβ1, the completion of the IL23:IL23R:IL12Rβ1 complex mediated by the initial formation of the IL23:1L23R complex which in turn binds to ILl2Rβ1.

The p40 subunit of the IL23 and IL12 cytokines provides the majority of binding sites for IL12Rβ1. In addition to forming a subunit of IL12 and IL23, p40 alone has significant bioactivity. P40 is reported to exist as both a monomer and a disulfide linked homodimer and which has a chemo attractant role for macrophages mediated by IL12Rβ1 alone. Gillesssen, et al (1995) European J. Immuno 25(1):200-206. The p40 homodimer is reported as a IL12 antagonist and its binding to IL12Rb1 is postulated to sequester the IL12Rb1 on the cell surface and suppressing the internalization or endocytosis of IL12Rβ1. Kundu, et al. (2017) PNASUSA 114(43):1148211487. Neutralization of p40 has been identified as reducing acute and chmnic GVHD through reducing Th1 and Th17 differentiation. This is in contrast to reports that IL12 can both exacerbate and suppress GVHD in various contexts but that targeting p40 has been p40 can be efficacious in reducing GVHD severity in experimental and clinical settings. In short, the activity of IL12 is a function of the competitive interaction of the IL12, p40 monomer and p40 homodimer with the IL12 receptor, in particular IL12Rβ1. Consequently, molecules which interfere in the association of p40 with IL12Rβ1 may be useful in the modulation of IL12 activity.

IL23 is a heterodimeric cytokine comprise of the p19 and p40 subunits produced by dendritic cells, macrophages and neutrophils. IL23 binds to the IL23 receptor, a heterodimeic complex of IL12 receptor subunit beta-1 (IL12Rβ1 or IL12RB1) and IL23R receptor subunit IL23 binds IL23R with an affinity of 44 nM but binds to IL12Rβ1 with a significantly lower affinity of 2 μM. There is no apparent direct binding of IL23R to IL12Rβ1, the completion of the IL23 IL23R:IL12Rβ1 complex mediated by the initial formation of the IL23:IL23R complex which in turn binds to IL12Rβ1. The IL23R binds primarily to the p19 subunit of the IL23 cytokine while the p40 subunit binds primarily to IL12Rb1.

In addition to forming one of the components of the IL12 receptor, IL12Rβ1 is also a component of the IL23 receptor IL12Rβ1 (also known as CD212) is a constitutively expressed type I transmembrane protein that belongs to the hemopoietin receptor superfamily. IL12Rβ1 binds with low affinity to IL23. IL12Rβ1 is required for high-affinity binding to the IL12p40 subunit and it is associated with the Janus kinase (Jak) family member Tyk-2 The binding IL12p40 and IL12p35 to IL12Rβ1 and IL12Rβ2, respectively results in the activation of the Tyk-2 and Jak-2 Janus kinases, induces STAT4 phosphorylation which in turn regulates IFNgmama gene transcription. The p40 subunit of the IL23 and IL12 cytokines provides the majority of binding sites for IL12Rβ1.

An antibody against p40, which blocks both IL23 and IL12 demonstrated activity in clinical trials of Crohn's disease. Selective blockade of the IL23R receptor activity by molecules that selectively bind to IL23R inhibit the by IL23p19/IL23R may provide more limited organ specific targeting o inflammatory disease without more widespread effects associate with inhibition of the p40/IL12 pathway. Additionally it has been shown that anti-IL23R, but not anti-IL23p19, partially suppressed lung metastases in tumor-bearing mice neutralized for IL12p40. Additionally it has been shown that IL23R has tumor-promoting effects that are partially independent of IL23p19. Consequently, the blockade of IL23R activity by molecules that selectively bind to IL23R is potentially effective in the suppression of tumor metastases. Yan, et al (2018) Cancer Immunol Res 6(8); 978-87.

Although monoclonal antibodies are the most widely used reagents for the detection and quantification of proteins, monoclonal antibodies are large molecules of about 150 kDa and it sometimes limits their use in assays with several reagents competing for close epitopes recognition. A unique class of immunoglobulin containing a heavy chain domain and lacking a light chain domain (commonly referred to as heavy chain” antibodies (HCAbs) is present in camelids, including dromedary camels, Bactrian camels, wild Bactrian camels, llamas, alpacas, vicuñas, and guanacos as well as cartilaginous fishes such as sharks. The isolated variable domain region of HCAbs is known as a VHH (an abbreviation for “variable-heavy-heavy” reflecting their architecture) or Nanobody® (Ablynx). Single domain VHH antibodies possesses the advantage of small size (˜12-14 kD), approximately one-tenth the molecular weight a conventional mammalian IgG class antibody) which facilitates the binding of these VHH molecules to antigenic determinants of the target which may be inaccessible to a conventional monoclonal IgG format (Ingram et al., 2018). Furthermore, VHH single domain antibodies are frequently characterized by high thermal stability facilitating pharmaceutical distribution to geographic areas where maintenance of the cold chain is difficult or impossible. These properties, particularly in combination with simple phage display discovery methods that do not require heavy/light chain pairing (as is the case with IgG antibodies) and simple manufacture (e.g., in bacterial expression systems) make VHH single domain antibodies useful in a variety of applications including the development of imaging and therapeutic agents that selectively bind cell surface exposed proteins.

### SUMMARY OF THE DISCLOSURE

The present disclosure provides compositions useful in the pairing of cellular receptors to generate desirable effects useful in treatment of disease in mammalian subjects.

The present disclosure provides binding molecules that comprise a first domain that binds to IL12Rβ1 of the IL23 receptor and a second domain that binds to IL23R of the IL23 receptor, such that upon contacting with a cell expressing IL12Rβ1 and IL23R, the IL-23 receptor binding molecule causes the functional association of IL12Rβ1 and IL23R, thereby resulting in functional dimerization of the receptors and downstream signaling.

Several advantages flow from the binding molecules described herein. The natural ligand of the IL23 receptor, IL23, causes IL12Rβ1 and IL23R to come into proximity (i.e., by their simultaneous binding of IL23). However, when IL23 is used as a therapeutic in mammalian, particularly human, subjects, it may also trigger a number of adverse and undesirable effects by a variety of mechanisms including the presence of IL12Rβ1 and I23R on other cell types and the binding to IL12Rβ31 and IL23R on the other cell types may result in undesirable effects and/or undesired signaling on cells expressing IL12Rβ31 and IL23R. The present disclosure is directed to methods and compositions that modulate the multiple effects of IL12Rβ1 and IL23R binding so that desired therapeutic signaling occurs, particularly in a desired cellular or tissue subtype, while minimizing undesired activity and/or intracellular signaling.

In some embodiments, the IL23 receptor binding molecules described herein are partial agonists of the. In some embodiments, the binding molecules described herein are designed such that the binding molecules are full agonists. In some embodiments, the binding molecules described herein are designed such that the binding molecules are super agonists.

In some embodiments, the binding molecules provide the maximal desired IL23 intracellular signaling from binding to IL12Rβ1 and IL23R on the desired cell types, while providing significantly less IL23 signaling on other undesired cell types. This can be achieved, for example, by selection of binding molecules having differing affinities or causing different Eₘₐₓ for IL12Rβ1 and IL23R as compared to the affinity of IL23 for IL12Rβ1 and IL23R Because different cell types respond to the binding of ligands to its cognate receptor with different sensitivity, by modulating the affinity of the dimeric ligand (or its individual binding moieties) for the IL23 receptor relative to wild-type IL23 binding facilitates the stimulation of desired activities while reducing undesired activities on non-target cells.

The present disclosure provides bivalent binding molecules that are agonists of the IL23 receptor, the bivalent binding molecule comprising:
a first single domain antibody (sdAb) that specifically binds to the extracellular domain of IL12Rβ1 of the IL23R (an “anti-IL12Rβ1 sdAb”), and
a second single domain antibody that specifically binds to extracellular domain IL23R of the IL23R ((an “anti-IL23R sdAb”),
   wherein the anti-IL12Rβ31 sdAb and anti-IL23R sdAb stably associated and first wherein contacting a cell expressing IL12Rβ1 and IL23R with an effective amount of the bivalent binding molecule results the dimerization of IL12Rβ1 and IL23R and results in intraceullar signaling characteristic of the IL23R receptor when activated by its natural cognate IL23. In some embodiments, one or both of the sdAbs is a an scFv. In some embodiments, one or both of the sdAbs is a VHH.

In some embodiments, one sdAb of the bivalent binding molecule is an scFv and the other sdAb is a VHH.

In some embodiments, the first and second sdAbs are covalently bound via a chemical linkage.

In some embodiments, the first and second sdAbs are provided as single continuous polypeptide.

In some embodiments, the first and second sdAbs are provided as single continuous polypeptide optionally comprising an intervening polypeptide linker between the amino acid sequences of the first and second sdAbs.

In some embodiments the bivalent binding molecule optionally comprising a linker, is optionally expressed as a fusion protein with an additional amino acid sequence. In some embodiments, the additional amino acid sequence is a purification handle such as a chelating peptide or an additional protein such as a subunit of an Fc molecule.

The disclosure also provides an expression vector comprising a nucleic acid encoding the bispecific binding molecule operably linked to one or more expression control sequences. The disclosure also provides an isolated host cell comprising the expression vector expression vector comprising a nucleic acid encoding the bispecific binding molecule operably linked to one or more expression control sequences functional in the host cell.

In another aspect, the disclosure provides a pharmaceutical composition comprising the IL-23 receptor binding molecule described herein and a pharmaceutically acceptable carrier.

In another aspect, the disclosure provides a method of treating an autoimmune or inflammatory disease, disorder, or condition or a viral infection in a subject in need thereof, comprising administering to the subject a therapeutically effective amount of an IL-23 receptor binding molecule described herein or a pharmaceutical composition described herein.

### BRIEF DESCRIPTION OF THE FIGURES

FIG. 1 of the attached drawings provides a schematic representation of one embodiment of the bivalent binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) interaction of a bivalent binding molecule and a second first receptor subunit comprising an extracellular domain (7), and transmembrane domain (8) and an intracellular domain (9) wherein the intracellular domain of the first receptor (6) and the intracellular domain of the second receptor (9) on of a bivalent binding molecule are within a proximal distance (11).

FIG. 2 of the attached drawings provides a schematic representation of two illustrative configurations of bivalent binding molecules of the present disclosure. Panel A provides a schematic representation of an illustrative single polypeptide chain bivalent binding molecule comprising, from amino to carboxy, a first single domain antibody (1) and a second single domain antibody (3) and a linker (2). Panel B provides a schematic representation of a bivalent binding molecule comprising a first single domain antibody (1) and a second single domain antibody (3) and a linker (2) and a knob-into-hole Fc domain, the Fc domain comprising a first subunit which is a Fc knob (13) and a second subunit which is a Fc hole (14) wherein the bivalent binding molecule is covalently linked to an Fc domain subunit via a IgG hinge sequence (12).

FIG. 3 of the attached drawings provides a schematic representations of two illustrative configurations of bivalent binding molecules of the present disclosure. Panel A provides a schematic representation of an illustrative bivalent binding molecule construct comprising two bivalent binding molecules each attached to a subunit of a knob-into-hole Fc domain, the construct comprising two polypeptide chains, the first polypeptide chain comprising, from amino to carboxy, a first single domain antibody (1), a linker (2) and a second single domain antibody (3), a IgG hinge sequence (12) and a Fc knob subunit (13) and a second polypeptide chain comprising, from amino to carboxy, a first single domain antibody (1), a linker (2) and a second single domain antibody (3), a IgG hinge sequence (12) and a Fc hole subunit (14) wherein the first and second polypeptides are in stable associate via the interaction of the knob-into-hole Fc domain. Panel B provides schematic representation of a an alternative arrangement of a bivalent binding molecule construct comprising two polypeptides a first polypeptide chain comprising, from amino to carboxy, a first single domain antibody (1), a linker (2) and a second single domain antibody (3), an IgG hinge sequence (12) and a Fc knob subunit (13) and a second polypeptide chain comprising, from amino to carboxy, a first second domain antibody (3), a linker (2) and a first single domain antibody (1), a IgG hinge sequence (12) and a Fc hole subunit (14), wherein the first and second polypeptides are in stable association via the interaction of the knob-into-hole Fc domain.

FIG. 4, Panel A provides alternative schematic representations of configurations of the bivalent binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG hinge sequence (12) and a Fc hole subunit (13), wherein the first and second single domain antibodies are in stable associate via the interaction of the knob-into-hole Fc domain.

FIG. 4, Panel B provides a schematic representation of a binding molecule the binding domains are single domain antibodies associated via transition metal coordinate covalent complex. As illustrated, the binding molecules comprises two polypeptide subunits: the first subunit comprising a first single domain antibody (1) is attached via a first linker (15) to a first chelating peptide (17) and second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

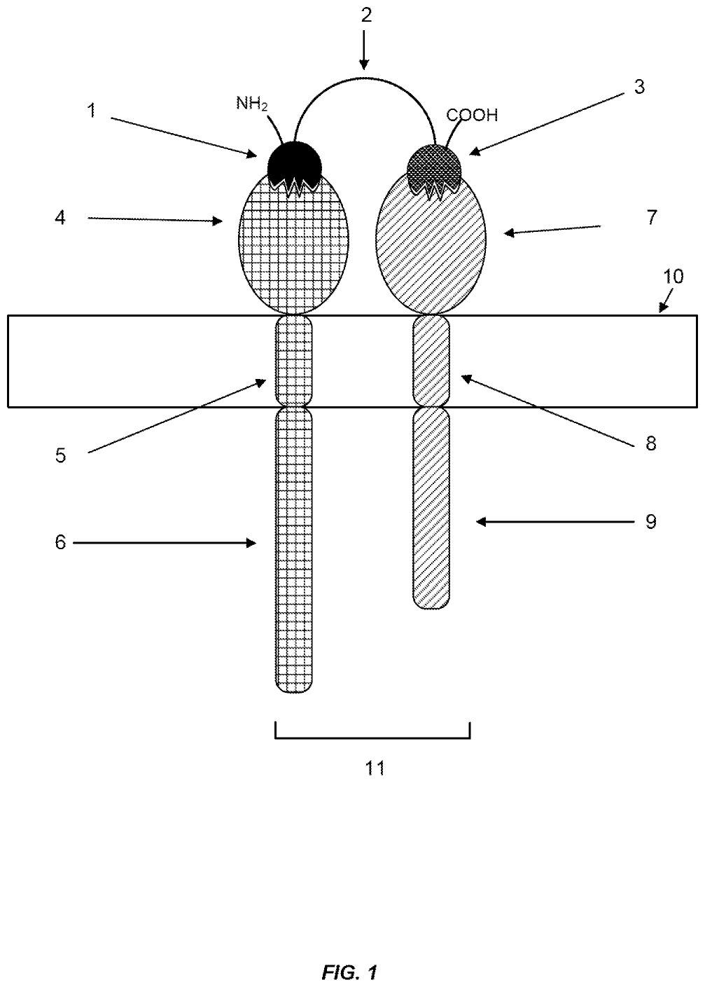

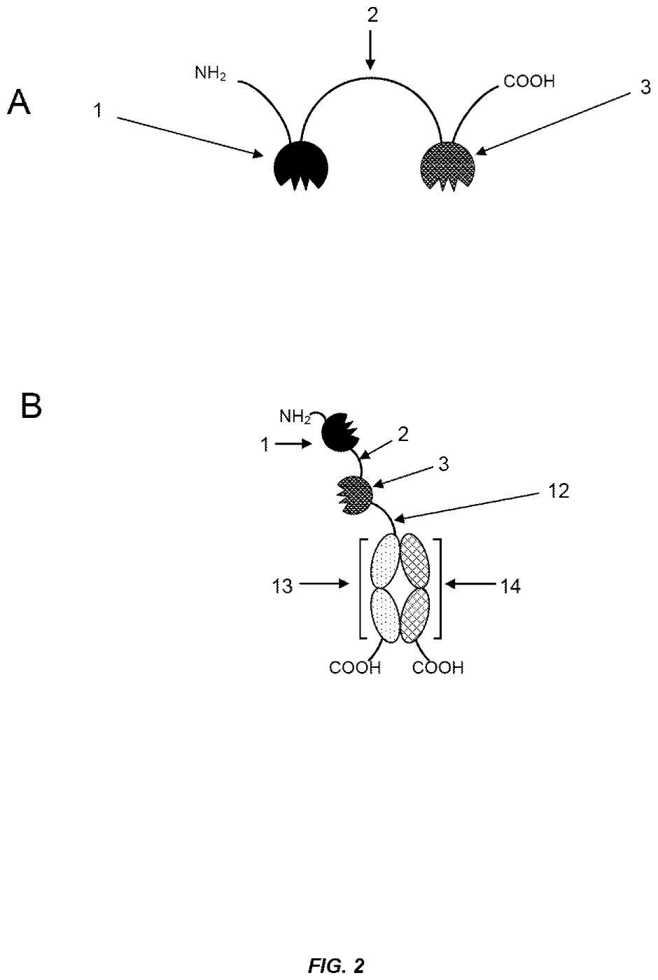

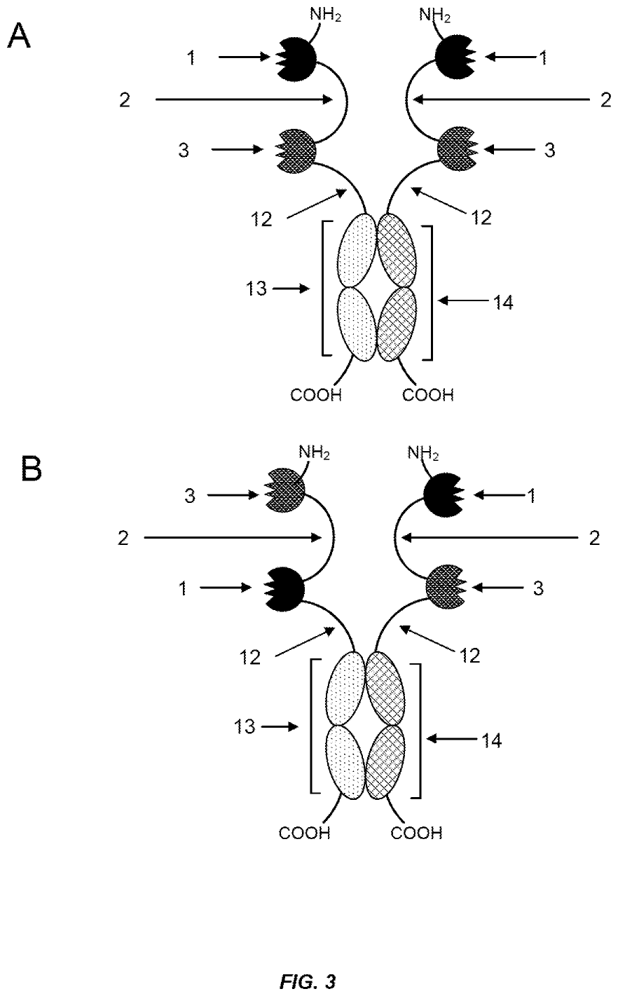

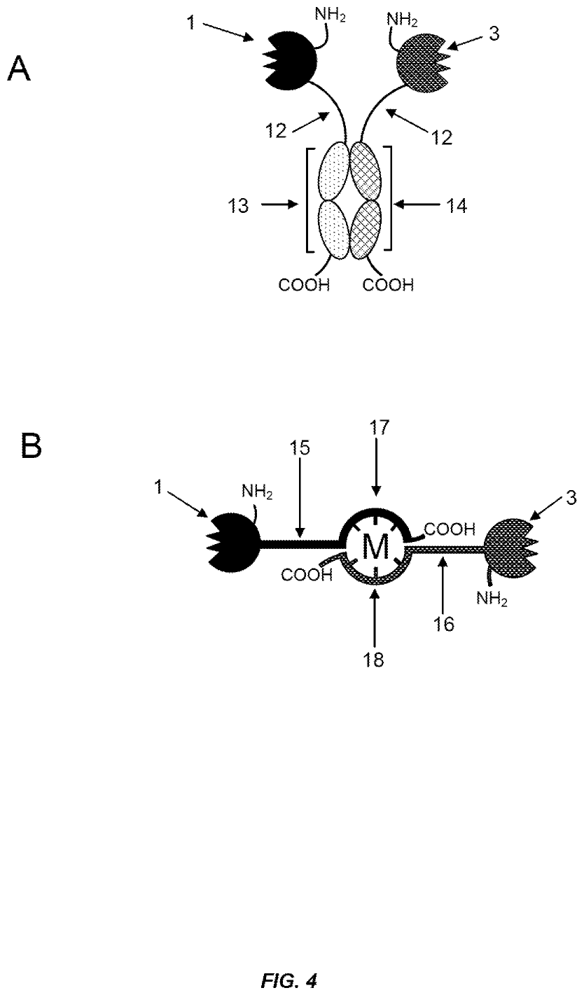

### DETAILED DESCRIPTION OF THE INVENTION

To facilitate the understanding of present disclosure, certain terms and phrases are defined below as well as throughout the specification. The definitions provided herein are non-limiting and should be read in view of the knowledge of one of skill in the art would know.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; g=microgram; mg=milligram; g=gram; kg=kilogram; dl or dL=deciliter; l or L=microliter, ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-bufferedsaline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g an ortholog, an IL2 ortholog, an engineered cell expressing an orthogonal receptor, an engineered cell expressing an orthogonal IL2 receptor, a CAR-T cell expressing an orthogonal IL2 receptor, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, inhalation and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (KD), a ratio of the dissociation rate constant between the molecule and its target (Koff) and the association rate constant between the molecule and its target (Kon).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by receptor understudy, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway, or cell.

Antibody: As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to target molecule and (b) immunoglobulin derivatives including but not limited to IgG (1-4) deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, and camelids antibodies (e.g., human antibodies). The term “antibody” encompasses antibodies isolatable from natural sources or from animals following immunization with an antigen and as well as engineered antibodies including monoclonal antibodies, bispecific antibodies, trispecific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries.

Binding molecule: As used herein, the term “binding molecule” refers to a bivalent molecule that can bind to the extracellular domain of two cell surface receptors. In some embodiments, a binding molecule specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

CDR: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat, et al., U.S. Dept. of Health and Human Services publication entitled “Sequences of proteins of immunological interest” (1991) (also referred to herein as “Kabat 1991” or “Kabat”); by Chothia, et al. (1987) J. Mol. Biol. 196:901-917 (also referred to herein as “Chothia”); and MacCallum, et al. (1996) J. Mol. Biol. 262:732-745, where the definitions include overlapping or subsets of amino acid residues when compared against each other. Nevertheless, application of either definition to refer to a CDR of an antibody or grafted antibodies or variants thereof is intended to be within the scope of the term as defined and used herein. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs2 and 3 in the variable region of an antibody follows Kabat numbering or simply, “Kabat.” The positioning of CDR1 in the variable region of an antibody follows a hybrid of Kabat and Chothia numbering schemes.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 10%. In particular embodiments, one measurement is comparable to a reference standard if it deviates by less than 15%, alternatively by less than 10%, or alternatively by less than 5% from the reference standard.

Effective Concentration (EC): As used herein, the terms “effective concentration” or its abbreviation “EC” are used interchangeably to refer to the concentration of an agent (e.g., an hIL2 mutein) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the with respect to particular parameter and test system.

Extracellular Domain: As used herein the term “extracellular domain” or its abbreviation “ECD” refers to the portion of a cell surface protein (e.g. a cell surface receptor) which is outside of the plasma membrane of a cell. The term “ECD” may include the extra-cytoplasmic portion of a transmembrane protein or the extra-cytoplasmic portion of a cell surface (or membrane associated protein).

Identity: As used herein, the term “percent (%) sequence identity” or “substantially identical” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%,75%, 80%,85%, 90%,91%, 92%, 93%, 94%, 95%, 96%, 97%, 98%, or 99% sequence identity to the reference sequence as determined with BLAST using standard parameters, as described below. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned. Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁵, and most preferably less than about 10⁻²⁰.

Intracellular Signaling: As used herein, the terms “intracellular signaling” and “downstream signaling” are used interchangeably to refer to the to the cellular signaling process that is caused by the interaction of the intracellular domains (ICDs) of two or more cell surface receptors that are in proximity of each other. In rececptor complexes via the JAK/STAT pathway, the association of the ICDS of the receptor subunits brings the JAK domains of the ICDs into proximit which initiates a phosphorylation cascade in which STAT molecules are phosphorylated and translocate to the nucleus associating with particular nucleic acid sequences resulting in the activation and expression of particular genes in the cell. The binding molecules of the present disclosure provide intraceullar signaling characteristic of the IL23R receptor when activated by its natural cognate IL23. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signalinging caused by the binding molecule, can also be measured.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polypeptide that may be employed to link two protein domains to provide space and/or flexibility between the two protein domains.

Modulate: As used herein, the terms “modulate”, “modulation” and the like refer to the ability of a test agent to affect a response, either positive or negative or directly or indirectly, in a system, including a biological system or biochemical pathway.

Multimerization: As used herein, the term “multimerization” refers to two or more cell surface receptors, or domains or subunits thereof, being brought in close proximity to each other such that the receptors, or domains or subunits thereof, can interact with each other and cause intracellular signaling.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. The terms “immediately N-terminal” or “immediately C-terminal” are used to refers to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the

Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. A In some embodiments, the IL-23 receptor binding molecule has a reduced Eₘₐₓ compared to the Eₘₐₓ caused by IL23. Eₘₐₓ reflects the maximum response level in a cell type that can be obtained by a ligand (e.g., a binding molecule described herein or the native cytokine (e.g., IL23)). In some embodiments, the IL-23 receptor binding molecule described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IL23. In other embodiments, the Eₘₐₓ of the IL-23 receptor binding molecule described herein is greater (e.g., at least 1%, 5%, 10%, 15%, 20%, 25%, 30%, 35%,40%, 45%, or 50% greater) than the Eₘₐₓ of the natural ligand, IL23. In some embodiments, by varying the linker length of the IL-23 receptor binding molecule, the Eₘₐₓ of the IL-23 receptor binding molecule can be changed. The IL-23 receptor binding molecule can cause Eₘₐₓ in the most desired cell types, and a reduced Eₘₐₓ in other cell types.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Proximity: As used herein, the term “proximity” refers to the spatial proximity or physical distance between two cell surface receptors, or domains or subunits thereof, after a binding molecule described herein binds to the two cell surface receptors, or domains or subunits thereof. In some embodiments, after the binding molecule binds to the cell surface receptors, or domains or subunits thereof, the spatial proximity between the cell surface receptors, or domains or subunits thereof, can be, e.g., less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the spatial proximity amounts to less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

Receptor: As used herein, the term“receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a multimeric complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lower case “r” (e.g., rhIL2) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors. In contrast, the terms “inhibition”, “down-regulation” and the like refer to the opposite effects.

Single Domain Antibody (sdAb): The term “single-domain antibody” or “sdAbs,” refers to an antibody having a single (only one) monomeric variable antibody domain. A sdAb is able to bind selectively to a specific antigen. A V_{H}H antibody, further defined below, is an example of a sdAb.

Specifically Binds: As used herein, the term “specifically bind” refers to the degree of selectivity or affinity for which one molecule binds to another. In the context of binding pairs (e.g., a binding molecule described herein/receptor, a ligand/receptor, antibody/antigen, antibody/ligand, antibody/receptor binding pairs), a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample.

Stably Associated: As used herein, the term “stably associated” or “in stable association with” are used to refer to the various means by which one molecule (e.g., a polypeptide) may be associated with another molecule over an extended period of time. The stable association of one molecule to another may be effected by a variety of means, including covalent bonding and non-covalent interactions. In some embodiments, stable association of two molecules may be effected by covalent bonds such as peptide bonds. In other embodiments, stable association of two molecules may be effected b non-covalent interactions. Examples of non-covalent interactions which may provide a stable association between two molecules include electrostatic interactions (e.g., hydrogen bonding, ionic bonding, halogen binding, dipole-dipole interactions, Van der Waals forces and π-effects including cation-π interactions, anion-π interactions and π-π interactions) and hydrophobilic/hydrophilic interactions. In some embodiments, the stable association of sdAbs of the bivalent binding molecules of the present disclosure may be effected by non-covalent interactions. In one embodiment, the non-covalent stable association of the sdAbs of the bivalent binding molecules may be achieved by conjugation of the sdAbs to “knob-into-hole” modified Fc monomers. An Fc “knob” monomer stably associates non-covalently with an Fc “hole” monomer. Conjugation of a first sdAb which specifically binds to the extracellular domain of a first subunit of a heterodimeric receptor to an “Fc knob” monomer and conjugation of an second sdAb which specifically binds to the extracellular domain of a second subunit of a heterodimeric receptor to an “Fc hole” monomer provides stable association of the first and second sdAbs. The knob-into-hole modification is more fully described in Ridgway, et al. (1996) Protein Engineering 9(7):617-621 and U.S. Pat. No. 5,731,168, issued Mar. 24, 1998, U.S. Pat. No. 7,642,228, issued Jan. 5, 2010, U.S. Pat. No. 7,695,936, issued Apr. 13, 2010, and U.S. Pat. No. 8,216,805, issued Jul. 10, 2012. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”) and ii) in the CH3 domain of a second heavy chain, an amino acid residue is replaced with an amino acid residue having a smaller side chain (e.g., alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 on one chain and Y349 on the second chain which results in a stabilizing disulfide bridge between the two antibody heavy chains in the Fc region (Carter, et al. (2001) Immunol Methods 248, 7-15). The knob-into-hole format is used to facilitate the expression of a first polypeptide (e.g., an IL23R binding sdAb) on a first Fc monomer with a “knob” modification and a second polypeptide on the second Fc monomer possessing a “hole” modification to facilitate the expression of heterodimeric polypeptide conjugates.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially: As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g., blood count), genomic data, protein expression data, immunohistochemistry, that the subject requires or will benefit from treatment. The term suffering from is typically used in conjunction with a particular disease state such as“suffering from a neoplastic disease” refers to a subject which has been diagnosed with the presence of a neoplasm.

Therapeutically Effective Amount: As used herein, the term The phrase “therapeutically effective amount” is used in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, modification of biomarker levels, increase in duration of survival, extended duration of progression free survival, extension of the time to progression, increased time to treatment failure, extended duration of event free survival, extension of time to next treatment, improvement objective response rate, improvement in the duration of response, and the like that that are relied upon by clinicians in the field for the assessment of an improvement in the condition of the subject in response to administration of an agent.

Treat: The terms “treat”, “treating”, treatment” and the like refer to a course of action (such as administering a binding molecule described herein, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

VHH: As used herein, the term “V_{H}H” is a type of sdAb that has a single monomeric heavy chain variable antibody domain. Such antibodies can be found in or produced from Camelid mammals (e.g., camels, llamas) which are naturally devoid of light chains V_{H}Hs can be obtained from immunization of camelids (including camels, llamas, and alpacas (see, e.g., Hamers-Casterman, et al. (1993) Nature 363:446-448) or by screening libraries (e.g., phage libraries) constructed in V_{H}H frameworks. Antibodies having a given specificity may also be derived from non-mammalian sources such as V_{H}Hs obtained from immunization of cartilaginous fishes including, but not limited to, sharks. In a particular embodiment, a V_{H}H in a bispecific V_{H}H² binding molecule described herein binds to a receptor (e.g., the first receptor or the second receptor of the natural or non-natural receptor pairs) if the equilibrium dissociation constant between the V_{H}H and the receptor is greater than about 10⁶ M, alternatively greater than about 108 M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. 1980 Analyt. Biochem. 107:220-239). Standardized protocols for the generation of single domain antibodies from camelids are well known in the scientific literature. See, e.g., Vincke, et al (2012) Chapter 8 in Methods in Molecular Biology, Walker, J. editor (Humana Press, Totowa N.J.). Specific binding may be assessed using techniques known in the art including but not limited to competition ELISA, BIACORE® assays and/or KINEXA® assays. In some embodiments, a V_{H}H described herein can be humanized to contain human framework regions. Examples of human germlines that could be used to create humanized V_{H}Hs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProtID: P01768), VH3-11 (e.g., UniProtID: P01762), and VH3-9 (e.g., UniProtID: P01782).

V_{H}H²: As used herein, the term “V_{H}H²” and “bispecific V_{H}H²” and “VHH dimer” refers to are used interchangeably to refer to a subtype of the binding molecules of the present disclosure wherein the first and second sdAbs are both VHHs and first V_{H}H binding to a first receptor, or domain or subunit thereof, and a second V_{H}H binding to a second receptor, or domain or subunit thereof.

Wild Type: As used herein, the term “wild type” or “WT” or “native” is used to refer to an amino acid sequence or a nucleotide sequence that is found in nature and that has not been altered by the hand of man.

### I. IL23 Receptor Binding Proteins

The present disclosure relates to synthetic mimetics of the naturally occurring IL23 which are agonists of the IL23R. The IL23R is a heterodimeric protein complex of IL12RB1 and IL23R. The binding of the IL23 results in dimerization IL12Rβ1 and IL23R and intracellular signaling in cells expressing IL12Rβ1 and IL23R characteristic of the binding of the naturally occurring IL23 for the IL23R. In some embodiments, the IL23R is the human IL23R and the IL23 is the human IL23. In some embodiments the IL23R is the murine IL23R and the IL23 is the murine IL23.

IL12Rβ1

IL12 is a heterodimeric cytokine comprise of the p35 and p40 subunits produced by dendritic cells, macrophages and neutrophils. The IL12 heterodimer is also referred to as p70. IL12 is typically identified as a T cell stimulating factor which can stimulate the proliferation and activity of T cells. IL12 stimulates the production of IFNgamma and TNFalpha and modulates the cytotoxic activity of NK and CD8+ cytotoxic T cells. IL12 is also involved in the immune cell differentiation in particular the differentiation of naïve T cells into Th1 (CD4+) cells. IL12 is also reported to provide anti-antiogenic activity. IL12 has been proposed for use in the treatment of a variety of neoplastic diseases, viral and bacterial infections.

IL12 binds to the IL12 receptor, a heterodimeric complex of IL12 receptor subunit beta-1 (IL12Rβ1 or IL12RB1) and IL12 receptor subunit beta-2 (IL12Rβ2 or IL12RB2). IL12Rβ1 and IL12Rβ2 are members of the class I cytokine receptor family and have homology to gp130. The expression of IL12Rβ1 and IL12Rβ2 are upregulated in response to IL2 with the majority of IL12Rβ2 is found on activated T cells.

IL12Rβ1 (also known as CD212) is a constitutively expressed type I transmembrane protein that belongs to the hemopoietin receptor superfamily. I2RP 1 binds with low affinity to IL12. IL12Rβ1 is required for high-affinity binding to the IL12p40 subunit and it is associated with the Janus kinase (Jak) family member Tyk-2. The binding IL12p40 and IL12p35 to IL12Rβ1 and IL12Rβ2, respectively results in the activation of the Tyk-2 and Jak-2 Janus kinases occurs. The phosphorylated intracellular signaling domain of IL12Rβ2 provides a binding site for STAT4, which are phosphorylated and translocate to the nucleus regulating IFNgamma gene transcription.

In addition to forming one of the components of the I2 receptor, ILl2R 1 is also a component of the IL23 receptor. The IL23 receptor is a heterodimer of IL23R and I2Rβ1. IL23 bin ds IL23R with an affinity of 44 nM but binds to IL12Rβ1 with a significantly lower affinity of 2 μM. There is no apparent direct binding of IL23R to IL12Rβ1, the completion of the IL23:IL23R:IL12Rβ1 complex mediated by the initial formation of the IL23:IL23R complex which in turn binds to IL12Rβ1.

The p40 subunit of the IL23 and IL12 cytokines provides the majority of binding sites for IL12Rβ1. In addition to forming a subunit of IL12 and IL23, p40 alone has significant bioactivity. P40 is reported to exist as both a monomer and a disulfide linked homodimer and which has a chemo attractant role for macrophages mediated by IL12Rβ1 alone. Gillesssen, et al (1995) European J. Immuno 25(1):200-206. The p40 homodimer is reported as a IL12 antagonist and its binding to IL12Rb1 is postulated to sequester the IL12Rb1 on the cell surface and suppressing the internalization or endocytosis of IL12Rβ1. Kundu, et al. (2017) PNASUSA 114(43):1148211487. Neutralization of p40 has been identified as reducing acute and chronic GVHD through reducing Th1 and Th17 differentiation. This is in contrast to reports that IL12 can both exacerbate and suppress GVHD in various contexts but that targeting p40 has been p40 can be efficacious in reducing GVHD severity in experimental and clinical settings. In short, the activity of IL12 is a function of the competitive interaction of the IL12, p40 monomer and p40 homodimer with the IL12 receptor, in particular IL12Rβ. Consequently, molecules which interfere in the association of p40 with IL12Rβ1 may be useful in the modulation of IL12 activity.

Human IL12RB1

In one embodiment, specifically bind to the extracellular domain of the human IL12RB1 receptor subunit (hIL12RB1). hIL12RB1 is expressed as a 662 amino acid precursor comprising a 23 amino acid N-terminal signal sequence which is post-translationally cleaved to provide an 639 amino acid mature protein. The canonical full-length acid hIL12RB1 precursor (including the signal peptide) is a 662 amino acid polypeptide having the amino acid sequence:

(SEQ ID NO: 1)
MEPLVTWVVPLLFLFLLSRQGAACRTSECCFQDPP
YPDADSGSASGPRDLRCYRISSDRYECSWQYEGPT
AGVSHFLRCCLSSGRCCYFAAGSATRLQFSDQAGV
SVLYTVTLWVESWARNQTEKSPEVTLQLYNSVKYE
PPLGDIKVSKLAGQLRMEWETPDNQVGAEVQFRHR
TPSSPWKLGDCGPQDDDTESCLCPLEMNVAQEFQL
RRRQLGSQGSSWSKWSSPVCVPPENPPQPQVRFSV
EQLGQDGRRRLTLKEQPTQLELPEGCQGLAPGTEV
TYRLQLHMLSCPCKAKATRTLHLGKMPYLSGAAYN
VAVISSNQFGPGLNQTWHIPADTHTEPVALNISVG
TNGTTMYWPARAQSMTYCIEWQPVGQDGGLATCSL
TAPQDPDPAGMATYSWSRESGAMGQEKCYYITIF
ASAHPEKLTLWSTVLSTYHFGGNASAAGTPHHVSV
KNHSLDSVSVDWAPSLLSTCPGVLKEYVVRCRDED
SKQVSEHPVQPTETQVTLSGLRAGVAYTVQVRADT
AWLRGVWSQPQRFSIEVQVSDWLIFFASLGSFLSI
LLVGVLGYLGLNRAARHLCPPLPTPCASSAIEFPG
GKETWQWINPVDFQEEASLQEALVVEMSWDKGERT
EPLEKTELPEGAPELALDTELSLEDGDRCKAKM.

For purposes of the present disclosure, the numbering of amino acid residues of the human IL12RB1 polypeptides as described herein is made in accordance with the numbering of this canonical sequence (UniProt Reference No P42701, SEQ ID NO:1). Amino acids 1-23 of SEQ ID NO:1 are identified as the signal peptide of hIL12RB1, amino acids 24-545 of SEQ ID NO:1 are identified as the extracellular domain, amino acids 546-570 of SEQ ID NO:1 are identified as the transmembrane domain, and amino acids 571-662 of SEQ ID NO:1 are identified as the intracellular domain.

For the purposes of generating antibodies that bind to the ECD of IL12RB1, immunization may be performed with the extracellular domain of the hIL12RB1. The extracellular domain of hIL12RB1 is a 522 amino acid polypeptide of the sequence:

(SEQ ID NO: 2)
CRTSECCFQDPPYPDADSGSASGPRDLRCYRISSD
RYECSWQYEGPTAGVSHFLRCCLSSGRCCYFAAGS
ATRLQFSDQAGVSVLYTVTLWVESWARNQTEKSPE
VTLQLYNSVKYEPPLGDIKVSKLAGQLRMEWETPD
NQVGAEVQFRHRTPSSPWKLGDCGPQDDDTESCLC
PLEMNVAQEFQLRRRQLGSQGSSWSKWSSPVCVPP
ENPPQPQVRFSVEQLGQDGRRRLTLKEQPTQLELP
EGCQGLAPGTEVTYRLQLHMLSCPCKAKATRTLHL
GKMPYLSGAAYNVAVISSNQFGPGLNQTWHIPADT
HTEPVALNISVGTNGTTMYWPARAQSMTYCIEWQP
VGQDGGLATCSLTAPQDPDPAGMATYSWSRESGAM
GQEKCYYITIFASAHPEKLTLWSTVLSTYHFGGNA
SAAGTPHHVSV
KNHSLDSVSVDWAPSLLSTCPGVLKEYVVRCRDED
SKQVSEHPVQPTETQVTLSGLRAGVAYTVQVRADT
AWLRGVWSQPQRFSIEVQVSD.

Mouse IL12RB1

In one embodiment, specifically bind to the extracellular domain of the mouse or murine IL12RB1 receptor subunit (mIL12RB1). mIL12RB1 is expressed as a 738 amino acid precursor comprising a 19 amino acid N-terminal signal sequence which is post-translationally cleaved to provide a 719 amino acid mature protein. The canonical full-length acid mIL12RB1 precursor (including the 24 amino acid signal peptide) is a 738 amino acid polypeptide having the amino acid sequence:

(SEQ ID NO: 3)
MDMMGLAGTSKHITFLLLCQLGASGPGDGCCVEKT
SFPEGASGSPLGPRNLSCYRVSKTDYECSWQYDGP
EDNVSHVLWCCFVPPNHTHTGQERCRYFSSGPDRT
VQFWEQDGIPVLSKVNFWVESRLGNRTMKSQKISQ
YLYNWTKTTPPLGHIKVSQSHRQLRMDWNVSEEAG
AEVQFRRRMPTTNWTLGDCGPQVNSGSGVLGDIRG
SMSESCLCPSENMAQEIQIRRRRRLSSGAPGGPWS
DWSMPVCVPPEVLPQAKIKFLVEPLNQGGRRRLTM
QGQSPQLAVPEGCRGRPGAQVKKHLVLVRMLSCRC
QAQTSKTVPLGKKLNLSGATYDLNVLAKTRFGRST
IQKWHLPAQELTETRALNVSVGGNMTSMQWAAQAP
GTTYCLEWQPWFQHRNHTHCTLIVPEEEDPAKMVT
HSWSSKPTLEQEECYRITVFASKNPKNPMLWATVL
SSYYFGGNASRAGTPRHVSVRNQTGDSVSVEWTAS
QLSTCPGVLTQYVVRCEAEDGAWESEWLVPPTKTQ
VTLDGLRSRVMYKVQVRADTARLPGAWSHPQRFSF
EVQISRLSIIFASLGSFASVLLVGSLGYIGLNR
AAWHLCPPLPTPC
GSTAVEFPGSQGKQAWQWCNPEDFPEVLYPRDALV
VEMPGDRGDGTESPQAAPECALDTRRPLETQRQRQ
VQALSEARRLGLAREDCPRGDLAHVTLPLLLGGVT
QGASVLDDLWRTHKTAEPGPPTLGQEA

For purposes of the present disclosure, the numbering of amino acid residues of the mIL12RB1 polypeptides as described herein is made in accordance with the numbering of this canonical sequence (UniProt Reference No. Q60837, SEQ ID NO:3). Amino acids 1-19 of SEQ ID NO:3 are identified as the signal peptide of mIL12RB1, amino acids 20-565 of SEQ ID NO:3 are identified as the extracellular domain, amino acids 566-591 of SEQ ID NO:3 are identified as the transmembrane domain, and amino acids 592-738 of SEQ ID NO:3 are identified as the intracellular domain.

For the purposes of generating antibodies that bind to the ECD of IL12RB1, immunization may be performed with the extracellular domain of the mIL12RB1. The extracellular domain of the mIL12RB1 receptor is a 546 amino acid polypeptide of the sequence:

(SEQ ID NO: 4)
QLGASGPGDGCCVEKTSFPEGASGSPLGPRNLSCY
RVSKTDYECSWQYDGPEDNVSHVLWCCFVPPNHTH
TGQERCRYFSSGPDRTVQFWEQDGIPVLSKVNFWV
ESRLGNRTMKSQKISQYLYNWTKTTPPLGHIKVSQ
SHRQLRMDWNVSEEAGAEVQFRRRMPTTNWTLGDC
GPQVNSGSGVLGDIRGSMSESCLCPSENMAQEIQI
RRRRRLSSGAPGGPWSDWSMPVCVPPEVLPQAKIK
FLVEPLNQGGRRRLTMQGQSPQLAVPEGCRGRPGA
QVKKHLVLVRMLSCRCQAQTSKTVPLGKKLNLSGA
TYDLNVLAKTRFGRSTIQKWHLPAQELTETRALNV
SVGGNMTSMQWAAQAPGTTYCLEWQPWFQHRNHTH
CTLIVPEEEDPAKMVTHSWSSKPTLEQEECYRITV
FASKNPKNPMLWATVLSSYYFGGNASRAGTPRHVS
VRNQTGDSVSVEWTASQLSTCPGVLTQYVVRCEAE
DGAWESEWLVPPTKTQVTLDGLRSRVMYKVQVRAD
TARLPGAWSHPQRFSFEVQIS

### IL23R

IL23 is a heterodimeric cytokine comprise of the p19 and p40 subunits produced by dendritic cells, macrophages and neutrophils. IL23 binds to the IL23 receptor, a heterodimeric complex of IL12 receptor subunit beta-I (IL12Rβ1 or IL12RB1) and IL23R receptor subunit. IL23 binds IL23R with an affinity of 44 nM but binds to IL12Rβ1 with a significantly lower affinity of 2 μM. There is no apparent direct binding of IL23R to IL12Rβ1, the completion of the IL23:IL23R:IL12Rβ1 complex mediated by the initial formation of the IL23:IL23R complex which in turn binds to I2RP 1. The IL23R binds primarily to the p19 subunit of the IL23 cytokine while the p40 subunit binds primarily to IL12Rb1.

In addition to forming one of the components of the IL12 receptor, IL12Rβ1 is also a component of the IL23 receptor. IL12Rβ1 (also known as CD212) is a constitutively expressed type I transmembrane protein that belongs to the hemopoietin receptor superfamily. IL12Rβ1 binds with low affinity to IL23. IL12Rβ1 is required for high-affinity binding to the IL12p40 subunit and it is associated with the Janus kinase (Jak) family member Tyk-2. The binding IL12p40 and IL12p35 to IL12Rβ1 and IL12Rβ2, respectively results in the activation of the Tyk-2 and Jak-2 Janus kinases, induces STAT4 phosphorylation which in turn regulates IFNgmama gene transcription. The p40 subunit of the IL23 and IL12 cytokines provides the majority of binding sites for IL12Rβ1.

An antibody against p40, which blocks both IL23 and IL12 demonstrated activity in clinical trials of Crohn's disease. Selective blockade of the IL23R receptor activity by molecules that selectively bind to IL23R inhibit the by IL23p19/IL23R may provide more limited organ specific targeting o inflammatory disease without more widespread effects associate with inhibition of the p40/IL12 pathway. Additionally it has been shown that anti-IL23R, but not anti-IL23p19, partially suppressed lung metastases in tumor-bearing mice neutralized for IL12p40. Additionally it has been shown that IL23R has tumor-promoting effects that are partially independent of IL23p19. Consequently, the blockade of IL23R activity by molecules that selectively bind to IL23R is potentially effective in the suppression of tumor metastases. Yan, et al (2018) Cancer Immunol Res 6(8); 978-87.

Human IL23R

In one embodiment, specifically bind to the extracellular domain of the human IL23R receptor subunit (hIL23R). hIL23R is expressed as a 629 amino acid precursor comprising a 23 amino acid N-terminal signal sequence which is post-translationally cleaved to provide an 606 amino acid mature protein. The canonical full-length acid hIL23R precursor (including the signal peptide) is a 629 amino acid polypeptide having the amino acid sequence:

(SEQ ID NO: 5)
MNQVTIQWDAVIALYILFSWCHGGITNINCSGHIW
VEPATIFKMGMNISIYCQAAIKNCQPRKLHFYKNG
IKERFQITRINKTTARLWYKNFLEPHASMYCTAEC
PKHFQETLICGKDISSGYPPDIPDEVTCVIYEYSG
NMTCTWNAGKLTYIDTKYVVHVKSLETEEEQQYLT
SSYINISTDSLQGGKKYLVWVQAANALGMEESKQL
QIHLDDIVIPSAAVISRAETINATVPKTIIYWDSQ
TTIEKVSCEMRYKATTNQTWNVKEFDTNFTYVQQS
EFYLEPNIKYVFQVRCQETGKRYWQPWSSLFFHKT
PETVPQVTSKAFQHDTWNSGLTVASISTGHLTSDN
RGDIGLLLGMIVFAVMLSILSLIGIFNRSFRTGIK
RRILLLIPKWLYEDIPNMKNSNVVKMLQENSELMN
NNSSEQVLYVDPMITEIKEIFIPEHKPTDYKKENT
GPLETRDYPQNSLFDNTTVVYIPDLNTGYKPQISN
FLPEGSHLSNNNEITSLTLKPPVDSLDSGNNPRLQ
KHPNFAFSVSSVNSLSNTIFLGELSLILNQGECSS
PDIQNSVEEETTMLLENDSPSETIPEQTLLPDEFV
SCLGIVNEELPSINTYFPQNILESHFNRISLLEK.

For purposes of the present disclosure, the numbering of amino acid residues of the human IL23R polypeptides as described herein is made in accordance with the numbering of this canonical sequence (UniProt Reference No Q5VWK5, SEQ ID NO:1). Amino acids 1-23 of SEQ ID NO:5 are identified as the signal peptide of hIL23R, amino acids 24-355 of SEQ ID NO:5 are identified as the extracellular domain, amino acids 356-376 of SEQ ID NO:5 are identified as the transmembrane domain, and amino acids 377-629 of SEQ ID NO:5 are identified as the intracellular domain.

For the purposes of generating antibodies that bind to the ECD of IL23R, immunization may be performed with the extracellular domain of the hIL23R. The extracellular domain of hIL23R is a 332 amino acid polypeptide of the sequence:

(SEQ ID NO: 6)
GITNINCSGHIWVEPATIFKMGMNISIYCQAAIKN
CQPRKLHFYKNGIKERFQITRINKTTARLWYKNFL
EPHASMYCTAECPKHFQETLICGKDISSGYPPDIP
DEVTCVIYEYSGNMTCTWNAGKLTYIDTKYVVHVK
SLETEEEQQYLTSSYINISTDSLQGGKKYLVWVQA
ANALGMEESKQLQIHLDDIVIPSAAVISRAETINA
TVPKTIIYWDSQTTIEKVSCEMRYKATTNQTWNVK
EFDTNFTYVQQSEFYLEPNIKYVFQVRCQETGKRY
WQPWSSLFFHKTPETVPQVTSKAFQHDTWNSGLTV
ASISTGHLTSDNRGDIG.

Mouse IL23R

In one embodiment, specifically bind to the extracellular domain of the mouse or murine IL23R receptor subunit (mIL23R). mIL23R is expressed as a 644 amino acid precursor comprising a 23 amino acid N-terminal signal sequence which is post-translationally cleaved to provide a 621 amino acid mature protein. The canonical full-length acid mIL23R precursor (including the 23 amino acid signal peptide) is a 644 amino acid polypeptide having the amino acid sequence:

(SEQ ID NO: 7)
MSHLTLQLHVVIALYVLFRWCHGGITSINCSGDMW
VEPGEIFQMGMNVSIYCQEALKHCRPRNLYFYKNG
FKEEFDITRINRTTARIWYKGFSEPHAYMHCTAEC
PGHFQETLICGKDISSGHPPDAPSNLTCVIYEYSG
NMTCTWNTGKPTYIDTKYIVHVKSLETEEEQQYLA
SSYVKISTDSLQGSRKYLVWVQAVNSLGMENSQQL
HVHLDDIVIPSASIISRAETTNDTVPKTIVYWKSK
TMIEKVFCEMRYKTTTNQTWSVKEFDANFTYVQQS
EFYLEPDSKYVFQVRCQETGKRNWQPWSSPFVHQT
SQETGKRNWQPWSSPFVHQTSQTVSQVTAKSSHEP
QKMEMLSATIFRGHPASGNHQDIGLLSGMVFLAIM
LPIFSLIGIFNRSLRIGIKRKVLLMIPKWLYEDIP
NMENSNVAKLLQEKSVFENDNASEQALYVDPVLTE
ISEISPLEHKPTDYKEERLTGLLETRDCPLGMLST
SSSVVYIPDLNTGYKPQVSNVPPGGNLFINRDERD
PTSLETTDDHFARLKTYPNFQFSASSMALLNKTLI
LDELCLVLNQGEFNSLDIKNSRQEETSIVLQSDSP
SETIPAQTLLSDEFVSCLAIGNEDLPSINSYFPQN
VLESHFSRISLFQK

For purposes of the present disclosure, the numbering of amino acid residues of the mIL23R polypeptides as described herein is made in accordance with the numbering of this canonical sequence (UniProt Reference No. Q8K4B4, SEQ ID NO:7). Amino acids 1-23 of SEQ ID NO:7 are identified as the signal peptide of mIL23R, amino acids 24-374 of SEQ ID NO: 7 are identified as the extracellular domain, amino acids 375-395 of SEQ ID NO: 7 are identified as the transmembrane domain, and amino acids 396-644 of SEQ ID NO:7 are identified as the intracellular domain.

For the purposes of generating antibodies that bind to the ECD of IL23R, immunization may be performed with the extracellular domain of the mIL23R. The extracellular domain of the mIL23R receptor is a 351 amino acid polypeptide of the sequence:

(SEQ ID NO: 8)
GITSINCSGDMWVEPGEIFQMGMNVSIYCQEALKH
CRPRNLYFYKNGFKEEFDITRINRTTARIWYKGFS
EPHAYMHCTAECPGHFQETLICGKDISSGHPPDAP
SNLTCVIYEYSGNMTCTWNTGKPTYIDTKYIVHVK
SLETEEEQQYLASSYVKISTDSLQGSRKYLVWVQA
VNSLGMENSQQLHVHLDDIVIPSASIISRAETTND
TVPKTIVYWKSKTMIEKVFCEMRYKTTTNQTWSVK
EFDANFTYVQQSEFYLEPDSKYVFQVRCQETGKRN
WQPWSSPFVHQTSQETGKRNWQPWSSPFVHQTSQT
VSQVTAKSSHEPQKMEMLSATIFRGHPASGNHQDI
G.

The IL23 receptor (IL23R) includes IL12Rβ1 subunit (IL12Rβ1) and IL23R subunit. Provided herein is an IL23R binding protein that specifically binds to IL12Rβ1 and IL23R In some embodiments, the IL23R binding protein binds to a mammalian cell expressing both IL12Rβ1 and IL23R. In some embodiments, the IL23R binding protein can be a bispecific V_{H}H² as described below. In other embodiments, the IL23R binding protein can include a first domain that is a V_{H}H and a second domain which can be a fragment of IL23 or, for example, a scFv.

The IL23R binding protein can be a bispecific V_{H}H² that has a first V_{H}H binding to IL12Rβ1 (an anti-IL12Rβ1 V_{H}H antibody) and a second V_{H}H binding to IL23R (an anti-IL23R V_{H}H antibody) and causes the dimerization of the two receptor subunits and downstream signaling when bound to a cell expressing IL12Rβ1 and IL23R, e.g., a T cell (e.g., a CD8⁺ T cell or a CD4⁺ T cell), a macrophage, and/or a Treg cell.

A linker can be used to join the anti-IL12Rβ1 V_{H}H antibody and the anti-IL23R V_{H}H antibody. For example, a linker can simply be a covalent bond or a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). A peptide linker joining the anti-IL12Rβ1 V_{H}H antibody and the anti-IL23R V_{H}H antibody can be a flexible glycine-serine linker. A linker can also be a chemical linker, such as a synthetic polymer, e.g., a polyethylene glycol (PEG) polymer.

The anti-IL12Rβ1 V_{H}H antibody can have a sequence having at least 90% (e.g., 91%, 92%, 93%, 94%, 95%, 96%, 97%, 98%, 99%, or 100%) sequence identity to the sequence of any sequence of Table 6 or 7 or having CDR1, 2, and 3 of a row of Table 2 or 3.

The anti-IL23R V_{H}H antibody can have a sequence having at least 90% (e.g., 91%, 92%, 93%, 94%, 95%, 96%, 97%, 98%, 99%, or 100%) sequence identity to the sequence of any sequence of Table 8 or 9 or having CDR1, 2, and 3 of a row of Table 4 or 5.

In some embodiments, the IL23R binding protein has a reduced Eₘₐₓ compared to the Eₘₐₓ caused by IL23. Eₘₐₓ reflects the maximum response level in a cell type that can be obtained by a ligand (e.g., a binding protein described herein or the native cytokine (e.g., IL23)). In some embodiments, the IL23R binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IL23. In some embodiments, by varying the linker length of the IL23R binding protein, the Eₘₐₓ of the IL23R binding protein can be changed. The IL23R binding protein can cause Eₘₐₓ in the most desired cell types (e.g., CD8⁺ T cells), and a reduced Eₘₐₓ in other cell types (e.g., marcophages). In some embodiments, the Eₘₐₓ in macrophages caused by an IL23R binding protein described herein is between 1% and 100% (e.g., between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ in T cells (e.g., CD8⁺ T cells) caused by the IL23R binding protein. In other embodiments, the Eₘₐₓ of the IL23R binding protein described herein is greater (e.g., at least 1%, 5%, 10%, 15%, 20%, 25%, 30%, 35%, 40%, 45%, or 50% greater) than the Eₘₐₓ of the natural ligand, IL23.

An IL23 receptor binding protein described herein are useful in wound healing Particularly, the IL23 receptor binding protein described herein plays an important role in initiating wound healing, e.g., healing of keratinocyte layer of the skin. The IL23 receptor binding protein binds to and activates CD8⁺ T cells, CD4⁺ T cells, macrophages, and/or Treg cells. The IL23 receptor binding protein can trigger different levels of downstream signaling in different cell types. For example, by varying the length of the linker between the anti-IL12Rβ1 V_{H}H antibody and the anti-IL23R V_{H}H antibody in the IL23R binding protein, the IL23R binding protein can cause a higher level of downstream signaling in desired cell types compared to undesired cell types. In some embodiments the IL23 receptor binding protein can be a partial agonist that selectively activate T cells (e.g., CD8⁺ T cells) over macrophages. In other embodiments, different anti-IL12Rβ1 V_{H}H antibodies with different binding affinities and different anti-IL23R V_{H}H antibodies with different binding affinities can be combined to make different IL23 receptor binding proteins. Further, the orientation of the two antibodies in the binding protein can also be changed to make a different binding protein (i.e., anti-IL12Rβ1 V_{H}H antibody-linker-anti-IL23R V_{H}H antibody, or anti-IL23R V_{H}H antibody-linker-anti-IL12Rβ1 V_{H}H antibody). Different IL23R binding proteins can be screened to find the ideal binding protein that causes a higher level of downstream signaling in desired cell types compared to undesired cell types. In some embodiments, the level of downstream signaling in T cells (e.g., CD8⁺ T cells) is at least 1.1, 1.5, 2, 3, 5, or 10 times of the level of downstream signaling in macrophages.

### II. Bispecific Binding Molecules

The present disclosure provides disclosure provides bivalent binding molecules that are agonists of the IL23 receptor, the bivalent binding molecule comprising:
a first single domain antibody (sdAb) that specifically binds to the extracellular domain of IL12Rβ1 of the IL23 receptor (an “anti-IL12Rβ1 sdAb”), and
a second single domain antibody that specifically binds to extracellular domain IL23R of the IL23 receptor ((an “anti-IL23R sdAb”),
   wherein the anti-IL12Rβ1 sdAb and anti-IL23R sdAb are stably associated and first wherein contacting a cell expressing IL12Rβ1 and IL23R with an effective amount of the bivalent binding molecule results the dimerization of IL12Rβ1 and IL23R and results in intraceullar signaling characteristic of the IL23R receptor when activated by its natural cognate IL23. In some embodiments, one or both of the sdAbs is a an scFv. In some embodiments, one or both of the sdAbs is a VHH.

### Single Domain Antibody Is A VHH

In some embodiments, the single domain antibody is a VHH. A V_{H}H is a type of single-domain antibody (sdAb) containing a single monomeric variable antibody domain. Like a full-length antibody, itis able to bind selectively to a specific antigen. The complementary determining regions (CDRs) of V_{H}Hs are within a single-domain polypeptide. V_{H}Hs can be engineered from heavy-chain antibodies found in camelids. An exemplary V_{H}H has a molecular weight of approximately 12-15 kDa which is much smaller than traditional mammalian antibodies (150-160 kDa) composed of two heavy chains and two light chains. V_{H}Hs can be found in or produced from Camelidae mammals (e.g., camels, llamas, dromedaiy, alpaca, and guanaco) which are naturally devoid of light chains. Descriptions of sdAbs and V_{H}HS can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018.

### Exemplary Anti IL12Rβ1 Single Domain Antibodies

Table 2 and 3 provide CDRs useful in the preparation of anti-IL12Rβ1 sdAbs for incorporation into the bivalent binding molecules of the present disclosure. In some embodiments, the anti-IL12Rβ1 sdAbs is a single domain antibody comprising, reference to the CDRs provided in Table 2 or 3: a CDR1 having 0, 1, 2, or 3 amino acid changes relative to the sequence depicted in the table; a CDR2 having 0, 1, 2, or 3 amino acid changes relative to the sequence depicted in the table; and a CDR3 having 0, 1, 2, or 3 amino acid changes relative to the sequence depicted in the table. In further embodiments, the anti IL23R VHH antibody comprises a CDR1 having 0, 1, 2, or 3 amino acid changes relative to a CDR1 in Table 4 or 5; a CDR2 having 0, 1, 2, or 3 amino acid changes relative to the sequence depicted in the table and a CDR3 having 0, 1, 2, or 3 amino acid changes relative to the sequence depicted in the table.

### Anti IL23R VHH Dimer Bispecific Binding Molecules

A. “Forward Orientation”

In some embodiments, the bivalent IL-23 receptor binding molecule comprises a polypeptide of the structure:

H₂N-[anti-IL12Rβ1 sdAb]-[L]ₓ-[anti-IL23R sdAb]-[TAG]_{y}-COOH

wherein and L is a polypeptide linker of 1-50 amino acids and x=0 or 1, and TAG is a chelating peptide or a subunit of an Fc domain and y=0 or 1.

In some embodiments, a bivalent IL-23 receptor binding molecule of the foregoing structure comprises a polypeptide from amino to carboxy terminus:
(a) an anti-IL12Rβ1 sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 in a row in Table 2 or 3.
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to CDR2 in the same row in Table 2 or 3; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to CDR3 in the same row in Table 2 or 3;
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an anti-IL23R sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of CDR1 in a row in Table 4 or 5;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of CDR2 in the same row in Table 4 or 5; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of CDR3 in the same row in Table 4 or 5.

### B. “Reverse Orientation”

In some embodiments, the bivalent IL-23 receptor binding molecule comprises a polypeptide of the structure:

H₂N-[anti-IL23R sdAb]-[L]ₓ-[anti-IL12Rβ1 sdAb]-[TAG]_{y}-COOH

wherein and L is a polypeptide linker of 1-50 amino acids and x=0 or 1, and TAG is a chelating peptide or a subunit of an Fc domain and y=0 or 1.

In some embodiments, a bivalent IL-23 receptor binding molecule of the foregoing structure comprises a polypeptide from amino to carboxy terminus:
(a) an anti-IL23R sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of CDR1 in a row in Table 4 or 5;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of CDR2 in the same row in Table 4 or 5; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of CDR3 in the same row in Table 4 or 5.
(b) polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an anti-IL12Rβ1 sdAb comprising:
an anti-IL12Rβ1 sdAb comprising:
a. a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to a CDR1 in a row in Table 2 or 3.
b. a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to CDR2 in the same row in Table 2 or 3; and
c. a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to CDR3 in the same row in Table 2 or 3.

### III. Linkers

A linker can be used to join the anti-IL12Rβ1 sdAb and the anti-IL12Rβ1 sdAb antibody. A linker is a linkage between two linker is a linkage between the two sdAbs in the binding molecule, e.g., protein domains. For example, a linker can simply be a covalent bond or a peptide linker. In some embodiments, the sdAbs in a binding molecule are joined directly (i.e., via a covalent bond). In a bispecific V_{H}H² binding molecule described herein, a linker is a linkage between the two V_{H}Hs in the binding molecule. A In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids).

Examples of flexible linkers include glycine polymers (G)n, glycine-alanine polymers, alanine-serine polymers, glycine-serine polymers (for example, (GmSo)n (SEQ ID NO: 168), (GSGGS)n (SEQ ID NO: 169), (GmSoGm)n (SEQ ID NO: 170), (GmSoGmSoGm)n (SEQ ID NO: 171), (GSGGSm)n (SEQ ID NO: 172), (GSGSmG)n (SEQ ID NO: 173) and (GGGSm)n (SEQ ID NO: 174), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Exemplary flexible linkers include, but are not limited to GGGS (SEQ ID NO:13), GGGGS (SEQ ID NO: 14), GGSG (SEQ ID NO: 15), GGSGG (SEQ ID NO: 16), GSGSG (SEQ ID NO: 17), GSGGG (SEQ ID NO: 18), GGGSG (SEQ ID NO: 19) and GSSSG (SEQ ID NO: 20). In yet other embodiments, a peptide linker can contain 4 to 20 amino acids including motifs of GGSG (SEQ ID NO:15), e.g., GGSGGGSG (SEQ ID NO:21), GGSGGGSGGGSG (SEQ ID NO:22), GGSGGGSGGGSGGGSG (SEQ ID NO:23), or GGSGGGSGGGSGGGSGGGSG (SEQ ID NO:24). In other embodiments, a peptide linker can contain motifs of GGSG (SEQ ID NO:15), e.g., GGSGGGSG (SEQ ID NO:21), GGSGGGSGGGSG (SEQ ID NO:22), GGSGGGSGGGSGGGSG (SEQ ID NO:23), or GGSGGGSGGGSGGGSGGGSG (SEQ ID NO:24)

A linker can also be a chemical linker, such as a synthetic polymer, e.g., a polyethylene glycol (PEG) polymer.

The length of the linker between two sdAb in a binding molecule can be used to modulate the proximity of the two sdAb of the binding molecule. By varying the length of the linker, the overall size and length of the binding molecule can be tailored to bind to specific cell receptors or domains or subunits thereof. For example, if the binding molecule is designed to bind to two receptors or domains or subunits thereof that are located close to each other on the same cell, then a short linker can be used. In another example, if the binding molecule is designed to bind to two receptors or domains or subunits there of that are located on two different cells, then a long linker can be used.

In some embodiments, a linker joins the C-terminus of the anti-IL12Rβ1 sdAb in the binding molecule to the N-terminus of the anti-IL23R sdAb in the binding molecule. In other embodiments, a linker joins the C-terminus of the anti-IL23R sdAb in the binding molecule to the N-terminus of the anti-IL12Rβ1 sdAb in the binding molecule.

Modulation of sdAb Binding Affinity:

In some embodiments, the activity and/or specificity of the bivalent IL-23 receptor binding molecule of the present disclosure may be modulated by the respective binding affinities of the sdAbs for their respective receptor subunits.

It will be appreciated by one of skill in the art that the binding of the first sdAb of the bivalent IL-23 receptor binding molecule to the first receptor subunit ECD on the cell surface will enhance the probability of a binding interaction between the second sdAb of the bivalent IL-23 receptor binding molecule with the ECD of the second receptor subunit. This cooperative binding effect may result in a bivalent IL-23 receptor binding molecule which has a very high affinity for the receptor and a very slow “off rate” from the receptor. Typical VHH single domain antibodies have an affinity for their targets of from about 10⁻⁵ M to about 10⁻¹⁰ M In those instances such slow off-rate kinetics are desirable in the bivalent IL-23 receptor binding molecule, the selection of sdAbs having high affinities (about 10⁻⁷ M to about 10⁻¹⁰ M) for incorporation into the bivalent IL-23 receptor binding molecule are favored.

Naturally occurring cytokine ligands for typically do not exhibit a similar affinity for each subunit of a heterodimeric receptor. Consequently, in designing a bivalent IL-23 receptor binding molecule which is a mimetic of the cognate cytokine IL23 as contemplated by some embodiments of the present disclosure, selection of sdAbs for the first and second IL23R receptor subunit have an affinity similar to (e.g., having an affinity about 10 fold, alternatively about 20 fold, or alternatively about 50 fold higher or lower than) the cognate IL23 for the respective receptor subunit may be used.

In some embodiments, the bivalent IL-23 receptor binding molecules of the present disclosure are partial agonists of the IL23R receptor. As such, the activity of the bivalent binding molecule may be modulated by selecting sdAb which have greater or lesser affinity for either one or both of the IL23R receptor subunits. As some heterodimeric cytokine receptors are comprised of a “proprietary subunit” (i.e., a subunit which is not naturally a subunit of another multimeric receptor) and a second “common” subunit (such as CD132) which is a shared component of multiple cytokine receptors), selectivity for the formation of such receptor may be enhanced by employing first sdAb which has a higher affinity for the proprietary receptor subunit and second sdAB which exhibits a lower affinity for the common receptor subunit. Additionally, the common receptor subunit may be expressed on a wider variety of cell types than the proprietary receptor subunit. In some embodiments wherein the receptor is a heterodimeric receptor comprising a proprietary subunit and a common subunit, the first sdAb of the bivalent IL-23 receptor binding molecule exhibits a significantly greater (more than 10 times greater, alternatively more than 100 times greater, alternatively more than 1000 times greater) affinity for the proprietary receptor than the second sdAb of the bivalent IL-23 receptor binding molecule for the common receptor subunit. In one embodiment, the present disclosure provides a bivalent IL-23 receptor binding molecule wherein the affinity of the anti-IL12Rβ1 sdAb of has an affinity of more than 10 times greater, alternatively more than 100 times greater, alternatively more than 1000 times greater) affinity anti-IL23R sdAb common receptor subunit. In one embodiment, the present disclosure provides a bivalent IL-23 receptor binding molecule wherein the affinity of the anti-IL23R sdAb of has an affinity of more than 10 times greater, alternatively more than 100 times greater, alternatively more than 1000 times greater) affinity anti-IL12Rβ1 sdAb common receptor subunit.

### III. Modifications to Extend Duration of Action In Vivo

The IL23 receptor bivalent binding molecule described herein can be modified to provide for an extended lifetime in vivo and/or extended duration of action in a subject. In some embodiments, the binding molecule can be conjugated to carrier molecules to provide desired pharmacological properties such as an extended half-life. In some embodiments, the binding molecule can be covalently linked to the Fc domain of IgG, albumin, or other molecules to extend its half-life, e.g., by pegylation, glycosylation, and the like as known in the art. In some embodiments, the IL23 receptor bivalent binding molecule modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the IL23 receptor bivalent binding molecule to provide an extended duration of action in a mammalian subject include (but are not limited to);
conjugation of the IL23 receptor bivalent binding molecule to one or more carrier molecules,
conjugation IL23 receptor bivalent binding molecule to protein carriers molecules, optionally in the form of a fusion protein with additional polypeptide sequences (e.g, IL23 receptor bivalent binding molecule-Fc fusions) and
conjugation to polymers, (e.g. water soluble polymers to provide a PEGylated IL23 receptor bivalent binding molecule).

It should be noted that the more than one type of modification that provides for an extended duration of action in a mammalian subject may be employed with respect to a given IL23 receptor bivalent binding molecule. For example, IL23 receptor bivalent binding molecule of the present disclosure may comprise both amino acid substitutions that provide for an extended duration of action as well as conjugation to a carrier molecule such as a polyethylene glycol (PEG) molecule.

### Protein Carrier Molecules:

Examples of protein carrier molecules which may be covalently attached to the IL23 receptor bivalent binding molecule to provide an extended duration of action in vivo include, but are not limited to albumins, antibodies and antibody fragments such and Fc domains of IgG molecules.

### Fc Fusions:

In some embodiments, the IL23 receptor bivalent binding molecule is conjugated to a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

### Linkage of Bivalent Binding Molecule to Fc

The linkage of the IL23 receptor bivalent binding molecule to the Fc subunit may incorporate a linker molecule as described below between the bivalent sdAb and Fc subunit. In some embodiments, the IL23 receptor bivalent binding molecule is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the IL23R bivalent binding is linked to an Fc domain using an human IgG1 hinge domain.

Knob-into-Hole Fc Format

In some embodiments, when the IL23 receptor bivalent binding molecule described herein is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”), and ii) in the CH3 domain of a second heavy chain, an amino acid residue is replaced with an amino acid residue having a smaller side chain (e.g., alanine or threonine), thereby generating a cavity (“hole”) at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fc region (Carter, et al. (2001) Immunol Methods 248, 7-15).

The knob-into-hole format can be used to facilitate the expression of a first polypeptide on a first Fc monomer with a “knob” modification and a second polypeptide on the second Fc monomer possessing a “hole” modification to facilitate the expression of heterodimeric polypeptide conjugates.

### Albumin Carrier Molecules

In some embodiments, the IL23 receptor bivalent binding molecule conjugated to an is albumin molecule (e.g., human serum albumin) which is known in the art to facilitate extended exposure in vivo. In one embodiment of the invention, the IL23 receptor bivalent binding molecule is conjugated to albumin via chemical linkage or expressed as a fusion protein with an albumin molecule referred to herein as an IL23 receptor bivalent binding molecule albumin fusion.” The term “albumin” as used in the context αβhIL2 mutein albumin fusions include albumins such as human serum albumin (HSA), cyno serum albumin, and bovine serum albumin (BSA). In some embodiments, the HSA the HSA comprises a C34S or K573P amino acid substitution relative to the wild-type HSA sequence According to the present disclosure, albumin can be conjugated to a IL23 receptor bivalent binding molecule at the carboxyl terminus, the amino terminus, both the carboxyl and amino termini, and internally (see, e.g., U.S. Pat. Nos. 5,876,969 and 7,056,701). In the HAS IL23 receptor bivalent binding molecule contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a IL23 receptor bivalent binding molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the IL23 receptor bivalent binding molecule and the albumin molecule the IL23 receptor bivalent binding molecule—albumin complex may be provided as a fusion protein comprising an albumin polypeptide sequence and an IL23 receptor bivalent binding molecule recombinantly expressed in a host cell as a single polypeptide chain, optionally comprising a linker molecule between the albumin and IL23 receptor bivalent binding molecule. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein solated from the host cell culture by techniques well known in the art.

### Polymeric Carriers

In some embodiments, extended in vivo duration of action of the IL23 receptor bivalent binding molecule may be achieved by conjugation to one or more polymeric carrier molecules such as XTEN polymers or water soluble polymers.

XTEN Conjugates

The IL23 receptor bivalent binding molecule may further comprise an XTEN polymer. The XTEN polymer may be is conjugated (either chemically or as a fusion protein) the αβhIL2 mutein provides extended duration of akin to PEGylation and may be produced as a recombinant fusion protein in E. coli. XTEN polymers suitable for use in conjunction with the IL23 receptor bivalent binding molecule of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE | DOI:10.1371/journal.pone.0157193 Jun. 13, 2016. The XTEN polymer may fusion protein may incorporate a protease sensitive cleavage site between the XTEN polypeptide and the hIL2 mutein such as an MMP-2 cleavage site.

Water Soluble Polymers

In some embodiments, the IL23 receptor bivalent binding molecule can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol,), polysaccharides), poly-alpha-hydroxy acid), polyvinyl alcohol (PVA), polyphosphazene, polyoxazolines (POZ), poly(N-acryloylmorpholine), or a combination thereof.

In some embodiments, IL23 receptor bivalent binding molecule can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some embodiments, selective PEGylation of the IL23 receptor bivalent binding molecule, for example, by the incorporation of non-natural amino acids having side chains to facilitate selective PEG conjugation, may be employed. Specific PEGylation sites can be chosen such that PEGylation of the binding molecule does not affect its binding to the target receptors.

In some instances, the sequences of IL23 receptor bivalent binding molecules of the present disclosure possess an N-terminal glutamine (“1Q”) residue. N-terminal glutamine residues have been observed to spontaneously cyclyize to form pyroglutamate (pE) at or near physiological conditions. (See e.g., Liu, et al (2011) J. Biol. Chem. 286(13): 11211-11217). In some embodiments, the formation of pyroglutamate complicates N-terminal PEG conjugation particularly when aldehyde chemistry is used for N-terminal PEGylation. Consequently, when PEGylating the IL-23 receptor binding molecules of the present disclosure, particularly when aldehyde chemistry is to be employed, the IL-23 receptor binding molecules possessing an amino acid at position 1 (e.g., 1Q) are substituted at position 1 with an alternative amino acid or are deleted at position 1 (e.g., des-1Q). In some embodiments, the IL-23 receptor binding molecules of the present disclosure comprise an amino acid substitution selected from the group Q1E and Q1D.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios.

For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbonst

Two widely used first generation activated monomethoxy PEGs (mPEGs) are succinimdyl carbonate PEG (SC-PEG; see, e.g., Zalipsky, et al. (1992) Biotehnol. Appl Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, N.Y. 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the IL23 receptor bivalent binding molecule and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942):834-840.

Fatty Acid Carriers

In some embodiments an IL23 receptor bivalent binding molecule having an extended duration of action in a mammalian subject and useful in the practice of the present disclosure is achieved by covalent attachment of the IL23 receptor bivalent binding molecule to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the IL23 receptor bivalent binding molecule is acetylated at the N-terminus by enzymatic reaction with N-terminal acetyltransferase and, for example, acetyl CoA. Alternatively, or in addition to N-terminal acetylation, the IL23 receptor bivalent binding molecule is acetylated at one or more lysine residues, e.g., by enzymatic reaction with a lysine acetyltransferase. See, for example Choudhary et al. (2009) Science 325 (5942):834L2 ortho840.

### Modifications to Provide Additional Functions

In some embodiments, embodiment, the IL23 receptor bivalent binding molecule may comprise a functional domain of a chimeric polypeptide. IL23 receptor bivalent binding molecule fusion proteins of the present disclosure may be readily produced by recombinant DNA methodology by techniques known in the art by constructing a recombinant vector comprising a nucleic acid sequence comprising a nucleic acid sequence encoding the IL23 receptor bivalent binding molecule in frame with a nucleic acid sequence encoding the fusion partner either at the N-terminus or C-terminus of the IL23 receptor bivalent binding molecule, the sequence optionally further comprising a nucleic acid sequence in frame encoding a linker or spacer polypeptide.

FLAG Tags

In other embodiments, the IL23 receptor bivalent binding molecule can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

### Chelating Peptides

In some embodiments, the IL23 receptor bivalent binding molecule (including fusion proteins of the IL23 receptor bivalent binding molecule) of the present disclosure are may be covalently bonded via a peptide bond to one or more transition metal chelating polypeptide sequences. The association of the IL23 receptor bivalent binding molecule with chelating peptide provides multiple utilities including: the purification of the IL23 receptor bivalent binding molecule using immobilized metal affinity chromatography (IMAC) as described in Smith, et al. U.S. Pat. No. 4,569,794 issued Feb. 11, 1986; immobilization of the IL23 receptor bivalent binding molecule on nitrilotriacetic acid (NTA) modified surface plasmon resonance sensor chips (e.g., Sensor Chip NTA available from Cytiva Global Life Science Solutions USA LLC, Marlborough Mass. as catalog number BR100407) as described in Nieba, et al. (1997) Analytical Biochemistry 252(2):217-228, or to form kinetically inert or kinetically labile complexes between the IL23 receptor bivalent binding molecule and a transition metal ion as described in Anderson, et al. (U.S. Pat. No. 5,439,829 issued Aug. 8, 1995 and Hale, J. E (1996) Analytical Biochemistry 231(1):46-49. Examples of transition metal chelating polypeptides useful in the practice of the present disclosure are described in Smith, et al. supra and Dobeli, et al. U.S. Pat. No. 5,320,663 issued May 10, 1995 the entire teachings of which are hereby incorporated by reference. Particular transition metal chelating polypeptides useful in the practice of the present disclosure are peptides comprising 3-6 contiguous histidine residues (SEQ ID NO: 389) such as a six-histidine peptide (His)₆ (SEQ ID NO: 175) anTd are frequently referred to in the art as “His-tags.” In some embodiments, a purification handle is a polypeptide having the sequence Ala-Ser-His-His-His-His-His-His (“ASH6”) (SEQ ID NO: 176) or Gly-Ser-His-His-His-His-His-His-His-His (“GSH8”) (SEQ ID NO: 177).

### Targeting Moieties:

In some embodiments, IL23 receptor bivalent binding molecule is conjugated to molecule which provides (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between IL23 receptor bivalent binding molecule sequence and the sequence of the targeting domain of the fusion protein.

In other embodiments, a chimeric polypeptide including a IL23 receptor bivalent binding molecule and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting moiety is an antibody that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Ra2, CD19, mesothelin, Her2, EpCam, Muc1, ROR1, CD133, CEA, EGRFRVIII, PSCA, GPC3, Pan-ErbB and FAP.

### Recombinant Production

Alternatively, the IL-23 receptor binding molecules of the present disclosure are produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide.

Construction of Nucleic Acid Sequences Encoding the IL-23 Receptor Binding Molecule

In some embodiments, the IL-23 receptor binding molecule is produced by recombinant methods using a nucleic acid sequence encoding the IL-23 receptor binding molecule (or fusion protein comprising the IL-23 receptor binding molecule). The nucleic acid sequence encoding the desired αβhIL-23 receptor binding molecule can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of IL-2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IL-23 receptor binding molecule (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL-23 receptor binding molecule may be obtained from various commercial sources that provide custom made nucleic acid sequences. Amino acid sequence variants of the IL-23 receptor binding molecules of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding a IL-23 receptor binding molecule and expressing those sequences in a suitably transformed host include, but are not limited to, using a PCR-assisted mutagenesis technique. Mutations that consist of deletions or additions of amino acid residues to a IL-23 receptor binding molecule can also be made with standard recombinant techniques. In the event of a deletion or addition, the nucleic acid molecule encoding a IL-23 receptor binding molecule is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A IL-23 receptor binding molecule of the present disclosure may be produced recombinantly not only directly, but also as a fusion polypeptide with a heterologous polypeptide, e.g. a signal sequence or other polypeptide having a specific cleavage site at the N-terminus or C-terminus of the mature IL-23 receptor binding molecule. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the IL-23 receptor binding molecule from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the IL-23 receptor binding molecule into the culture medium as described in Singh, U.S. Pat. No. 7,198,919 B1 issued Apr. 3, 2007.

In the event the IL-23 receptor binding molecule to be expressed is to be expressed as a chimera (e.g., a fusion protein comprising a IL-23 receptor binding molecule and a heterologous polypeptide sequence), the chimeric protein can be encoded by a hybrid nucleic acid molecule comprising a first sequence that encodes all or part of the IL-23 receptor binding molecule and a second sequence that encodes all or part of the heterologous polypeptide. For example, subject IL-23 receptor binding molecules described herein may be fused to a hexa-/octa-histidine tag (SEQ ID NOS 175 and 390, respectively) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IL-23 receptor binding molecule. For example, the N-terminus may be linked to a targeting domain and the C-terminus linked to a hexa-histidine tag (SEQ ID NO: 175) purification handle.

The complete amino acid sequence of the polypeptide (or fusion/chimera) to be expressed can be used to construct a back-translated gene. A DNA oligomer containing a nucleotide sequence coding a IL-23 receptor binding molecule can be synthesized. For example, several small oligonucleotides coding for portions of the desired polypeptide can be synthesized and then ligated. The individual oligonucleotides typically contain 5′ or 3′ overhangs for complementary assembly.

Codon Optimization:

In some embodiments, the nucleic acid sequence encoding the IL-23 receptor binding molecule may be “codon optimized” to facilitate expression in a particular host cell type.

Techniques for codon optimization in a wide variety of expression systems, including mammalian, yeast and bacterial host cells, are well known in the and there are online tools to provide for a codon optimized sequences for expression in a variety of host cell types. See e.g Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

Expression Vectors:

Once assembled (by synthesis, site-directed mutagenesis or another method), the nucleic acid sequence encoding an a IL-23 receptor binding molecule will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Regulatory Control Sequences:

Expression vectors for a IL-23 receptor binding molecules of the present disclosure contain a regulatory sequence that is recognized by the host organism and is operably linked to nucleic acid sequence encoding the IL-23 receptor binding molecule. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego Calif. USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject a IL-23 receptor binding molecule, particularly as regards potential secondary structures.

Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox vims, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovims enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing restriction mapping, and expression of a biologically active polypeptide in a suitable host.

### Host Cells:

The present disclosure further provides prokaryotic or eukaryotic cells that contain and express a nucleic acid molecule that encodes a IL-23 receptor binding molecule. A cell of the present disclosure is a transfected cell, i.e., a cell into which a nucleic acid molecule, for example a nucleic acid molecule encoding a mutant IL-2 polypeptide, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant IL-23 receptor binding molecule can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989)Virology 170:31-39)); yeast cells (examples of vectors for expression in yeast S. cerenvisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo ratliver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The IL-23 receptor binding molecule may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, a IL-23 receptor binding molecule obtained will be glycosylated or unglycosylated depending on the host organism used to produce the mutein. If bacteria are chosen as the host then the a IL-23 receptor binding molecule produced will be unglycosylated. Eukaryotic cells, on the other hand, will typically result in glycosylation of the IL-23 receptor binding molecule.

In some embodiments, itis possible that an amino acid sequence (particularly a CDR sequence) of an sdAb to be incorporated into a bivalent IL-23 receptor binding molecule may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce a IL-23 receptor binding molecule disclosed herein. The expression vector comprising a nucleic acic sequence encoding IL-23 receptor binding molecule is introduced into the prokaryotic or eukaryotic host cells via conventional transformation or transfection techniques.

Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced IL-23 receptor binding molecule polypeptides can be recovered from the culture medium as a secreted polypeptide if a secretion leader sequence is employed. Alternatively, the IL-23 receptor binding molecule polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly IL-23 receptor binding molecule by the transformed host can be purified according to any suitable method. Recombinant IL-23 receptor binding molecules can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant a IL-23 receptor binding molecule can be purified from the expression system using routine biochemical procedures, and can be used, e.g., as therapeutic agents, as described herein.

In some embodiments, where the IL-23 receptor binding molecule is expressed with a purification tag as discussed above, this purification handle may be used for isolation of the IL-23 receptor binding molecule from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the IL-23 receptor binding molecules recovered can be assayed for activating by any suitable method known in the art and may be evaluated as substantially purified forms or as part of the cell lysate or cell medium when secretion leader sequences are employed for expression.

### Pharmaceutical Formulations

In some embodiments, the subject IL-23 receptor binding molecule (and/or nucleic acids encoding the IL-23 receptor binding molecule or recombinant cells incorporating a nucleic acid sequence and modified to express the IL-23 receptor binding molecule) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IL-23 receptor binding molecule is to be administered to the subject in need of treatment or prophyaxis.

Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor ELTM (BASF, Parsippany, N.J.) or phosphate buffered saline (PBS).

Buffers:

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Preservatives:

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

Tonicity Agents:

In many cases, it will be preferable to include isotonic agents, for example, sugars, polyalcohols such as mannitol, sorbitol, sodium chloride in the composition.

### Routes of Administration

In some embodiments of the therapeutic methods of the present disclosure involve the administration of a pharmaceutical formulation comprising a IL-23 receptor binding molecule (and/or nucleic acids encoding the IL-23 receptor binding molecule or recombinantly modified host cells expressing the IL-23 receptor binding molecule) to a subject in need of treatment. The pharmaceutical formulation comprising a IL-23 receptor binding molecules of the present disclosure may be administered to a subject in need of treatment or prophyaxis by a variety of routes of administration, including parenteral administration, oral, topical, or inhalation routes.

Parenteral Administration:

In some embodiments, the methods of the present disclosure involve the parenteral administration of a pharmaceutical formulation comprising a IL-23 receptor binding molecule (and/or nucleic acids encoding the IL-23 receptor binding molecule or recombinantly modified host cells expressing the IL-23 receptor binding molecule) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration.

Oral Administration:

In some embodiments, the methods of the present disclosure involve the oral administration of a pharmaceutical formulation comprising a IL-23 receptor binding molecule (and/or nucleic acids encoding the IL-23 receptor binding molecule or recombinantly modified host cells expressing the IL-23 receptor binding molecule) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

Inhalation Formulations:

In some embodiments, the methods of the present disclosure involve the inhaled administration of a pharmaceutical formulation comprising a IL-23 receptor binding molecule (and/or nucleic acids encoding the IL-23 receptor binding molecule or recombinantly modified host cells expressing the IL-23 receptor binding molecule) to a subject in need of treatment. In the event of administration by inhalation, subject IL-23 receptor binding molecules, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Mucosal and Transdermal Formulations:

In some embodiments, the methods of the present disclosure involve the mucosal or transdermal administration of a pharmaceutical formulation comprising a IL-23 receptor binding molecule (and/or nucleic acids encoding the IL-23 receptor binding molecule or recombinantly modified host cells expressing the IL-23 receptor binding molecule) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

Extended Release and Depot Formulations:

In some embodiments of the method of the present disclosure, the IL-23 receptor binding molecule is administered to a subject in need of treatment in a formulation to provide extended release of the IL-23 receptor binding molecule agent. Examples of extended release formulations of the injectable compositions can be brought about by including in the composition an agent which delays absorption, for example, aluminum monostearate and gelatin. In one embodiment, the subject IL-23 receptor binding molecules or nucleic acids are prepared with carriers that will protect the IL-23 receptor binding molecules against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Administration of Nucleic Acids Encoding the IL23R Binding Molecule:

In some embodiments of the method of the present disclosure, delivery of the IL-23 receptor binding molecule to a subject in need of treatment is achieved by the administration of a nucleic acid encoding the IL-23 receptor binding molecule. Methods for the administration nucleic acid encoding the IL-23 receptor binding molecule to a subject is achieved by transfection or infection using methods known in the art, including but not limited to the methods described in McCaffrey et al. (Nature (2002)418:6893), Xia et al. (Nature Biotechnol. (2002) 20:1006-1010), or Putnam (Am. J. Health Syst. Pharm. (1996) 53: 151-160 erratum at Am. J. Health Syst. Pharm. (1996) 53:325). In some embodiments, the IL23 receptor binding molecule is administered to a subject by the administration of a pharmaceutically acceptable formulation of recombinant expression vector comprising a nucleic acid sequence encoding the IL23 receptor binding molecule operably linked to one or more expression control sequences operable in a mammalian subject. In some embodiments, the expression control sequence may be selected that is operable in a limited range of cell types (or single cell type) to facilitate the selective expression of the IL-23 receptor binding molecule in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovims is a replication competent adenovirus. In some embodiments the adenovirus is a replication competent recombinant virus engineered to selectively replicate in the target cell type.

In some embodiments, particularly for administration of IL23 receptor binding molecules to the subject, particular for treatment of diseases of the intestinal tract or bacterial infections in a subject, the nucleic acid encoding the IL23 receptor binding molecule may be delivered to the subject by the administration of a recombinantly modified bacteriophage vector encoding the IL23 receptor binding molecule. As used herein, the terms ‘procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the IL23 receptor binding molecule to procaryotic cells in the subject while avoiding expression in mammalian cells. A wide variety of bacteriophages capable of selection abroad range of bacterial cells have been identified and characterized extensively in the scientific literature. In some embodiments, the phage is modified to remove adjacent motifs (PAM). Elimination of the of Cas9 sequences from the phage genome reduces ability of the Cas9 endonuclease of the target procaryotic cell to neutralize the invading phage encoding the IL23 receptor binding molecule.

Administration of Recombinantly Modified Cells Expressing the IL23 Receptor Binding Molecule:

In some embodiments of the method of the present disclosure, delivery of the IL23 receptor binding molecule to a subject in need of treatment is achieved by the administration of recombinant host cells modified to express the IL23 receptor binding molecule may be administered in the therapeutic and prophylactic applications described herein. In some embodiments, the recombinant host cells are mammalian cells, e.g., human cells.

In some embodiments, the nucleic acid sequence encoding the IL23 receptor binding molecule (or vectors comprising same) may be maintained extrachromosomally in the recombinantly modified host cell for administration. In other embodiments, the nucleic acid sequence encoding the IL23 receptor binding molecule may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009)Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005)Nature Biotechnology 23(3):967-973, a TALEN-nuclease, a Cas9 endonuclease from CRISPR system as or a modified restriction endonuclease to extended sequence specificity (Eisenschmidt, et al. 2005; 33(22): 7039-7047).

In some embodiments, particularly for administration of IL-23 receptor binding molecules to the intestinal tract, the IL23 receptor binding molecule may be delivered to the subject by a recombinantly modified procaryotic cell (e.g., Lactobacillus lacti). The use of engineered procaryotic cells for the delivery of recombinant proteins to the intestinal tract are known in the art. See, e.g. Lin, et al. (2017) Microb Cell Fact 16:148. In some embodiments, the engineered bacterial cell expressing the IL-23 receptor binding molecule may be administered orally, typically in aqueous suspension, or rectally (e.g. enema).

### Therapeutic Applications

The present disclosure further provides methods of treating a subject suffering from a disease disorder or condition by the administration of a therapeutically effective amount of an IL23 receptor binding molecule (or nucleic acid encoding an IL23 receptor binding molecule including recombinant viruses encoding the IL23 receptor binding molecule) of the present disclosure.

In another aspect, the disclosure provides a method of treating an autoimmune or inflammatory disease, disorder, or condition, a neoplastic disease, or a viral infection in a subject in need thereof, comprising administering to the subject a therapeutically effective amount of an IL23 receptor binding protein described herein or a pharmaceutical composition described herein.

In some embodiments, the method further comprises administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody. In certain embodiments, the therapeutic antibody is an antibody that binds a protein selected from the group consisting of BLyS, CD11a, CD20, CD25, CD3, CD52, IgEIL12/IL23, IL17a, IL1β, IL4Rα, IL5, IL6R, integrin-α4 β7, RANKL, TNFα, VEGF-A, and VLA-4.

In certain embodiments, the disease, disorder, or condition is selected from viral infections, Heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, Pityriasis rubra pilaris, Pityriasis rosacea, parapsoriasis, Pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis, seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

The present disclosure provides methods of use of IL-23 receptor binding molecules in the treatment of subjects suffering from a neoplastic disease disorder or condition by the administration of a therapeutically effective amount of a IL-23 receptor binding molecule (or nucleic acid encoding a IL-23 receptor binding molecule including recombinant vectors encoding IL-23 receptor binding molecules, and eucaryotic and procaryotic cells modified to express a IL-23 receptor binding molecule) as described herein.

Neoplasms Amenable to Treatment:

The compositions and methods of the present disclosure are useful in the treatment of subject suffering from a neoplastic disease characterized by the presence neoplasms, including benign and malignant neoplasms, and neoplastic disease.

Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

Assessing Anti-Neoplastic Efficacy:

The determination of efficacy of the methods of the present disclosure in the treatment of cancer is generally associated with the achievement of one or more art recognized parameters such as reduction in lesions particularly reduction of metastatic lesion, reduction in metastasis, reduction in tumor volume, improvement in ECOG score, and the like. Determining response to treatment can be assessed through the measurement of biomarker that can provide reproducible information useful in any aspect of IL-23 receptor binding molecule therapy, including the existence and extent of a subject's response to such therapy and the existence and extent of untoward effects caused by such therapy. By way of example, but not limitation, biomarkers include enhancement of IFNγ, and upregulation of granzyme A, granzyme B, and perforin; increase in CD8⁺ T-cell number and function; enhancement of IFNγ, an increase in ICOS expression on CD8⁺ T-cells, enhancement of IL-10 expressing T_{Reg} cells. The response to treatment may be characterized by improvements in conventional measures of clinical efficacy may be employed such as Complete Response (CR), Partial Response (PR), Stable Disease (SD) and with respect to target lesions, Complete Response (CR),” Incomplete Response/Stable Disease (SD) as defined by RECIST as well as immune-related Complete Response (irCR), immune-related Partial Response (irPR), and immune-related Stable Disease (irSD) as defined Immune-Related Response Criteria (irRC) are considered by those of skill in the art as evidencing efficacy in the treatment of neoplastic disease in mammalian (e.g. human) subjects.

Maintenance of Serum Concentration:

In some embodiments of the invention the present disclosure provides methods and compositions for the treatment and/or prevention of neoplastic diseases, disorders or conditions by the administration of a therapeutically effective amount of an IL-23 receptor binding molecules the serum concentration of the IL-23 receptor binding molecule is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the therapeutically effective concentration with respect to such IL-23 receptor binding molecule.

Combination of IL-23 Receptor Binding Molecules with Supplementary Therapeutic Agents:

The present disclosure provides for the use of the IL-23 receptor binding molecules of the present disclosure in combination with one or more additional active agents (“supplementary agents”). Such further combinations are referred to interchangeably as “supplementary combinations” or “supplementary combination therapy” and those therapeutic agents that are used in combination with IL-23 receptor binding molecules of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the IL-23 receptor binding molecules.

Chemotherapeutic Agents:

In some embodiments, the supplementary agent is a chemotherapeutic agent. In some embodiments the supplementary agent is a “cocktail” of multiple chemotherapeutic agents. In some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethyl olomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A₂, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; best rabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT 11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent isone or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foreoing as practied in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the IL-23 receptor binding molecule is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11 SI 15), IDO inhibitors such as epacadostat, and oncolytic viruses such as talimogene laherparepvec (T-VEC).

Anti-Tumor Antigen Antibody Therapeutics as Supplementary Agents

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Ley, VEGF (e.g. bevacizumab), VEGFR, VEGFR2 (e.g. ramucirumab), PDGFRα (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin αVβ3, and integrin α4 β1.

Examples of antibody therapeutics which are FDA approved and may be used as supplementary agents for use in the treatment of neoplastic disease include those provided in the Table below.

TABLE
Approved Antineoplastic Disease Antibodies and Indications

| Name | Tradename(s) | Target; format | Indication |
|---|---|---|---|
| [fam]- | Enhertu | HER2; Humanized IgG1 ADC | HER2 + breast cancer |
| trastuzumab deruxtecan | | | |
| Enfortumab vedotin | Padcev | Nectin-4; Human IgG1 ADC | Urothelia 1 cancer |
| Polatuzumab vedotin | Polivy | CD79b; Humanized IgG1 ADC | Diffuse large B-cell lymphoma |
| Cemiplimab | Libtayo | PD-1; Human mAb | Cutaneous squamous cell |
| | | | carcinoma |
| Moxetumomab pasudotox | Lumoxiti | CD22; Murine | Hairy cell leukemia |
| | | IgG1 dsFv immunotoxin | |
| Mogamuizumab | Poteligeo | CCR4; Humanized IgG1 | Cutaneous T cell lymphoma |
| Tildrakizumab | Ilumya | IL23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, ADC | Hematological malignancy |
| ozogamicin | | | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL-17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell lung |
| | | | cancer |
| Blinatumomab | Blincyto | CD19, CD3; Murine bispecific | Acute lymphoblastic leukemia |
| | | tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL-6; Chimeric IgG1 | Castleman disease |
| Obinutuzumab | Gazyva | CD20; Humanized IgG1; | Chronic lymphocytic leukemia |
| | | Glycoengineered | |
| Ado-trastuzumab | Kadcyla | HER2; Humanized IgG1, ADC | Breast cancer |
| emtansine | | | |
| Pertuzumab | Perjeta | HER2; Humanized IgG1 | Breast Cancer |
| Brentuximab vedotin | Adcetris | CD30; Chimeric IgG1, ADC | Hodgkin lymphoma, systemic |
| | | | anaplastic large cell lymphoma |
| Ipilimumab | Yervoy | CTLA-4; Human IgG1 | Metastatic melanoma |
| Ofatumumab | Arzerra | CD20; Human IgG1 | Chronic lymphocytic leukemia |
| Certolizumab pegol | Cimzia | TNF; Humanized Fab, | Crohn disease |
| | | pegylated | |
| Catumaxomab | Removab | EPCAM/CD3; Rat/mouse | Malignantascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab-I131 | Bexxar | CD20; Murine IgG2a | Non-Hodgkin lymphoma |
| Ibritumomab tiuxetan | Zevalin | CD20; Murine IgG1 | Non-Hodgkin lymphoma |
| Gemtuzumab | Mylotarg | CD33; Humanized IgG4, ADC | Acute myeloid leukemia |
| ozogamicin | | | |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

Physical Methods

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising a IL-23 receptor binding molecule and one or more supplementary agents. In some embodiments, the present disclosure further contemplates the use of a IL-23 receptor binding molecule in combination with surgery (e.g. tumor resection). In some embodiments, the present disclosure further contemplates the use of a IL-23 receptor binding molecule in combination with bone marrow transplantation, peripheral blood stem cell transplantation or other types of transplantation therapy.

Combination with Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

Immune checkpoint modulators include but are not limited to immune checkpoint antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H⁵) receptor, LAG3 (lymphocyte activation gene 3; also known as CD233) and CTLA4 (cytotoxic T-lymphocyte associated antigen 4; also known as CD152). In some embodiments, immune checkpoint modulators are agonists that trigger the checkpoint pathway resulting stimulation of the immune response. Examples of such agonist immune checkpoint modulators include, but is not limited to, agonist that modulate the binding of ICOSL to ICOS(CD278), B7-H6 to NKp30, CD155 to CD96, OX40L to OX40, CD70 to CD27, CD40 to CD40L, and GITRL to GITR. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor). The term PD1 pathway inhibitors includes monoclonal antibodies that interfere with the binding of PD1 to PDL1 and/or PDL2. Examples of commercially available PD1 pathway inhibitors useful as supplementary agents in the treatment of neoplastic disease include antibodies that interfere with the binding of PD1 to PDL1 and/or PDL2 including but not limited to nivolumab (Opdivo®, BMS-936558, MDX1106, commercially available from BristolMyers Squibb, Princeton N.J.), pembrolizumab (Keytruda® MK-3475, lambrolizumab, commercially available from Merck and Company, Kenilworth N.J.), and atezolizumab (Tecentriq®, Genentech/Roche, South San Francisco Calif.). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline), aptamers (Wang, et al. (2018) 145:125-130), peptide PD1 pathway inhibitors (Sasikumar, et al., U.S. Pat. No. 9,422,339 issued Aug. 23, 2016, and Sasilkumar, et al., U.S. Pat. No. 8,907,053 issued Dec. 9, 2014), small molecules (CA-170, AUPM-170, Aurigene/Curis; Sasikumar, et al., 1,2,4-oxadiazole and thiadiazole compounds as immunomodulators (PCT/IB2016/051266 filed Mar. 7, 2016, published as WO2016142833A1 Sep. 15, 2016) and Sasikumar, et al PCT/IB2016/051343 filed Mar. 9, 2016 and published as WO2016142886A2), BMS-1166 and Chupak L S and Zheng X. (2015) WO 2015/034820 A1, EP3041822 B1 granted Aug. 9, 2017; WO2015034820 A1; and Chupak, et al. 2015)WO 2015/160641 A2. WO 2015/160641 A2, Chupak, et al. Sharpe, et al. WO 2011082400 A2 published Jul. 7, 2011; U.S. Pat. No. 7,488,802 issued Feb. 10, 2009;

In some embodiments, the IL-23 receptor binding molecule is administered in combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009)

In some embodiments, the IL-23 receptor binding molecule is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in PCT International Patent Publication No. WO 2016/144803 published Sep. 15, 2016; Lifke, et al. United States Patent Publication No. US 20160257749 A1 published Sep. 8, 2016 (F. Hoffman-LaRoche); Karunsky, U.S. Pat. No. 9,631,026 issued Apr. 27, 2017; Karunsky, Sabatos-Peyton, et al. U.S. Pat. No. 8,841,418 issued Sep. 23, 2014; U.S. Pat. No. 9,605,070; Takayanagi, et al., U.S. Pat. No. 8,552,156 issued Oct. 8, 2013.

In some embodiments, the IL-23 receptor binding molecule is administered in combination with an inhibitor of both LAG3 and PD1 as the blockade of LAG3 and PD1 has been suggested to synergistically reverse anergy among tumor-specific CD8+ T-cells and virus-specific CD8+ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392.

In some embodiments, the IL-23 receptor binding molecule is administered in combination with an A2aR inhibitor. A2aR inhibits T-cell responses by stimulating CD4⁺ T-cells towards developing into T_{Reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the IL-23 receptor binding molecules for use in the treatment disorders such as cancer and Parkinson's disease.

In some embodiments, the IL-23 receptor binding molecule is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g. cancer) in a mammalian subject by the administration of a IL-23 receptor binding molecule in combination with an agent(s) that modulate at least one immune checkpoint pathway including immune checkpoint pathway modulators that modulate two, three or more immune checkpoint pathways.

In some embodiments the IL-23 receptor binding molecule is administered in combination with an immune checkpoint modulator that modulates multiple immune checkpoint pathways. Multiple immune checkpoint pathways may be modulated by the administration of multi-functional molecules which act as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of IL-23 receptor binding molecules in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the IL-23 receptor binding molecule may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a IL-23 receptor binding molecule of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

Cell Therapy Agents and Methods as Supplementary Agents:

In some embodiments, the methods of the disclosure may include the combination of the administration of a IL-23 receptor binding molecules with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of an CD25 biased IL-23 receptor binding molecule as described herein.

In some embodiments of the methods of the present disclosure, the supplementary agent is a “chimeric antigen receptor T-cell” and “CAR-T cell” are used interchangeably to refer to a T-cell that has been recombinantly modified to express a chimeric antigen receptor. As used herein, the terms As used herein, the terms “chimeric antigen receptor” and “CAR” are used interchangeably to refer to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS (USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis). In some embodiments, the CAR-T possesses a CAR specifically binds to a cell surface molecule associated with a tumor cell is selected from the group consisting of GD2, BCMA, CD19, CD33, CD38, CD70, GD2, IL3Rα2, CD19, mesothelin, Her2, EpCam, Muc1, ROR1, CD133, CEA, EGRFRVIII, PSCA, GPC3, Pan-ErbB and FAP.

Physical Methods:

In some embodiments, the supplementary agent is a anti-neoplastic physical methods including but not limited to radiotherapy, cryotherapy, hyperthermic therapy, surgery, laser ablation, and proton therapy.

Use in Combination with Supplementary Agents:

In some embodiments of the therapeutic uses of the compositions of the present disclosure, the administration of a therapeutically effective amount of an IL23 receptor binding molecule (or nucleic acid encoding an IL23 receptor binding molecule including recombinant viruses encoding the IL23 receptor binding molecule) are administered in combination with one or more additional active agents (“supplementary agents”).

As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g., IL23 receptor binding molecule) is considered to be administered in combination with a second agent (e.g. a therapeutic autoimmune antibody such as Humira®) if the biological effect resulting from the administration of the first agent persists in the subject at the time of administration of the second agent such that the therapeutic effects of the first agent and second agent overlap. For example, the therapeutic antibodies are sometimes administered by IV infusion every two weeks while the IL23 receptor binding molecules of the present disclosure may be administered more frequently, e.g. daily, BID, or weekly. However, the administration of the first agent (e.g. entaercept) provides a therapeutic effect over an extended time and the administration of the second agent (e.g. an IL23 receptor binding molecule) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the IL23 receptor binding molecule and the supplementary agent(s) are administered or applied sequentially, e.g., where one agent is administered prior to one or more other agents. In other embodiments, the IL23 receptor binding molecule and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

Supplementary agents may administered or introduced separately, for example, formulated separately for separate administration (e.g., as may be provided in a kit) and/or therapies that can be administered or introduced in combination with the IL23 receptor binding molecules.

### Prophylactic Applications

In some embodiments where the IL23 receptor binding molecule is used in prophylaxis of disease, the supplementary agent may be a vaccine. The IL23 receptor binding molecule of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IL23 receptor binding molecule of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, humanpapilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### Examples

### Example 1—VHH Generation

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7th immunization.

After immunization, the library was constructed. Briefly, RNA was extracted from blood and transcribed to cDNA. The VHH regions were obtained via two-step PCR, which fragment about 400 bp. The PCR outcomes and the vector of pMECS phagemid were digested with Pst I and Not I, subsequently, ligated to pMECS/Nb recombinant. After ligation, the products were transformed into Escherichia coli (E. coli) TG1 cells by electroporation. Then, the transformants were enriched in growth medium and planted on plates. Finally, the library size was estimated by counting the number of colonies.

Library biopanning was conducted to screen candidates against the antigens after library construction. Phage display technology was applied in this procedure. Positive colonies were identified by PE-ELISA.

### Example 2—Recombinant Production and Purification

Codon optimized DNA inserts were cloned into modified pcDNA3.4 (Genscript) for small scale expression in HEK293 cells in 24 well plates. The binding molecules were purified in substantial accordance with the following procedure. Using a Hamilton Star automated system, 96×4 mL of supernatants in 4×24-well blocks were re-arrayed into 4×96-well, 1 mL blocks. PhyNexus micropipette tips (Biotage, San Jose Calif.) holding 80 μL of Ni-Excel IMAC resin (Cytiva) are equilibrated wash buffer: PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM imidazole. PhyNexus tips were regenerated in 3×1 mL blocks of 0.5 M sodium hydroxide.

The purified protein eluates were quantified using a Biacore® T200 as in substantial accordance with the following procedure. 10 uL of the first 96×0.36 mL eluates were transferred to a Biacore®96-well microplate and diluted to 60 uL in HBS-EP+ buffer (10 mM Hepes pH 7.4, 150 mM NaCl, 1 mM EDTA, 0.05% Tween 20). Each of the 96 samples was injected on a CM5 series S chip previously functionalized with anti-histidine capture antibody (Cytiva): injection is performed for 18 seconds at 5 μL/min. Capture levels were recorded 60 seconds after buffer wash. A standard curve of known VHH concentrations (270, 90, 30, 10, 3.3, 1.1 μg/mL) was acquired in each of the 4 Biacore chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 kDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent The final, normalized plate was sterile filtered using 0.22 μm filter plates (Corning).

### Experimental

The single domain antibodies of the present disclosure were obtained from camels by immunization with an extracellular domain of a IL-23 receptor. IL-23 VHH molecules of the present disclosure of the present disclosure were generated in substantial accordance with the teaching of the Examples. Briefly, a camel was sequentially immunized with the ECD of the human IL-23 and mouse IL-23 over a period several weeks of by the subcutaneous an adjuvanted composition containing a recombinantly produced fusion proteins comprising the extracellular domain of the IL-23, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of biopanning of the phage library were conducted to identify VHHs that bound to the ECD of IL-23 (human or mouse as appropriate). Individual phage clones were isolated for periplasmic extract ELISA (PE-ELISA) in a 96-well plate format and selective binding confirmed by colorimetric determination. The IL-23 binding molecules that demonstrated specific binding to the IL-23 antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence. The VHH molecules demonstrating specific binding to the hIL-23 ECD antigen (anti-human IL-23 VHHs) and the CDRs isolated from such VHHs are provided in Table 8 and the CDRs isolated from such VHHs are provided in Table 4. The VHH molecules demonstrating specific binding to the mIL-23 ECD antigen (anti-mouse IL-23 VHHs) are provided in Table 9 and the CDRs isolated from such VHHs are provided in Table 3. Nucleic acid sequences encoding the VHHs of Table 8 and 9 are provided in Tables 12 and 13 respectively.

In some instances, due to sequence or structural similarities between the extracellular domains of IL-23 receptors from various mammalian species, immunization with an antigen derived from a IL-23 of a first mammalian species (e.g., the hIL-23 ECD) may provide antibodies which specifically bind to IL-23 receptors of one or more additional mammalian species. Such antibodies are termed “cross reactive.” For example, immunization of a camelid with a human derived antigen (e.g., the hIL-23-ECD) may generate antibodies that are cross-reactive the murine and human receptors. Evaluation of cross-reactivity of antibody with respect to the receptors derived from other mammalian species may be readily determined by the skilled artisan, for example using the methods relating to evaluation of binding affinity and/or specific binding described elsewhere herein such as flow cytometry or SPR. Consequently, the use of the term “human IL-23 VHH” or “hIL-23 VHH” merely denotes that the species of the IL-23 antigen used for immunization of the camelid from which the VHH was derived was the human IL-23 (e.g., the hIL-23, ECD) but should not be understood as limiting with respect to the specific binding affinity of the VHH for hIL-23 molecules of other mammalian species. Similarly, the use of the term “mouse IL-23 VHH” or “mIL-23” merely denotes that the species of the IL-23 antigen used for immunization of the camelid from which the VHH was derived was the murine IL-23 (e.g., the mIL-23 ECD) but should not be understood as limiting with respect to the specific binding affinity of the VHH for IL-23 molecules of other mammalian species.

The single domain antibodies of the present disclosure were obtained from camels by immunization with an extracellular domain of a IL12Rb1 receptor. IL12Rb1 VHH molecules of the present disclosure of the present disclosure were generated in substantial accordance with the teaching of the Examples. Briefly, a camel was sequentially immunized with the ECD of the human IL12Rb1 and mouse IL12Rb1 over a period several weeks of by the subcutaneous an adjuvanted composition containing a recombinantly produced fusion proteins comprising the extracellular domain of the IL12Rb1, the human IgG1 hinge domain and the human IgG1 heavy chain Fc. Following immunization, RNAs extracted from a blood sample of appropriate size VHH-hinge-CH2-CH3 species were transcribed to generate DNA sequences, digested to identify the approximately 400 bp fragment comprising the nucleic acid sequence encoding the VHH domain was isolated. The isolated sequence was digested with restriction endonucleases to facilitate insertion into a phagemid vector for in frame with a sequence encoding a his-tag and transformed into E. coli to generate a phage library. Multiple rounds of biopanning of the phage library were conducted to identify VHHs that bound to the ECD of IL12Rb1 (human or mouse as appropriate). Individual phage clones were isolated for periplasmic extract ELISA (PE-ELISA) in a 96-well plate format and selective binding confirmed by colorimetric determination. The IL12Rb1 binding molecules that demonstrated specific binding to the IL12Rb1 antigen were isolated and sequenced and sequences analyzed to identify VHH sequences, CDRs and identify unique VHH clonotypes. As used herein, the term “clonotypes” refers a collection of binding molecules that originate from the same B-cell progenitor cell, in particular collection of antigen binding molecules that belong to the same germline family, have the same CDR3 lengths, and have 70% or greater homology in CDR3 sequence. The VHH molecules demonstrating specific binding to the hIL12Rb1 ECD antigen (anti-human IL12Rb1 VHHs) and the CDRs isolated from such VHHs are provided in Table 6 and the CDRs isolated from such VHHs are provided in Table 2. The VHH molecules demonstrating specific binding to the mIL12Rb1 ECD antigen (anti-mouse IL12Rb1 VHHs) are provided in Table 7 and the CDRs isolated from such VHHs are provided in Table 3. Nucleic acid sequences encoding the VHHs of Table 2 and 3 are provided in Tables 10 and 11 respectively.

In some instances, due to sequence or structural similarities between the extracellular domains of IL12Rb1 receptors from various mammalian species, immunization with an antigen derived from a IL12Rb1 of a first mammalian species (e.g., the hIL12Rb1-ECD) may provide antibodies which specifically bind to IL12Rb1 receptors of one or more additional mammalian species. Such antibodies are termed “cross reactive.” For example, immunization of a camelid with a human derived antigen (e.g., the hIL12Rb1-ECD) may generate antibodies that are cross-reactive the murine and human receptors. Evaluation of cross-reactivity of antibody with respect to the receptors derived from other mammalian species may be readily determined by the skilled artisan, for example using the methods relating to evaluation of binding affinity and/or specific binding described elsewhere herein such as flow cytometry or SPR. Consequently, the use of the term “human IL12Rb1 VHH” or “hIL12Rb1 VHH” merely denotes that the species of the IL12Rb1 antigen used for immunization of the camelid from which the VHH was derived was the human IL12Rb1 (e.g., the hIL12Rb1, ECD) but should not be understood as limiting with respect to the specific binding affinity of the VHH for hIL12Rb1 molecules of other mammalian species. Similarly, the use of the term “mouse IL12Rb1 VHH” or “mIL12Rb” merely denotes that the species of the IL12Rb1 antigen used for immunization of the camelid from which the VHH was derived was the murine IL12Rb1 (e.g., the mIL12Rb1 ECD) but should not be understood as limiting with respect to the specific binding affinity of the VHH for IL12Rb1 molecules of other mammalian species.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

Tables

TABLE 2
anti-human IL12RB1 sdAb CDR Sequences

| | | SEQ ID | | SEQ ID | | SEQ ID |
|---|---|---|---|---|---|---|
| Name | CDR1 | NO : | CDR2 | NO: | CDR3 | NO: |
| hIL12Rb1 | YGYCGYDMS | 25. | LITSDRSISYEDSVKA | 47. | SAAARESSWCRSRYRVAS | 69. |
| _VHH1 | | | | | | |
| hIL12Rb1 | YTYSSAFMA | 26. | AIYTRDGGTVYADSVKG | 48. | KIPQPGRASLLDSQTYDY | 70. |
| _VHH2 | | | | | | |
| hIL12Rb1 | YSYCGYDMM | 27. | LITSDYSIRYEDSVEG | 49. | STAARESSWCRSRYRVAS | 71. |
| _VHH3 | | | | | | |
| hIL12Rb1 | YGYCGYDMS | 28. | LITSDRIASYEDSVKG | 50. | SAAARENSWCRSRYRVAS | 72. |
| _VHH4 | | | | | | |
| hIL12Rb1 | YGYCGYDMS | 29. | LITSDRSVSYEDSVKG | 51. | STAARENNWCRSRYRIAY | 73. |
| _VHH5 | | | | | | |
| hIL12Rb1 | YTYTNNFMA | 30. | AIYTGDGYAYYFYSVKG | 52. | MERRIGTRRMTENAEYKY | 74. |
| _VHH6 | | | | | | |
| hIL12Rb1 | YDYCGYDVR | 31. | GIDSDGSTSYADSVKG | 53. | ESPAGESAWCRNFRGMDY | 75. |
| _VHH7 | | | | | | |
| hIL12Rb1 | YSYCGYDMM | 32. | LITSDYSIRYEDSVEG | 54. | STAARESSWCRSRYRVAS | 76. |
| _VHH8 | | | | | | |
| hIL12Rb1 | YSYCGYDMM | 33. | LITSDYSIRYEDSVEG | 55. | STAARESGWCRSRYRVAS | 77 . |
| _VHH9 | | | | | | |
| hIL12Rb1 | YDYCGYDVR | 34. | GIDSDGSTSYADSVKG | 56. | ESPAGESAWCRNFRGMDY | 78. |
| _VHH10 | | | | | | |
| hIL12Rb1 | YDYCGYDVR | 35. | GIDSDGSTSYADSVKG | 57. | ESPAGESAWCRNFRGMDY | 79. |
| _VHH11 | | | | | | |
| hIL12Rb1 | YTYSSAFMA | 36. | AIYTRDGGTVYADSVKG | 58 . | KMPQPGRASLLDSQTYDY | 80. |
| _VHH12 | | | | | | |
| hIL12Rb1 | YGYCGYDMS | 37. | LITSERVISYEDSVKG | 59. | SAAARESSWCRSRYRVAS | 81. |
| _VHH13 | | | | | | |
| hIL12Rb1 | YDYCGYDVR | 38. | GIDSDGSTSYADSVKG | 60. | ESPAGESAWCRNFRGMDY | 82. |
| _VHH14 | | | | | | |
| hIL12Rb1 | YDYCGYDVR | 39. | GINSDGSTSYADSVKG | 61. | ESPAGESAWCRNFRGMDY | 83. |
| _VHH15 | | | | | | |
| hIL12Rb1 | YTYSSAFMA | 40. | AMYTRDGGTVYADSVKG | 62. | KIPQPGRASLLDSQTYDY | 84. |
| _VHH16 | | | | | | |
| hIL12Rb1 | YGYCGYDMS | 41. | LITSDRSVSYEDSVKG | 63. | STAARENNWCRSRYRIAS | 85. |
| _VHH17 | | | | | | |
| hIL12Rb1 | YTYTNNFMA | 42. | AIYTGDGYAYYFDSVKG | 64. | MERRSGRRRMTENAEYKY | 86. |
| _VHH18 | | | | | | |
| hIL12Rb1 | YDYCGYDVR | 43. | GINSDGSTSYADSVKG | 65. | EGPAGESAWCRNFRGMDY | 87. |
| _VHH19 | | | | | | |
| hIL12Rb1 | YTYSSAFMA | 44. | AIYTRDGSPVYADSLKG | 66. | KIPEPGRISLLDSQTYDY | 88. |
| _VHH20 | | | | | | |
| hIL12Rb1 | YTYSSAFMA | 45. | AMYTRDGGTVYADSVKG | 67. | KIPQPGRASLLDSQTYDY | 89. |
| _VHH21 | | | | | | |
| hIL12Rb1 | YTYSSAFMA | 46. | AIYTRDGGTVYADSVKG | 68. | KIPQPGRASLLDSQTYDY | 90. |
| _VHH22 | | | | | | |

TABLE 3
anti-mIL12RB1 sdAb CDRs MOUSE
mIL12RB1_VHH CDRs

| | SEQ | | SEQ | | SEQ |
|---|---|---|---|---|---|
| CDR1 | ID | CDR2 | ID | CDR3 | ID |
| (AA Seq) | NO | (AA Seq) | NO | (AA Seq) | NO |
| YTYSSAFMA | 26 | AIYTRDGGTVYADSVKG | 48 | KIPQPGRASLLDSQTYDY | 70 |
| YDYCGYDVR | 31 | GIDSDGSTSYADSVKG | 53 | ESPAGESAWCRNFRGMDY | 75 |
| YSYCGYDMM | 27 | LITSDYSIRYEDSVEG | 49 | STAARESSWCRSRYRVAS | 71 |
| YTYTNNFMA | 30 | AIYTGDGYAYYFDSVKG | 64 | MERRSGRRRMTENAEYKY | 86 |
| FTIDDSEMG | 178 | SGSSDDDTYYVDSVKG | 179 | GPTYPPKDGDCAH | 180 |
| YTYSSAFMA | 26 | AIYTRDGSPVYADSLKG | 66 | KIPEPGRISLLDSQTYDY | 88 |
| YDYCGYDVR | 31 | GIDSDGSTSYADSVKG | 53 | ESPAGESAWCRNFRGMDY | 75 |
| YGYCGYDMS | 25 | LITSERVISYEDSVKG | 59 | SAAARESSWCRSRYRVAS | 69 |
| YGYCGYDMS | 25 | LITSDRSISYEDSVKA | 47 | SAAARESSWCRSRYRVAS | 69 |
| YDYCGYDVR | 31 | GIDSDGSTSYADSVKG | 53 | ESPAGESAWCRNFRGMDY | 75 |
| YSYCGYDMM | 27 | LITSDYSIRYEDSVEG | 49 | STAARESSWCRSRYRVAS | 71 |
| YTYTNNFMA | 30 | AIYTGDGYAYYFYSVKG | 52 | MERRIGTRRMTENAEYKY | 74 |
| YSYCGYDMM | 27 | LITSDYSIRYEDSVEG | 49 | STAARESGWCRSRYRVAS | 77 |
| YDYCGYDVR | 31 | GINSDGSTSYADSVKG | 61 | ESPAGESAWCRNFRGMDY | 75 |
| YGYCGYDMS | 25 | LITSDRSVSYEDSVKG | 51 | STAARENNWCRSRYRIAY | 73 |
| YDYCGYDVR | 31 | GINSDGSTSYADSVKG | 61 | EGPAGESAWCRNFRGMDY | 87 |
| YTYSSAFMA | 26 | AMYTRDGGTVYADSVKG | 62 | KIPQPGRASLLDSQTYDY | 70 |
| YTYSSAFMA | 26 | AMYTRDGGTVYADSVKG | 62 | KIPQPGRASLLDSQTYDY | 70 |
| YGYCGYDMS | 25 | LITSDRSVSYEDSVKG | 51 | STAARENNWCRSRYRIAS | 85 |
| YGYCGYDMS | 25 | LITSDRIASYEDSVKG | 50 | SAAARENSWCRSRYRVAS | 72 |
| YDYCGYDVR | 31 | GIDSDGSTSYADSVKG | 53 | ESPAGESAWCRNFRGMDY | 75 |
| YTYSSAFMA | 26 | AIYTRDGGTVYADSVKG | 48 | KIPQPGRASLLDSQTYDY | 70 |
| YTYSSAFMA | 26 | AIYTRDGGTVYADSVKG | 48 | KMPQPGRASLLDSQTYDY | 80 |

TABLE 4
anti-human IL23R sdAb CDR Sequences

| | | SEQ ID | | SEQ ID | | SEQ ID |
|---|---|---|---|---|---|---|
| Name | CDR1 | NO: | CDR2 | NO: | CDR3 | NO: |
| hIL23R_VHH1 | YTYCSYDMS | 181 | AFNSDGTTSY | 192 | DPHVQSSGGY | 206 |
| | | | ADSVKG | | CPPY | |
| hIL23R_VHH2 | YTYCSYDMS | 181 | SFNSDGSTSY | 193 | DPHADWGAPC | 207 |
| | | | ADSVKG | | GGDY | |
| hIL23R_VHH3 | YTYCTYDMT | 182 | GIHSDGTTSY | 194 | DPIATITRRC | 208 |
| | | | ADSVKG | | DSY | |
| hIL23R_VHH4 | STYCTYDMT | 183 | AINSDGSTSY | 195 | DPNSGWGAPC | 209 |
| | | | ADSVKG | | GGDY | |
| hIL23R_VHH5 | YTYCSYDMS | 181 | AIASDGSTSY | 196 | DPHVQSSGGY | 206 |
| | | | ADSLKG | | CPPY | |
| hIL23R_VHH6 | YTYCSYDMG | 184 | SINSDGTTSY | 197 | DPQTRPGKPC | 210 |
| | | | ADSVKG | | ADY | |
| hIL23R_VHH7 | YTYCNYDIA | 185 | AIASDGITSY | 198 | DPISTITRIC | 211 |
| | | | ADSVKG | | DPY | |
| hIL23R_VHH8 | YTYCSYDMK | 186 | GIDSDGSISY | 199 | EGTIPVGACP | 212 |
| | | | ADSVKG | | NY | |
| hIL23R_VHH9 | YTYCSYDMS | 181 | SINSDGTTSY | 197 | DPQTRPGKPC | 210 |
| | | | ADSVKG | | ADY | |
| hIL23R_VHH1 | YTYCNYDIA | 185 | AIASDGSTSY | 200 | DPIATMTRRC | 143 |
| 0 | | | ADSVKG | | DPY | |
| hIL23R_VHH1 | YTYCSYDMT | 187 | AIDSDGSTSY | 201 | DPIATISRRC | 213 |
| 1 | | | ADSVKG | | DSY | |
| hIL23R_VHH1 | YTSSSRCMG | 188 | RIYTPTRTTW | 202 | GASCAVDLFS | 214 |
| 2 | | | YADSVKG | | Y | |
| hIL23R_VHH1 | YTYCSYDMK | 186 | AIDSDGSTSY | 201 | EGTIPVGVCP | 215 |
| 3 | | | ADSVKG | | NY | |
| hIL23R_VHH1 | YTYCSYDMK | 186 | AIDSDGSTSY | 201 | EGTVPVGVCP | 216 |
| 4 | | | ADSVKG | | NY | |
| hIL23R_VHH1 | GTYTSRYMG | 189 | TIWPAGGNTV | 203 | AKYGGTSLAP | 217 |
| 5 | | | YADSVKG | | YTYNY | |
| hIL23R_VHH1 | YTYCNYDIA | 185 | AIASDGSTSY | 200 | DPIATMTRRC | 143 |
| 6 | | | ADSVKG | | DPY | |
| hIL23R_VHH1 | YTFSTMKYMG | 190 | AIWIAAGNTY | 204 | ARYGFVPSTW | 218 |
| 7 | | | YADSVKG | | YLPERYNY | |
| hIL23R_VHH1 | YTYCNYDIA | 185 | AIASDGSTSY | 200 | DPIATMTRRC | 143 |
| 8 | | | ADSVKG | | DPY | |
| hIL23R_VHH1 | YTSCSYDMS | 191 | AIHSDGTTSY | 205 | DPNYSDHVCP | 144 |
| 9 | | | ADSMKG | | PY | |

TABLE 5
anti- mIL23R sdAb CDRs MOUSE
mIL23R ECD Generated_VHH CDRs

| | SEQ | | SEQ | | SEQ |
|---|---|---|---|---|---|
| CDR1 | ID | CDR2 | ID | CDR3 | ID |
| (AA Seq) | NO | (AA Seq) | NO | (AA Seq) | NO |
| YTYSSCTMG | 219 | MLISDGSTFYADSVKG | 235 | ATLGSRTV | 249 |
| FTFRLAAMR | 220 | GIDSRGSTIYADSVKG | 236 | GVYGDTYS | 250 |
| NTYCEYNMS | 221 | GVDSDGSTRYSESVKG | 237 | YVCTFCSGNSCYYEYKYYY | 251 |
| YTYSNNCMG | 222 | NIYTGGGRTTYADSVKG | 238 | GSCGSARSEYSY | 252 |
| YTFCMA | 223 | RFYTRDGYTYYSDSVKG | 239 | DLARCSSNKNDFRY | 253 |
| YTSGNYWMG | 224 | TLWTGGASTFYGDSVKG | 240 | DPALRLGANILRPAEYKY | 254 |
| FTFSRSAMT | 225 | GIDSGGTTVYADSVKG | 241 | GLPWGNTWRT | 255 |
| YTYSSCTMG | 219 | MVFSDGSTFYADSVKG | 242 | ATLGSRTI | 256 |
| FTFRLTAMR | 226 | GIDSAGSTIYADSVKG | 243 | GVYGDTYS | 250 |
| DTYSSCTMG | 227 | MLMGDGSTFYADSVKG | 244 | ATLGSRTI | 256 |
| YTYSSCTMG | 219 | MLISDGSTFYADSVKG | 235 | ATLGSRTV | 249 |
| YTYSSCTMG | 219 | MLISDGSTFYADSVKG | 235 | ATLGSRTV | 249 |
| FTFRLAAMR | 220 | GIDSRGSTIYADSVKG | 236 | GVYGDTYS | 250 |
| FTFRTSAMT | 228 | GIDSGGTTVYADSVKG | 241 | GLPWGNTWRT | 255 |
| YTYSSCTMG | 219 | MVFSDGSTFYADSVKG | 242 | ATLGSRTI | 256 |
| DTYSSCTMG | 227 | MLMGDGSTFYADSVKG | 244 | ATLGSRTI | 256 |
| FTFRLTAMR | 226 | GIDSRGSTIYADSVKG | 236 | GVYGDTYS | 250 |
| FTFRLTAMR | 226 | GIDSRGSTIYADSVKG | 236 | GVYGDTYS | 250 |
| FTFRLSAMR | 229 | GIDSRGSTIYADSVKG | 236 | GVYGDTYS | 250 |
| FTFSSSAMT | 230 | GIDSGGTTVYADSVKG | 241 | GLPWGNTWRT | 255 |
| FTFSSSAMT | 230 | GIDSGGTTVYADSVKG | 241 | GLPWGNTWRT | 255 |
| YTFCMA | 223 | RFYTRDSYTYYSDSVKG | 245 | DLTRCSSNKNDFRY | 257 |
| FNFRLYAMR | 231 | GIDSGGSTIYADSVKG | 246 | GVYGDTYS | 250 |
| YTFCMA | 223 | RFYTRDGYTYYSGSVKG | 247 | DLTRCSSNKNDFRY | 257 |
| YTYSSCTMG | 219 | MLISDGSTFYADSVKG | 235 | ATLGSRTV | 249 |
| YTFCMA | 223 | RFYTRDGYTYYSDSVKG | 239 | DLTRCSSNKNDFRY | 257 |
| YTYSSCTMG | 219 | MLISDGSTFYADSVKG | 235 | ATLGSRTV | 249 |
| FTFRLTAMR | 226 | GIDSRGSTIYADSVKG | 236 | GVYGDTYS | 250 |
| FTFSTSAMT | 232 | GIDSGGTTVYADSVKG | 241 | GLPWGNTWRT | 255 |
| FTFRLTAMR | 226 | GIDSRGSTIYADSVKG | 236 | GVYGDTYS | 250 |
| FTFRLTAMR | 226 | GIDSRGSTIYADSVKG | 236 | GVYGDTYS | 250 |
| FTFSRSAMT | 225 | GIDSGGTTVYADSVKG | 241 | GLPWGNTWRT | 255 |
| FTFSSSAMT | 230 | GIDSGGTTVYADSVKG | 241 | GLPWGNTWRT | 255 |
| FTFRLTAMR | 226 | GIDSRGSTIYADSVKG | 236 | GVYGDTHS | 258 |
| FTFSSSAMT | 230 | GIDSGGTTVYADSVKG | 241 | GLPWGNTWRT | 255 |
| DTYSSCTMG | 227 | MVFSDGSTFYADSVKG | 242 | ATLGSRTI | 256 |
| FTFSSGAMT | 233 | GIDSGGTTVYADSVKG | 241 | GLPWGNTWRT | 255 |
| FTFSTSAMT | 232 | GIDSGGTTVYADSVKG | 241 | GLPWGNIWRT | 259 |
| FTFSRSAMT | 225 | GIDSGGTTVYADSVKG | 241 | GLPWGNTWRT | 255 |
| FTFRLTAMR | 226 | GIDSRGSTIYADSVKG | 236 | GVYGDTYS | 250 |
| FTFSTSAMT | 232 | GIDSGGTTVYADSVKG | 241 | GLPWGNTWRT | 255 |
| FTFSNYAMR | 234 | GIDSRGSTIYADSVKG | 236 | GVYGDTYS | 250 |
| YTFCMA | 223 | RFYTRDGYTYYSDSVKG | 239 | DLTRCSSNKNDFRY | 257 |
| FTFRLSAMR | 229 | GIDSRGSTIYADSVKG | 236 | GVYGDTYS | 250 |
| FTFRLSAMR | 229 | GIDSRGSTIYADSVEG | 248 | GVYGDTYS | 250 |
| FTFRLSAMR | 229 | GIDSRGSTIYADSVKG | 236 | GVYGDTYS | 250 |

TABLE 6
anti-human IL12RB1 sdAb VHH Amino Acid Sequences

| | | SEQ |
|---|---|---|
| Name | Sequence | ID NO |
| hIL12R | QVQLQESGGGSVQAGGSLRLSCVASGYGYCGYDMSWYRQAPGKER | 153 |
| b1_VHH | EFVALITSDRSISYEDSVKARFIISRDNAANTGYLDMTRLTPDDT | |
| 1 | AIYYCKTSAAARESSWCRSRYRVASWGQGTQVTVSS | |
| hIL12R | QVQLQESGGGSVQAGGSLRLSCTASGYTYSSAFMAWFRQAPGKER | 166 |
| b1_VHH | EGVAAIYTRDGGTVYADSVKGRFTISQDNAKNILYLQMNSLKAED | |
| 2 | TAMYYCAAKIPQPGRASLLDSQTYDYWGQGTQVTVSS | |
| hIL12R | QVQLQESGGGSVQAGGSLRLSCVASGYSYCGYDMMWYRQAPGKER | 147 |
| b1_VHH | EFVALITSDYSIRYEDSVEGRFSISRDNAKNTGYLLMSNLTPADT | |
| 3 | AIYYCKTSTAARESSWCRSRYRVASWGQGTQVTVSS | |
| hIL12R | QVQLQESGGGSVQAGGSLRLSCVASGYGYCGYDMSWYRQTPGKER | 164 |
| b1_VHH | EFVALITSDRIASYEDSVKGRFIISRDNAKNTGYLDMTRVTPDDT | |
| 4 | AIYYCKTSAAARENSWCRSRYRVASWGQGTQVTVSS | |
| hIL12R | QVQLQESGGGSVQAGGFLRLSCVASGYGYCGYDMSWYRQVPGKER | 159 |
| b1_VHH | EFVALITSDRSVSYEDSVKGRFSISRDNAKNTAYLEMNRLTPDDT | |
| 5 | AVYYCKTSTAARENNWCRSRYRIAYWGQGTQVTVSS | |
| hIL12R | QVQLQESGGGSVQAGGSLRLSCAASRYTYTNNFMAWFRQAPGKER | 156 |
| b1_VHH | EGVAAIYTGDGYAYYFYSVKGRFTISQDNDENMLYLQMNSLKPED | |
| 6 | TAMYYCAAMERRIGTRRMTENAEYKYWGQGTQVTVSS | |
| hIL12R | QVQLQESGGGSVQAGGSLRLSCAVSGYDYCGYDVRWYRRAPGKER | 151 |
| b1_VHH | EFVSGIDSDGSTSYADSVKGRFTISQDNAENTSYLHMFSLKPEDT | |
| 7 | AMYYCKTESPAGESAWCRNFRGMDYWGKGTQVTVSS | |
| hIL12R | QVQLQESGGGSVQAGGSLRLSCVASGYSYCGYDMMWYRQAPGKER | 147 |
| b1_VHH | EFVALITSDYSIRYEDSVEGRFSISRDNAKNTGYLLMSNLTPADT | |
| 8 | AIYYCKTSTAARESSWCRSRYRVASWGQGTQVTVSS | |
| hIL12R | QVQLQESGGGSVQAGGSLRLSCVASGYSYCGYDMMWYRQAPGKER | 157 |
| b1_VHH | EFVALITSDYSIRYEDSVEGRFSISRDNAKNTGYLLMSNLTPADT | |
| 9 | AIYYCKTSTAARESGWCRSRYRVASWGQGTQVTVSS | |
| hIL12R | QVQLQESGGGSVQAGGSLRLSCAVSGYDYCGYDVRWYRQAPGKER | 146 |
| b1_VHH | EFVSGIDSDGSTSYADSVKGRFTISQDNAENTSYLHMFSLKPEDT | |
| 10 | AMYYCKTESPAGESAWCRNFRGMDYWGKGTQVTVSS | |
| hIL12R | QVQLQESGGGSVQAGGSLRLSCAVSGYDYCGYDVRWYRQAPGKER | 146 |
| b1_VHH | EFVSGIDSDGSTSYADSVKGRFTISQDNAENTSYLHMFSLKPEDT | |
| 11 | AMYYCKTESPAGESAWCRNFRGMDYWGKGTQVTVSS | |
| hIL12R | QVQLQESGGGSVQAGGSLRLSCTASGYTYSSAFMAWFRQAPGKER | 167 |
| b1_VHH | EGVAAIYTRDGGTVYADSVKGRFTISQDNAKNTLYLQMNSLKPED | |
| 12 | TAMYYCAAKMPQPGRASLLDSQTYDYWGQGTQVTVSS | |
| hIL12R | QVQLQESGGGSVQAGGFLRLSCVASGYGYCGYDMSWYRQAPGKER | 152 |
| b1_VHH | EFVALITSERVISYEDSVKGRFSISRDNAENTGYLEMNRLTPDDT | |
| 13 | AIYYCKTSAAARESSWCRSRYRVASWGQGTQVTVSS | |
| hIL12R | QVQLQESGGGSVQAGGSLRLSCAVSGYDYCGYDVRWYRRAPGKER | 151 |
| b1_VHH | EFVSGIDSDGSTSYADSVKGRFTISQDNAENTSYLHMFSLKPEDT | |
| 14 | AMYYCKTESPAGESAWCRNFRGMDYWGKGTQVTVSS | |
| hIL12R | QVQLQESGGGSVQAGGSLRLSCAVSGYDYCGYDVRWYRQAPGKER | 158 |
| b1_VHH | EFVSGINSDGSTSYADSVKGRFTISQDNAENTSYLHMFSLKPEDT | |
| 15 | AMYYCKTESPAGESAWCRNFRGMDYWGKGTQVTVSS | |
| hIL12R | QVQLQESGGGSVQAGGSLRLSCTASGYTYSSAFMAWFRQAPGKER | 161 |
| b1_VHH | EGVAAMYTRDGGTVYADSVKGRFTISQDNAKNTLYLQIHTLKAED | |
| 16 | TAMYYCAAKIPQPGRASLLDSQTYDYWGQGTQVTVSS | |
| hIL12R | QVQLQESGGGSVQAGGFLRLSCVASGYGYCGYDMSWYRQVPGKER | 163 |
| b1_VHH | EFVALITSDRSVSYEDSVKGRFSISRDNAKNTAYLEMNRLTPDDT | |
| 17 | AIYYCKTSTAARENNWCRSRYRIASWGQGTQVTVSS | |
| hIL12R | QVQLQESGGGSVQAGGSLRLSCAASRYTYTNNFMAWFRQAPGKER | 148 |
| b1_VHH | EGVAAIYTGDGYAYYFDSVKGRFTISQDNDKNMLYLQMNSLKPED | |
| 18 | TAMYYCAAMERRSGRRRMTENAEYKYWGQGTQVTVSS | |
| hIL12R | QVQLQESGGGSVQAGGSLRLSCAVSGYDYCGYDVRWYRQAPGKER | 160 |
| b1_VHH | EFVSGINSDGSTSYADSVKGRFTISQDNAENTSYLHMFSLKPEDT | |
| 19 | AMYYCKTEGPAGESAWCRNFRGMDYWGKGTQVTVSS | |
| hIL12R | QVQLQESGGGSVQAGGSLRLSCTASGYTYSSAFMAWFRQAPGKER | 150 |
| b1_VHH | EGVAAIYTRDGSPVYADSLKGRFTISQDNAKNTLHLQMNSLKPED | |
| 20 | TAMYYCAAKIPEPGRISLLDSQTYDYWGHGTQVTVSS | |
| hIL12R | QVQLQESGGGSVQAGGSLRLSCTASGYTYSSAFMAWFRQAPGKER | 162 |
| b1_VHH | EGVAAMYTRDGGTVYADSVKGRFTISQDNAKNTLYLQMNSLKTED | |
| 21 | TAMYYCAAKIPQPGRASLLDSQTYDYWGQGTQVTVSS | |
| hIL12R | QVQLQESGGGSVQAGGSLRLSCTASGYTYSSAFMAWFRQAPGKER | 145 |
| b1_VHH | EGVAAIYTRDGGTVYADSVKGRFTISQDNAKNTLYLQMNSLKAED | |
| 22 | TAMYYCAAKIPQPGRASLLDSQTYDYWGQGTQVTVSS | |

TABLE 7
anti- mIL12RB sdAb VHH AMINO ACID SEQUENCE MOUSE
mIL12RB ECD Generated VHHs

| Name | VHH Amino Acid Sequence (CDRs underlined | SEQ ID NO |
|---|---|---|
| mIL12Rb1 | QVQLQESGGGSVQAGGSLRLSCTASGYTYSSAFMAWFRQAPGKE | 145 |
| _VHH1 | REGVAAIYTRDGGTVYADSVKGRFTISQDNAKNTLYLQMNSLKA | |
| | EDTAMYYCAAKIPQPGRASLLDSQTYDYWGQGTQVTVSS | |
| mIL12Rb1 | QVQLQESGGGSVQAGGSLRLSCAVSGYDYCGYDVRWYRQAPGKE | 146 |
| _VHH2 | REFVSGIDSDGSTSYADSVKGRFTISQDNAENTSYLHMFSLKPE | |
| | DTAMYYCKTESPAGESAWCRNFRGMDYWGKGTQVTVSS | |
| mIL12Rb1 | QVQLQESGGGSVQAGGSLRLSCVASGYSYCGYDMMWYRQAPGKE | 147 |
| _VHH3 | REFVALITSDYSIRYEDSVEGRFSISRDNAKNTGYLLMSNLTPA | |
| | DTAIYYCKTSTAARESSWCRSRYRVASWGQGTQVTVSS | |
| mIL12Rb1 | QVQLQESGGGSVQAGGSLRLSCAASRYTYTNNFMAWFRQAPGK | 148 |
| _VHH4 | EREGVAAIYTGDGYAYYFDSVKGRFTISQDNDKNMLYLQMNSLK | |
| | PEDTAMYYCAAMERRSGRRRMTENAEYKYWGQGTQVTVSS | |
| mIL12Rb1 | QVQLQESGGGSVQAGETLRLSCTVSGFTIDDSEMGWYRQAPGHE | 149 |
| _VHH5 | CELVASGSSDDDTYYVDSVKGRFTISLDNAKNMVYLQMNSLKPE | |
| | DTAVYYCATGPTYPPKDGDCAHWGQGTQVTVSS | |
| mIL12Rb1 | QVQLQESGGGSVQAGGSLRLSCTASGYTYSSAFMAWFRQAPGKE | 150 |
| _VHH6 | REGVAAIYTRDGSPVYADSLKGRFTISQDNAKNTLHLQMNSLKP | |
| | EDTAMYYCAAKIPEPGRISLLDSQTYDYWGHGTQVTVSS | |
| mIL12Rb1 | QVQLQESGGGSVQAGGSLRLSCAVSGYDYCGYDVRWYRRAPGKER | 151 |
| _VHH7 | EFVSGIDSDGSTSYADSVKGRFTISQDNAENTSYLHMFSLKPED | |
| | TAMYYCKTESPAGESAWCRNFRGMDYWGKGTQVTVSS | |
| mIL12Rb1 | QVQLQESGGGSVQAGGFLRLSCVASGYGYCGYDMSWYRQAPGKE | 152 |
| _VHH8 | REFVALITSERVISYEDSVKGRFSISRDNAENTGYLEMNRLTPD | |
| | DTAIYYCKTSAAARESSWCRSRYRVASWGQGTQVTVSS | |
| mIL12Rb1 | QVQLQESGGGSVQAGGSLRLSCVASGYGYCGYDMSWYRQAPGK | 153 |
| _VHH9 | EREFVALITSDRSISYEDSVKARFIISRDNAANTGYLDMTRLT | |
| | PDDTAIYYCKTSAAARESSWCRSRYRVASWGQGTQVTVSS | |
| mIL12Rb1 | QVQLQESGGGSVQAGGSLRLSCAVSGYDYCGYDVRWYRQAPGK | 154 |
| _VHH10 | EREFVSGIDSDGSTSYADSVKGRFTISQDNAENTSYLHMFSLK | |
| | PEDTAMYYCKTESPAGESAWCRNFRGMDYWGKGTQVTVSS | |
| mIL12Rb1 | QVQLQESGGGSVQAGGSLRLSCVASGYSYCGYDMMWYRQAPG | 155 |
| _VHH11 | KEREFVALITSDYSIRYEDSVEGRFSISRDNAKNTGYLLMSN | |
| | LTPADTAIYYCKTSTAARESSWCRSRYRVASWGQGTQVTVSS | |
| mIL12Rb1 | QVQLQESGGGSVQAGGSLRLSCAASRYTYTNNFMAWFRQAPGK | 156 |
| _VHH12 | EREGVAAIYTGDGYAYYFYSVKGRFTISQDNDENMLYLQMNSL | |
| | KPEDTAMYYCAAMERRIGTRRMTENAEYKYWGQGTQVTVSS | |
| mIL12Rb1 | QVQLQESGGGSVQAGGSLRLSCVASGYSYCGYDMMWYRQAPG | 157 |
| _VHH13 | KEREFVALITSDYSIRYEDSVEGRFSISRDNAKNTGYLLMSN | |
| | LTPADTAIYYCKTSTAARESGWCRSRYRVASWGQGTQVTVSS | |
| mIL12Rb1 | QVQLQESGGGSVQAGGSLRLSCAVSGYDYCGYDVRWYRQAPGK | 158 |
| _VHH14 | EREFVSGINSDGSTSYADSVKGRFTISQDNAENTSYLHMFSLK | |
| | TPEDAMYYCKTESPAGESAWCRNFRGMDYWGKGTQVTVSS | |
| mIL12Rb1 | QVQLQESGGGSVQAGGFLRLSCVASGYGYCGYDMSWYRQVPGK | 159 |
| _VHH15 | EREFVALITSDRSVSYEDSVKGRFSISRDNAKNTAYLEMNRLT | |
| | PDDTAVYYCKTSTAARENNWCRSRYRIAYWGQGTQVTVSS | |
| mIL12Rb1 | QVQLQESGGGSVQAGGSLRLSCAVSGYDYCGYDVRWYRQAPGK | 160 |
| _VHH16 | EREFVSGINSDGSTSYADSVKGRFTISQDNAENTSYLHMFSLK | |
| | PEDTAMYYCKTEGPAGESAWCRNFRGMDYWGKGTQVTVSS | |
| mIL12Rb1 | QVQLQESGGGSVQAGGSLRLSCTASGYTYSSAFMAWFRQAPGKE | 161 |
| _VHH17 | REGVAAMYTRDGGTVYADSVKGRFTISQDNAKNTLYLQIHTLKA | |
| | EDTAMYYCAAKIPQPGRASLLDSQTYDYWGQGTQVTVSS | |
| mIL12Rb1 | QVQLQESGGGSVQAGGSLRLSCTASGYTYSSAFMAWFRQAPGKE | 162 |
| _VHH18 | REGVAAMYTRDGGTVYADSVKGRFTISQDNAKNTLYLQMNSLK | |
| | TEDTAMYYCAAKIPQPGRASLLDSQTYDYWGQGTQVTVSS | |
| mIL12Rb1 | QVQLQESGGGSVQAGGFLRLSCVASGYGYCGYDMSWYRQVPGK | 163 |
| _VHH19 | EREFVALITSDRSVSYEDSVKGRFSISRDNAKNTAYLEMNRLT | |
| | PDDTAIYYCKTSTAARENNWCRSRYRIASWGQGTQVTVSS | |
| mIL12Rb1 | QVQLQESGGGSVQAGGSLRLSCVASGYGYCGYDMSWYRQTPGK | 164 |
| _VHH20 | EREFVALITSDRIASYEDSVKGRFIISRDNAKNTGYLDMTRVT | |
| | PDDTAIYYCKTSAAARENSWCRSRYRVASWGQGTQVTVSS | |
| mIL12Rb1 | QVQLQESGGGSVQAGGSLRLSCAVSGYDYCGYDVRWYRRAPGK | 165 |
| _VHH21 | EREFVSGIDSDGSTSYADSVKGRFTISQDNAENTSYLHMFSLK | |
| | PEDTAMYYCKTESPAGESAWCRNFRGMDYWGKGTQVTVSS | |
| mIL12Rb1 | QVQLQESGGGSVQAGGSLRLSCTASGYTYSSAFMAWFRQAPGKE | 166 |
| _VHH22 | REGVAAIYTRDGGTVYADSVKGRFTISQDNAKNILYLQMNSLKA | |
| | EDTAMYYCAAKIPQPGRASLLDSQTYDYWGQGTQVTVSS | |
| mIL12Rb1 | QVQLQESGGGSVQAGGSLRLSCTASGYTYSSAFMAWFRQAPGKE | 167 |
| _VHH23 | REGVAAIYTRDGGTVYADSVKGRFTISQDNAKNTLYLQMNSLKP | |
| | EDTAMYYCAAKMPQPGRASLLDSQTYDYWGQGTQVTVSS | |

TABLE 8
anti-human IL23R sdAb VHH Amino Acid Sequences

| | | SEQ | | SEQ | | SEQ | | SEQ |
|---|---|---|---|---|---|---|---|---|
| | | ID | | ID | | ID | | ID |
| Name | Sequence | NO: | CDR1 | NO: | CDR2 | NO: | CDR3 | NO: |
| hIL | QVQLQESGGGSVQAGGSLRLSCA | 260 | YTYCSYDM | 181 | AFNSDG | 192 | DPHV | 206 |
| 23R | ASGYTYCSYDMSWYRQAPGKKRE | | S | | TTSYAD | | QSSG | |
| _VH | FVSAFNSDGTTSYADSVKGRFTI | | | | SVKG | | GYCP | |
| H1 | SQDKAKNTVYLQMNSLKPEDTAM | | | | | | PY | |
| | YYCKTDPHVQSSGGYCPPYWGQG | | | | | | | |
| | TQVTVSS | | | | | | | |
| hIL | QVQLQESGGGSVQAGGSLRLSCA | 261 | YTYCSYDM | 181 | SFNSDG | 193 | DPHA | 207 |
| 23R | ASGYTYCSYDMSWYRQAPGKKRE | | S | | STSYAD | | DWGA | |
| _VH | FVSSFNSDGSTSYADSVKGRFTI | | | | SVKG | | PCGG | |
| H2 | SQDNAKNTVYLQMNSLKPEDTAM | | | | | | DY | |
| | YYCKTDPHADWGAPCGGDYWGQG | | | | | | | |
| | TQVTVSS | | | | | | | |
| hIL | QVQLQESGGGSVQAGESLRLSCA | 262 | YTYCTYDM | 182 | GIHSDG | 194 | DPIA | 208 |
| 23R | ASGYTYCTYDMTWYRQAPGKKRE | | T | | TTSYAD | | TITR | |
| _VH | FVSGIHSDGTTSYADSVKGRFTI | | | | SVKG | | RCDS | |
| H3 | SQDNAENTVYLQMNSLKPEDTAM | | | | | | Y | |
| | YYCKTDPIATITRRCDSYWGQGT | | | | | | | |
| | QVTVSS | | | | | | | |
| hIL | QVQLQESGGGSVQAGGSLRLSCA | 263 | STYCTYDM | 183 | AINSDG | 195 | DPNS | 209 |
| 23R | ASGSTYCTYDMTWYRQAPGKRRE | | T | | STSYAD | | GWGA | |
| _VH | FVSAINSDGSTSYADSVKGRFTI | | | | SVKG | | PCGG | |
| H4 | SQDNAKNTVYLQMNSLKPEDTAM | | | | | | DY | |
| | YYCKTDPNSGWGAPCGGDYWGQG | | | | | | | |
| | TQVTVSS | | | | | | | |
| hIL | QVQLQESGGGSVQAGGSLRLSCA | 264 | YTYCSYDM | 181 | AIASDG | 196 | DPHV | 206 |
| 23R | ASGYTYCSYDMSWYRQAPGKKRE | | S | | STSYAD | | QSSG | |
| _VH | FVSAIASDGSTSYADSLKGRFTI | | | | SLKG | | GYCP | |
| H5 | SQDNAKNTVYLQMNSLKPEDTAM | | | | | | PY | |
| | YYCKTDPHVQSSGGYCPPYWGQG | | | | | | | |
| | TQVTVSS | | | | | | | |
| hIL | QVQLQESGGGLVQPGGSLRLSCA | 265 | YTYCSYDM | 184 | SINSDG | 197 | DPQT | 210 |
| 23R | ASGYTYCSYDMGWYRQAPGKKRK | | G | | TTSYAD | | RPGK | |
| _VH | FVSSINSDGTTSYADSVKGRFTI | | | | SVKG | | PCAD | |
| H6 | SQDNAKNTVYLQMNSLKPEDTAM | | | | | | Y | |
| | YYCKTDPQTRPGKPCADYWGQGT | | | | | | | |
| | QVTVSS | | | | | | | |
| hIL | QVQLQESGGGSVQAGGSLRLSCA | 266 | YTYCNYDI | 185 | AIASDG | 198 | DPIS | 211 |
| 23R | ASGYTYCNYDIAWYRQAPGKERK | | A | | ITSYAD | | TITR | |
| _VH | FVSAIASDGITSYADSVKGRFTI | | | | SVKG | | ICDP | |
| H7 | SQDNAKNTVYLQMNSLKPEDTAM | | | | | | Y | |
| | YYCKTDPISTITRICDPYWGQGT | | | | | | | |
| | QVTVSS | | | | | | | |
| hIL | QVQLQESGGDSVQAGGSLRLSCA | 267 | YTYCSYDM | 186 | GIDSDG | 199 | EGTI | 212 |
| 23R | ASGYTYCSYDMKWYRQAPGKERE | | K | | SISYAD | | PVGA | |
| _VH | FVSGIDSDGSISYADSVKGRFTI | | | | SVKG | | CPNY | |
| H8 | SQDNAKNTVYLQMNSLKPEDTAM | | | | | | | |
| | YYCKTEGTIPVGACPNYWGQGTQ | | | | | | | |
| | VTVSS | | | | | | | |
| hIL | QVQLQESGGGLVQAGGSLRLSCA | 268 | YTYCSYDM | 181 | SINSDG | 197 | DPQT | 210 |
| 23R | ASGYTYCSYDMSWYRQAPGKERK | | S | | TTSYAD | | RPGK | |
| _VH | FVSSINSDGTTSYADSVKGRFTI | | | | SVKG | | PCAD | |
| H9 | SQDNAKNTVYLQMNSLKPEDTAM | | | | | | Y | |
| | YYCKTDPQTRPGKPCADYWGQGT | | | | | | | |
| | QVTVSS | | | | | | | |
| hIL | QVQLQESGGGSVQAGGSLKLSCA | 269 | YTYCNYDI | 185 | AIASDG | 200 | DPIA | 143 |
| 23R | ASGYTYCNYDIAWYRQAPGKERK | | A | | STSYAD | | TMTR | |
| _VH | FVSAIASDGSTSYADSVKGRFTI | | | | SVKG | | RCDP | |
| H10 | SQDNAKNTVYLQMNSLKPEDTAM | | | | | | Y | |
| | YYCKTDPIATMTRRCDPYWGQGT | | | | | | | |
| | QVTVSS | | | | | | | |
| hIL | QVQLQESGGGSVQAGGSLRLSCA | 270 | YTYCSYDM | 187 | AIDSDG | 201 | DPIA | 213 |
| 23R | ASGYTYCSYDMTWYRQAPGKKRE | | T | | STSYAD | | TISR | |
| _VH | FVSAIDSDGSTSYADSVKGRFTI | | | | SVKG | | RCDS | |
| H11 | SQDNAKNTVYLQMNSLKPEDTAM | | | | | | Y | |
| | YYCKTDPIATISRRCDSYWGQGT | | | | | | | |
| | QVTVSS | | | | | | | |
| hIL | QVQLQESGGGSVQAGGSLRLSCA | 271 | YTSSSRCM | 188 | RIYTPT | 202 | GASC | 214 |
| 23R | ASGYTSSSRCMGWFRQAPGKERE | | G | | RTTWYA | | AVDL | |
| _VH | GVARIYTPTRTTWYADSVKGRFT | | | | DSVKG | | FSY | |
| H12 | ISQDNAKNTVYLEMASLKPEDTA | | | | | | | |
| | KYFCAAGASCAVDLFSYWGQGTQ | | | | | | | |
| | VTVSS | | | | | | | |
| hIL | QVQLQESGGGSVQAGGSLRLSCA | 272 | YTYCSYDM | 186 | AIDSDG | 201 | EGTI | 215 |
| 23R | ASGYTYCSYDMKWYRQAPGKKRE | | K | | STSYAD | | PVGV | |
| _VH | FVSAIDSDGSTSYADSVKGRFTI | | | | SVKG | | CPNY | |
| H13 | SQDNAKNTVYLQMNSLKPEDTAM | | | | | | | |
| | YYCKTEGTIPVGVCPNYWGQGTQ | | | | | | | |
| | VTVSS | | | | | | | |
| hIL | QVQLQESGGGLVQPGGSLRLSCA | 273 | YTYCSYDM | 186 | AIDSDG | 201 | EGTV | 216 |
| 23R | ASGYTYCSYDMKWYRQAPGKKRE | | K | | STSYAD | | PVGV | |
| _VH | FVSAIDSDGSTSYADSVKGRFTI | | | | SVKG | | CPNY | |
| H14 | SQDNAKNTVYLQMNSLKPEDTAM | | | | | | | |
| | YYCKTEGTVPVGVCPNYWGQGTQ | | | | | | | |
| | VTVSS | | | | | | | |
| hIL | QVQLQESGGGSVQAGGSLRLSCA | 274 | GTYTSRYM | 189 | TIWPAG | 203 | AKYG | 217 |
| 23R | ASPGTYTSRYMGWFRQAPGKERE | | G | | GNTVYA | | GTSL | |
| _VH | GVATIWPAGGNTVYADSVKGRFT | | | | DSVKG | | APYT | |
| H15 | ISQDGAKKTVYLQMNSLKPEDTA | | | | | | YNY | |
| | MYYCAAAKYGGTSLAPYTYNYWG | | | | | | | |
| | QGTQVTVSS | | | | | | | |
| hIL | QVQLQESGGGSVEAGGALTLSCV | 275 | YTYCNYDI | 185 | AIASDG | 200 | DPIA | 143 |
| 23R | ASGYTYCNYDIAWYRQAPGKERK | | A | | STSYAD | | TMTR | |
| _VH | FVSAIASDGSTSYADSVKGRFTI | | | | SVKG | | RCDP | |
| H16 | SQDNAKNTVYLQMNSLKPEDTAM | | | | | | Y | |
| | YYCKTDPIATMTRRCDPYWGQGT | | | | | | | |
| | QVTVSS | | | | | | | |
| hIL | QVQLQESGGGSVQAGGSLRLSCT | 276 | YTFSTMKY | 190 | AIWIAA | 204 | ARYG | 218 |
| 23R | ASGYTFSTMKYMGWFRQAPGKER | | MG | | GNTYYA | | FVPS | |
| _VH | EGVAAIWIAAGNTYYADSVKGRF | | | | DSVKG | | TWYL | |
| H17 | TISQDNTKNTVYLQMNSLKPEDT | | | | | | PERY | |
| | ALYYCAAARYGFVPSTWYLPERY | | | | | | NY | |
| | NYWGQGTQVTVSS | | | | | | | |
| hIL | QVQLQESGGGSVQAGGSLRLACA | 277 | YTYCNYDI | 185 | AIASDG | 200 | DPIA | 143 |
| 23R | ASGYTYCNYDIAWYRQAPGKERK | | A | | STSYAD | | TMTR | |
| _VH | FVSAIASDGSTSYADSVKGRFTI | | | | SVKG | | RCDP | |
| H18 | SQDNAKNTVYLQMNSLKPEDTAM | | | | | | Y | |
| | YYCKTDPIATMTRRCDPYWGQGT | | | | | | | |
| | QVTVSS | | | | | | | |
| hIL | QVQLQESGGGSVQAGGSLRLSCA | 278 | YTSCSYDM | 191 | AIHSDG | 205 | DPNY | 144 |
| 23R | ASGYTSCSYDMSWYRQAPGKKRE | | S | | TTSYAD | | SDHV | |
| _VH | FVSAIHSDGTTSYADSMKGRFTI | | | | SMKG | | CPPY | |
| H19 | SQDNAKNTVYLQMNSLKPEDTAM | | | | | | | |
| | YYCKTDPNYSDHVCPPYWGRGTQ | | | | | | | |
| | VTVSS | | | | | | | |

TABLE 9
anti-mIL23R sdAb VHH AMINO ACID SEQUENCE MOUSE
mIL23R ECD Generated VHHs and CDRs

| Name | VHH Amino Acid Sequence (CDRs underlined | SEQ ID NO |
|---|---|---|
| mIL23R | QVQLQESGGGSVQAGGSLRLSCAASGYTYSSCTMGWYRQAPGKER | 97 |
| VHH1 | ELVSMLISDGSTFYADSVKGRFTFSQEYAKNTVYLQMNSLKPEDTA | |
| | MYYCGCATLGSRTVWGQGTQVTVSS | |
| mIL23R | QVQLQESGGGSVQAGGSLTLSCTAPGFTFRLAAMRWVRQAPGKGL | 98 |
| VHH2 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGSVQAGGSLRLSCTASVNTYCEYNMSWYRQAPGKE | 99 |
| VHH3 | REFVSGVDSDGSTRYSESVKGRFTISQDNAKNTMYLQMNGLKPEDT | |
| | AMYYCKTYVCTFCSGNSCYYEYKYYYEGQGTQVTVSS | |
| mIL23R | QVQLQESGGGSVQAGGSLRLSCAASGYTYSNNCMGWFRQAPGKD | 100 |
| VHH4 | RERIANIYTGGGRTTYADSVKGRFTISQDSAKSTVYLQMNSLKPEDT | |
| | AMYYCAAGSCGSARSEYSYWGQGTQVTVSS | |
| mIL23R | QVQLQESGGGSVQAGGSLRLSCAASGYTFCMAWFRQAPGKEREGV | 101 |
| VHH5 | ARFYTRDGYTYYSDSVKGRFTISQNNAKNTLYLQMNSLKSEDTAM | |
| | YYCAADLARCSSNKNDFRYWGQGTQVTVSS | |
| mIL23R | QVQLQESGGGSVQAGGSLRLSCAASGYTSGNYWMGWFRQAPGKE | 102 |
| VHH6 | REGVATLWTGGASTFYGDSVKGRFTISRDNFKNTLYLQMNSLKVE | |
| | DTAMYYCAADPALRLGANILRPAEYKYWGQGTQVTVSS | |
| mIL23R | QVQLQESGGGLVQPGGSLRLSCAASGFTFSRSAMTWVRQAPGKGL | 103 |
| VHH7 | DWVSGIDSGGTTVYADSVKGRFTISRDSAKNTLYLQMNSLKTEDTA | |
| | VYYCAIGLPWGNTWRTRGQGTQVTVSS | |
| mIL23R | QVQLQESGGGSVQAGGSLRLSCTASRYTYSSCTMGWYRQAPGKER | 104 |
| VHH8 | ELVSMVFSDGSTFYADSVKGRFTFSQENAKNTVYLQMNSLKPEDT | |
| | AMYYCGCATLGSRTIWGQGTQVTVSS | |
| mIL23R | QVQLQESGGGLVQPGGSLRLSCATSGFTFRLTAMRWVRQAPGKGV | 105 |
| VHH9 | EWVSGIDSAGSTIYADSVKGRFTISKDNAKNTLYLQMNSLKTEDTA | |
| | MYYCAQGVYGDTYSGSQGTQVTVSS | |
| mIL23R | QVQLQESGGGSVQAGGSLRLSCAASGDTYSSCTMGWYRQAPGKER | 106 |
| VHH10 | DLVSMLMGDGSTFYADSVKGRFTFSQENAKNTVYLQMNSLKPEDT | |
| | AMYYCGCATLGSRTIWGQGTQVTVSS | |
| mIL23R | QVQLQESGGGSVQAGGSLRLSCAASGYTYSSCTMGWYRQAPGKER | 107 |
| VHH11 | ELVSMLISDGSTFYADSVKGRFTFSQENAKSTVYLQMNSLKPEDTA | |
| | MYYCGCATLGSRTVWGQGTQVTVSS | |
| mIL23R | QVQLQESGGGSVQAGGSLRLSCAASGYTYSSCTMGWYRQAPGKER | 108 |
| VHH12 | ELVSMLISDGSTFYADSVKGRFTFSQENAKNTVYLQMNSLKPEDTA | |
| | MYYCGCATLGSRTVWGQGTQVTVSS | |
| mIL23R | QVQLQESGGGLVQPGGSLRLSCATSGFTFRLAAMRWVRQAPGKGL | 109 |
| VHH13 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGLVQPGGSLRLSCAASGFTFRTSAMTWVRQAPGKGL | 110 |
| VHH14 | DWVSGIDSGGTTVYADSVKGRFTISRDSAKNTLYLQMNSLKTEDTA | |
| | VYYCAMGLPWGNTWRTRGQGTQVTVSS | |
| mIL23R | QVQLQESGGGSVQAGGSLRLSCAASGYTYSSCTMGWYRQAPGKER | 111 |
| VHH15 | ELVSMVFSDGSTFYADSVKGRFTFSQENAKNTVYLQMNSLKPEDT | |
| | AMYYCGCATLGSRTIWGQGTQVTVSS | |
| mIL23R | QVQLQESGGGSVQAGGSLRLSCAASGDTYSSCTMGWYRQAPGKER | 112 |
| VHH16 | DLVSMLMGDGSTFYADSVKGRFTFSQENAKNTVYLQMNNLKPEDT | |
| | AMYYCGCATLGSRTIWGQGTQVTVSS | |
| mIL23R | QVQLQESGGGSVQAGGSLRLSCAASGFTFRLTAMRWVRQAPGKGL | 113 |
| VHH17 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGLVQPGGSLRLSCAASGFTFRLTAMRWVRQAPGKGL | 114 |
| VHH18 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGLVQPGGSLRLSCAASGFTFRLSAMRWVRQAPGKGL | 115 |
| VHH19 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGLVQPGGSLRLSCAASGFTFSSSAMTWVRQAPGKGL | 116 |
| VHH20 | DWVSGIDSGGTTVYADSVKGRATILKDNAKNTLYLQMNSLKTEDT | |
| | AVYYCATGLPWGNTWRTRGQGTQVTVSS | |
| mIL23R | QVQLQESGGGLVQPGGSLRLSCATSGFTFSSSAMTWVRQAPGKGL | 117 |
| VHH21 | DWVSGIDSGGTTVYADSVKGRFTISKDNAKNTLYLQMNSLKTEDT | |
| | AVYYCATGLPWGNTWRTTGQGTQVTVSS | |
| mIL23R | QVQLQESGGGSVQAGGSLRLSCAASGYTFCMAWFRQAPGKEREGV | 118 |
| VHH22 | ARFYTRDSYTYYSDSVKGRFTISQNNAKNTLYLQMNSLKSEDTAM | |
| | YYCAADLTRCSSNKNDFRYWGQGTQVTVSS | |
| mIL23R | QVQLQESGGGLVQPGGSLRLSCAASGFNFRLYAMRWVRQAPGKG | 119 |
| VHH23 | VEWVSGIDSGGSTIYADSVKGRFTISKDNAKNTLYLQLNSLKTEDT | |
| | AMYYCAQGVYGDTYSGSQGTQVTVSS | |
| mIL23R | QVQLQESGGGSVQAGGSLRLSCAVSGYTFCMAWFRQAPGKEREGV | 120 |
| VHH24 | ARFYTRDGYTYYSGSVKGRFTISQNNAKNTLYLQMNSLKSEDTAM | |
| | YYCAADLTRCSSNKNDFRYWGQGTQVTVSS | |
| mIL23R | QVQLQESGGGSVQAGGSLRLSCAASGYTYSSCTMGWYRQAPGKER | 121 |
| VHH25 | ELVSMLISDGSTFYADSVKGRFTFSQENAKNTVYLQMNSLKPEDTA | |
| | MYFCGCATLGSRTVWGQGTQVTVSS | |
| mIL23R | QVQLQESGGGSVQAGGSLRLSCAASGYTFCMAWFRQAPGKEREGV | 122 |
| VHH26 | ARFYTRDGYTYYSDSVKGRFTISQNNAKNTLYLQMNSLKSEDTAM | |
| | YYCAADLTRCSSNKNDFRYWGQGTQVTVSS | |
| mIL23R | QVQLQESGGGSVQAGGSLRLSCAASGYTYSSCTMGWYRQAPGKER | 123 |
| VHH27 | ELVSMLISDGSTFYADSVKGRFTSSQENAKNTVYLQMNSLKPEDTA | |
| | MYYCGCATLGSRTVWGQGTQVTVSS | |
| mIL23R | QVQLQESGGGLVQPGGSLRLSCAASGFTFRLTAMRWVRQAPGKGL | 124 |
| VHH28 | EWVSGIDSRGSTIYADSVKGRFTISRDNAKNTLYLQLNSLKTEDAA | |
| | MYYCAQGVYGDTYSGSQGTQVTVSS | |
| mIL23R | QVQLQESGGGLVQPGGSLRLSCAASGFTFSTSAMTWVRQAPGKGL | 125 |
| VHH29 | DWVSGIDSGGTTVYADSVKGRFTISKDNAKNTLYLQMNSLKTEDT | |
| | AVYYCATGLPWGNTWRTRGQGTQVTVSS | |
| mIL23R | QVQLQESGGGLVQPGGSLRLSCAASGFTFRLTAMRWVRQAPGKGL | 126 |
| VHH30 | EWVSGIDSRGSTIYADSVKGRFTISKDNAKNTLYLQLNSLKTEDTA | |
| | MYYCAQGVYGDTYSGSLGTQVTVSS | |
| mIL23R | QVQLQESGGGLVQPGGSLRLSCAASGFTFRLTAMRWVRQAPGKGL | 127 |
| VHH31 | EWVSGIDSRGSTIYADSVKGRFTISRDNAKNTLYLQLNSLKTEDTA | |
| | MYYCAQGVYGDTYSGSQGTQVTVSS | |
| mIL23R | QVQLQESGGGLVRPGGSLRLSCAASGFTFSRSAMTWVRQAPGKGL | 128 |
| VHH32 | DWVSGIDSGGTTVYADSVKGRFTISRDSAKNTLYLQMNSLKTEDTA | |
| | VYYCAIGLPWGNTWRTRGQGTQVTVSS | |
| mIL23R | QVQLQESGGGLVQPGGSLRLSCTTSGFTFSSSAMTWVRQAPGKGLD | 129 |
| VHH33 | WVSGIDSGGTTVYADSVKGRFTISKDNAKNTLYLQMNSLKTEDTA | |
| | VYYCATGLPWGNTWRTTGQGTQVTVSS | |
| mIL23R | QVQLQESGGGLVQPGGSLRLSCAASGFTFRLTAMRWVRQAPGKGL | 130 |
| VHH34 | EWVSGIDSRGSTIYADSVKGRFTISKDNAKNTLYLQLNSLKTEDTA | |
| | MYYCAQGVYGDTHSGSQGTQVTVSS | |
| mIL23R | QVQLQESGGGLVQPGGSLRLACSASGFTFSSSAMTWVRQAPGKGL | 131 |
| VHH35 | DWVSGIDSGGTTVYADSVKGRATILKDNAKNTLYLQMNSLKTEDT | |
| | AVYYCATGLPWGNTWRTRGQGTQVTVSS | |
| mIL23R | QVQLQESGGGSVQAGGSLRLSCAASGDTYSSCTMGWYRQAPGKER | 132 |
| VHH36 | DLVSMVFSDGSTFYADSVKGRFTFSQENAKNTVYLQMNSLKPEDT | |
| | AMYYCGCATLGSRTIWGQGTQVTVSS | |
| mIL23R | QVQLQESGGGLVQPGGSLRLSCATSGFTFSSGAMTWVRQAPGKGL | 133 |
| VHH37 | DWVSGIDSGGTTVYADSVKGRFTISKDNAKNTLYLQMNSLKTEDT | |
| | AVYYCATGLPWGNTWRTTGQGTQVTVSS | |
| mIL23R | QVQLQESGGGLVQPGGSLRLSCAASGFTFSTSAMTWVRQAPGKGL | 134 |
| VHH38 | DWVSGIDSGGTTVYADSVKGRFTISKDNAKNTLYLQMNSLKTEDT | |
| | AVYYCATGLPWGNIWRTRGQGTQVTVSS | |
| mIL23R | QVQLQESGGGLVQPGGSLRLSCAASGFTFSRSAMTWVRQAPGKGL | 135 |
| VHH39 | DWVSGIDSGGTTVYADSVKGRFTISRDSAKNTLYLQMNGLKTEDT | |
| | AVYYCAIGLPWGNTWRTRGQGTQVTVSS | |
| mIL23R | QVQLQESGGGLVQPGGSLRLSCAASGFTFRLTAMRWVRQAPGKGL | 136 |
| VHH40 | EWVSGIDSRGSTIYADSVKGRFTISKDNAKNTLYLQLNSLKSEDTA | |
| | MYYCAQGVYGDTYSGSQGTQVTVSS | |
| mIL23R | QVQLQESGGGLVQPGGSLRLTCAASGFTFSTSAMTWVRQAPGKGL | 137 |
| VHH41 | DWVSGIDSGGTTVYADSVKGRFTISKDNAKNTLYLQMNSLKTEDT | |
| | AVYYCATGLPWGNTWRTRGQGTQVTVSS | |
| mIL23R | QVQLQESGGGLVQPGGSLRLSCAASGFTFSNYAMRWVRQAPGKGL | 138 |
| VHH42 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGSVQAGGSLRLSCAASGYTFCMAWFRQAPGKEREGV | 139 |
| VHH43 | ARFYTRDGYTYYSDSVKGRFTISQDNAKNTLYLQMNSLKSEDTAM | |
| | YYCAADLTRCSSNKNDFRYWGQGTQVTVSS | |
| mIL23R | QVQLQESGGGLVQPGGSLRLSCAASGFTFRLSAMRWVRQAPGKGF | 140 |
| VHH44 | EWVSGIDSRGSTIYADSVKGRFTISKDNAKNTLYLQLNSLKTEDTA | |
| | MYYCAQGVYGDTYSGSQGTQVTVSS | |
| mIL23R | QVQLQESGGGLVQPGGSLRLSCAASGFTFRLSAMRWVRQAPGKGL | 141 |
| VHH45 | EWVSGIDSRGSTIYADSVEGRFTISKDNAKNTLYLQLNSLKTEDTA | |
| | MYYCAQGVYGDTYSGSQGTQVTVSS | |
| mIL23R | QVQLQESGGGLVQPGGSLRLSCAASGFTFRLSAMRWVRQAPGKGL | 142 |
| VHH46 | EWVSGIDSRGSTIYADSVKGRFTISKDDAKNTLYLQLNSLKTEDTA | |
| | MYYCAQGVYGDTYSGSQGTQVTVSS | |

TABLE 10
anti-human IL12RB1 sdAb VHH DNA Sequences

| Name | Sequence |
|---|---|
| hIL | CAGGTCCAGCTCCAGGAGTCTGGCGGTGGCTCAGTACAAGCTGGGGGCTCTCTGCGTTTGTCCT |
| 12R | GTGTGGCGAGCGGGTACGGATACTGTGGGTACGACATGAGTTGGTACAGACAGGCCCCTGGCAA |
| b1 | GGAACGTGAATTTGTGGCCCTCATCACTTCTGATCGCTCCATTAGCTACGAGGATTCTGTCAAA |
| _VHH | GCTCGCTTTATCATTTCCCGCGACAACGCCGCTAACACTGGTTATCTGGACATGACTAGACTGA |
| 1 | CCCCCGATGACACGGCCATTTACTATTGCAAGACCAGTGCAGCGGCCCGCGAATCTTCCTGGTG |
| | TCGCTCTCGCTACCGCGTGGCATCATGGGGCCAGGGTACTCAGGTCACCGTGTCTAGC (SEQ |
| | ID NO: 279) |
| hIL | CAAGTCCAACTCCAGGAGTCTGGTGGGGGCTCTGTTCAAGCTGGCGGGTCCCTGCGCCTTTCCT |
| 12R | GTACCGCCAGCGGCTACACGTACTCTAGCGCCTTCATGGCTTGGTTTCGGCAGGCCCCTGGAAA |
| b1 | AGAGAGAGAGGGAGTGGCAGCTATCTACACTCGTGACGGCGGAACCGTGTACGCTGATAGTGTC |
| _VHH | AAGGGCCGCTTCACCATTTCCCAGGATAATGCCAAGAATATCCTGTATCTCCAGATGAACTCCC |
| 2 | TTAAAGCCGAAGACACTGCGATGTACTATTGCGCAGCCAAAATCCCGCAGCCAGGCCGGGCTTC |
| | TTTGCTGGATAGCCAAACCTACGACTATTGGGGTCAAGGCACTCAGGTTACCGTGTCTTCC |
| | (SEQ ID NO: 280) |
| hIL | CAGGTCCAGCTTCAGGAGAGCGGCGGAGGCTCCGTGCAGGCTGGGGGATCTTTGAGACTCAGCT |
| 12R | GCGTGGCCAGTGGCTACTCTTACTGTGGGTACGACATGATGTGGTATCGCCAAGCGCCGGGCAA |
| b1 | GGAACGTGAGTTCGTGGCGCTCATCACTTCCGACTACTCAATTCGTTACGAGGATTCCGTTGAG |
| _VHH | GGCCGCTTCAGCATTTCTCGTGACAACGCGAAGAACACAGGATACTTGCTGATGAGTAACCTCA |
| 3 | CCCCCGCCGATACCGCTATTTATTACTGCAAGACAAGTACAGCTGCCAGGGAGAGCAGTTGGTG |
| | TCGGTCTCGCTATCGTGTGGCCTCCTGGGGACAGGGCACCCAAGTAACCGTGTCATCA (SEQ |
| | ID NO: 281) |
| hIL | CAGGTGCAGCTCCAGGAATCTGGTGGGGGCAGTGTTCAGGCTGGTGGCAGCCTGAGACTTAGCT |
| 12R | GCGTGGCTTCTGGCTATGGTTACTGTGGGTACGACATGAGCTGGTATCGGCAGACCCCCGGAAA |
| b1 | GGAGCGGGAGTTCGTAGCGCTCATCACAAGTGACCGCATCGCCTCCTATGAAGACTCCGTTAAG |
| _VHH | GGTCGCTTTATCATTAGCCGGGACAATGCCAAGAACACAGGTTACCTCGATATGACTCGGGTCA |
| 4 | CACCTGACGATACCGCTATCTATTACTGCAAGACTTCTGCGGCTGCCCGTGAAAACAGCTGGTG |
| | CCGCTCAAGATACCGGGTGGCCTCCTGGGGACAGGGAACTCAGGTCACCGTCTCTAGC (SEQ |
| | ID NO: 282) |
| hIL | CAGGTGCAGTTGCAGGAGAGCGGAGGCGGATCTGTGCAGGCCGGTGGATTTCTGCGGCTGTCTT |
| 12R | GCGTGGCGAGCGGCTATGGCTATTGCGGATACGACATGAGCTGGTATCGCCAGGTTCCGGGTAA |
| b1 | GGAGCGTGAGTTCGTCGCTCTGATTACCTCTGATCGCTCTGTGTCCTATGAGGACTCCGTTAAG |
| _VHH | GGTAGATTCTCTATCTCTCGCGATAATGCTAAGAACACAGCCTACCTGGAGATGAACAGACTGA |
| 5 | CCCCCGACGATACCGCTGTCTATTACTGTAAGACCTCCACAGCCGCTCGCGAGAATAACTGGTG |
| | CCGCTCTCGCTATAGAATCGCCTATTGGGGTCAGGGTACACAAGTTACCGTATCCTCC (SEQ |
| | ID NO: 283) |
| hIL | CAGGTGCAGTTGCAGGAGAGTGGCGGGGGCTCTGTTCAGGCTGGTGGATCATTGCGTCTGAGCT |
| 12R | GTGCTGCCTCCCGCTACACCTACACTAATAACTTCATGGCTTGGTTTAGACAAGCTCCTGGCAA |
| b1 | GGAACGCGAAGGCGTTGCCGCGATTTATACCGGAGACGGTTACGCATATTACTTCTATTCCGTG |
| _VHH | AAGGGCCGCTTCACAATCTCCCAGGATAACGACGAAAATATGCTCTACTTGCAGATGAACTCCC |
| 6 | TCAAACCTGAGGACACGGCAATGTACTATTGTGCGGCTATGGAGCGCCGTATCGGAACTCGCCG |
| | TATGACCGAAAACGCTGAGTATAAGTATTGGGGACAAGGAACCCAGGTGACCGTATCCTCC |
| | (SEQ ID NO: 284) |
| hIL | CAGGTCCAGTTGCAGGAGTCTGGTGGCGGAAGCGTGCAGGCTGGGGGCAGCCTCAGGCTGTCCT |
| 12R | GTGCTGTGTCCGGGTACGACTACTGCGGCTACGACGTGCGCTGGTATCGCCGTGCCCCCGGCAA |
| 01 | GGAGAGGGAGTTCGTCTCCGGGATTGATTCCGATGGCTCTACCAGTTACGCAGATTCCGTCAAG |
| _VHH | GGTCGTTTTACCATTAGTCAGGATAACGCTGAGAACACAAGCTATCTGCACATGTTCTCACTGA |
| 7 | AGCCTGAGGATACGGCCATGTACTATTGCAAGACTGAGTCCCCCGCAGGTGAATCCGCCTGGTG |
| | TCGTAACTTTCGCGGCATGGACTACTGGGGAAAGGGCACCCAGGTCACTGTGTCTTCT (SEQ |
| | ID NO: 285) |
| hIL | CAGGTGCAGCTCCAGGAATCAGGCGGTGGGTCCGTGCAGGCAGGAGGGAGTCTGCGCCTGTCCT |
| 12R | GTGTGGCCTCCGGTTACAGCTACTGCGGCTACGATATGATGTGGTATAGGCAAGCTCCAGGGAA |
| b1 | GGAGCGTGAGTTCGTGGCCCTTATCACATCTGACTATTCCATCCGCTACGAGGACTCCGTGGAG |
| _VHH | GGAAGATTTTCAATCTCCAGAGACAACGCAAAGAACACCGGATACCTCCTGATGTCTAACCTGA |
| 8 | CCCCAGCCGACACGGCAATCTATTACTGTAAAACCTCCACAGCAGCGAGGGAGTCCAGCTGGTG |
| | CAGGTCCAGATACCGTGTTGCCTCCTGGGGACAGGGCACTCAGGTGACGGTGAGTTCT (SEQ |
| | ID NO: 286) |
| hIL | CAGGTGCAGCTCCAGGAGTCCGGTGGCGGGAGCGTGCAGGCTGGCGGATCTCTGCGGCTCAGTT |
| 12R | GCGTCGCCTCAGGGTATTCCTATTGTGGCTACGATATGATGTGGTATCGTCAGGCCCCCGGCAA |
| b1 | GGAGCGCGAGTTCGTCGCCCTGATTACAAGCGATTATTCAATCCGTTATGAAGATTCCGTGGAG |
| _VHH | GGGCGCTTCTCCATCAGTCGCGACAACGCCAAAAACACTGGCTACCTTCTGATGTCAAACCTGA |
| 9 | CTCCCGCTGACACCGCGATCTACTATTGTAAAACCTCAACGGCTGCCCGCGAGTCCGGCTGGTG |
| | CCGGTCTAGGTATCGTGTGGCCAGCTGGGGGCAGGGCACTCAGGTCACCGTGTCATCC (SEQ |
| | ID NO: 287) |
| hIL | CAGGTCCAGCTGCAAGAATCCGGTGGAGGCTCTGTGCAGGCGGGTGGGTCCCTGCGCCTGTCTT |
| 12R | GCGCCGTGTCTGGCTATGATTATTGCGGATATGACGTGCGCTGGTATCGCCAGGCTCCCGGCAA |
| b1 | GGAACGCGAGTTTGTCTCTGGGATTGACTCAGACGGCAGCACTAGCTATGCCGACTCCGTGAAA |
| _VHH | GGTCGCTTCACCATTTCCCAAGACAACGCCGAGAATACCAGCTATCTGCACATGTTCAGCCTCA |
| 10 | AACCTGAAGATACTGCCATGTATTACTGTAAGACGGAGAGTCCCGCAGGCGAATCCGCTTGGTG |
| | TCGGAATTTCAGGGGAATGGACTACTGGGGCAAGGGTACTCAAGTGACCGTAAGCTCT (SEQ |
| | ID NO: 288) |
| hIL | CAGGTGCAGCTCCAGGAGAGCGGCGGAGGCTCCGTGCAGGCGGGCGGGAGCCTGCGTCTGTCTT |
| 12R | GTGCCGTATCTGGCTATGACTATTGCGGTTACGACGTTCGCTGGTACAGGCAGGCTCCGGGCAA |
| b1 | GGAGCGTGAGTTTGTCAGCGGGATTGACAGTGACGGCTCCACCTCTTATGCGGATTCCGTGAAG |
| _VHH | GGACGCTTCACAATTTCCCAGGATAACGCAGAGAACACCTCCTACCTCCACATGTTCAGCCTCA |
| 11 | AACCCGAAGATACTGCTATGTATTACTGTAAAACAGAGAGCCCAGCCGGGGAGTCTGCTTGGTG |
| | TCGTAACTTTCGCGGCATGGACTACTGGGGCAAGGGAACCCAGGTGACCGTCTCTTCC (SEQ |
| | ID NO: 289) |
| hIL | CAGGTGCAACTCCAAGAGAGCGGAGGCGGGAGTGTTCAGGCCGGGGGCTCTCTGCGGCTGTCCT |
| 12R | GCACCGCCTCTGGTTACACCTACTCCAGCGCCTTCATGGCCTGGTTCCGGCAGGCACCTGGCAA |
| b1 | GGAACGCGAAGGCGTAGCCGCTATCTATACGCGCGATGGGGGTACAGTTTATGCTGATAGCGTT |
| _VHH | AAAGGACGCTTCACTATCTCCCAGGACAACGCCAAAAACACCCTGTACTTGCAGATGAACTCCC |
| 12 | TCAAACCTGAAGATACGGCGATGTACTATTGTGCGGCAAAGATGCCTCAGCCCGGACGCGCAAG |
| | TCTGCTTGACTCTCAAACTTATGATTACTGGGGCCAAGGGACTCAGGTGACCGTTAGCTCC |
| | (SEQ ID NO: 290) |
| hIL | CAGGTGCAGTTGCAGGAAAGCGGCGGTGGCTCAGTCCAGGCCGGGGGCTTCTTGCGCTTGAGTT |
| 12R | GCGTGGCGAGCGGATATGGCTACTGTGGCTACGATATGAGCTGGTATCGTCAGGCTCCGGGCAA |
| b1 | GGAACGTGAGTTCGTCGCGCTCATCACTAGCGAAAGAGTCATCTCCTACGAAGACTCCGTTAAG |
| _VHH | GGCCGCTTTTCCATTTCTCGCGACAACGCCGAGAACACGGGCTACCTTGAAATGAATAGACTGA |
| 13 | CTCCCGACGATACTGCCATCTACTATTGCAAGACAAGCGCCGCTGCACGCGAGTCCTCTTGGTG |
| | CAGGTCTCGCTACCGCGTGGCTTCTTGGGGGCAGGGGACCCAGGTGACCGTATCATCC (SEQ |
| | ID NO: 291) |
| hIL | CAGGTTCAACTCCAGGAGTCCGGGGGCGGTTCCGTGCAGGCTGGGGGCTCCCTTAGACTTAGCT |
| 12R | GTGCCGTGTCTGGATACGATTACTGTGGGTATGACGTGCGGTGGTACAGACGCGCTCCGGGAAA |
| b1 | GGAACGCGAGTTCGTGAGCGGAATTGATTCCGATGGCAGCACCTCCTATGCGGATTCTGTGAAG |
| _VHH | GGCCGCTTCACTATCTCTCAAGACAACGCCGAGAACACTAGCTACCTGCACATGTTCAGTCTGA |
| 14 | AACCGGAGGATACCGCGATGTATTACTGTAAGACCGAGTCTCCTGCTGGAGAGAGCGCGTGGTG |
| | CAGAAACTTCCGTGGAATGGACTATTGGGGTAAAGGAACTCAGGTGACTGTGTCCAGT (SEQ |
| | ID NO: 292) |
| hIL | CAAGTGCAGCTCCAGGAATCTGGAGGCGGAAGCGTACAGGCCGGTGGCTCACTCCGGCTTTCTT |
| 12R | GCGCTGTGTCAGGTTACGACTATTGTGGATATGATGTCCGGTGGTATAGGCAAGCGCCGGGAAA |
| b1 | GGAGCGCGAGTTCGTGAGCGGTATCAACTCTGACGGCTCCACCTCCTACGCCGACTCTGTCAAG |
| _VHH | GGCCGCTTTACAATTTCTCAGGACAACGCAGAGAACACCTCTTACCTGCACATGTTCAGCTTGA |
| 15 | AGCCGGAGGACACCGCGATGTACTATTGTAAGACTGAGTCCCCCGCTGGAGAGTCTGCATGGTG |
| | CCGTAATTTTCGCGGCATGGACTATTGGGGGAAAGGTACTCAGGTTACCGTAAGCTCA (SEQ |
| | ID NO: 293) |
| hIL | CAGGTACAGCTCCAGGAGAGTGGAGGCGGGTCAGTGCAGGCCGGGGGCTCACTGCGCTTGAGCT |
| 12R | GCACCGCGAGCGGTTACACCTACAGCTCCGCATTCATGGCTTGGTTCAGGCAAGCCCCAGGCAA |
| b1 | GGAGCGCGAGGGCGTGGCTGCCATGTATACCCGCGACGGGGGCACCGTGTATGCCGATTCCGTG |
| _VHH | AAGGGCCGTTTCACCATCTCCCAGGATAACGCTAAGAACACCCTCTACCTCCAGATCCACACTC |
| 16 | TCAAAGCCGAAGACACGGCTATGTACTATTGCGCCGCGAAGATCCCTCAACCTGGCAGGGCAAG |
| | CCTTCTGGACTCCCAGACGTATGACTATTGGGGCCAGGGGACTCAGGTTACAGTGTCCAGC |
| | (SEQ ID NO: 294) |
| hIL | CAGGTGCAGCTCCAGGAATCCGGCGGTGGGTCTGTGCAGGCAGGGGGTTTTCTCCGCTTGAGCT |
| 12R | GTGTGGCTAGTGGATACGGTTATTGTGGATACGACATGAGCTGGTATCGCCAAGTACCGGGCAA |
| b1 | GGAGCGTGAGTTTGTGGCCCTCATCACCTCTGATCGCTCCGTGTCTTATGAGGACAGCGTGAAG |
| _VHH | GGCCGCTTCAGCATCAGTCGCGACAACGCCAAGAACACCGCTTATCTGGAAATGAACAGACTCA |
| 17 | CCCCGGATGACACAGCTATCTACTATTGCAAGACCTCCACAGCGGCCAGAGAGAATAACTGGTG |
| | CCGGTCCCGCTACCGCATCGCGTCCTGGGGCCAGGGCACCCAGGTGACTGTCTCCTCT (SEQ |
| | ID NO: 295) |
| hIL | CAGGTGCAGTTGCAGGAGTCTGGAGGGGGCAGCGTGCAGGCCGGAGGCTCCCTCCGCCTCAGCT |
| 12R | GCGCGGCCTCCCGGTACACCTACACCAATAACTTCATGGCATGGTTCAGGCAGGCCCCAGGAAA |
| b1 | GGAGCGTGAGGGGGTCGCCGCAATCTATACCGGAGACGGCTACGCCTATTACTTTGACTCCGTT |
| _VHH | AAAGGGCGTTTCACCATCAGTCAAGACAACGACAAAAACATGCTCTACCTCCAGATGAATAGCT |
| H | TGAAGCCGGAGGATACCGCAATGTACTATTGTGCCGCGATGGAGAGACGCTCCGGTCGGCGTCG |
| 18 | CATGACTGAAAATGCCGAGTACAAGTACTGGGGGCAGGGGACTCAGGTGACCGTGAGCAGC |
| | (SEQ ID NO: 296) |
| hIL | CAAGTTCAGCTCCAGGAGAGTGGAGGCGGTTCCGTACAGGCTGGCGGAAGTCTGCGCCTCTCCT |
| 12R | GCGCCGTCTCCGGTTACGACTATTGTGGGTACGACGTGCGCTGGTATAGACAGGCTCCTGGAAA |
| b1 | GGAGCGTGAGTTTGTGAGTGGCATCAACTCCGACGGTAGCACCTCCTATGCTGATTCTGTGAAG |
| _VHH | GGTCGCTTTACAATCTCACAGGACAACGCCGAAAACACTTCCTATCTGCACATGTTCAGCCTCA |
| 19 | AGCCCGAAGACACCGCAATGTACTATTGTAAGACTGAAGGTCCAGCTGGCGAGAGTGCATGGTG |
| | CAGGAATTTTAGGGGCATGGACTACTGGGGCAAGGGCACCCAGGTCACCGTGTCTTCA (SEQ |
| | ID NO: 297) |
| hIL | CAGGTGCAGTTGCAGGAATCAGGAGGCGGTTCTGTGCAGGCCGGAGGCAGCCTGCGTCTGAGCT |
| 12R | GCACCGCTTCTGGGTACACCTACTCAAGTGCCTTCATGGCCTGGTTTCGGCAAGCGCCCGGCAA |
| b1 | GGAACGCGAGGGAGTTGCGGCCATCTACACCAGGGACGGCAGTCCCGTGTACGCTGACTCCCTG |
| _VHH | AAGGGCCGTTTCACCATCAGCCAGGATAACGCAAAGAACACCCTGCACCTCCAGATGAACAGCC |
| 20 | TGAAACCTGAGGACACAGCTATGTATTACTGCGCGGCCAAAATCCCTGAGCCTGGAAGAATCAG |
| | CCTCCTTGACTCCCAGACCTACGACTACTGGGGTCACGGCACTCAGGTGACTGTGTCTTCT |
| | (SEQ ID NO: 298) |
| hIL | CAGGTTCAACTCCAAGAGTCTGGAGGCGGGTCCGTGCAGGCTGGGGGCTCCCTCAGACTGTCCT |
| 12R | GTACTGCGTCAGGGTACACCTACAGCTCCGCTTTCATGGCTTGGTTCCGGCAAGCTCCGGGCAA |
| b1 | GGAGCGCGAGGGCGTGGCCGCGATGTATACCCGCGACGGTGGCACCGTGTACGCCGACTCTGTT |
| _VHH | AAAGGCCGCTTCACCATCTCCCAGGATAACGCCAAGAACACCCTGTACCTCCAGATGAACTCTT |
| 21 | TGAAGACCGAGGATACCGCTATGTACTATTGCGCCGCAAAAATTCCCCAGCCGGGCCGTGCTTC |
| | CCTTCTGGACAGCCAAACCTATGATTACTGGGGCCAGGGCACACAGGTGACCGTGTCCTCC |
| | (SEQ ID NO: 299) |
| hIL | CAGGTGCAACTTCAGGAATCTGGCGGTGGCAGCGTGCAGGCTGGTGGCTCCCTGCGCCTGAGCT |
| 12R | GTACTGCTTCCGGCTACACATACTCTAGTGCGTTCATGGCCTGGTTCAGGCAAGCTCCGGGAAA |
| b1 | GGAGCGCGAGGGTGTGGCGGCCATTTATACACGCGACGGAGGCACCGTGTACGCTGACTCTGTC |
| _VHH | AAGGGCCGCTTCACCATCTCACAGGACAATGCAAAAAATACCCTCTACCTTCAGATGAACAGCC |
| 22 | TGAAGGCAGAGGACACAGCAATGTATTACTGTGCAGCCAAGATCCCACAACCCGGACGCGCGTC |
| | CCTCCTGGATTCACAGACCTACGACTACTGGGGCCAGGGCACGCAGGTTACTGTATCAAGC |
| | (SEQ ID NO: 300) |

TABLE 11
anti-mILRb1 sdAb VHH DNA SEQUENCE MOUSE

| Name | DNA Sequence |
|---|---|
| mIL12Rb1 | CAGGTGCAGCTCCAGGAAAGCGGGGGAGGTTCCGTCCAGGCC |
| _VHH1 | GGTGGCTCCCTCCGCCTGTCATGCACAGCGAGCGGTTACACGT |
| | ATAGCTCCGCCTTTATGGCCTGGTTTAGACAGGCCCCAGGGAA |
| | AGAACGTGAGGGAGTGGCTGCAATTTACACCCGCGATGGCGG |
| | GACTGTITACGCCGATAGCGTCAAGGGTCGCTTTACCATCAGC |
| | CAGGACAACGCTAAAAACACCCTCTATCTCCAGATGAATAGC |
| | CTGAAGGCCGAGGACACTGCGATGTATTACTGCGCCGCTAAG |
| | ATCCCTCAACCTGGCCGCGCCAGCTTGCTGGATAGCCAGACAT |
| | ACGATTACTGGGGTCAGGGAACACAAGTGACGGTCAGCAGC |
| | (SEQ ID NO: 301) |
| mIL12Rb1 | CAGGTGCAGCTCCAGGAGAGCGGCGGGGGCTCCGTACAGGCC |
| _VHH2 | GGTGGATCACTCCGCCTGAGCTGTGCTGTGAGCGGGTACGAC |
| | TATTGCGGATACGACGTGCGCTGGTATCGCCAAGCTCCAGGG |
| | AAGGAAAGGGAGTTCGTGAGCGGAATTGATTCCGATGGCTCC |
| | ACCAGTTATGCCGACTCCGTTAAAGGAAGGTTTACCATCTCCC |
| | AAGATAACGCCGAGAACACCTCCTATCTGCATATGTTTTCCCT |
| | GAAACCCGAGGATACCGCTATGTATTACTGTAAGACAGAGAG |
| | CCCTGCCGGAGAGTCCGCCTGGTGCCGCAACTTTCGGGGCAT |
| | GGACTACTGGGGAAAGGGCACCCAGGTGACAGTGTCTAGC |
| | (SEQ ID NO: 302) |
| mIL12Rb1 | CAGGTGCAGCTGCAAGAATCAGGAGGTGGATCTGTGCAAGCT |
| _VHH3 | GGGGGCTCTTTGCGCCTGTCCTGTGTCGCCTCCGGCTATAGCT |
| | ATTGCGGCTATGACATGATGTGGTACAGGCAAGCCCCAGGTA |
| | AGGAGAGGGAGTTTGTGGCTCTCATCACCTCCGACTACAGCA |
| | TTCGCTATGAAGATAGTGTCGAGGGACGCTTCTCCATTTCTCG |
| | CGACAACGCGAAGAACACTGGCTATTTGCTGATGAGTAACCT |
| | CACCCCCGCCGACACCGCGATCTACTATTGCAAAACATCTACC |
| | GCCGCTCGGGAAAGTAGCTGGTGTAGGTCACGTTATAGGGTC |
| | GCTTCCTGGGGTCAGGGCACGCAGGTGACCGTCTCATCC (SEQ |
| | ID NO: 303) |
| mIL12Rb1 | CAGGTGCAGTTGCAGGAGAGCGGAGGCGGATCTGTGCAGGCA |
| _VHH4 | GGCGGAAGCCTCCGCCTGTCTTGCGCCGCTTCCCGGTACACCT |
| | ACACAAATAACTTTATGGCATGGTTCCGCCAAGCGCCCGGCA |
| | AGGAGCGCGAGGGTGTCGCGGCCATTTACACAGGTGATGGCT |
| | ACGCCTATTACTTCGACTCCGTGAAAGGCAGGTTCACGATCTC |
| | CCAGGATAACGACAAGAATATGTTGTATCTTCAGATGAACTCT |
| | CTGAAACCTGAGGACACCGCTATGTACTATTGTGCAGCTATGG |
| | AACGCAGGTCAGGCAGGCGCAGGATGACCGAGAACGCCGAG |
| | TACAAGTACTGGGGCCAGGGCACCCAGGTGACCGTGTCTTCA |
| | (SEQ ID NO: 304) |
| mIL12Rb1 | CAGGTGCAGCTCCAGGAGTCTGGAGGCGGTTCCGTCCAGGCC |
| _VHH5 | GGGGAAACGCTCCGGCTTAGCTGCACCGTCTCCGGTTTCACCA |
| | TTGATGACTCCGAAATGGGTTGGTATCGCCAAGCGCCCGGCC |
| | ATGAGTGCGAACTGGTGGCCAGCGGAAGTTCCGACGATGACA |
| | CCTATTACGTGGACTCAGTGAAGGGTCGCTTTACGATCTCTCT |
| | GGATAACGCCAAAAACATGGTGTACCTCCAGATGAACTCACT |
| | CAAGCCAGAGGATACAGCAGTITATTACTGTGCCACTGGACC |
| | TACATACCCTCCCAAGGATGGTGACTGCGCACACTGGGGTCA |
| | AGGCACCCAGGTCACTGTCTCCTCC (SEQ ID NO: 305) |
| mIL12Rb1 | CAAGTCCAGCTCCAGGAGTCTGGGGGAGGCTCAGTGCAAGCT |
| _VHH6 | GGTGGATCTCTTCGCCTGTCTTGCACCGCTTCTGGGTACACCT |
| | ATAGCTCTGCCTTCATGGCCTGGTTTAGGCAAGCGCCTGGCAA |
| | GGAGCGGGAGGGCGTCGCCGCTATCTACACCCGCGACGGCAG |
| | TCCGGTTTATGCCGACTCCCTGAAGGGTAGATTTACTATCTCT |
| | CAGGATAATGCAAAGAATACGCTGCACTTGCAGATGAACTCC |
| | CTCAAACCCGAGGACACGGCCATGTATTACTGTGCTGCAAAA |
| | ATCCCAGAGCCTGGTCGGATCTCCCTCCTGGATTCACAGACCT |
| | ACGACTACTGGGGCCACGGCACCCAGGTGACAGTCTCTTCC |
| | (SEQ ID NO: 306) |
| mIL12Rb1 | CAGGTGCAGCTCCAGGAGTCCGGTGGCGGAAGCGTGCAGGCC |
| _VHH7 | GGTGGCTCCCTGCGGTTGAGTTGCGCGGTCTCAGGTTACGATT |
| | ATTGTGGCTACGACGTGCGCTGGTATAGACGCGCTCCTGGCA |
| | AGGAGCGTGAGTTCGTGTCTGGCATCGACTCCGATGGCTCTAC |
| | TTCATACGCTGATTCCGTCAAAGGCCGTTTCACCATCTCTCAG |
| | GATAACGCCGAGAACACCTCCTACCTTCACATGTTCTCTCTGA |
| | AGCCCGAGGATACTGCAATGTATTACTGTAAGACTGAGTCTCC |
| | TGCCGGAGAATCCGCCTGGTGTCGTAACTTTCGTGGCATGGAC |
| | TACTGGGGTAAGGGAACCCAGGTGACTGTATCTTCC (SEQ ID |
| | NO: 307) |
| mIL12Rb1 | CAGGTCCAGTTGCAGGAGTCTGGTGGAGGCTCCGTCCAAGCT |
| _VHH8 | GGGGGCTTTCTTAGGCTGTCATGTGTGGCATCCGGCTATGGGT |
| | ATTGTGGCTATGATATGTCCTGGTATAGACAAGCGCCCGGCA |
| | AGGAGCGCGAGTTCGTGGCGCTGATTACCAGCGAGCGCGTTA |
| | TCAGCTACGAGGACTCCGTCAAAGGCAGATTCTCCATCTCACG |
| | CGACAACGCCGAGAACACAGGCTATCTGGAAATGAATCGTTT |
| | GACACCTGATGACACCGCTATCTACTATTGCAAGACCTCTGCG |
| | GCTGCGCGTGAGTCTAGCTGGTGCCGTTCCCGCTATAGAGTGG |
| | CTTCTTGGGGTCAGGGAACCCAGGTGACAGTCTCCAGC (SEQ |
| | ID NO: 308) |
| mIL12Rb1 | CAGGTACAGCTCCAGGAGTCTGGAGGCGGGAGCGTGCAGGCA |
| _VHH9 | GGCGGTTCCCTGCGTCTGTCCTGCGTCGCCTCTGGGTATGGGT |
| | ACTGCGGCTACGATATGTCCTGGTATCGTCAGGCTCCCGGCAA |
| | AGAAAGAGAGTTCGTAGCCCTCATCACATCTGACCGGAGCAT |
| | TTCCTACGAAGACTCCGTCAAGGCCCGCTTCATTATCTCACGG |
| | GATAACGCAGCCAACACCGGATACCTGGACATGACTCGCCTG |
| | ACCCCCGATGACACTGCTATCTATTACTGCAAGACGAGCGCG |
| | GCAGCTCGCGAGAGTTCTTGGTGCCGGTCCCGGTACAGGGTG |
| | GCGTCCTGGGGCCAGGGGACTCAGGTCACCGTCTCCTCC (SEQ |
| | ID NO: 309) |
| mIL12Rb1 | CAGGTGCAACTCCAGGAGAGTGGAGGTGGCTCAGTACAGGCC |
| _VHH10 | GGGGGAAGCCTCCGTCTGAGCTGTGCCGTGTCCGGCTACGATT |
| | ACTGTGGTTACGACGTGCGGTGGTATCGCCAGGCCCCTGGTA |
| | AGGAAAGAGAGTTCGTGTCCGGCATCGACAGCGATGGTAGCA |
| | CATCTTACGCCGACTCCGTGAAGGGCCGCTTCACAATCTCCCA |
| | GGACAACGCCGAAAACACGTCTTACCTCCATATGTTTTCCCTG |
| | AAACCTGAAGACACCGCTATGTATTACTGCAAGACCGAGTCT |
| | CCCGCTGGCGAGTCAGCATGGTGTAGGAACTTTCGCGGCATG |
| | GACTATTGGGGTAAGGGCACCCAGGTGACGGTGAGTTCT (SEQ |
| | ID NO: 310) |
| mIL12Rb1 | CAGGTGCAGCTCCAGGAAAGCGGCGGGGGAAGCGTGCAGGC |
| _VHH11 | AGGAGGCTCCCTTCGGTTGAGCTGCGTGGCCAGCGGCTACAG |
| | CTACTGCGGCTACGACATGATGTGGTATCGCCAAGCTCCGGG |
| | GAAGGAGCGCGAGTTCGTCGCCCTCATCACCAGTGATTATTCT |
| | ATCCGCTACGAAGACTCTGTGGAAGGTAGGTTCTCCATTAGCA |
| | GAGACAACGCAAAGAACACTGGATACCTGCTTATGAGCAACC |
| | TCACACCCGCCGACACTGCCATCTACTATTGTAAGACCTCTAC |
| | CGCCGCTCGCGAAAGCTCCTGGTGCAGGTCCCGCTATCGCGTG |
| | GCCAGTTGGGGTCAGGGAACCCAGGTGACGGTATCTAGC |
| | (SEQ ID NO: 311) |
| mIL12Rb1 | CAGGTTCAGTTGCAGGAGTCTGGAGGTGGCAGTGTGCAAGCT |
| _VHH12 | GGAGGCTCCCTCCGCCTGAGTTGCGCTGCCAGCAGATATACCT |
| | ATACGAATAACTTTATGGCTTGGTTTAGACAGGCCCCCGGTAA |
| | AGAGCGGGAAGGTGTGGCCGCGATTTACACCGGCGATGGCTA |
| | CGCCTATTACTTITACAGCGTGAAGGGACGTTTCACCATTICT |
| | CAGGATAACGATGAAAACATGCTGTATCTCCAAATGAACTCT |
| | CTGAAGCCTGAAGACACCGCTATGTATTACTGCGCGGCTATG |
| | GAGCGCAGGATCGGAACAAGACGCATGACTGAGAACGCTGA |
| | GTATAAATATTGGGGACAAGGCACACAGGTGACAGTTAGCTC |
| | C (SEQ ID NO: 312) |
| mIL12Rb1 | CAGGTCCAACTCCAGGAGTCCGGGGGAGGGTCTGTGCAGGCG |
| _VHH13 | GGTGGCTCCCTGCGCCTGAGCTGTGTCGCGTCTGGTTACTCCT |
| | ACTGTGGATATGATATGATGTGGTATAGACAGGCCCCAGGTA |
| | AGGAGCGCGAGTTTGTGGCCCTGATTACCAGCGACTACAGTA |
| | TCCGCTATGAGGATTCCGTGGAGGGCCGCTTCTCTATCTCACG |
| | CGACAACGCCAAGAATACAGGCTACCTCCTGATGAGCAACCT |
| | GACCCCTGCCGACACAGCCATTTATTACTGCAAGACCTCCACC |
| | GCCGCGCGTGAATCCGGCTGGTGCAGGTCACGCTATCGTGTC |
| | GCCAGCTGGGGTCAGGGGACACAGGTGACGGTGTCATCT |
| | (SEQ ID NO: 313) |
| mIL12Rb1 | CAAGTGCAGTIGCAAGAATCAGGAGGCGGGTCCGTGCAGGCG |
| _VHH14 | GGCGGATCTCTGCGTCTGTCTTGTGCTGTCTCCGGTTATGACT |
| | ACTGTGGTTACGACGTGCGCTGGTATCGCCAGGCCCCTGGTAA |
| | GGAACGTGAGTTCGTGAGCGGGATCAATAGCGACGGCTCCAC |
| | CTCTTATGCCGACAGTGTGAAGGGTAGGTTTACCATCAGTCAA |
| | GACAACGCCGAGAACACATCCTACCTTCATATGTTCTCTCTCA |
| | AGCCTGAGGATACCGCAATGTACTATTGCAAGACGGAGTCCC |
| | CAGCAGGTGAGTCCGCTTGGTGCAGAAACTTTCGCGGCATGG |
| | ATTATTGGGGGAAGGGAACCCAGGTCACCGTGTCTTCC (SEQ |
| | ID NO: 314) |
| mIL12Rb1 | CAGGTGCAACTTCAGGAATCCGGTGGCGGATCTGTTCAGGCT |
| _VHH15 | GGCGGATTCCTGCGCCTGTCTTGCGTGGCCAGTGGCTACGGCT |
| | ACTGCGGCTATGATATGTCATGGTATCGCCAAGTGCCCGGCA |
| | AGGAGCGCGAGTTTGTAGCCCTCATCACATCTGATCGTTCTGT |
| | CAGCTACGAAGACAGTGTCAAGGGCCGCTTTTCCATCAGCCG |
| | CGATAATGCGAAGAACACGGCCTACCTGGAGATGAACAGACT |
| | GACACCGGATGACACCGCTGTATATTACTGTAAGACCTCAAC |
| | GGCTGCCAGAGAGAATAATTGGTGCCGTTCTCGCTACCGCATC |
| | GCTTATTGGGGCCAGGGAACACAGGTCACAGTCTCCTCC (SEQ |
| | ID NO: 315) |
| mIL12Rb1 | CAGGTGCAACTCCAGGAGAGCGGGGGAGGTTCCGTTCAGGCC |
| _VHH16 | GGGGGTTCCCTCAGATTGTCTTGTGCCGTCTCCGGGTACGATT |
| | ACTGTGGCTATGACGTGCGCTGGTATCGGCAGGCTCCTGGGA |
| | AGGAGCGGGAGTTCGTGAGTGGCATTAACTCAGACGGGTCTA |
| | CCTCCTATGCCGACAGCGTTAAGGGCAGGTTTACTATCAGTCA |
| | GGACAATGCGGAGAATACCAGTTACCTGCACATGTTCAGCCT |
| | CAAGCCCGAGGATACCGCCATGTATTACTGCAAGACAGAGGG |
| | TCCAGCTGGCGAGTCCGCATGGTGCCGCAACTTCAGGGGTAT |
| | GGACTACTGGGGCAAGGGTACTCAGGTGACTGTGTCCTCT |
| | (SEQ ID NO: 316) |
| mIL12Rb1 | CAGGTGCAGTTGCAGGAGTCAGGCGGGGGCTCTGTCCAGGCT |
| _VHH17 | GGGGGCTCTCTGAGACTGTCTTGTACTGCGTCTGGTTACACGT |
| | ACAGTTCTGCCTTTATGGCCTGGTTTCGGCAAGCGCCCGGAAA |
| | GGAGCGCGAGGGTGTTGCTGCCATGTATACCCGTGATGGCGG |
| | AACCGTCTACGCAGATTCTGTTAAGGGTCGTTTCACAATCTCC |
| | CAGGACAATGCGAAAAATACCCTCTATCTCCAGATCCACACC |
| | TTGAAGGCTGAGGACACCGCGATGTATTACTGTGCTGCCAAG |
| | ATCCCGCAGCCTGGCCGCGCTTCCCTGCTCGACAGCCAGACAT |
| | ACGACTACTGGGGTCAGGGCACACAGGTTACCGTGAGTAGT |
| | (SEQ ID NO: 317) |
| mIL12Rb1 | CAAGTCCAACTCCAGGAAAGCGGAGGTGGCAGCGTCCAGGCC |
| _VHH18 | GGGGGCTCTCTGAGACTGTCTTGTACCGCTTCCGGCTATACAT |
| | ATTCCTCTGCCTTTATGGCATGGTTCCGCCAAGCGCCAGGCAA |
| | GGAGCGCGAGGGCGTCGCCGCTATGTATACCAGAGACGGAGG |
| | CACCGTCTACGCTGACAGCGTCAAGGGACGCTTCACAATCTCC |
| | CAGGACAACGCCAAGAATACTTTGTATCTCCAGATGAATAGC |
| | CTCAAGACGGAGGACACCGCAATGTATTACTGCGCTGCAAAA |
| | ATCCCTCAGCCAGGTCGCGCCTCCCTCCTGGACAGTCAGACCT |
| | ATGATTATTGGGGCCAGGGGACCCAGGTGACTGTCTCCTCC |
| | (SEQ ID NO: 318) |
| mIL12Rb1 | CAGGTACAGTTGCAGGAGTCCGGCGGAGGCAGCGTTCAGGCC |
| _VHH19 | GGTGGCTTCCTGAGGCTGTCCTGCGTCGCCAGCGGCTATGGAT |
| | ATTGCGGCTACGATATGTCCTGGTACAGACAGGTCCCTGGGA |
| | AAGAACGCGAGTTCGTGGCTCTTATCACATCCGACAGGTCCGT |
| | GTCCTATGAGGACTCTGTCAAGGGCCGTTTCAGCATCAGCCGT |
| | GACAACGCAAAAAACACGGCTTACTTGGAGATGAACCGGCTT |
| | ACCCCCGACGATACCGCGATTTATTACTGCAAGACCAGCACA |
| | GCAGCCAGGGAAAATAATTGGTGTCGGAGCCGTTATCGTATC |
| | GCCTCTTGGGGACAGGGAACCCAGGTGACTGTCTCCTCA (SEQ |
| | ID NO: 319) |
| mIL12Rb1 | CAGGTGCAGCTCCAGGAGTCCGGCGGAGGCTCAGTACAAGCT |
| _VHH20 | GGCGGTTCACTCAGGTTGAGTTGTGTCGCCAGTGGCTACGGCT |
| | ATTGTGGCTATGATATGTCTTGGTATCGCCAGACCCCCGGCAA |
| | GGAGCGTGAGTTCGTGGCACTCATCACGTCCGACCGGATCGC |
| | CTCTTACGAAGACTCTGTCAAGGGCCGTTTTATTATCAGCCGC |
| | GACAACGCAAAAAACACTGGTTATCTCGACATGACTCGGGTG |
| | ACCCCCGATGACACTGCCATCTACTATTGCAAAACCTCTGCTG |
| | CGGCCCGCGAGAACTCCTGGTGCCGTAGTCGCTACCGCGTCG |
| | CCTCCTGGGGACAGGGTACACAGGTGACCGTTAGCTCC (SEQ |
| | ID NO: 320) |
| mIL12Rb1 | CAGGTCCAACTGCAAGAGTCTGGCGGTGGCTCCGTGCAGGCT |
| _VHH21 | GGCGGTAGTCTGCGCCTGTCTTGTGCAGTCAGCGGGTACGACT |
| | ACTGCGGTTATGATGTCAGATGGTATCGCCGTGCTCCCGGCAA |
| | GGAACGCGAGTTCGTCTCTGGCATTGACTCCGACGGCTCTACC |
| | TCCTATGCCGATAGCGTAAAGGGAAGGTTCACCATCAGCCAG |
| | GACAACGCTGAGAACACCAGCTACTTGCACATGTTCTCCCTTA |
| | AACCTGAGGACACAGCTATGTATTACTGTAAAACTGAGAGCC |
| | CGGCTGGCGAGAGCGCCTGGTGTCGCAACTTTCGTGGCATGG |
| | ACTACTGGGGTAAGGGCACCCAGGTTACTGTCTCTAGT (SEQ |
| | ID NO: 321) |
| mIL12Rb1 | CAGGTGCAACTTCAGGAGAGCGGTGGCGGTTCAGTGCAGGCT |
| _VHH22 | GGGGGAAGCCTGCGCCTGTCTTGCACCGCTTCCGGCTACACCT |
| | ATTCCAGTGCCTTCATGGCCTGGTTCCGCCAGGCCCCTGGAAA |
| | GGAACGCGAAGGCGTGGCTGCCATTTATACACGGGATGGGGG |
| | AACCGTCTACGCGGACTCCGTCAAGGGAAGATTCACCATTAG |
| | CCAGGATAATGCTAAGAACATCCTGTACCTCCAGATGAACTC |
| | CCTCAAAGCCGAGGATACTGCTATGTACTATTGTGCCGCTAAG |
| | ATTCCGCAGCCAGGCCGGGCATCCCTCCTGGACAGCCAGACC |
| | TATGACTACTGGGGACAGGGGACCCAGGTGACCGTGTCTTCC |
| | (SEQ ID NO: 322) |
| mIL12Rb1 | CAGGTGCAGCTCCAGGAGTCCGGCGGTGGCAGTGTCCAGGCA |
| _VHH23 | GGAGGCAGTCTGCGTCTGTCTTGCACTGCCTCAGGCTACACAT |
| | ACTCAAGCGCATTCATGGCCTGGTTCAGGCAGGCCCCTGGGA |
| | AGGAGCGCGAGGGTGTGGCAGCTATCTACACCCGCGATGGCG |
| | GTACTGTGTACGCCGATAGTGTCAAGGGGCGCTTTACCATTTC |
| | TCAGGACAACGCGAAGAACACCCTGTACTTGCAGATGAACAG |
| | CCTGAAGCCGGAGGATACTGCTATGTATTACTGCGCCGCAAA |
| | AATGCCCCAGCCGGGCCGCGCGTCTTTGCTGGATTCCCAGACA |
| | TACGACTACTGGGGGCAGGGCACCCAGGTTACGGTTAGCTCC |
| | (SEQ ID NO: 323) |

TABLE 12
anti-human IL23R sdAb VHH DNA Sequences

| Name | Sequence |
|---|---|
| hIL | CAGGTTCAGCTGCAAGAGAGCGGGGGTGGGTCTGTGCAGGCTGGTGGCAGCTTGCGCCTTAGTT |
| 23R | GCGCGGCTTCTGGTTATACTTATTGTTCCTACGATATGTCATGGTATCGTCAGGCTCCTGGCAA |
| _VH | GAAACGGGAGTTCGTCTCTGCCTTCAACTCCGATGGCACCACTAGCTATGCAGATTCTGTGAAA |
| H1 | GGCAGATTCACCATCTCTCAGGACAAGGCCAAGAATACCGTGTACCTCCAGATGAACAGCCTGA |
| | AGCCAGAGGATACCGCTATGTACTATTGCAAGACAGATCCTCACGTGCAATCCTCTGGTGGCTA |
| | CTGTCCGCCCTACTGGGGCCAGGGCACACAGGTAACGGTTAGTTCC (SEQ ID NO: 324) |
| hIL | CAGGTGCAGCTCCAGGAATCTGGCGGAGGTTCCGTGCAGGCCGGTGGCAGCCTGAGGCTCAGCT |
| 23R | GCGCCGCGTCCGGGTATACCTACTGTTCCTACGATATGTCCTGGTATCGGCAGGCTCCGGGTAA |
| _VH | AAAGAGAGAGTTTGTGTCCAGCTTTAACAGCGACGGCAGTACATCTTACGCTGACTCCGTGAAG |
| H2 | GGTCGCTTCACCATTAGCCAGGATAACGCAAAAAACACAGTGTACCTTCAGATGAACAGTCTGA |
| | AGCCAGAGGACACCGCCATGTATTACTGCAAGACGGACCCGCACGCTGATTGGGGTGCCCCTTG |
| | CGGGGGCGATTATTGGGGCCAAGGCACCCAGGTGACTGTTTCTTCC (SEQ ID NO: 325) |
| hIL | CAGGTGCAGCTCCAGGAATCTGGGGGCGGTTCTGTGCAAGCGGGCGAGAGCCTGAGACTGAGCT |
| 23R | GCGCCGCGAGCGGCTACACCTACTGTACCTATGACATGACTTGGTACAGACAGGCCCCCGGAAA |
| _VH | AAAGCGCGAGTTCGTCAGCGGTATCCATAGCGACGGTACTACCTCTTACGCAGATTCCGTGAAA |
| H3 | GGCCGCTTCACAATTTCTCAGGACAATGCCGAAAACACCGTGTACCTCCAGATGAACTCCCTGA |
| | AGCCAGAGGATACCGCGATGTATTACTGCAAGACAGACCCCATCGCCACCATCACCCGCCGGTG |
| | CGACTCATACTGGGGGCAGGGCACTCAGGTCACAGTCTCATCT (SEQ ID NO: 326) |
| hIL | CAGGTGCAACTTCAGGAATCAGGAGGCGGTAGTGTGCAAGCGGGCGGAAGTCTGCGCCTGAGCT |
| 23R | GCGCTGCCTCCGGGAGCACGTATTGTACGTATGATATGACGTGGTACAGGCAGGCCCCTGGCAA |
| _VH | GCGCAGGGAGTTCGTGAGTGCAATCAACTCAGATGGCAGCACCTCTTATGCTGACAGCGTGAAA |
| H4 | GGGAGATTCACTATCTCCCAGGACAACGCAAAAAACACCGTCTATCTCCAGATGAACTCTCTGA |
| | AGCCCGAAGACACCGCGATGTATTACTGTAAGACTGATCCTAACAGCGGATGGGGCGCTCCTTG |
| | CGGTGGCGATTATTGGGGACAGGGCACCCAAGTGACAGTTAGCAGC (SEQ ID NO: 327) |
| hIL | CAGGTGCAGCTTCAGGAAAGCGGTGGGGGCTCCGTCCAGGCAGGCGGTTCCCTTCGCCTTTCTT |
| 23R | GTGCCGCTTCTGGTTATACTTACTGTTCATACGATATGTCTTGGTATCGCCAGGCTCCCGGCAA |
| _VH | AAAGCGTGAGTTCGTCTCTGCCATCGCCTCCGATGGCTCAACGTCCTACGCGGACAGTCTCAAG |
| H5 | GGTCGCTTCACCATTTCCCAGGATAATGCAAAGAACACCGTGTATCTCCAGATGAACTCACTGA |
| | AGCCCGAAGACACAGCCATGTACTATTGCAAGACTGACCCACACGTACAGTCTTCCGGCGGATA |
| | CTGCCCACCTTACTGGGGACAGGGAACCCAGGTGACAGTGAGTTCT (SEQ ID NO: 328) |
| hIL | CAGGTGCAGCTTCAGGAAAGTGGCGGGGGTCTGGTGCAGCCGGGCGGGTCCCTCCGGCTGTCCT |
| 23R | GTGCTGCCAGCGGCTACACCTATTGCAGCTATGATATGGGCTGGTATCGCCAGGCCCCTGGAAA |
| _VH | AAAGAGAAAGTTTGTGTCCAGCATTAACAGCGATGGGACCACTTCTTACGCTGACAGTGTTAAA |
| H6 | GGGCGTTTCACGATCTCCCAGGACAACGCTAAAAACACCGTGTATCTCCAGATGAATAGCCTGA |
| | AGCCCGAGGACACCGCAATGTATTACTGTAAAACTGACCCCCAGACACGTCCCGGTAAGCCATG |
| | TGCTGATTATTGGGGCCAGGGGACCCAGGTGACCGTCAGCTCC (SEQ ID NO: 329) |
| hIL | CAGGTGCAGCTCCAGGAGTCCGGTGGCGGGTCTGTCCAAGCTGGCGGTTCCCTTCGCCTGTCCT |
| 23R | GTGCAGCCAGTGGATATACGTATTGCAACTACGACATCGCCTGGTATAGACAGGCCCCTGGCAA |
| _VH | AGAGCGCAAGTTCGTATCCGCAATCGCCAGTGACGGTATCACCTCTTATGCTGACTCTGTGAAG |
| H7 | GGTCGGTTCACTATCTCCCAGGATAACGCTAAGAACACAGTCTACCTCCAGATGAACAGCTTGA |
| | AGCCGGAGGACACTGCGATGTACTATTGCAAGACTGATCCGATTTCCACCATCACAAGGATCTG |
| | CGACCCGTACTGGGGCCAGGGCACCCAAGTGACTGTGTCATCA (SEQ ID NO: 330) |
| hIL | CAAGTGCAGTTGCAGGAGAGCGGTGGCGATTCTGTGCAGGCCGGAGGCTCCCTCCGCCTGTCCT |
| 23R | GTGCCGCTTCAGGCTACACGTATTGTTCTTATGATATGAAGTGGTATCGCCAGGCCCCAGGTAA |
| _VH | GGAACGCGAGTTCGTCAGCGGTATTGATTCCGACGGCAGTATTAGCTACGCCGACTCCGTGAAA |
| H8 | GGCCGCTTCACAATTAGCCAGGACAACGCGAAAAACACCGTGTACCTCCAGATGAACTCTCTGA |
| | AGCCAGAGGATACCGCCATGTACTATTGCAAGACTGAGGGCACTATCCCCGTAGGTGCATGTCC |
| | TAACTACTGGGGCCAGGGCACTCAGGTAACCGTCAGTAGC (SEQ ID NO: 331) |
| hIL | CAAGTCCAGCTCCAGGAGAGCGGCGGTGGCTTGGTGCAGGCCGGTGGCTCCCTGAGACTGAGCT |
| 23R | GTGCAGCCTCCGGGTATACATATTGCAGCTACGACATGAGTTGGTATCGCCAGGCACCGGGCAA |
| _VH | GGAGAGAAAGTTTGTGTCCTCTATCAATTCAGATGGCACAACCTCCTACGCCGACTCAGTCAAG |
| H9 | GGTCGTTTCACTATTTCTCAGGACAACGCTAAGAACACCGTGTACCTCCAGATGAACAGCCTGA |
| | AGCCTGAGGATACGGCCATGTACTATTGCAAAACTGACCCCCAGACTAGACCTGGCAAGCCGTG |
| | CGCGGACTATTGGGGTCAGGGCACGCAAGTCACCGTGTCCTCA (SEQ ID NO: 332) |
| hIL | CAGGTGCAGTTGCAGGAGAGCGGCGGAGGGTCTGTGCAGGCTGGAGGTAGCCTGAAGCTGTCCT |
| 23R | GTGCTGCCAGCGGCTACACCTACTGTAACTACGATATCGCGTGGTATCGGCAAGCGCCCGGCAA |
| _VH | AGAGCGTAAGTTCGTGTCCGCTATCGCCTCCGATGGCTCTACTTCCTATGCCGACAGCGTTAAA |
| H10 | GGTCGCTTCACCATCTCCCAAGACAACGCCAAAAATACAGTGTATCTTCAGATGAACTCTCTGA |
| | AACCCGAGGATACTGCGATGTATTACTGCAAGACTGATCCAATCGCTACCATGACCAGGCGCTG |
| | CGACCCTTACTGGGGCCAGGGCACCCAGGTGACGGTATCCTCA (SEQ ID NO: 333) |
| hIL | CAAGTCCAGCTTCAGGAGTCTGGTGGGGGCTCTGTCCAGGCCGGGGGCTCTCTGAGACTGTCTT |
| 23R | GTGCAGCCTCCGGGTACACCTACTGTTCCTATGACATGACTTGGTATCGTCAAGCACCTGGCAA |
| _VH | AAAGCGTGAGTTCGTGTCTGCCATCGACTCCGACGGCTCTACCTCCTACGCCGACTCTGTGAAA |
| H11 | GGTAGGTTCACAATCTCCCAGGATAACGCAAAGAATACTGTGTACTTGCAGATGAACTCCTTGA |
| | AGCCCGAGGATACTGCCATGTACTATTGCAAGACAGACCCTATTGCTACTATCTCTCGTAGGTG |
| | TGATAGTTACTGGGGACAAGGCACCCAGGTTACCGTATCCAGT (SEQ ID NO: 334) |
| hIL | CAGGTGCAGCTCCAAGAATCTGGTGGAGGGTCAGTGCAGGCCGGAGGCAGCCTGCGCCTGTCTT |
| 23R | GCGCTGCAAGCGGTTACACCAGCTCCTCTCGCTGTATGGGATGGTTCCGGCAAGCTCCGGGCAA |
| _VH | GGAAAGGGAAGGAGTCGCTCGTATCTACACCCCAACCAGAACTACGTGGTACGCCGATAGCGTC |
| H12 | AAGGGGCGCTTCACCATCAGCCAGGATAACGCCAAGAATACCGTGTACCTGGAGATGGCCAGCC |
| | TCAAGCCAGAGGACACGGCGAAGTATTTTTGCGCTGCCGGGGCGTCCTGCGCCGTGGATTTGTT |
| | CTCTTACTGGGGTCAGGGCACTCAGGTCACCGTGTCAAGC (SEQ ID NO: 335) |
| hIL | CAGGTTCAGCTCCAGGAGTCCGGCGGTGGCTCTGTGCAGGCCGGTGGCTCCCTGAGACTGTCCT |
| 23R | GCGCGGCTTCAGGATACACGTACTGCTCCTATGATATGAAGTGGTATCGTCAGGCTCCAGGCAA |
| _VH | AAAGAGGGAGTTCGTGAGCGCGATTGATTCCGATGGGAGTACCTCCTACGCGGACTCTGTGAAG |
| H13 | GGACGCTTTACTATTTCCCAGGACAACGCGAAGAATACGGTCTACCTGCAAATGAACTCCCTCA |
| | AGCCGGAGGATACCGCTATGTATTACTGTAAAACCGAGGGAACAATTCCTGTCGGCGTCTGCCC |
| | TAATTATTGGGGGCAGGGCACACAAGTGACTGTCTCCTCC (SEQ ID NO: 336) |
| hIL | CAAGTCCAGCTGCAAGAGTCTGGTGGGGGCCTGGTGCAACCAGGGGGCAGCTTGAGACTCTCCT |
| 23R | GCGCTGCCAGCGGGTATACATACTGTAGCTATGATATGAAGTGGTACAGGCAAGCCCCTGGCAA |
| _VH | AAAGCGCGAGTTCGTGTCCGCCATCGACTCCGACGGTTCCACTAGCTACGCGGATTCCGTGAAG |
| H14 | GGAAGGTTCACTATTTCTCAGGATAACGCCAAGAACACCGTCTACCTCCAGATGAACTCCCTGA |
| | AGCCAGAGGACACCGCCATGTACTATTGTAAGACCGAGGGCACAGTGCCTGTGGGCGTCTGTCC |
| | AAATTATTGGGGTCAAGGCACCCAGGTCACAGTATCCTCT (SEQ ID NO: 337) |
| hIL | CAAGTGCAGCTGCAAGAGAGCGGTGGCGGGTCCGTGCAAGCAGGTGGCTCCTTGCGCCTGTCCT |
| 23R | GCGCTGCCAGCCCCGGCACCTACACATCCCGTTATATGGGATGGTTTCGCCAGGCACCTGGAAA |
| _VH | GGAACGCGAGGGGGTTGCGACTATCTGGCCCGCTGGCGGTAACACCGTTTACGCCGATAGCGTG |
| H15 | AAAGGGCGCTTCACCATTAGTCAAGACGGGGCCAAAAAGACCGTGTACCTCCAGATGAACTCCC |
| | TGAAACCTGAAGACACTGCTATGTACTATTGTGCCGCAGCTAAGTACGGCGGGACTAGCCTGGC |
| | TCCTTACACATATAACTACTGGGGCCAAGGCACCCAGGTGACAGTCTCTTCT (SEQ ID |
| | NO: 338) |
| hIL | CAGGTCCAACTCCAGGAATCCGGTGGGGGCAGCGTCGAGGCCGGGGGCGCACTCACCCTCTCCT |
| 23R | GCGTCGCAAGCGGCTATACGTACTGTAACTACGACATTGCTTGGTATCGCCAGGCCCCAGGCAA |
| _VH | GGAGCGCAAGTTCGTTTCCGCCATCGCCTCTGATGGAAGCACAAGTTACGCAGATTCCGTGAAA |
| H16 | GGCCGGTTCACAATCTCACAGGACAACGCTAAGAACACCGTCTACTTGCAGATGAACAGTCTGA |
| | AGCCCGAAGACACCGCCATGTATTACTGCAAGACCGATCCCATCGCCACTATGACACGTCGCTG |
| | TGACCCCTACTGGGGCCAAGGCACTCAGGTGACCGTGAGTTCC (SEQ ID NO: 339) |
| hIL | CAGGTACAGCTCCAAGAATCTGGCGGTGGCTCCGTGCAGGCCGGTGGCTCCTTGCGTCTGTCCT |
| 23R | GCACCGCCAGTGGATATACTTTCTCCACCATGAAGTACATGGGATGGTTCCGCCAGGCTCCGGG |
| _VH | AAAGGAGAGGGAGGGCGTTGCGGCCATTTGGATCGCCGCTGGCAACACTTATTACGCCGATTCC |
| H17 | GTGAAAGGCCGCTTTACCATTTCCCAGGACAACACAAAGAACACCGTTTACCTCCAGATGAATA |
| | GCCTGAAGCCAGAGGATACCGCCCTCTACTATTGCGCGGCAGCCAGGTACGGCTTTGTCCCCAG |
| | CACTTGGTATCTCCCCGAGCGTTACAACTATTGGGGCCAGGGAACTCAGGTGACTGTCAGTTCC |
| | (SEQ ID NO: 340) |
| hIL | CAGGTGCAGTTGCAGGAGTCCGGCGGTGGGTCTGTGCAGGCGGGCGGGAGCCTGCGCTTGGCCT |
| 23R | GCGCCGCAAGCGGTTATACCTACTGCAATTACGACATCGCGTGGTATCGGCAGGCCCCCGGTAA |
| _VH | GGAGCGTAAGTTCGTGTCCGCCATCGCGTCTGACGGAAGCACCTCTTATGCCGATAGCGTGAAA |
| H18 | GGAAGATTTACAATCTCCCAGGATAACGCCAAAAACACCGTCTATTTGCAGATGAATAGTCTGA |
| | AGCCAGAGGACACGGCTATGTATTACTGCAAGACCGATCCAATCGCTACCATGACCAGGCGCTG |
| | CGACCCATATTGGGGCCAGGGCACGCAGGTCACCGTATCCTCC (SEQ ID NO: 341) |
| hIL | CAGGTCCAGTTGCAGGAAAGCGGCGGTGGATCAGTCCAGGCTGGTGGGAGTCTGCGTCTGAGCT |
| 23R | GTGCTGCCAGCGGTTATACATCCTGCTCTTACGATATGTCTTGGTACAGACAGGCCCCTGGCAA |
| _VH | GAAAAGAGAGTTCGTTTCCGCCATTCATAGTGATGGCACAACCTCCTACGCCGACAGCATGAAG |
| H19 | GGGAGGTTCACTATCTCCCAGGATAATGCTAAGAATACCGTTTATCTCCAGATGAACTCACTGA |
| | AACCGGAGGACACCGCAATGTACTATTGCAAGACGGACCCTAACTACTCAGACCACGTGTGCCC |
| | GCCTTACTGGGGACGCGGTACTCAAGTGACTGTGTCAAGC |
| | (SEQ ID NO: 342) |

TABLE 13
anti-mIL23R sdAb VHH DNA SEQUENCE MOUSE

| Name | DNA Sequence |
|---|---|
| mIL23R | CAAGTGCAGCTGCAAGAATCTGGCGGAGGCTTGTGCAAGCTGGC |
| VHH1 | GGTTCCTTGCGTCTGAGCTGCGCCGCATCTGGGTATACCTACAGT |
| | AGCTGCACAATGGGTTGGTATAGACAAGCGCCCGGTAAGGAGCG |
| | GGAACTGGTGTCCATGCTGATCTCTGACGGCAGTACTTTTTACGC |
| | CGACTCCGTGAAGGGCAGATTCACATTCTCCCAGGAGTACGCCA |
| | AGAACACCGTCTACCTTCAGATGAACAGTCTGAAGCCTGAGGAC |
| | ACCGCTATGTACTATTGCGGCTGCGCAACGCTCGGCTCCCGCACG |
| | GTCTGGGGTCAGGGCACCCAGGTCACCGTGTCCTCT (SEQ ID NO: |
| | 343) |
| mIL23R | CAGGTTCAGCTTCAGGAGTCTGGTGGCGGTTCCGTACAGGCCGG |
| VHH2 | GGGCTCCCTTACTCTCAGCTGTACCGCGCCGGGTTTTACTTTCCG |
| | GTTGGCGGCCATGCGCTGGGTTCGCCAGGCTCCGGGAAAAGGAC |
| | TGGAGTGGGTTAGCGGCATTGATTCCAGGGGCTCCACCATCTATG |
| | CCGACTCTGTGAAGGGGAGGTTC ACAATT AGCAAAGATAATGCC |
| | AAAAACACTCTTTACCTTCAGCTCAATTCTTTGAAAACTGAGGAT |
| | ACTGCAATGTATTACTGCGCCCAGGGCGTATACGGCGACACTTAC |
| | TCCGGGAGCCAAGGAACACAGGTGACTGTATCTTCT (SEQ ID NO: |
| | 344) |
| mIL23R | CAGGTCCAGTTGCAAGAGTCAGGGGGTGGGTCCGTACAGGCAGG |
| VHH3 | AGGTTCCCTCCGCCTCAGCTGCACTGCCTCTGTCAACACATACTG |
| | CGAATACAATATGTCCTGGTATCGGCAGGCCCCTGGCAAGGAGA |
| | GAGAGTTCGTGTCCGGCGTTGACTCTGATGGTTCCACCCGCTACA |
| | GCGAGAGCGTTAAGGGGCGCTTCACCATCTCCCAGGACAACGCC |
| | AAGAACACTATGTACCTCCAGATGAATGGTCTGAAGCCCGAGGA |
| | CACCGCTATGTACTATTGTAAGACATACGTTTGTACCTTCTGTTC |
| | AGGCAACAGCTGCTACTATGAATATAAGTACTATTACGAGGGCC |
| | AGGGAACGCAGGTTACCGTATCCTCT (SEQ ID NO: 345) |
| mIL23R | CAGGTGCAACTCCAGGAGTCCGGCGGGGGTAGCGTCCAGGCTGG |
| VHH4 | GGGCTCCCTCCGCCTGAGTTGTGCTGCCAGCGGTTATACTTACTC |
| | TAATAATTGCATGGGCTGGTTTAGGCAAGCTCCGGGCAAGGACC |
| | GCGAGAGAATTGCCAATATCTACACAGGAGGTGGCAGAACTACC |
| | TACGCAGATAGTGTGAAGGGCCGGTTCACCATTTCTCAGGACAG |
| | TGCGAAGTCCACTGTGTACCTCCAGATGAACTCACTGAAGCCGG |
| | AGGACACCGCGATGTATTACTGCGCCGCTGGCTCCTGTGGGAGC |
| | GCTCGTTCCGAATACTCATACTGGGGCCAGGGCACCCAGGTGAC |
| | CGTGTCCTCC (SEQ ID NO: 346) |
| mIL23R | CAAGTGCAGCTCCAGGAATCTGGGGGCGGGTCTGTCCAAGCTGG |
| VHH5 | CGGGAGCCTCCGCCTGAGTTGTGCTGCCTCTGGGTACACCTTTTG |
| | TATGGCTTGGTTCCGCCAAGCGCCTGGGAAGGAACGCGAGGGTG |
| | TCGCACGCTTCTATACACGTGATGGATACACAT ATTACTCTGACA |
| | GCGTTAAGGGCAGATTCACTATCTCTCAGAATAACGCTAAGAAT |
| | ACCCTCTACTTGCAGATGAACTCTCTGAAAAGCGAGGACACCGC |
| | TATGTACTATTGCGCAGCGGATTTGGCCCGCTGTTCCAGCAACAA |
| | GAATGACTTTCGTTACTGGGGTCAGGGGACACAGGTGACAGTTA |
| | GTAGC (SEQ ID NO: 347) |
| mIL23R | CAGGTCCAGCTCCAGGAGTCAGGAGGTGGCTCCGTTCAGGCTGG |
| VHH6 | TGGCTCCCTCCGGCTGTCCTGTGCCGCAAGCGGATACACGTCTGG |
| | AAACTACTGGATGGGATGGTTCCGTCAAGCCCCCGGCAAAGAAC |
| | GCGAGGGCGTGGCTACTCTGTGGACTGGTGGAGCCTCAACCTTCT |
| | ACGGCGACTCTGTTAAGGGCCGTTTCACCATTAGTCGCGATAACT |
| | TCAAAAACACACTCTACCTTCAGATGAACTCCCTGAAGGTCGAG |
| | GATACAGCCATGTATTACTGCGCCGCTGACCCTGCCCTGCGTCTG |
| | GGAGCTAACATCCTGCGCCCTGCTGAATACAAATATTGGGGTCA |
| | AGGGACACAGGTGACTGTCAGCTCA (SEQ ID NO: 348) |
| mIL23R | CAGGTGCAGCTCCAGGAGTCCGGCGGTGGCCTGGTACAGCCCGG |
| VHH7 | TGGCAGCTTGCGCCTGAGCTGCGCCGCTTCTGGATTTACATTCTC |
| | CCGCAGCGCCATGACATGGGTTCGCCAGGCTCCAGGCAAGGGCC |
| | TCGACTGGGTGTCCGGCATTGACAGTGGCGGAACTACCGTGTAC |
| | GCAGATTCTGTTAAGGGAAGATTCACCATCTCCCGCGACTCCGCC |
| | AAGAACACCCTGTACTTGCAAATGAACAGCCTCAAGACAGAAGA |
| | CACAGCCGTGTATTACTGTGCCATCGGACTGCCGTGGGGCAACA |
| | CATGGCGTACCAGGGGACAGGGCACACAGGTGACAGTCTCCTCA |
| | (SEQ ID NO: 349) |
| mIL23R | CAGGTGCAGTTGCAGGAGTCCGGCGGAGGCAGCGTGCAAGCCGG |
| VHH8 | AGGTAGCCTCCGCCTGAGCTGCACAGCGAGCCGCTACACCTATA |
| | GCAGTTGCACTATGGGTTGGTATCGTCAGGCCCCCGGCAAGGAG |
| | AGGGAACTCGTGTCAATGGTGTTTTCTGACGGTTCCACCTTCTAC |
| | GCCGACTCCGTTAAAGGTCGGTTCACCTTCTCTCAGGAAAACGCC |
| | AAAAACACCGTGTACCTCCAGATGAACTCCCTGAAACCCGAGGA |
| | TACCGCTATGTACTATTGCGGATGCGCTACACTCGGCTCAAGAAC |
| | TATCTGGGGCCAAGGCACTCAGGTGACTGTCTCCTCC (SEQ ID |
| | NO: 350) |
| mIL23R | CAGGTGCAGCTCCAGGAAAGCGGAGGTGGCCTCGTGC AGCCTGG |
| VHH9 | TGGCTCCCTGAGACTGTCTTGCGCAACATCTGGATTCACCTTCAG |
| | GCTCACTGCTATGCGTTGGGTGAGACAAGCCCCAGGGAAGGGCG |
| | TCGAATGGGTGTCTGGAATCGACTCCGCTGGCTCTACGATCTACG |
| | CCGACAGCGTGAAGGGCCGCTTCACTATCTCCAAAGATAATGCT |
| | AAGAACACTCTGTATCTGCAAATGAACAGCCTCAAAACCGAAGA |
| | CACAGCTATGTACTATTGTGCGCAGGGTGTCTACGGTGATACCTA |
| | CAGCGGTTCTCAGGGCACTCAGGTGACGGTGTCCAGC (SEQ ID |
| | NO: 351) |
| mIL23R | CAGGTCCAGCTCCAGGAGAGCGGCGGGGGCAGCGTGCAGGCTGG |
| VHH10 | GGGTTCCCTGAGACTGTCCTGTGCTGCCTCCGGCGATACCTACAG |
| | TTCATGTACTATGGGCTGGTATCGCCAGGCTCCGGGCAAGGAGC |
| | GCGACCTCGTGAGCATGTTGATGGGCGATGGTAGCACCTTTTACG |
| | CCGATAGTGTGAAGGGCCGTTTCACCTTTAGCCAGGAGAACGCT |
| | AAAAACACTGTGTACTTGCAGATGAACAGCTTGAAGCCCGAGGA |
| | CACTGCAATGTATTACTGTGGCTGCGCCACGCTGGGCTCCCGCAC |
| | AATTTGGGGCCAGGGCACCCAGGTGACCGTTTCTAGC (SEQ ID |
| | NO: 352) |
| mIL23R | CAGGTCCAGCTCCAGGAAAGCGGCGGTGGAAGCGTGCAGGCCGG |
| VHH11 | TGGCAGTCTTCGCCTGTCTTGCGCTGCGTCTGGTTACACCTACTCC |
| | TCATGCACTATGGGTTGGTACAGGCAGGCCCCAGGGAAGGAGCG |
| | CGAGCTGGTATCCATGCTCATTTCTGACGGGTCCACCTTCTACGC |
| | CGATTCTGTCAAGGGCAGGTTTACCTTTTCCCAGGAAAACGCCAA |
| | GTCCACTGTCTATCTGCAAATGAACTCCTTGAAGCCCGAAGACAC |
| | TGCCATGTATTACTGTGGCTGTGCGACGCTTGGCTCAAGGACGGT |
| | GTGGGGCCAGGGCACGCAGGTAACCGTTTCCAGC (SEQ ID NO: |
| | 353) |
| mIL23R | CAGGTGCAGCTCCAAGAGTCTGGCGGAGGCTCCGTGCAGGCTGG |
| VHH12 | CGGGAGCCTGCGCTTGTCCTGCGCAGCCAGCGGCTATACCTACTC |
| | CTCTTGTACTATGGGCTGGTATAGGCAAGCGCCTGGCAAGGAGC |
| | GCGAACTCGTCAGCATGTTGATCTCTGACGGAAGCACCTTCTACG |
| | CTGATTCTGTGAAGGGGCGTTTTACCTTCAGCCAGGAGAACGCTA |
| | AGAACACCGTGTACCTCCAGATGAACTCTCTGAAACCTGAAGAT |
| | ACCGCTATGTACTATTGCGGATGCGCTACCCTGGGTTCTAGGACC |
| | GTTTGGGGTCAGGGAACACAGGTGAC AGTATCTTCC (SEQ ID NO: |
| | 354) |
| mIL23R | CAGGTCCAGCTCCAGGAATCTGGCGGGGGCCTCGTGCAACCAGG |
| VHH13 | GGGTTCCCTGAGATTGTCTTGCGCAACCAGTGGTTTTACCTTCCG |
| | CCTCGCTGCCATGCGTTGGGTGCGGCAAGCGCCCGGCAAGGGCC |
| | TGGAGTGGGTGTCTGGGATTGATTCTCGGGGCTCCACAATCTACG |
| | CGGACAGCGTCAAGGGTCGCTTCACTATCTCTAAGGACAATGCT |
| | AAGAACACTCTGTACCTTCAGTTGAACTCTCTGAAAACCGAGGAT |
| | ACCGCTATGTATTACTGTGCTCAGGGAGTGTATGGCGATACATAC |
| | TCCGGCTCCCAGGGGACGCAAGTGACTGTGAGTTCT (SEQ ID NO: |
| | 355) |
| mIL23R | CAGGTGCAGCTTCAGGAGTCCGGCGGGGGCCTGGTTCAGCCCGG |
| VHH14 | TGGGTCCCTGCGCCTGTCATGCGCAGCTTCCGGCTTCACCTTTAG |
| | GACATCTGCCATGACTTGGGTGCGTCAGGCTCCTGGCAAGGGCCT |
| | CGACTGGGTGAGCGGCATCGACAGCGGGGGAACCACAGTGTATG |
| | CCGACTCCGTCAAGGGACGCTTCACCATTAGCCGCGACTCCGCCA |
| | AGAACACCCTCTACCTTCAGATGAACAGCCTGAAGACGGAAGAC |
| | ACCGCCGTTTATTACTGCGCAATGGGGCTGCCTTGGGGCAACACC |
| | TGGAGGACTCGGGGCCAGGGAACTCAGGTGACCGTGTCTTCC |
| | (SEQ ID NO: 356) |
| mIL23R | CAGGTGCAGCTTCAGGAGTCAGGCGGGGGCAGCGTGCAGGCCGG |
| VHH15 | AGGCTCCTTGAGGCTGAGTTGCGCGGCCAGCGGCTACACATATTC |
| | TAGCTGCACAATGGGGTGGTATCGCCAGGCACCCGGAAAGGAGA |
| | GGGAACTCGTGTCTATGGTGTTCTCTGACGGCTCCACATTCTACG |
| | CCGATTCTGTGAAGGGCCGGTTTACCTTCTCACAGGAGAATGCCA |
| | AAAACACCGTGTATCTCCAGATGAACTCTTTGAAGCCAGAGGAC |
| | ACAGCCATGTATTACTGTGGATGCGCTACCCTGGGCTCCCGTACC |
| | ATCTGGGGTCAGGGCACCCAGGTGACTGTCAGCTCT (SEQ ID NO: |
| | 357) |
| mIL23R | CAAGTCCAGCTCCAGGAGAGCGGGGGCGGTTCCGTCCAGGCGGG |
| VHH16 | CGGAAGCCTCCGCCTTTCATGTGCAGCTAGTGGCGACACGTACA |
| | GCTCCTGTACTATGGGCTGGTACAGGCAGGCCCCAGGTAAGGAG |
| | CGCGATCTGGTGTCTATGCTGATGGGCGACGGCAGTACCTTTTAC |
| | GCTGATAGCGTCAAGGGCCGTTTCACCTTTTCTCAGGAGAACGCC |
| | AAGAATACCGTCTATCTTCAAATGAATAACCTCAAGCCAGAAGA |
| | TACTGCTATGTACTATTGTGGTTGTGCCACCCTGGGGTCCAGAAC |
| | AATCTGGGGACAGGGCACCCAGGTCACTGTGTCCTCT (SEQ ID |
| | NO: 358) |
| mIL23R | CAAGTCCAGCTTCAGGAGTCTGGCGGGGGCTCAGTGCAAGCAGG |
| VHH17 | AGGTAGCCTGAGGCTGAGCTGCGCTGCCAGTGGTTTTACTTTCCG |
| | CCTCACCGCCATGCGCTGGGTGCGCCAGGCCCCCGGCAAGGGCC |
| | TGGAGTGGGTGAGCGGAATCGACTCCAGGGGCAGCACTATTTAT |
| | GCCGACTCAGTGAAGGGGAGATTTACTATCTCCAAGGACAATGC |
| | AAAAAACACCCTTTACCTTCAACTGAACTCTTTGAAGACCGAGG |
| | ACACGGCCATGTATTACTGCGCACAGGGAGTCTACGGGGACACC |
| | TACTCTGGCTCTCAGGGCACCCAGGTCACTGTGTCTAGC (SEQ ID |
| | NO: 359) |
| mIL23R | CAAGTCCAGCTCCAGGAGAGCGGCGGGGGCCTGGTGCAGCCCGG |
| VHH18 | TGGCTCTTTGAGGCTCAGCTGTGCTGCCTCCGGCTTCACATTCCG |
| | CCTGACTGCAATGCGTTGGGTGAGGCAGGCTCCTGGCAAGGGTC |
| | TGGAGTGGGTCTCTGGTATCGACAGTAGAGGCTCCACCATCTACG |
| | CAGATAGCGTAAAGGGACGCTTCACCATCTCCAAAGATAACGCT |
| | AAGAACACCCTCTACCTCCAGCTTAACAGCCTGAAGACCGAGGA |
| | CACAGCTATGTACTATTGTGCACAAGGCGTCTACGGCGATACCTA |
| | TTCCGGTTCCCAGGGCACTCAGGTGACCGTCTCCTCC (SEQ ID NO: |
| | 360) |
| mIL23R | CAGGTTCAGCTTCAGGAGAGCGGCGGTGGCCTGGTCCAACCTGG |
| VHH19 | GGGAAGCCTCCGTCTGAGCTGCGCCGCATCTGGATTCACCTTTAG |
| | GCTGTCAGCTATGCGCTGGGTCCGTCAGGCCCCAGGGAAGGGCC |
| | TGGAATGGGTTAGCGGGATCGACTCTCGCGGGTCTACGATTTATG |
| | CCGACTCAGTCAAGGGGCGCTTCACGATCTCTAAGGACAACGCT |
| | AAGAACACCCTGTACTTGCAGCTGAACAGCCTGAAGACCGAGGA |
| | TACGGCTATGTATTACTGTGCGCAGGGGGTCTACGGGGACACCT |
| | ACTCAGGATCACAGGGCACCCAAGTGACCGTGAGTTCC (SEQ ID |
| | NO: 361) |
| mIL23R | CAGGTACAGCTCCAGGAGTCCGGCGGAGGCCTTGTGCAGCCTGG |
| VHH20 | TGGCTCCTTGAGACTGAGCTGTGCCGCTTCCGGTTTTACATTCTCC |
| | AGCTCAGCCATGACATGGGTGAGACAGGCCCCTGGAAAGGGACT |
| | GGACTGGGTTTCTGGCATTGACTCAGGGGGCACGACCGTGTATG |
| | CTGACTCTGTTAAGGGCCGCGCCACCATCCTCAAGGACAACGCT |
| | AAGAACACACTCTACCTCCAGATGAACTCCCTGAAGACTGAGGA |
| | CACAGCTGTCTACTATTGTGCTACTGGTCTGCCTTGGGGTAACAC |
| | CTGGCGGACCAGGGGCCAGGGCACTCAGGTGACTGTCTCCTCA |
| | (SEQ ID NO: 362) |
| mIL23R | CAGGTGCAGCTCCAGGAGTCAGGCGGTGGACTCGTTCAGCCGGG |
| VHH21 | TGGCTCCCTGCGCCTCAGTTGTGCGACCTCTGGCTTTACCTTCTCC |
| | AGCTCCGCTATGACCTGGGTGAGGCAAGCACCTGGGAAGGGCCT |
| | CGATTGGGTCTCCGGCATTGATTCTGGAGGCACCACTGTCTACGC |
| | CGACAGCGTGAAGGGCAGATTCACAATCAGTAAGGACAACGCTA |
| | AGAACACTCTGTACCTGCAAATGAACAGCCTGAAGACCGAGGAC |
| | ACCGCTGTTTATTACTGCGCAACGGGACTGCCTTGGGGTAATACT |
| | TGGAGGACTACCGGCCAGGGAACTCAGGTGACTGTGAGTTCC |
| | (SEQ ID NO: 363) |
| mIL23R | CAGGTGCAGCTCCAGGAATCCGGTGGAGGCTCCGTGCAAGCGGG |
| VHH22 | CGGGTCCCTGCGCCTCAGCTGTGCAGCTTCTGGCTATACCTTCTG |
| | CATGGCCTGGTTTCGCCAGGCCCCTGGGAAGGAGAGGGAGGGGG |
| | TGGCCCGCTTTTACACTAGAGACAGCTATACTTACTATAGCGACT |
| | CCGTGAAGGGGCGCTTTACGATTAGCCAGAATAACGCCAAGAAT |
| | ACCTTGTACCTCCAGATGAATAGTCTGAAGTCCGAGGACACCGC |
| | CATGTATTACTGTGCTGCCGACCTTACGAGGTGCAGCTCCAATAA |
| | GAACGACTTCCGCTACTGGGGCCAGGGTACTCAGGTCACTGTGTC |
| | CAGC (SEQ ID NO: 364) |
| mIL23R | CAAGTGCAGCTCCAGGAAAGCGGAGGCGGTCTGGTCCAACCAGG |
| VHH23 | AGGGTCCCTGCGTCTGTCCTGCGCGGCCTCCGGCTTTAATTTCAG |
| | ACTGTATGCGATGCGTTGGGTTCGTCAAGCGCCCGGTAAGGGCG |
| | TGGAGTGGGTGTCCGGTATCGACTCAGGAGGCTCTACCATCTATG |
| | CTGACTCTGTGAAGGGCCGCTTTACCATCAGCAAGGACAACGCT |
| | AAAAATACCCTGTACTTGCAGCTGAACTCTCTGAAAACCGAGGA |
| | CACTGCCATGTATTACTGCGCCCAGGGTGTGTACGGCGACACCTA |
| | CTCTGGTTCCCAAGGCACCCAGGTGACGGTCTCCTCC (SEQ ID |
| | NO: 365) |
| mIL23R | CAAGTGCAGCTCCAGGAGAGCGGTGGCGGTTCTGTGCAAGCGGG |
| VHH24 | TGGGTCCCTGCGGCTGAGCTGCGCTGTGTCTGGTTATACCTTCTG |
| | TATGGCCTGGTTCCGCCAGGCTCCGGGAAAGGAGCGCGAAGGCG |
| | TGGCTCGGTTCTACACCAGAGACGGTTACACATACTATTCCGGCA |
| | GCGTGAAGGGCAGGTTCACGATCAGCCAGAATAACGCTAAAAAC |
| | ACCCTGTACCTGCAAATGAACAGCCTGAAGAGCGAGGATACCGC |
| | GATGTATTACTGCGCAGCCGACTTGACCAGATGCTCTTCCAACAA |
| | AAACGACTTCCGCTACTGGGGTCAGGGCACCCAAGTCACTGTGT |
| | CCTCC (SEQ ID NO: 366) |
| mIL23R | CAAGTCCAGCTCCAGGAGAGTGGAGGCGGAAGCGTGCAGGCCG |
| VHH25 | GTGGCTCCCTGAGACTTTCATGCGCAGCGTCCGGCTATACATATT |
| | CTTCCTGCACTATGGGCTGGTACAGACAAGCGCCGGGCAAGGAG |
| | CGTGAGTTGGTGAGTATGCTCATCAGCGATGGCAGTACCTTTTAT |
| | GCGGACTCTGTCAAGGGCCGCTTCACCTTCTCTCAAGAGAACGCT |
| | AAAAACACAGTTTACCTCCAGATGAACTCCCTGAAGCCCGAAGA |
| | CACTGCCATGTATTTTTGTGGGTGTGCCACTCTTGGCTCCAGGAC |
| | GGTGTGGGGCCAGGGCACCCAGGTTACCGTGAGCAGT (SEQ ID |
| | NO: 367) |
| mIL23R | CAGGTCCAGCTGCAAGAGTCTGGAGGGGGCAGCGTGCAGGCTGG |
| VHH26 | CGGTTCTCTGCGCCTGAGCTGCGCTGCGAGTGGGTACACTTTCTG |
| | TATGGCATGGTTTCGCCAAGCTCCCGGTAAGGAGCGCGAAGGTG |
| | TGGCCCGCTTTTATACTAGGGACGGTTACACATATTACTCAGACT |
| | CTGTGAAGGGCCGTTTTACCATTTCCCAAAACAATGCAAAAAAC |
| | ACCCTGTACCTTCAGATGAACTCTCTCAAAAGCGAGGATACTGCT |
| | ATGTATTACTGCGCCGCAGACCTGACCAGATGTTCATCCAATAAG |
| | AATGACTTCCGCTACTGGGGCCAAGGGACCCAGGTGACCGTGAG |
| | CAGT (SEQ ID NO: 368) |
| mIL23R | CAGGTCCAGCTGCAAGAATCTGGTGGGGGTTCCGTGCAAGCCGG |
| VHH27 | AGGCAGCCTGAGGCTCAGCTGCGCCGCAAGCGGATACACATATT |
| | CCTCTTGCACTATGGGTTGGTATCGCCAGGCCCCAGGCAAGGAA |
| | CGTGAGCTGGTCTCTATGCTCATCAGTGATGGCAGCACCTTTTAC |
| | GCTGATTCTGTGAAGGGCAGATTTACCTCTTCCCAGGAGAACGCT |
| | AAAAACACTGTGTACCTTCAGATGAACAGCCTGAAGCCAGAGGA |
| | CACCGCGATGTACTATTGCGGCTGCGCAACCCTGGGGAGCAGGA |
| | CAGTGTGGGGGCAGGGCACACAGGTGACCGTCTCCTCA (SEQ ID |
| | NO: 369) |
| mIL23R | CAGGTCCAGCTGCAAGAGAGCGGAGGCGGGCTCGTGCAGCCGGG |
| VHH28 | TGGCAGCCTGCGCCTTTCCTGCGCTGCGTCTGGCTTCACCTTCAG |
| | GCTCACCGCTATGAGATGGGTTAGACAAGCTCCCGGCAAGGGTC |
| | TGGAATGGGTGAGCGGCATCGACAGCAGAGGTAGCACGATCTAC |
| | GCTGATTCCGTCAAGGGACGGTTCACAATTTCCAGAGACAACGC |
| | CAAGAACACACTGTACCTTCAGTTGAACTCCCTGAAGACCGAAG |
| | ACGCCGCTATGTACTATTGTGCGCAGGGCGTGTACGGCGATACCT |
| | ACTCAGGCTCCCAGGGCACCCAGGTAACGGTGAGTTCC (SEQ ID |
| | NO: 370) |
| mIL23R | CAGGTACAGCTGCAAGAGAGTGGCGGAGGCCTCGTTCAACCCGG |
| VHH29 | AGGGAGTCTGCGCCTGTCTTGTGCTGCCTCCGGCTTCACCTTTTCC |
| | ACTTCCGCTATGACCTGGGTCAGGCAGGCCCCCGGCAAGGGACT |
| | GGACTGGGTAAGTGGCATCGACTCCGGTGGCACTACCGTGTACG |
| | CGGACTCCGTGAAAGGCCGCTTCACTATTAGCAAGGACAACGCT |
| | AAAAACACACTCTACCTCCAGATGAACAGCCTCAAGACAGAGGA |
| | CACTGCCGTGTACTATTGCGCGACCGGCCTGCCTTGGGGCAACAC |
| | CTGGCGCACAAGAGGTCAAGGGACACAGGTCACTGTGAGCAGC |
| | (SEQ ID NO: 371) |
| mIL23R | CAGGTGCAGCTGCAAGAAAGCGGAGGCGGACTTGTTCAGCCTGG |
| VHH30 | GGGCTCCTTGCGGCTGTCCTGTGCTGCCTCAGGCTTTACTTTTCGT |
| | CTGACAGCCATGCGGTGGGTGCGGCAGGCCCCTGGCAAGGGTCT |
| | CGAATGGGTTTCCGGTATTGACTCTCGCGGCTCTACTATCTACGC |
| | CGACTCTGTGAAGGGCCGTTTCACCATCTCCAAGGATAATGCCAA |
| | AAACACGCTGTACTTGCAGCTTAATAGCTTGAAGACCGAGGATA |
| | CGGCCATGTACTATTGTGCTCAGGGCGTTTACGGCGACACTTATT |
| | CTGGCTCCCTTGGCACGCAGGTCACGGTTTCTAGC (SEQ ID NO: |
| | 372) |
| mIL23R | CAGGTGCAGCTCCAGGAGAGCGGAGGCGGACTGGTGCAGCCAG |
| VHH31 | GTGGCAGCCTGAGGCTCTCCTGTGCGGCCTCAGGTTTTACCTTTC |
| | GCCTGACAGCCATGCGGTGGGTCAGACAAGCGCCTGGGAAAGGT |
| | CTGGAGTGGGTGTCTGGTATCGACTCTCGCGGTTCCACCATCTAC |
| | GCCGATTCTGTGAAGGGGCGCTTTACAATTAGCCGCGACAACGC |
| | CAAGAACACCCTGTACCTCCAGCTCAATTCCCTGAAGACCGAGG |
| | ACACCGCGATGTACTATTGTGCGCAAGGCGTCTATGGGGATACCT |
| | ATAGCGGTTCTCAGGGAACCCAGGTGACTGTTTCCAGC (SEQ ID |
| | NO: 373) |
| mIL23R | CAGGTGCAGCTCCAGGAATCTGGGGGAGGCCTGGTTCGCCCTGG |
| VHH32 | GGGTAGCCTGAGACTGAGCTGTGCAGCCTCTGGATTCACTTTCTC |
| | CCGTTCCGCAATGACCTGGGTCCGCCAGGCCCCAGGCAAGGGGT |
| | TGGATTGGGTGTCTGGCATTGATTCCGGGGGCACCACTGTGTACG |
| | CGGACTCCGTGAAGGGCCGCTTCACCATCAGCCGCGATAGCGCC |
| | AAAAACACGCTGTATCTCCAGATGAACAGCCTGAAGACCGAGGA |
| | CACTGCCGTCTACTATTGTGCTATCGGCCTGCCCTGGGGCAACAC |
| | ATGGCGTACACGCGGTCAGGGCACGCAGGTGACCGTGTCTTCT |
| | (SEQ ID NO: 374) |
| mIL23R | CAGGTCCAGCTTCAGGAAAGCGGAGGCGGACTGGTGCAGCCCGG |
| VHH33 | AGGCAGTCTGCGTCTCAGCTGTACGACCAGCGGGTTTACTTTCTC |
| | TAGTAGCGCAATGACTTGGGTGAGGCAGGCTCCGGGCAAGGGTC |
| | TGGACTGGGTCAGCGGTATCGACAGCGGCGGGACGACTGTGTAT |
| | GCCGATTCAGTGAAAGGACGGTTCACTATCTCAAAGGACAACGC |
| | CAAAAACACACTGTACCTTCAGATGAACTCCCTGAAGACCGAAG |
| | ACACAGCGGTGTATTACTGCGCCACAGGGTTGCCTTGGGGCAAC |
| | ACTTGGCGCACCACTGGACAAGGGACGCAGGTGACCGTTTCCTC |
| | T (SEQ ID NO: 375) |
| mIL23R | CAGGTGCAGCTCCAAGAGAGTGGCGGAGGCCTGGTGCAGCCCGG |
| VHH34 | TGGCTCTCTGAGGTTGTCTTGTGCTGCCTCTGGCTTTACCTTCAGA |
| | CTGACAGCCATGCGCTGGGTCCGCCAGGCTCCTGGTAAGGGACT |
| | GGAGTGGGTAAGCGGTATCGACTCCAGAGGGAGCACCATCTATG |
| | CTGATTCCGTTAAGGGACGGTTCACCATCTCTAAGGATAATGCCA |
| | AGAACACCCTGTATCTCCAGTTGAACTCCCTGAAAACCGAGGAC |
| | ACCGCGATGTACTATTGCGCACAGGGCGTGTATGGCGACACTCA |
| | CAGCGGCTCTCAAGGCACCCAGGTGACCGTGTCTTCC (SEQ ID |
| | NO: 376) |
| mIL23R | CAGGTCCAGCTCCAAGAATCCGGCGGAGGGCTGGTACAGCCAGG |
| VHH35 | AGGCAGTCTTAGGCTGGCTTGCTCTGCGTCCGGCTTCACATTTTC |
| | CAGCTCTGCCATGACCTGGGTGCGCCAGGCACCCGGAAAGGGCC |
| | TGGACTGGGTGAGCGGGATTGATAGCGGAGGCACCACGGTGTAT |
| | GCTGACAGTGTAAAAGGACGCGCCACTATCCTGAAGGACAATGC |
| | CAAGAACACCCTCTATTTGCAGATGAACAGCCTGAAGACTGAAG |
| | ATACTGCTGTGTATTACTGTGCAACGGGCCTGCCTTGGGGAAACA |
| | CTTGGCGGACGCGGGGCCAGGGCACGCAGGTGACCGTGTCTTCC |
| | (SEQ ID NO: 377) |
| mIL23R | CAGGTTCAGCTGCAAGAATCTGGTGGCGGAAGCGTGCAAGCGGG |
| VHH36 | TGGCTCTCTTCGTCTCTCTTGTGCTGCATCCGGCGACACCTACAG |
| | CTCCTGCACAATGGGGTGGTATCGTCAGGCCCCTGGCAAGGAGC |
| | GGGATCTGGTCAGCATGGTCTTCTCTGACGGCAGCACATTCTACG |
| | CTGACTCCGTCAAGGGACGTTTCACCTTCTCTCAGGAGAACGCGA |
| | AGAATACTGTGTATCTTCAGATGAACAGCCTGAAGCCGGAGGAT |
| | ACAGCAATGTATTACTGCGGTTGCGCGACCCTGGGTAGCAGGAC |
| | CATCTGGGGTCAAGGCACCCAGGTGACAGTGTCCTCC (SEQ ID |
| | NO: 378) |
| mIL23R | CAGGTCCAGCTTCAGGAATCAGGAGGCGGGCTTGTGCAGCCGGG |
| VHH37 | AGGCAGCCTGCGCCTGTCCTGCGCAACCTCCGGCTTTACCTTCTC |
| | CAGCGGAGCCATGACCTGGGTGCGGCAGGCCCCCGGTAAGGGCC |
| | TGGATTGGGTGTCTGGCATCGACTCCGGCGGAACCACTGTGTACG |
| | CTGATTCTGTGAAGGGTCGCTTCACAATTAGTAAGGACAACGCTA |
| | AGAACACCCTGTACCTCCAGATGAACTCATTGAAGACAGAGGAT |
| | ACCGCCGTGTACTATTGTGCAACCGGCCTCCCCTGGGGGAACACC |
| | TGGCGCACCACTGGTCAGGGAACACAAGTAACCGTGAGCAGC |
| | (SEQ ID NO: 379) |
| mIL23R | CAGGTCCAGCTCCAGGAGAGTGGCGGGGGACTCGTGCAGCCTGG |
| VHH38 | TGGCTCTTTGCGCCTGAGCTGCGCGGCAAGCGGATTTAC ATTTTC |
| | CACCAGTGCTATGACCTGGGTGCGCCAGGCTCCCGGCAAGGGAC |
| | TGGACTGGGTAAGCGGTATTGATTCCGGCGGAACGACTGTGTAC |
| | GCTGATAGCGTAAAGGGCCGCTTTACCATCAGC AAAGACAACGC |
| | CAAAAATACCCTTTACCTGCAAATGAACTCTTTGAAGACGGAGG |
| | ACACCGCTGTGTATTACTGCGCCACTGGCCTCCCTTGGGGCAACA |
| | TCTGGAGAACCCGTGGTCAGGGCACCCAGGTTACCGTGTCCTCC |
| | (SEQ ID NO: 380) |
| mIL23R | CAGGTCCAACTCCAGGAGTCCGGCGGAGGCTTGGTGCAGCCTGG |
| VHH39 | AGGCTCTCTGCGGCTGTCCTGCGCCGCATCAGGTTTTACGTTTTCT |
| | CGGTCTGCCATGACCTGGGTCAGACAGGCACCAGGCAAGGGCCT |
| | GGATTGGGTGTCCGGTATTGACTCTGGTGGCACTACCGTGTATGC |
| | CGACTCCGTTAAGGGCCGTTTCACCATCTCCAGGGACTCTGCCAA |
| | GAACACATTGTATTTGCAAATGAACGGCCTCAAAACTGAGGACA |
| | CCGCAGTCTACTATTGTGCAATCGGGCTTCCGTGGGGCAACACGT |
| | GGAGAACCAGGGGCCAGGGGACTCAGGTCACCGTGTCATCC |
| | (SEQ ID NO: 381) |
| mIL23R | CAGGTCCAGCTCCAGGAGTCAGGTGGAGGCCTGGTGCAACCCGG |
| VHH40 | AGGCTCCCTCCGCCTGTCCTGTGCAGCCTCCGGCTTTACCTTTCGC |
| | CTGACCGCGATGAGGTGGGTTCGGCAGGCCCCTGGCAAAGGGCT |
| | GGAGTGGGTTAGCGGCATCGACTCCAGGGGCTCCACCATTTACG |
| | CCGACTCTGTCAAGGGGCGTTTCACCATTTCTAAGGACAACGCTA |
| | AGAATACCCTCTACCTCCAGCTCAACTCCCTGAAGAGTGAAGAC |
| | ACCGCCATGTATTACTGTGCCCAGGGCGTCTACGGAGACACTTAC |
| | AGCGGGTCCCAGGGTACTCAGGTGACCGTGTCTTCC (SEQ ID NO: |
| | 382) |
| mIL23R | CAAGTCCAGTTGCAGGAGTCAGGAGGTGGCCTGGTGCAACCCGG |
| VHH41 | TGGCTCCCTCCGTCTGACCTGCGCTGCGTCTGGTTTCACTTTCTCA |
| | ACTTCAGCTATGACATGGGTCCGCCAGGCACCGGGGAAGGGCCT |
| | CGACTGGGTATCTGGGATCGACAGCGGAGGCACCACTGTCTATG |
| | CCGATTCCGTGAAAGGACGCTTCACTATTAGCAAGGACAACGCT |
| | AAAAACACCCTGTATTTGCAGATGAAT AGCCTCAAAACTGAAGA |
| | TACTGCCGTTTACTATTGCGCCACTGGCCTCCCCTGGGGCAACAC |
| | CTGGCGCACAAGGGGTCAGGGTACTCAGGTAACCGTGTCCTCT |
| | (SEQ ID NO: 383) |
| mIL23R | CAGGTGCAGCTGCAAGAGTCCGGTGGCGGTCTGGTGCAGCCCGG |
| VHH42 | AGGCAGTCTGAGGCTCTCCTGCGCTGCCTCTGGATTCACCTTCAG |
| | CAACTACGCTATGCGCTGGGTGCGGCAGGCCCCCGGCAAGGGCC |
| | TGGAGTGGGTCAGTGGCATCGACAGTCGCGGAAGTACTATTTAT |
| | GCCGACTCCGTGAAGGGAAGGTTCACTATTTCCAAGGACAACGC |
| | CAAAAACACTCTGTACTTGCAGCTGAACTCCTTGAAGACTGAGG |
| | ACACTGCCATGTACTATTGCGCCCAGGGAGTCTATGGGGACACCT |
| | ATTCCGGGAGCCAGGGCACTCAGGTGACCGTGTCAAGT (SEQ ID |
| | NO: 384) |
| mIL23R | CAGGTTCAGCTCCAGGAATCCGGTGGAGGGTCCGTGCAGGCCGG |
| VHH43 | GGGCAGCCTGAGACTGTCCTGTGCTGCCTCTGGTTATACGTTTTG |
| | CATGGCGTGGTTCCGGCAGGCTCCTGGAAAAGAGCGCGAGGGCG |
| | TGGCAAGATTCTACACTAGAGATGGTTACACCTATTACTCCGACT |
| | CTGTCAAGGGGAGGTTTACCATCTCTCAGGACAACGCCAAGAAC |
| | ACTTTGTACCTCCAGATGAACTCCCTGAAGTCTGAGGACACCGCC |
| | ATGTATTACTGTGCAGCCGATCTGACCCGGTGC AGTTCCAACAAG |
| | AACGATTTCCGCTATTGGGGCCAGGGCACACAGGTCACAGTCTC |
| | CTCC (SEQ ID NO: 385) |
| mIL23R | CAAGTACAGCTCCAAGAGTCTGGGGGAGGTCTTGTGCAGCCCGG |
| VHH44 | AGGCTCTTTGCGTCTGTCATGTGCGGCCAGCGGATTCACATTCAG |
| | GCTGTCTGCAATGCGTTGGGTGCGCCAAGCGCCTGGCAAGGGGT |
| | TTGAATGGGTGTCTGGAATTGATTCCCGTGGCTCTACCATCTATG |
| | CCGATTCTGTTAAAGGCCGCTTTACCATCTCCAAGGATAACGCAA |
| | AAAACACACTGTACTTGCAGCTGAATAGCCTGAAAACTGAGGAC |
| | ACCGCTATGTATTACTGCGCTCAGGGAGTGTATGGCGACACTTAT |
| | TCCGGCAGCCAGGGCACTCAGGTGACAGTTAGCTCC (SEQ ID NO: |
| | 386) |
| mIL23R | CAGGTGCAGCTCCAGGAGTCCGGCGGTGGACTCGTGCAACCCGG |
| VHH45 | CGGTTCCCTTAGATTGTCTTGCGCCGCTTCAGGTTTTACCTTTCGC |
| | TTGTCCGCTATGCGGTGGGTTCGCCAAGCGCCAGGAAAAGGCCT |
| | GGAGTGGGTCTCCGGTATTGATTCCAGAGGCTCCACCATCTACGC |
| | CGACTCTGTCGAGGGCAGGTTCACCATCAGCAAGGACAACGCAA |
| | AGAACACCCTGTATCTTCAGCTTAATAGTCTGAAGACCGAGGAC |
| | ACTGCGATGTATTACTGCGCTCAGGGAGTGTACGGTGATACCTAC |
| | TCCGGCTCCCAGGGAACTCAGGTGACCGTCTCCAGC (SEQ ID NO: |
| | 387) |
| mIL23R | CAAGTTCAGTTGCAGGAGAGCGGAGGGGGCCTGGTTCAGCCGGG |
| VHH46 | AGGCTCCCTGAGGCTGTCCTGCGCTGCGAGTGGCTTCACTTTTAG |
| | GTTGTCCGCTATGCGCTGGGTGCGCCAGGCTCCTGGGAAGGGTCT |
| | GGAGTGGGTGTCTGGGATTGACTCCAGAGGTAGTACCATTTACG |
| | CCGACTCCGTCAAGGGACGCTTCACCATCTCCAAGGACGATGCC |
| | AAGAACACCCTGTATCTCCAGCTGAACTCACTCAAGACCGAAGA |
| | CACGGCAATGTATTACTGTGCCCAGGGTGTGTATGGTGACACTTA |
| | CTCTGGCTCTCAGGGCACTCAAGTGACCGTTTCTTCC (SEQ ID NO: |
| | 388) |

1. An IL-23 receptor binding molecule that specifically binds to IL-12Rb1 and IL-23R,
wherein the binding molecule causes the multimerization of IL-12Rb1 and IL-23R when bound to IL-12Rb1 and IL-23R, and
wherein the binding molecule comprises a single-domain antibody (sdAb) that specifically binds to IL-12Rb1 and a sdAb that specifically binds to IL-23R.

2. The IL-23R binding molecule of claim 1, wherein the anti-IL-12Rb1 sdAb is a V_{H}H antibody and/or the anti-IL-23R sdAb is a V_{H}H antibody.

3. The IL-23 receptor binding molecule of claim 1, wherein the anti-IL-12Rb1 sdAb and the anti-IL-23R sdAb are joined by a peptide linker.

4. The IL-23 receptor binding molecule of claim 3, wherein the peptide linker comprises between 1 and 50 amino acids.

5. The IL-23 receptor binding molecule of claim 4, wherein the peptide linker comprises a sequence of GGGS (SEQ ID NO: 13).

6. The IL-23 receptor binding molecule of claim 2, wherein the anti-IL-12Rb1 sdAb comprises one or more CDRs in a row of Table 2 or 3 wherein each CDR independently comprises 0, 1, 2, or 3 amino acid changes relative to the sequence of Table 2 or 3.

7. The IL-23 receptor binding molecule of claim 2, wherein the anti-IL-23R sdAb comprises one or more CDRs (e.g., CDR1, CDR2, and CDR3) in a row of Table 4 or 5 wherein each CDR independently comprises 0, 1, 2, or 3 amino acid changes relative to the sequence of Table 4 or 5.

8. The IL-23 receptor binding molecule of claim 2, wherein the IL-23 receptor binding molecule comprises
an anti-IL-12Rb1 sdAb comprising a CDR1, a CDR2, and a CDR3 in a row of Table 2 or 3, and
an anti-IL-23R sdAb a CDR1, a CDR2, and a CDR3 in a row of Table 4 or 5.

9. The IL-23 receptor binding molecule of claim 1, wherein the binding molecule comprises an anti-IL-12Rb1 sdAb linked to the N-terminus of a linker and an anti-IL-23R sdAb linked to the C-terminus of the linker.

10. The IL-23 receptor binding molecule of claim 1, wherein the binding molecule comprises an anti-IL-23R sdAb linked to the N-terminus of a linker and an anti-IL-12Rb1 sdAb linked to the C-terminus of the linker.

11. The IL-23 receptor binding molecule of claim 9, wherein the anti-IL-12Rb1 sdAb comprises a sequence having at least 90% sequence identity to a sequence of Table 6 or 7.

12. The IL-23 receptor binding molecule of claim 9, wherein the anti-IL-12Rb1 sdAb comprises a sequence of Table 6 or 7.

13. The TL-23 receptor binding molecule of claim 9, wherein the anti-IL-23R sdAb comprises a sequence having at least 90% sequence identity to a sequence of Table 8 or 9.

14. The IL-23 receptor binding molecule of claim 9, wherein the anti-IL-23R sdAb comprises a sequence of Table 8 or 9.

15. The IL-23 receptor binding molecule of claim 9, wherein each of the anti-IL-12Rb1 sdAb comprises a sequence having at least 90% sequence identity to a sequence of Table 6 or 7 and the anti-IL-23R sdAb comprises a sequence having at least 90% sequence identity to a sequence of Table 8 or 9.

16. An isolated nucleic acid encoding the IL-23 receptor binding molecule of claim 1.

17. An expression vector comprising the nucleic acid of claim 16.

18. An isolated host cell comprising the vector of claim 17.

19. A pharmaceutical composition comprising the IL-23 receptor binding molecule of claim 1 and a pharmaceutically acceptable carrier.

20. A method of treating an autoimmune or inflammatory disease, disorder, or condition or a viral infection in a subject in need thereof, comprising administering to the subject a therapeutically effective amount of an IL-23 receptor binding molecule of claim 1 or a pharmaceutical composition of claim 19.

21. The method of claim 20, further comprising administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody.

22. The method of claim 21, wherein the therapeutic antibody is an antibody that binds a protein selected from the group consisting of BLyS, CD11a, CD20, CD25, CD3, CD52, IgEIL12/IL23, IL17a, IL1β, IL4Rα, IL5, IL6R, integrin-α4β7, RANKL, TNFα, VEGF-A, and VLA-4.

23. The method of claim 20, wherein the disease, disorder, or condition is selected from a neoplastic disease, viral infections, Heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, Pityriasis rubra pilaris, Pityriasis rosacea, parapsoriasis, Pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis; seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.


SEQUENCE LISTING (390 sequences)

SEQ ID NO: 1 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 2 - protein, 522 aa, Homo sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 3 - protein, 738 aa, Mus musculus
MDMMGLAGTSKHITFLLLCQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAKIKFLVEPLNQGGRRRLTMQGQSPQLAVPEGCRGRPGAQVKKHLVLVRMLSCRCQAQTSKTVPLGKKLNLSGATYDLNVLAKTRFGRSTIQKWHLPAQELTETRALNVSVGGNMTSMQWAAQAPGTTYCLEWQPWFQHRNHTHCTLIVPEEEDPAKMVTHSWSSKPTLEQEECYRITVFASKNPKNPMLWATVLSSYYFGGNASRAGTPRHVSVRNQTGDSVSVEWTASQLSTCPGVLTQYVVRCEAEDGAWESEWLVPPTKTQVTLDGLRSRVMYKVQVRADTARLPGAWSHPQRFSFEVQISRLSIIFASLGSFASVLLVGSLGYIGLNRAAWHLCPPLPTPCGSTAVEFPGSQGKQAWQWCNPEDFPEVLYPRDALVVEMPGDRGDGTESPQAAPECALDTRRPLETQRQRQVQALSEARRLGLAREDCPRGDLAHVTLPLLLGGVTQGASVLDDLWRTHKTAEPGPPTLGQEA

SEQ ID NO: 4 - protein, 546 aa, Mus musculus
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAKIKFLVEPLNQGGRRRLTMQGQSPQLAVPEGCRGRPGAQVKKHLVLVRMLSCRCQAQTSKTVPLGKKLNLSGATYDLNVLAKTRFGRSTIQKWHLPAQELTETRALNVSVGGNMTSMQWAAQAPGTTYCLEWQPWFQHRNHTHCTLIVPEEEDPAKMVTHSWSSKPTLEQEECYRITVFASKNPKNPMLWATVLSSYYFGGNASRAGTPRHVSVRNQTGDSVSVEWTASQLSTCPGVLTQYVVRCEAEDGAWESEWLVPPTKTQVTLDGLRSRVMYKVQVRADTARLPGAWSHPQRFSFEVQIS

SEQ ID NO: 5 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 6 - protein, 332 aa, Homo sapiens
GITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIG

SEQ ID NO: 7 - protein, 644 aa, Mus musculus
MSHLTLQLHVVIALYVLFRWCHGGITSINCSGDMWVEPGEIFQMGMNVSIYCQEALKHCRPRNLYFYKNGFKEEFDITRINRTTARIWYKGFSEPHAYMHCTAECPGHFQETLICGKDISSGHPPDAPSNLTCVIYEYSGNMTCTWNTGKPTYIDTKYIVHVKSLETEEEQQYLASSYVKISTDSLQGSRKYLVWVQAVNSLGMENSQQLHVHLDDIVIPSASIISRAETTNDTVPKTIVYWKSKTMIEKVFCEMRYKTTTNQTWSVKEFDANFTYVQQSEFYLEPDSKYVFQVRCQETGKRNWQPWSSPFVHQTSQETGKRNWQPWSSPFVHQTSQTVSQVTAKSSHEPQKMEMLSATIFRGHPASGNHQDIGLLSGMVFLAIMLPIFSLIGIFNRSLRIGIKRKVLLMIPKWLYEDIPNMENSNVAKLLQEKSVFENDNASEQALYVDPVLTEISEISPLEHKPTDYKEERLTGLLETRDCPLGMLSTSSSVVYIPDLNTGYKPQVSNVPPGGNLFINRDERDPTSLETTDDHFARLKTYPNFQFSASSMALLNKTLILDELCLVLNQGEFNSLDIKNSRQEETSIVLQSDSPSETIPAQTLLSDEFVSCLAIGNEDLPSINSYFPQNVLESHFSRISLFQK

SEQ ID NO: 8 - protein, 351 aa, Mus musculus
GITSINCSGDMWVEPGEIFQMGMNVSIYCQEALKHCRPRNLYFYKNGFKEEFDITRINRTTARIWYKGFSEPHAYMHCTAECPGHFQETLICGKDISSGHPPDAPSNLTCVIYEYSGNMTCTWNTGKPTYIDTKYIVHVKSLETEEEQQYLASSYVKISTDSLQGSRKYLVWVQAVNSLGMENSQQLHVHLDDIVIPSASIISRAETTNDTVPKTIVYWKSKTMIEKVFCEMRYKTTTNQTWSVKEFDANFTYVQQSEFYLEPDSKYVFQVRCQETGKRNWQPWSSPFVHQTSQETGKRNWQPWSSPFVHQTSQTVSQVTAKSSHEPQKMEMLSATIFRGHPASGNHQDIG

SEQ ID NO: 9-12 - skipped (4 entries)

SEQ ID NO: 13 - protein, 4 aa, Artificial Sequence
  Description of Artificial Sequence: Synthetic peptide
GGGS

SEQ ID NO: 14 - protein, 5 aa, Artificial Sequence
  Description of Artificial Sequence: Synthetic peptide
GGGGS

SEQ ID NO: 15 - protein, 4 aa, Artificial Sequence
  Description of Artificial Sequence: Synthetic peptide
GGSG

SEQ ID NO: 16 - protein, 5 aa, Artificial Sequence
  Description of Artificial Sequence: Synthetic peptide
GGSGG

SEQ ID NO: 17 - protein, 5 aa, Artificial Sequence
  Description of Artificial Sequence: Synthetic peptide
GSGSG

SEQ ID NO: 18 - protein, 5 aa, Artificial Sequence
  Description of Artificial Sequence: Synthetic peptide
GSGGG

SEQ ID NO: 19 - protein, 5 aa, Artificial Sequence
  Description of Artificial Sequence: Synthetic peptide
GGGSG

SEQ ID NO: 20 - protein, 5 aa, Artificial Sequence
  Description of Artificial Sequence: Synthetic peptide
GSSSG

SEQ ID NO: 21 - protein, 8 aa, Artificial Sequence
  Description of Artificial Sequence: Synthetic peptide
GGSGGGSG

SEQ ID NO: 22 - protein, 12 aa, Artificial Sequence
  Description of Artificial Sequence: Synthetic peptide
GGSGGGSGGGSG

SEQ ID NO: 23 - protein, 16 aa, Artificial Sequence
  Description of Artificial Sequence: Synthetic peptide
GGSGGGSGGGSGGGSG

SEQ ID NO: 24 - protein, 20 aa, Artificial Sequence
  Description of Artificial Sequence: Synthetic peptide
GGSGGGSGGGSGGGSGGGSG

SEQ ID NO: 25 - protein, 9 aa, Artificial Sequence
  Description of Artificial Sequence: Synthetic peptide
YGYCGYDMS

SEQ ID NO: 26 - protein, 9 aa, Artificial Sequence
  Description of Artificial Sequence: Synthetic peptide
YTYSSAFMA

SEQ ID NO: 27 - protein, 9 aa, Artificial Sequence
  Description of Artificial Sequence: Synthetic peptide
YSYCGYDMM

SEQ ID NO: 28 - protein, 9 aa, Artificial Sequence
  Description of Artificial Sequence: Synthetic peptide
YGYCGYDMS

SEQ ID NO: 29 - protein, 9 aa, Artificial Sequence
  Description of Artificial Sequence: Synthetic peptide
YGYCGYDMS

SEQ ID NO: 30 - protein, 9 aa, Artificial Sequence
  Description of Artificial Sequence: Synthetic peptide
YTYTNNFMA

SEQ ID NO: 31 - protein, 9 aa, Artificial Sequence
  Description of Artificial Sequence: Synthetic peptide
YDYCGYDVR

SEQ ID NO: 32 - protein, 9 aa, Artificial Sequence
  Description of Artificial Sequence: Synthetic peptide
YSYCGYDMM

SEQ ID NO: 33 - protein, 9 aa, Artificial Sequence
  Description of Artificial Sequence: Synthetic peptide
YSYCGYDMM

SEQ ID NO: 34 - protein, 9 aa, Artificial Sequence
  Description of Artificial Sequence: Synthetic peptide
YDYCGYDVR

SEQ ID NO: 35 - protein, 9 aa, Artificial Sequence
  Description of Artificial Sequence: Synthetic peptide
YDYCGYDVR

SEQ ID NO: 36 - protein, 9 aa, Artificial Sequence
  Description of Artificial Sequence: Synthetic peptide
YTYSSAFMA

SEQ ID NO: 37 - protein, 9 aa, Artificial Sequence
  Description of Artificial Sequence: Synthetic peptide
YGYCGYDMS

SEQ ID NO: 38 - protein, 9 aa, Artificial Sequence
  Description of Artificial Sequence: Synthetic peptide
YDYCGYDVR

SEQ ID NO: 39 - protein, 9 aa, Artificial Sequence
  Description of Artificial Sequence: Synthetic peptide
YDYCGYDVR

SEQ ID NO: 40 - protein, 9 aa, Artificial Sequence
  Description of Artificial Sequence: Synthetic peptide
YTYSSAFMA

SEQ ID NO: 41 - protein, 9 aa, Artificial Sequence
  Description of Artificial Sequence: Synthetic peptide
YGYCGYDMS

SEQ ID NO: 42 - protein, 9 aa, Artificial Sequence
  Description of Artificial Sequence: Synthetic peptide
YTYTNNFMA

SEQ ID NO: 43 - protein, 9 aa, Artificial Sequence
  Description of Artificial Sequence: Synthetic peptide
YDYCGYDVR

SEQ ID NO: 44 - protein, 9 aa, Artificial Sequence
  Description of Artificial Sequence: Synthetic peptide
YTYSSAFMA

SEQ ID NO: 45 - protein, 9 aa, Artificial Sequence
  Description of Artificial Sequence: Synthetic peptide
YTYSSAFMA

SEQ ID NO: 46 - protein, 9 aa, Artificial Sequence
  Description of Artificial Sequence: Synthetic peptide
YTYSSAFMA

SEQ ID NO: 47 - protein, 16 aa, Artificial Sequence
  Description of Artificial Sequence: Synthetic peptide
LITSDRSISYEDSVKA

SEQ ID NO: 48 - protein, 17 aa, Artificial Sequence
  Description of Artificial Sequence: Synthetic peptide
AIYTRDGGTVYADSVKG

SEQ ID NO: 49 - protein, 16 aa, Artificial Sequence
  Description of Artificial Sequence: Synthetic peptide
LITSDYSIRYEDSVEG

SEQ ID NO: 50 - protein, 16 aa, Artificial Sequence
  Description of Artificial Sequence: Synthetic peptide
LITSDRIASYEDSVKG

SEQ ID NO: 51 - protein, 16 aa, Artificial Sequence
  Description of Artificial Sequence: Synthetic peptide
LITSDRSVSYEDSVKG

SEQ ID NO: 52 - protein, 17 aa, Artificial Sequence
  Description of Artificial Sequence: Synthetic peptide
AIYTGDGYAYYFYSVKG

SEQ ID NO: 53 - protein, 16 aa, Artificial Sequence
  Description of Artificial Sequence: Synthetic peptide
GIDSDGSTSYADSVKG

SEQ ID NO: 54 - protein, 16 aa, Artificial Sequence
  Description of Artificial Sequence: Synthetic peptide
LITSDYSIRYEDSVEG

SEQ ID NO: 55 - protein, 16 aa, Artificial Sequence
  Description of Artificial Sequence: Synthetic peptide
LITSDYSIRYEDSVEG

SEQ ID NO: 56 - protein, 16 aa, Artificial Sequence
  Description of Artificial Sequence: Synthetic peptide
GIDSDGSTSYADSVKG

SEQ ID NO: 57 - protein, 16 aa, Artificial Sequence
  Description of Artificial Sequence: Synthetic peptide
GIDSDGSTSYADSVKG

SEQ ID NO: 58 - protein, 17 aa, Artificial Sequence
  Description of Artificial Sequence: Synthetic peptide
AIYTRDGGTVYADSVKG

SEQ ID NO: 59 - protein, 16 aa, Artificial Sequence
  Description of Artificial Sequence: Synthetic peptide
LITSERVISYEDSVKG

SEQ ID NO: 60 - protein, 16 aa, Artificial Sequence
  Description of Artificial Sequence: Synthetic peptide
GIDSDGSTSYADSVKG

SEQ ID NO: 61 - protein, 16 aa, Artificial Sequence
  Description of Artificial Sequence: Synthetic peptide
GINSDGSTSYADSVKG

SEQ ID NO: 62 - protein, 17 aa, Artificial Sequence
  Description of Artificial Sequence: Synthetic peptide
AMYTRDGGTVYADSVKG

SEQ ID NO: 63 - protein, 16 aa, Artificial Sequence
  Description of Artificial Sequence: Synthetic peptide
LITSDRSVSYEDSVKG

SEQ ID NO: 64 - protein, 17 aa, Artificial Sequence
  Description of Artificial Sequence: Synthetic peptide
AIYTGDGYAYYFDSVKG

SEQ ID NO: 65 - protein, 16 aa, Artificial Sequence
  Description of Artificial Sequence: Synthetic peptide
GINSDGSTSYADSVKG

SEQ ID NO: 66 - protein, 17 aa, Artificial Sequence
  Description of Artificial Sequence: Synthetic peptide
AIYTRDGSPVYADSLKG

SEQ ID NO: 67 - protein, 17 aa, Artificial Sequence
  Description of Artificial Sequence: Synthetic peptide
AMYTRDGGTVYADSVKG

SEQ ID NO: 68 - protein, 17 aa, Artificial Sequence
  Description of Artificial Sequence: Synthetic peptide
AIYTRDGGTVYADSVKG

SEQ ID NO: 69 - protein, 18 aa, Artificial Sequence
  Description of Artificial Sequence: Synthetic peptide
SAAARESSWCRSRYRVAS

SEQ ID NO: 70 - protein, 18 aa, Artificial Sequence
  Description of Artificial Sequence: Synthetic peptide
KIPQPGRASLLDSQTYDY

SEQ ID NO: 71 - protein, 18 aa, Artificial Sequence
  Description of Artificial Sequence: Synthetic peptide
STAARESSWCRSRYRVAS

SEQ ID NO: 72 - protein, 18 aa, Artificial Sequence
  Description of Artificial Sequence: Synthetic peptide
SAAARENSWCRSRYRVAS

SEQ ID NO: 73 - protein, 18 aa, Artificial Sequence
  Description of Artificial Sequence: Synthetic peptide
STAARENNWCRSRYRIAY

SEQ ID NO: 74 - protein, 18 aa, Artificial Sequence
  Description of Artificial Sequence: Synthetic peptide
MERRIGTRRMTENAEYKY

SEQ ID NO: 75 - protein, 18 aa, Artificial Sequence
  Description of Artificial Sequence: Synthetic peptide
ESPAGESAWCRNFRGMDY

SEQ ID NO: 76 - protein, 18 aa, Artificial Sequence
  Description of Artificial Sequence: Synthetic peptide
STAARESSWCRSRYRVAS

SEQ ID NO: 77 - protein, 18 aa, Artificial Sequence
  Description of Artificial Sequence: Synthetic peptide
STAARESGWCRSRYRVAS

SEQ ID NO: 78 - protein, 18 aa, Artificial Sequence
  Description of Artificial Sequence: Synthetic peptide
ESPAGESAWCRNFRGMDY

SEQ ID NO: 79 - protein, 18 aa, Artificial Sequence
  Description of Artificial Sequence: Synthetic peptide
ESPAGESAWCRNFRGMDY

SEQ ID NO: 80 - protein, 18 aa, Artificial Sequence
  Description of Artificial Sequence: Synthetic peptide
KMPQPGRASLLDSQTYDY

SEQ ID NO: 81 - protein, 18 aa, Artificial Sequence
  Description of Artificial Sequence: Synthetic peptide
SAAARESSWCRSRYRVAS

SEQ ID NO: 82 - protein, 18 aa, Artificial Sequence
  Description of Artificial Sequence: Synthetic peptide
ESPAGESAWCRNFRGMDY

SEQ ID NO: 83 - protein, 18 aa, Artificial Sequence
  Description of Artificial Sequence: Synthetic peptide
ESPAGESAWCRNFRGMDY

SEQ ID NO: 84 - protein, 18 aa, Artificial Sequence
  Description of Artificial Sequence: Synthetic peptide
KIPQPGRASLLDSQTYDY

SEQ ID NO: 85 - protein, 18 aa, Artificial Sequence
  Description of Artificial Sequence: Synthetic peptide
STAARENNWCRSRYRIAS

SEQ ID NO: 86 - protein, 18 aa, Artificial Sequence
  Description of Artificial Sequence: Synthetic peptide
MERRSGRRRMTENAEYKY

SEQ ID NO: 87 - protein, 18 aa, Artificial Sequence
  Description of Artificial Sequence: Synthetic peptide
EGPAGESAWCRNFRGMDY

SEQ ID NO: 88 - protein, 18 aa, Artificial Sequence
  Description of Artificial Sequence: Synthetic peptide
KIPEPGRISLLDSQTYDY

SEQ ID NO: 89 - protein, 18 aa, Artificial Sequence
  Description of Artificial Sequence: Synthetic peptide
KIPQPGRASLLDSQTYDY

SEQ ID NO: 90 - protein, 18 aa, Artificial Sequence
  Description of Artificial Sequence: Synthetic peptide
KIPQPGRASLLDSQTYDY

SEQ ID NO: 91-96 - skipped (6 entries)

SEQ ID NO: 97 - protein, 116 aa, Artificial Sequence
  Description of Artificial Sequence: Synthetic polypeptide
QVQLQESGGGSVQAGGSLRLSCAASGYTYSSCTMGWYRQAPGKERELVSMLISDGSTFYADSVKGRFTFSQEYAKNTVYLQMNSLKPEDTAMYYCGCATLGSRTVWGQGTQVTVSS

SEQ ID NO: 98 - protein, 116 aa, Artificial Sequence
  Description of Artificial Sequence: Synthetic polypeptide
QVQLQESGGGSVQAGGSLTLSCTAPGFTFRLAAMRWVRQAPGKGLEWVSGIDSRGSTIYADSVKGRFTISKDNAKNTLYLQLNSLKTEDTAMYYCAQGVYGDTYSGSQGTQVTVSS

SEQ ID NO: 99 - protein, 127 aa, Artificial Sequence
  Description of Artificial Sequence: Synthetic polypeptide
QVQLQESGGGSVQAGGSLRLSCTASVNTYCEYNMSWYRQAPGKEREFVSGVDSDGSTRYSESVKGRFTISQDNAKNTMYLQMNGLKPEDTAMYYCKTYVCTFCSGNSCYYEYKYYYEGQGTQVTVSS

SEQ ID NO: 100 - protein, 121 aa, Artificial Sequence
  Description of Artificial Sequence: Synthetic polypeptide
QVQLQESGGGSVQAGGSLRLSCAASGYTYSNNCMGWFRQAPGKDRERIANIYTGGGRTTYADSVKGRFTISQDSAKSTVYLQMNSLKPEDTAMYYCAAGSCGSARSEYSYWGQGTQVTVSS

SEQ ID NO: 101 - protein, 120 aa, Artificial Sequence
  Description of Artificial Sequence: Synthetic polypeptide
QVQLQESGGGSVQAGGSLRLSCAASGYTFCMAWFRQAPGKEREGVARFYTRDGYTYYSDSVKGRFTISQNNAKNTLYLQMNSLKSEDTAMYYCAADLARCSSNKNDFRYWGQGTQVTVSS

SEQ ID NO: 102 - protein, 127 aa, Artificial Sequence
  Description of Artificial Sequence: Synthetic polypeptide
QVQLQESGGGSVQAGGSLRLSCAASGYTSGNYWMGWFRQAPGKEREGVATLWTGGASTFYGDSVKGRFTISRDNFKNTLYLQMNSLKVEDTAMYYCAADPALRLGANILRPAEYKYWGQGTQVTVSS

SEQ ID NO: 103 - protein, 118 aa, Artificial Sequence
  Description of Artificial Sequence: Synthetic polypeptide
QVQLQESGGGLVQPGGSLRLSCAASGFTFSRSAMTWVRQAPGKGLDWVSGIDSGGTTVYADSVKGRFTISRDSAKNTLYLQMNSLKTEDTAVYYCAIGLPWGNTWRTRGQGTQVTVSS

SEQ ID NO: 104 - protein, 116 aa, Artificial Sequence
  Description of Artificial Sequence: Synthetic polypeptide
QVQLQESGGGSVQAGGSLRLSCTASRYTYSSCTMGWYRQAPGKERELVSMVFSDGSTFYADSVKGRFTFSQENAKNTVYLQMNSLKPEDTAMYYCGCATLGSRTIWGQGTQVTVSS

SEQ ID NO: 105 - protein, 116 aa, Artificial Sequence
  Description of Artificial Sequence: Synthetic polypeptide
QVQLQESGGGLVQPGGSLRLSCATSGFTFRLTAMRWVRQAPGKGVEWVSGIDSAGSTIYADSVKGRFTISKDNAKNTLYLQMNSLKTEDTAMYYCAQGVYGDTYSGSQGTQVTVSS

SEQ ID NO: 106 - protein, 116 aa, Artificial Sequence
  Description of Artificial Sequence: Synthetic polypeptide
QVQLQESGGGSVQAGGSLRLSCAASGDTYSSCTMGWYRQAPGKERDLVSMLMGDGSTFYADSVKGRFTFSQENAKNTVYLQMNSLKPEDTAMYYCGCATLGSRTIWGQGTQVTVSS

SEQ ID NO: 107 - protein, 116 aa, Artificial Sequence
  Description of Artificial Sequence: Synthetic polypeptide
QVQLQESGGGSVQAGGSLRLSCAASGYTYSSCTMGWYRQAPGKERELVSMLISDGSTFYADSVKGRFTFSQENAKSTVYLQMNSLKPEDTAMYYCGCATLGSRTVWGQGTQVTVSS

SEQ ID NO: 108 - protein, 116 aa, Artificial Sequence
  Description of Artificial Sequence: Synthetic polypeptide
QVQLQESGGGSVQAGGSLRLSCAASGYTYSSCTMGWYRQAPGKERELVSMLISDGSTFYADSVKGRFTFSQENAKNTVYLQMNSLKPEDTAMYYCGCATLGSRTVWGQGTQVTVSS

SEQ ID NO: 109 - protein, 116 aa, Artificial Sequence
  Description of Artificial Sequence: Synthetic polypeptide
QVQLQESGGGLVQPGGSLRLSCATSGFTFRLAAMRWVRQAPGKGLEWVSGIDSRGSTIYADSVKGRFTISKDNAKNTLYLQLNSLKTEDTAMYYCAQGVYGDTYSGSQGTQVTVSS

SEQ ID NO: 110 - protein, 118 aa, Artificial Sequence
  Description of Artificial Sequence: Synthetic polypeptide
QVQLQESGGGLVQPGGSLRLSCAASGFTFRTSAMTWVRQAPGKGLDWVSGIDSGGTTVYADSVKGRFTISRDSAKNTLYLQMNSLKTEDTAVYYCAMGLPWGNTWRTRGQGTQVTVSS

SEQ ID NO: 111 - protein, 116 aa, Artificial Sequence
  Description of Artificial Sequence: Synthetic polypeptide
QVQLQESGGGSVQAGGSLRLSCAASGYTYSSCTMGWYRQAPGKERELVSMVFSDGSTFYADSVKGRFTFSQENAKNTVYLQMNSLKPEDTAMYYCGCATLGSRTIWGQGTQVTVSS

SEQ ID NO: 112 - protein, 116 aa, Artificial Sequence
  Description of Artificial Sequence: Synthetic polypeptide
QVQLQESGGGSVQAGGSLRLSCAASGDTYSSCTMGWYRQAPGKERDLVSMLMGDGSTFYADSVKGRFTFSQENAKNTVYLQMNNLKPEDTAMYYCGCATLGSRTIWGQGTQVTVSS

SEQ ID NO: 113 - protein, 116 aa, Artificial Sequence
  Description of Artificial Sequence: Synthetic polypeptide
QVQLQESGGGSVQAGGSLRLSCAASGFTFRLTAMRWVRQAPGKGLEWVSGIDSRGSTIYADSVKGRFTISKDNAKNTLYLQLNSLKTEDTAMYYCAQGVYGDTYSGSQGTQVTVSS

SEQ ID NO: 114 - protein, 116 aa, Artificial Sequence
  Description of Artificial Sequence: Synthetic polypeptide
QVQLQESGGGLVQPGGSLRLSCAASGFTFRLTAMRWVRQAPGKGLEWVSGIDSRGSTIYADSVKGRFTISKDNAKNTLYLQLNSLKTEDTAMYYCAQGVYGDTYSGSQGTQVTVSS

SEQ ID NO: 115 - protein, 116 aa, Artificial Sequence
  Description of Artificial Sequence: Synthetic polypeptide
QVQLQESGGGLVQPGGSLRLSCAASGFTFRLSAMRWVRQAPGKGLEWVSGIDSRGSTIYADSVKGRFTISKDNAKNTLYLQLNSLKTEDTAMYYCAQGVYGDTYSGSQGTQVTVSS

SEQ ID NO: 116 - protein, 118 aa, Artificial Sequence
  Description of Artificial Sequence: Synthetic polypeptide
QVQLQESGGGLVQPGGSLRLSCAASGFTFSSSAMTWVRQAPGKGLDWVSGIDSGGTTVYADSVKGRATILKDNAKNTLYLQMNSLKTEDTAVYYCATGLPWGNTWRTRGQGTQVTVSS

SEQ ID NO: 117 - protein, 118 aa, Artificial Sequence
  Description of Artificial Sequence: Synthetic polypeptide
QVQLQESGGGLVQPGGSLRLSCATSGFTFSSSAMTWVRQAPGKGLDWVSGIDSGGTTVYADSVKGRFTISKDNAKNTLYLQMNSLKTEDTAVYYCATGLPWGNTWRTTGQGTQVTVSS

SEQ ID NO: 118 - protein, 120 aa, Artificial Sequence
  Description of Artificial Sequence: Synthetic polypeptide
QVQLQESGGGSVQAGGSLRLSCAASGYTFCMAWFRQAPGKEREGVARFYTRDSYTYYSDSVKGRFTISQNNAKNTLYLQMNSLKSEDTAMYYCAADLTRCSSNKNDFRYWGQGTQVTVSS

SEQ ID NO: 119 - protein, 116 aa, Artificial Sequence
  Description of Artificial Sequence: Synthetic polypeptide
QVQLQESGGGLVQPGGSLRLSCAASGFNFRLYAMRWVRQAPGKGVEWVSGIDSGGSTIYADSVKGRFTISKDNAKNTLYLQLNSLKTEDTAMYYCAQGVYGDTYSGSQGTQVTVSS

SEQ ID NO: 120 - protein, 120 aa, Artificial Sequence
  Description of Artificial Sequence: Synthetic polypeptide
QVQLQESGGGSVQAGGSLRLSCAVSGYTFCMAWFRQAPGKEREGVARFYTRDGYTYYSGSVKGRFTISQNNAKNTLYLQMNSLKSEDTAMYYCAADLTRCSSNKNDFRYWGQGTQVTVSS

SEQ ID NO: 121 - protein, 116 aa, Artificial Sequence
  Description of Artificial Sequence: Synthetic polypeptide
QVQLQESGGGSVQAGGSLRLSCAASGYTYSSCTMGWYRQAPGKERELVSMLISDGSTFYADSVKGRFTFSQENAKNTVYLQMNSLKPEDTAMYFCGCATLGSRTVWGQGTQVTVSS

SEQ ID NO: 122 - protein, 120 aa, Artificial Sequence
  Description of Artificial Sequence: Synthetic polypeptide
QVQLQESGGGSVQAGGSLRLSCAASGYTFCMAWFRQAPGKEREGVARFYTRDGYTYYSDSVKGRFTISQNNAKNTLYLQMNSLKSEDTAMYYCAADLTRCSSNKNDFRYWGQGTQVTVSS

SEQ ID NO: 123 - protein, 116 aa, Artificial Sequence
  Description of Artificial Sequence: Synthetic polypeptide
QVQLQESGGGSVQAGGSLRLSCAASGYTYSSCTMGWYRQAPGKERELVSMLISDGSTFYADSVKGRFTSSQENAKNTVYLQMNSLKPEDTAMYYCGCATLGSRTVWGQGTQVTVSS

SEQ ID NO: 124 - protein, 116 aa, Artificial Sequence
  Description of Artificial Sequence: Synthetic polypeptide
QVQLQESGGGLVQPGGSLRLSCAASGFTFRLTAMRWVRQAPGKGLEWVSGIDSRGSTIYADSVKGRFTISRDNAKNTLYLQLNSLKTEDAAMYYCAQGVYGDTYSGSQGTQVTVSS

SEQ ID NO: 125 - protein, 118 aa, Artificial Sequence
  Description of Artificial Sequence: Synthetic polypeptide
QVQLQESGGGLVQPGGSLRLSCAASGFTFSTSAMTWVRQAPGKGLDWVSGIDSGGTTVYADSVKGRFTISKDNAKNTLYLQMNSLKTEDTAVYYCATGLPWGNTWRTRGQGTQVTVSS

SEQ ID NO: 126 - protein, 116 aa, Artificial Sequence
  Description of Artificial Sequence: Synthetic polypeptide
QVQLQESGGGLVQPGGSLRLSCAASGFTFRLTAMRWVRQAPGKGLEWVSGIDSRGSTIYADSVKGRFTISKDNAKNTLYLQLNSLKTEDTAMYYCAQGVYGDTYSGSLGTQVTVSS

SEQ ID NO: 127 - protein, 116 aa, Artificial Sequence
  Description of Artificial Sequence: Synthetic polypeptide
QVQLQESGGGLVQPGGSLRLSCAASGFTFRLTAMRWVRQAPGKGLEWVSGIDSRGSTIYADSVKGRFTISRDNAKNTLYLQLNSLKTEDTAMYYCAQGVYGDTYSGSQGTQVTVSS

SEQ ID NO: 128 - protein, 118 aa, Artificial Sequence
  Description of Artificial Sequence: Synthetic polypeptide
QVQLQESGGGLVRPGGSLRLSCAASGFTFSRSAMTWVRQAPGKGLDWVSGIDSGGTTVYADSVKGRFTISRDSAKNTLYLQMNSLKTEDTAVYYCAIGLPWGNTWRTRGQGTQVTVSS

SEQ ID NO: 129 - protein, 118 aa, Artificial Sequence
  Description of Artificial Sequence: Synthetic polypeptide
QVQLQESGGGLVQPGGSLRLSCTTSGFTFSSSAMTWVRQAPGKGLDWVSGIDSGGTTVYADSVKGRFTISKDNAKNTLYLQMNSLKTEDTAVYYCATGLPWGNTWRTTGQGTQVTVSS

SEQ ID NO: 130 - protein, 116 aa, Artificial Sequence
  Description of Artificial Sequence: Synthetic polypeptide
QVQLQESGGGLVQPGGSLRLSCAASGFTFRLTAMRWVRQAPGKGLEWVSGIDSRGSTIYADSVKGRFTISKDNAKNTLYLQLNSLKTEDTAMYYCAQGVYGDTHSGSQGTQVTVSS

SEQ ID NO: 131 - protein, 118 aa, Artificial Sequence
  Description of Artificial Sequence: Synthetic polypeptide
QVQLQESGGGLVQPGGSLRLACSASGFTFSSSAMTWVRQAPGKGLDWVSGIDSGGTTVYADSVKGRATILKDNAKNTLYLQMNSLKTEDTAVYYCATGLPWGNTWRTRGQGTQVTVSS

SEQ ID NO: 132 - protein, 116 aa, Artificial Sequence
  Description of Artificial Sequence: Synthetic polypeptide
QVQLQESGGGSVQAGGSLRLSCAASGDTYSSCTMGWYRQAPGKERDLVSMVFSDGSTFYADSVKGRFTFSQENAKNTVYLQMNSLKPEDTAMYYCGCATLGSRTIWGQGTQVTVSS

SEQ ID NO: 133 - protein, 118 aa, Artificial Sequence
  Description of Artificial Sequence: Synthetic polypeptide
QVQLQESGGGLVQPGGSLRLSCATSGFTFSSGAMTWVRQAPGKGLDWVSGIDSGGTTVYADSVKGRFTISKDNAKNTLYLQMNSLKTEDTAVYYCATGLPWGNTWRTTGQGTQVTVSS

SEQ ID NO: 134 - protein, 118 aa, Artificial Sequence
  Description of Artificial Sequence: Synthetic polypeptide
QVQLQESGGGLVQPGGSLRLSCAASGFTFSTSAMTWVRQAPGKGLDWVSGIDSGGTTVYADSVKGRFTISKDNAKNTLYLQMNSLKTEDTAVYYCATGLPWGNIWRTRGQGTQVTVSS

SEQ ID NO: 135 - protein, 118 aa, Artificial Sequence
  Description of Artificial Sequence: Synthetic polypeptide
QVQLQESGGGLVQPGGSLRLSCAASGFTFSRSAMTWVRQAPGKGLDWVSGIDSGGTTVYADSVKGRFTISRDSAKNTLYLQMNGLKTEDTAVYYCAIGLPWGNTWRTRGQGTQVTVSS

SEQ ID NO: 136 - protein, 116 aa, Artificial Sequence
  Description of Artificial Sequence: Synthetic polypeptide
QVQLQESGGGLVQPGGSLRLSCAASGFTFRLTAMRWVRQAPGKGLEWVSGIDSRGSTIYADSVKGRFTISKDNAKNTLYLQLNSLKSEDTAMYYCAQGVYGDTYSGSQGTQVTVSS

SEQ ID NO: 137 - protein, 118 aa, Artificial Sequence
  Description of Artificial Sequence: Synthetic polypeptide
QVQLQESGGGLVQPGGSLRLTCAASGFTFSTSAMTWVRQAPGKGLDWVSGIDSGGTTVYADSVKGRFTISKDNAKNTLYLQMNSLKTEDTAVYYCATGLPWGNTWRTRGQGTQVTVSS

SEQ ID NO: 138 - protein, 116 aa, Artificial Sequence
  Description of Artificial Sequence: Synthetic polypeptide
QVQLQESGGGLVQPGGSLRLSCAASGFTFSNYAMRWVRQAPGKGLEWVSGIDSRGSTIYADSVKGRFTISKDNAKNTLYLQLNSLKTEDTAMYYCAQGVYGDTYSGSQGTQVTVSS

SEQ ID NO: 139 - protein, 120 aa, Artificial Sequence
  Description of Artificial Sequence: Synthetic polypeptide
QVQLQESGGGSVQAGGSLRLSCAASGYTFCMAWFRQAPGKEREGVARFYTRDGYTYYSDSVKGRFTISQDNAKNTLYLQMNSLKSEDTAMYYCAADLTRCSSNKNDFRYWGQGTQVTVSS

SEQ ID NO: 140 - protein, 116 aa, Artificial Sequence
  Description of Artificial Sequence: Synthetic polypeptide
QVQLQESGGGLVQPGGSLRLSCAASGFTFRLSAMRWVRQAPGKGFEWVSGIDSRGSTIYADSVKGRFTISKDNAKNTLYLQLNSLKTEDTAMYYCAQGVYGDTYSGSQGTQVTVSS

SEQ ID NO: 141 - protein, 116 aa, Artificial Sequence
  Description of Artificial Sequence: Synthetic polypeptide
QVQLQESGGGLVQPGGSLRLSCAASGFTFRLSAMRWVRQAPGKGLEWVSGIDSRGSTIYADSVEGRFTISKDNAKNTLYLQLNSLKTEDTAMYYCAQGVYGDTYSGSQGTQVTVSS

SEQ ID NO: 142 - protein, 116 aa, Artificial Sequence
  Description of Artificial Sequence: Synthetic polypeptide
QVQLQESGGGLVQPGGSLRLSCAASGFTFRLSAMRWVRQAPGKGLEWVSGIDSRGSTIYADSVKGRFTISKDDAKNTLYLQLNSLKTEDTAMYYCAQGVYGDTYSGSQGTQVTVSS

SEQ ID NO: 143 - protein, 13 aa, Artificial Sequence
  Description of Artificial Sequence: Synthetic peptide
DPIATMTRRCDPY

SEQ ID NO: 144 - protein, 12 aa, Artificial Sequence
  Description of Artificial Sequence: Synthetic peptide
DPNYSDHVCPPY

SEQ ID NO: 145 - protein, 127 aa, Artificial Sequence
  Description of Artificial Sequence: Synthetic polypeptide
QVQLQESGGGSVQAGGSLRLSCTASGYTYSSAFMAWFRQAPGKEREGVAAIYTRDGGTVYADSVKGRFTISQDNAKNTLYLQMNSLKAEDTAMYYCAAKIPQPGRASLLDSQTYDYWGQGTQVTVSS

SEQ ID NO: 146 - protein, 126 aa, Artificial Sequence
  Description of Artificial Sequence: Synthetic polypeptide
QVQLQESGGGSVQAGGSLRLSCAVSGYDYCGYDVRWYRQAPGKEREFVSGIDSDGSTSYADSVKGRFTISQDNAENTSYLHMFSLKPEDTAMYYCKTESPAGESAWCRNFRGMDYWGKGTQVTVSS

SEQ ID NO: 147 - protein, 126 aa, Artificial Sequence
  Description of Artificial Sequence: Synthetic polypeptide
QVQLQESGGGSVQAGGSLRLSCVASGYSYCGYDMMWYRQAPGKEREFVALITSDYSIRYEDSVEGRFSISRDNAKNTGYLLMSNLTPADTAIYYCKTSTAARESSWCRSRYRVASWGQGTQVTVSS

SEQ ID NO: 148 - protein, 127 aa, Artificial Sequence
  Description of Artificial Sequence: Synthetic polypeptide
QVQLQESGGGSVQAGGSLRLSCAASRYTYTNNFMAWFRQAPGKEREGVAAIYTGDGYAYYFDSVKGRFTISQDNDKNMLYLQMNSLKPEDTAMYYCAAMERRSGRRRMTENAEYKYWGQGTQVTVSS

SEQ ID NO: 149 - protein, 121 aa, Artificial Sequence
  Description of Artificial Sequence: Synthetic polypeptide
QVQLQESGGGSVQAGETLRLSCTVSGFTIDDSEMGWYRQAPGHECELVASGSSDDDTYYVDSVKGRFTISLDNAKNMVYLQMNSLKPEDTAVYYCATGPTYPPKDGDCAHWGQGTQVTVSS

SEQ ID NO: 150 - protein, 127 aa, Artificial Sequence
  Description of Artificial Sequence: Synthetic polypeptide
QVQLQESGGGSVQAGGSLRLSCTASGYTYSSAFMAWFRQAPGKEREGVAAIYTRDGSPVYADSLKGRFTISQDNAKNTLHLQMNSLKPEDTAMYYCAAKIPEPGRISLLDSQTYDYWGHGTQVTVSS

SEQ ID NO: 151 - protein, 126 aa, Artificial Sequence
  Description of Artificial Sequence: Synthetic polypeptide
QVQLQESGGGSVQAGGSLRLSCAVSGYDYCGYDVRWYRRAPGKEREFVSGIDSDGSTSYADSVKGRFTISQDNAENTSYLHMFSLKPEDTAMYYCKTESPAGESAWCRNFRGMDYWGKGTQVTVSS

SEQ ID NO: 152 - protein, 126 aa, Artificial Sequence
  Description of Artificial Sequence: Synthetic polypeptide
QVQLQESGGGSVQAGGFLRLSCVASGYGYCGYDMSWYRQAPGKEREFVALITSERVISYEDSVKGRFSISRDNAENTGYLEMNRLTPDDTAIYYCKTSAAARESSWCRSRYRVASWGQGTQVTVSS

SEQ ID NO: 153 - protein, 126 aa, Artificial Sequence
  Description of Artificial Sequence: Synthetic polypeptide
QVQLQESGGGSVQAGGSLRLSCVASGYGYCGYDMSWYRQAPGKEREFVALITSDRSISYEDSVKARFIISRDNAANTGYLDMTRLTPDDTAIYYCKTSAAARESSWCRSRYRVASWGQGTQVTVSS

SEQ ID NO: 154 - protein, 126 aa, Artificial Sequence; identical to SEQ ID NO: 146
  Description of Artificial Sequence: Synthetic polypeptide

SEQ ID NO: 155 - protein, 126 aa, Artificial Sequence; identical to SEQ ID NO: 147
  Description of Artificial Sequence: Synthetic polypeptide

SEQ ID NO: 156 - protein, 127 aa, Artificial Sequence
  Description of Artificial Sequence: Synthetic polypeptide
QVQLQESGGGSVQAGGSLRLSCAASRYTYTNNFMAWFRQAPGKEREGVAAIYTGDGYAYYFYSVKGRFTISQDNDENMLYLQMNSLKPEDTAMYYCAAMERRIGTRRMTENAEYKYWGQGTQVTVSS

SEQ ID NO: 157 - protein, 126 aa, Artificial Sequence
  Description of Artificial Sequence: Synthetic polypeptide
QVQLQESGGGSVQAGGSLRLSCVASGYSYCGYDMMWYRQAPGKEREFVALITSDYSIRYEDSVEGRFSISRDNAKNTGYLLMSNLTPADTAIYYCKTSTAARESGWCRSRYRVASWGQGTQVTVSS

SEQ ID NO: 158 - protein, 126 aa, Artificial Sequence
  Description of Artificial Sequence: Synthetic polypeptide
QVQLQESGGGSVQAGGSLRLSCAVSGYDYCGYDVRWYRQAPGKEREFVSGINSDGSTSYADSVKGRFTISQDNAENTSYLHMFSLKPEDTAMYYCKTESPAGESAWCRNFRGMDYWGKGTQVTVSS

SEQ ID NO: 159 - protein, 126 aa, Artificial Sequence
  Description of Artificial Sequence: Synthetic polypeptide
QVQLQESGGGSVQAGGFLRLSCVASGYGYCGYDMSWYRQVPGKEREFVALITSDRSVSYEDSVKGRFSISRDNAKNTAYLEMNRLTPDDTAVYYCKTSTAARENNWCRSRYRIAYWGQGTQVTVSS

SEQ ID NO: 160 - protein, 126 aa, Artificial Sequence
  Description of Artificial Sequence: Synthetic polypeptide
QVQLQESGGGSVQAGGSLRLSCAVSGYDYCGYDVRWYRQAPGKEREFVSGINSDGSTSYADSVKGRFTISQDNAENTSYLHMFSLKPEDTAMYYCKTEGPAGESAWCRNFRGMDYWGKGTQVTVSS

SEQ ID NO: 161 - protein, 127 aa, Artificial Sequence
  Description of Artificial Sequence: Synthetic polypeptide
QVQLQESGGGSVQAGGSLRLSCTASGYTYSSAFMAWFRQAPGKEREGVAAMYTRDGGTVYADSVKGRFTISQDNAKNTLYLQIHTLKAEDTAMYYCAAKIPQPGRASLLDSQTYDYWGQGTQVTVSS

SEQ ID NO: 162 - protein, 127 aa, Artificial Sequence
  Description of Artificial Sequence: Synthetic polypeptide
QVQLQESGGGSVQAGGSLRLSCTASGYTYSSAFMAWFRQAPGKEREGVAAMYTRDGGTVYADSVKGRFTISQDNAKNTLYLQMNSLKTEDTAMYYCAAKIPQPGRASLLDSQTYDYWGQGTQVTVSS

SEQ ID NO: 163 - protein, 126 aa, Artificial Sequence
  Description of Artificial Sequence: Synthetic polypeptide
QVQLQESGGGSVQAGGFLRLSCVASGYGYCGYDMSWYRQVPGKEREFVALITSDRSVSYEDSVKGRFSISRDNAKNTAYLEMNRLTPDDTAIYYCKTSTAARENNWCRSRYRIASWGQGTQVTVSS

SEQ ID NO: 164 - protein, 126 aa, Artificial Sequence
  Description of Artificial Sequence: Synthetic polypeptide
QVQLQESGGGSVQAGGSLRLSCVASGYGYCGYDMSWYRQTPGKEREFVALITSDRIASYEDSVKGRFIISRDNAKNTGYLDMTRVTPDDTAIYYCKTSAAARENSWCRSRYRVASWGQGTQVTVSS

SEQ ID NO: 165 - protein, 126 aa, Artificial Sequence; identical to SEQ ID NO: 151
  Description of Artificial Sequence: Synthetic polypeptide

SEQ ID NO: 166 - protein, 127 aa, Artificial Sequence
  Description of Artificial Sequence: Synthetic polypeptide
QVQLQESGGGSVQAGGSLRLSCTASGYTYSSAFMAWFRQAPGKEREGVAAIYTRDGGTVYADSVKGRFTISQDNAKNILYLQMNSLKAEDTAMYYCAAKIPQPGRASLLDSQTYDYWGQGTQVTVSS

SEQ ID NO: 167 - protein, 127 aa, Artificial Sequence
  Description of Artificial Sequence: Synthetic polypeptide
QVQLQESGGGSVQAGGSLRLSCTASGYTYSSAFMAWFRQAPGKEREGVAAIYTRDGGTVYADSVKGRFTISQDNAKNTLYLQMNSLKPEDTAMYYCAAKMPQPGRASLLDSQTYDYWGQGTQVTVSS

SEQ ID NO: 168 - protein, 800 aa, Artificial Sequence
  Description of Artificial Sequence: Synthetic polypeptide
  SITE (1)..(20): This region may encompass 1-20 residues
  SITE (21)..(40): This region may encompass 1-20 residues
  SITE (41)..(60): This region may encompass 1-20 residues
  SITE (61)..(80): This region may encompass 1-20 residues
  SITE (81)..(100): This region may encompass 1-20 residues
  SITE (101)..(120): This region may encompass 1-20 residues
  SITE (121)..(140): This region may encompass 1-20 residues
  SITE (141)..(160): This region may encompass 1-20 residues
  SITE (161)..(180): This region may encompass 1-20 residues
  SITE (181)..(200): This region may encompass 1-20 residues
  SITE (201)..(220): This region may encompass 1-20 residues
  SITE (221)..(240): This region may encompass 1-20 residues
  SITE (241)..(260): This region may encompass 1-20 residues
  SITE (261)..(280): This region may encompass 1-20 residues
  SITE (281)..(300): This region may encompass 1-20 residues
  SITE (301)..(320): This region may encompass 1-20 residues
  SITE (321)..(340): This region may encompass 1-20 residues
  SITE (341)..(360): This region may encompass 1-20 residues
  SITE (361)..(380): This region may encompass 1-20 residues
  SITE (381)..(400): This region may encompass 1-20 residues
  SITE (401)..(420): This region may encompass 1-20 residues
  SITE (421)..(440): This region may encompass 1-20 residues
  SITE (441)..(460): This region may encompass 1-20 residues
  SITE (461)..(480): This region may encompass 1-20 residues
  SITE (481)..(500): This region may encompass 1-20 residues
  SITE (501)..(520): This region may encompass 1-20 residues
  SITE (521)..(540): This region may encompass 1-20 residues
  SITE (541)..(560): This region may encompass 1-20 residues
  SITE (561)..(580): This region may encompass 1-20 residues
  SITE (581)..(600): This region may encompass 1-20 residues
  SITE (601)..(620): This region may encompass 1-20 residues
  SITE (621)..(640): This region may encompass 1-20 residues
  SITE (641)..(660): This region may encompass 1-20 residues
  SITE (661)..(680): This region may encompass 1-20 residues
  SITE (681)..(700): This region may encompass 1-20 residues
  SITE (701)..(720): This region may encompass 1-20 residues
  SITE (721)..(740): This region may encompass 1-20 residues
  SITE (741)..(760): This region may encompass 1-20 residues
  SITE (761)..(780): This region may encompass 1-20 residues
  SITE (781)..(800): This region may encompass 1-20 residues
  SITE (1)..(800): This sequence may encompass 1-20 “(Gly)m(Ser)o” repeating units, wherein m = 1 to 20, o = 1 to 20
  See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 169 - protein, 100 aa, Artificial Sequence
  Description of Artificial Sequence: Synthetic polypeptide
  SITE (1)..(100): This sequence may encompass 1-20 “Gly Ser Gly Gly Ser” repeating units
  See specification as filed for detailed description of substitutions and preferred embodiments
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 170 - protein, 1200 aa, Artificial Sequence
  Description of Artificial Sequence: Synthetic polypeptide
  SITE (1)..(20): This region may encompass 1-20 residues
  SITE (21)..(40): This region may encompass 1-20 residues
  SITE (41)..(60): This region may encompass 1-20 residues
  SITE (61)..(80): This region may encompass 1-20 residues
  SITE (81)..(100): This region may encompass 1-20 residues
  SITE (101)..(120): This region may encompass 1-20 residues
  SITE (121)..(140): This region may encompass 1-20 residues
  SITE (141)..(160): This region may encompass 1-20 residues
  SITE (161)..(180): This region may encompass 1-20 residues
  SITE (181)..(200): This region may encompass 1-20 residues
  SITE (201)..(220): This region may encompass 1-20 residues
  SITE (221)..(240): This region may encompass 1-20 residues
  SITE (241)..(260): This region may encompass 1-20 residues
  SITE (261)..(280): This region may encompass 1-20 residues
  SITE (281)..(300): This region may encompass 1-20 residues
  SITE (301)..(320): This region may encompass 1-20 residues
  SITE (321)..(340): This region may encompass 1-20 residues
  SITE (341)..(360): This region may encompass 1-20 residues
  SITE (361)..(380): This region may encompass 1-20 residues
  SITE (381)..(400): This region may encompass 1-20 residues
  SITE (401)..(420): This region may encompass 1-20 residues
  SITE (421)..(440): This region may encompass 1-20 residues
  SITE (441)..(460): This region may encompass 1-20 residues
  SITE (461)..(480): This region may encompass 1-20 residues
  SITE (481)..(500): This region may encompass 1-20 residues
  SITE (501)..(520): This region may encompass 1-20 residues
  SITE (521)..(540): This region may encompass 1-20 residues
  SITE (541)..(560): This region may encompass 1-20 residues
  SITE (561)..(580): This region may encompass 1-20 residues
  SITE (581)..(600): This region may encompass 1-20 residues
  SITE (601)..(620): This region may encompass 1-20 residues
  SITE (621)..(640): This region may encompass 1-20 residues
  SITE (641)..(660): This region may encompass 1-20 residues
  SITE (661)..(680): This region may encompass 1-20 residues
  SITE (681)..(700): This region may encompass 1-20 residues
  SITE (701)..(720): This region may encompass 1-20 residues
  SITE (721)..(740): This region may encompass 1-20 residues
  SITE (741)..(760): This region may encompass 1-20 residues
  SITE (761)..(780): This region may encompass 1-20 residues
  SITE (781)..(800): This region may encompass 1-20 residues
  SITE (801)..(820): This region may encompass 1-20 residues
  SITE (821)..(840): This region may encompass 1-20 residues
  SITE (841)..(860): This region may encompass 1-20 residues
  SITE (861)..(880): This region may encompass 1-20 residues
  SITE (881)..(900): This region may encompass 1-20 residues
  SITE (901)..(920): This region may encompass 1-20 residues
  SITE (921)..(940): This region may encompass 1-20 residues
  SITE (941)..(960): This region may encompass 1-20 residues
  SITE (961)..(980): This region may encompass 1-20 residues
  SITE (981)..(1000): This region may encompass 1-20 residues
  SITE (1001)..(1020): This region may encompass 1-20 residues
  SITE (1021)..(1040): This region may encompass 1-20 residues
  SITE (1041)..(1060): This region may encompass 1-20 residues
  SITE (1061)..(1080): This region may encompass 1-20 residues
  SITE (1081)..(1100): This region may encompass 1-20 residues
  SITE (1101)..(1120): This region may encompass 1-20 residues
  SITE (1121)..(1140): This region may encompass 1-20 residues
  SITE (1141)..(1160): This region may encompass 1-20 residues
  SITE (1161)..(1180): This region may encompass 1-20 residues
  SITE (1181)..(1200): This region may encompass 1-20 residues
  SITE (1)..(1200): This sequence may encompass 1-20 “(Gly)m(Ser)o(Gly)m” repeating units, wherein m = 1 to 20, o = 1 to 20
  See specification as filed for detailed description of substitutions and preferred embodiments
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 171 - protein, 2000 aa, Artificial Sequence
  Description of Artificial Sequence: Synthetic polypeptide
  SITE (1)..(20): This region may encompass 1-20 residues
  SITE (21)..(40): This region may encompass 1-20 residues
  SITE (41)..(60): This region may encompass 1-20 residues
  SITE (61)..(80): This region may encompass 1-20 residues
  SITE (81)..(100): This region may encompass 1-20 residues
  SITE (101)..(120): This region may encompass 1-20 residues
  SITE (121)..(140): This region may encompass 1-20 residues
  SITE (141)..(160): This region may encompass 1-20 residues
  SITE (161)..(180): This region may encompass 1-20 residues
  SITE (181)..(200): This region may encompass 1-20 residues
  SITE (201)..(220): This region may encompass 1-20 residues
  SITE (221)..(240): This region may encompass 1-20 residues
  SITE (241)..(260): This region may encompass 1-20 residues
  SITE (261)..(280): This region may encompass 1-20 residues
  SITE (281)..(300): This region may encompass 1-20 residues
  SITE (301)..(320): This region may encompass 1-20 residues
  SITE (321)..(340): This region may encompass 1-20 residues
  SITE (341)..(360): This region may encompass 1-20 residues
  SITE (361)..(380): This region may encompass 1-20 residues
  SITE (381)..(400): This region may encompass 1-20 residues
  SITE (401)..(420): This region may encompass 1-20 residues
  SITE (421)..(440): This region may encompass 1-20 residues
  SITE (441)..(460): This region may encompass 1-20 residues
  SITE (461)..(480): This region may encompass 1-20 residues
  SITE (481)..(500): This region may encompass 1-20 residues
  SITE (501)..(520): This region may encompass 1-20 residues
  SITE (521)..(540): This region may encompass 1-20 residues
  SITE (541)..(560): This region may encompass 1-20 residues
  SITE (561)..(580): This region may encompass 1-20 residues
  SITE (581)..(600): This region may encompass 1-20 residues
  SITE (601)..(620): This region may encompass 1-20 residues
  SITE (621)..(640): This region may encompass 1-20 residues
  SITE (641)..(660): This region may encompass 1-20 residues
  SITE (661)..(680): This region may encompass 1-20 residues
  SITE (681)..(700): This region may encompass 1-20 residues
  SITE (701)..(720): This region may encompass 1-20 residues
  SITE (721)..(740): This region may encompass 1-20 residues
  SITE (741)..(760): This region may encompass 1-20 residues
  SITE (761)..(780): This region may encompass 1-20 residues
  SITE (781)..(800): This region may encompass 1-20 residues
  SITE (801)..(820): This region may encompass 1-20 residues
  SITE (821)..(840): This region may encompass 1-20 residues
  SITE (841)..(860): This region may encompass 1-20 residues
  SITE (861)..(880): This region may encompass 1-20 residues
  SITE (881)..(900): This region may encompass 1-20 residues
  SITE (901)..(920): This region may encompass 1-20 residues
  SITE (921)..(940): This region may encompass 1-20 residues
  SITE (941)..(960): This region may encompass 1-20 residues
  SITE (961)..(980): This region may encompass 1-20 residues
  SITE (981)..(1000): This region may encompass 1-20 residues
  SITE (1001)..(1020): This region may encompass 1-20 residues
  SITE (1021)..(1040): This region may encompass 1-20 residues
  SITE (1041)..(1060): This region may encompass 1-20 residues
  SITE (1061)..(1080): This region may encompass 1-20 residues
  SITE (1081)..(1100): This region may encompass 1-20 residues
  SITE (1101)..(1120): This region may encompass 1-20 residues
  SITE (1121)..(1140): This region may encompass 1-20 residues
  SITE (1141)..(1160): This region may encompass 1-20 residues
  SITE (1161)..(1180): This region may encompass 1-20 residues
  SITE (1181)..(1200): This region may encompass 1-20 residues
  SITE (1201)..(1220): This region may encompass 1-20 residues
  SITE (1221)..(1240): This region may encompass 1-20 residues
  SITE (1241)..(1260): This region may encompass 1-20 residues
  SITE (1261)..(1280): This region may encompass 1-20 residues
  SITE (1281)..(1300): This region may encompass 1-20 residues
  SITE (1301)..(1320): This region may encompass 1-20 residues
  SITE (1321)..(1340): This region may encompass 1-20 residues
  SITE (1341)..(1360): This region may encompass 1-20 residues
  SITE (1361)..(1380): This region may encompass 1-20 residues
  SITE (1381)..(1400): This region may encompass 1-20 residues
  SITE (1401)..(1420): This region may encompass 1-20 residues
  SITE (1421)..(1440): This region may encompass 1-20 residues
  SITE (1441)..(1460): This region may encompass 1-20 residues
  SITE (1461)..(1480): This region may encompass 1-20 residues
  SITE (1481)..(1500): This region may encompass 1-20 residues
  SITE (1501)..(1520): This region may encompass 1-20 residues
  SITE (1521)..(1540): This region may encompass 1-20 residues
  SITE (1541)..(1560): This region may encompass 1-20 residues
  SITE (1561)..(1580): This region may encompass 1-20 residues
  SITE (1581)..(1600): This region may encompass 1-20 residues
  SITE (1601)..(1620): This region may encompass 1-20 residues
  SITE (1621)..(1640): This region may encompass 1-20 residues
  SITE (1641)..(1660): This region may encompass 1-20 residues
  SITE (1661)..(1680): This region may encompass 1-20 residues
  SITE (1681)..(1700): This region may encompass 1-20 residues
  SITE (1701)..(1720): This region may encompass 1-20 residues
  SITE (1721)..(1740): This region may encompass 1-20 residues
  SITE (1741)..(1760): This region may encompass 1-20 residues
  SITE (1761)..(1780): This region may encompass 1-20 residues
  SITE (1781)..(1800): This region may encompass 1-20 residues
  SITE (1801)..(1820): This region may encompass 1-20 residues
  SITE (1821)..(1840): This region may encompass 1-20 residues
  SITE (1841)..(1860): This region may encompass 1-20 residues
  SITE (1861)..(1880): This region may encompass 1-20 residues
  SITE (1881)..(1900): This region may encompass 1-20 residues
  SITE (1901)..(1920): This region may encompass 1-20 residues
  SITE (1921)..(1940): This region may encompass 1-20 residues
  SITE (1941)..(1960): This region may encompass 1-20 residues
  SITE (1961)..(1980): This region may encompass 1-20 residues
  SITE (1981)..(2000): This region may encompass 1-20 residues
  SITE (1)..(2000): This sequence may encompass 1-20 “(Gly)m(Ser)o(Gly)m(Ser)o(Gly)m” repeating units, wherein m = 1 to 20, o = 1 to 20
  See specification as filed for detailed description of substitutions and preferred embodiments
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 172 - protein, 480 aa, Artificial Sequence
  Description of Artificial Sequence: Synthetic polypeptide
  SITE (5)..(24): This region may encompass 1-20 residues
  SITE (29)..(48): This region may encompass 1-20 residues
  SITE (53)..(72): This region may encompass 1-20 residues
  SITE (77)..(96): This region may encompass 1-20 residues
  SITE (101)..(120): This region may encompass 1-20 residues
  SITE (125)..(144): This region may encompass 1-20 residues
  SITE (149)..(168): This region may encompass 1-20 residues
  SITE (173)..(192): This region may encompass 1-20 residues
  SITE (197)..(216): This region may encompass 1-20 residues
  SITE (221)..(240): This region may encompass 1-20 residues
  SITE (245)..(264): This region may encompass 1-20 residues
  SITE (269)..(288): This region may encompass 1-20 residues
  SITE (293)..(312): This region may encompass 1-20 residues
  SITE (317)..(336): This region may encompass 1-20 residues
  SITE (341)..(360): This region may encompass 1-20 residues
  SITE (365)..(384): This region may encompass 1-20 residues
  SITE (389)..(408): This region may encompass 1-20 residues
  SITE (413)..(432): This region may encompass 1-20 residues
  SITE (437)..(456): This region may encompass 1-20 residues
  SITE (461)..(480): This region may encompass 1-20 residues
  SITE (1)..(480): This sequence may encompass 1-20 “(Gly)(Ser)(Gly)(Gly)(Ser)m” repeating units, wherein m = 1 to 20
  See specification as filed for detailed description of substitutions and preferred embodiments
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 173 - protein, 480 aa, Artificial Sequence
  Description of Artificial Sequence: Synthetic polypeptide
  SITE (4)..(23): This region may encompass 1-20 residues
  SITE (28)..(47): This region may encompass 1-20 residues
  SITE (52)..(71): This region may encompass 1-20 residues
  SITE (76)..(95): This region may encompass 1-20 residues
  SITE (100)..(119): This region may encompass 1-20 residues
  SITE (124)..(143): This region may encompass 1-20 residues
  SITE (148)..(167): This region may encompass 1-20 residues
  SITE (172)..(191): This region may encompass 1-20 residues
  SITE (196)..(215): This region may encompass 1-20 residues
  SITE (220)..(239): This region may encompass 1-20 residues
  SITE (244)..(263): This region may encompass 1-20 residues
  SITE (268)..(287): This region may encompass 1-20 residues
  SITE (292)..(311): This region may encompass 1-20 residues
  SITE (316)..(335): This region may encompass 1-20 residues
  SITE (340)..(359): This region may encompass 1-20 residues
  SITE (364)..(383): This region may encompass 1-20 residues
  SITE (388)..(407): This region may encompass 1-20 residues
  SITE (412)..(431): This region may encompass 1-20 residues
  SITE (436)..(455): This region may encompass 1-20 residues
  SITE (460)..(479): This region may encompass 1-20 residues
  SITE (1)..(480): This sequence may encompass 1-20 “(Gly)(Ser)(Gly)(Ser)m(Gly)” repeating units, wherein m = 1 to 20
  See specification as filed for detailed description of substitutions and preferred embodiments
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 174 - protein, 460 aa, Artificial Sequence
  Description of Artificial Sequence: Synthetic polypeptide
  SITE (4)..(23): This region may encompass 1-20 residues
  SITE (27)..(46): This region may encompass 1-20 residues
  SITE (50)..(69): This region may encompass 1-20 residues
  SITE (73)..(92): This region may encompass 1-20 residues
  SITE (96)..(115): This region may encompass 1-20 residues
  SITE (119)..(138): This region may encompass 1-20 residues
  SITE (142)..(161): This region may encompass 1-20 residues
  SITE (165)..(184): This region may encompass 1-20 residues
  SITE (188)..(207): This region may encompass 1-20 residues
  SITE (211)..(230): This region may encompass 1-20 residues
  SITE (234)..(253): This region may encompass 1-20 residues
  SITE (257)..(276): This region may encompass 1-20 residues
  SITE (280)..(299): This region may encompass 1-20 residues
  SITE (303)..(322): This region may encompass 1-20 residues
  SITE (326)..(345): This region may encompass 1-20 residues
  SITE (349)..(368): This region may encompass 1-20 residues
  SITE (372)..(391): This region may encompass 1-20 residues
  SITE (395)..(414): This region may encompass 1-20 residues
  SITE (418)..(437): This region may encompass 1-20 residues
  SITE (441)..(460): This region may encompass 1-20 residues
  SITE (1)..(460): This sequence may encompass 1-20 “(Gly)(Gly)(Gly)(Ser)m” repeating units, wherein m = 1 to 20
  See specification as filed for detailed description of substitutions and preferred embodiments
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 175 - protein, 6 aa, Artificial Sequence
  Description of Artificial Sequence: Synthetic 6xHis tag
HHHHHH

SEQ ID NO: 176 - protein, 8 aa, Artificial Sequence
  Description of Artificial Sequence: Synthetic peptide
ASHHHHHH

SEQ ID NO: 177 - protein, 10 aa, Artificial Sequence
  Description of Artificial Sequence: Synthetic peptide
GSHHHHHHHH

SEQ ID NO: 178 - protein, 9 aa, Artificial Sequence
  Description of Artificial Sequence: Synthetic peptide
FTIDDSEMG

SEQ ID NO: 179 - protein, 16 aa, Artificial Sequence
  Description of Artificial Sequence: Synthetic peptide
SGSSDDDTYYVDSVKG

SEQ ID NO: 180 - protein, 13 aa, Artificial Sequence
  Description of Artificial Sequence: Synthetic peptide
GPTYPPKDGDCAH

SEQ ID NO: 181 - protein, 9 aa, Artificial Sequence
  Description of Artificial Sequence: Synthetic peptide
YTYCSYDMS

SEQ ID NO: 182 - protein, 9 aa, Artificial Sequence
  Description of Artificial Sequence: Synthetic peptide
YTYCTYDMT

SEQ ID NO: 183 - protein, 9 aa, Artificial Sequence
  Description of Artificial Sequence: Synthetic peptide
STYCTYDMT

SEQ ID NO: 184 - protein, 9 aa, Artificial Sequence
  Description of Artificial Sequence: Synthetic peptide
YTYCSYDMG

SEQ ID NO: 185 - protein, 9 aa, Artificial Sequence
  Description of Artificial Sequence: Synthetic peptide
YTYCNYDIA

SEQ ID NO: 186 - protein, 9 aa, Artificial Sequence
  Description of Artificial Sequence: Synthetic peptide
YTYCSYDMK

SEQ ID NO: 187 - protein, 9 aa, Artificial Sequence
  Description of Artificial Sequence: Synthetic peptide
YTYCSYDMT

SEQ ID NO: 188 - protein, 9 aa, Artificial Sequence
  Description of Artificial Sequence: Synthetic peptide
YTSSSRCMG

SEQ ID NO: 189 - protein, 9 aa, Artificial Sequence
  Description of Artificial Sequence: Synthetic peptide
GTYTSRYMG

SEQ ID NO: 190 - protein, 10 aa, Artificial Sequence
  Description of Artificial Sequence: Synthetic peptide
YTFSTMKYMG

SEQ ID NO: 191 - protein, 9 aa, Artificial Sequence
  Description of Artificial Sequence: Synthetic peptide
YTSCSYDMS

SEQ ID NO: 192 - protein, 16 aa, Artificial Sequence
  Description of Artificial Sequence: Synthetic peptide
AFNSDGTTSYADSVKG

SEQ ID NO: 193 - protein, 16 aa, Artificial Sequence
  Description of Artificial Sequence: Synthetic peptide
SFNSDGSTSYADSVKG

SEQ ID NO: 194 - protein, 16 aa, Artificial Sequence
  Description of Artificial Sequence: Synthetic peptide
GIHSDGTTSYADSVKG

SEQ ID NO: 195 - protein, 16 aa, Artificial Sequence
  Description of Artificial Sequence: Synthetic peptide
AINSDGSTSYADSVKG

SEQ ID NO: 196 - protein, 16 aa, Artificial Sequence
  Description of Artificial Sequence: Synthetic peptide
AIASDGSTSYADSLKG

SEQ ID NO: 197 - protein, 16 aa, Artificial Sequence
  Description of Artificial Sequence: Synthetic peptide
SINSDGTTSYADSVKG

SEQ ID NO: 198 - protein, 16 aa, Artificial Sequence
  Description of Artificial Sequence: Synthetic peptide
AIASDGITSYADSVKG

SEQ ID NO: 199 - protein, 16 aa, Artificial Sequence
  Description of Artificial Sequence: Synthetic peptide
GIDSDGSISYADSVKG

SEQ ID NO: 200 - protein, 16 aa, Artificial Sequence
  Description of Artificial Sequence: Synthetic peptide
AIASDGSTSYADSVKG

SEQ ID NO: 201 - protein, 16 aa, Artificial Sequence
  Description of Artificial Sequence: Synthetic peptide
AIDSDGSTSYADSVKG

SEQ ID NO: 202 - protein, 17 aa, Artificial Sequence
  Description of Artificial Sequence: Synthetic peptide
RIYTPTRTTWYADSVKG

SEQ ID NO: 203 - protein, 17 aa, Artificial Sequence
  Description of Artificial Sequence: Synthetic peptide
TIWPAGGNTVYADSVKG

SEQ ID NO: 204 - protein, 17 aa, Artificial Sequence
  Description of Artificial Sequence: Synthetic peptide
AIWIAAGNTYYADSVKG

SEQ ID NO: 205 - protein, 16 aa, Artificial Sequence
  Description of Artificial Sequence: Synthetic peptide
AIHSDGTTSYADSMKG

SEQ ID NO: 206 - protein, 14 aa, Artificial Sequence
  Description of Artificial Sequence: Synthetic peptide
DPHVQSSGGYCPPY

SEQ ID NO: 207 - protein, 14 aa, Artificial Sequence
  Description of Artificial Sequence: Synthetic peptide
DPHADWGAPCGGDY

SEQ ID NO: 208 - protein, 13 aa, Artificial Sequence
  Description of Artificial Sequence: Synthetic peptide
DPIATITRRCDSY

SEQ ID NO: 209 - protein, 14 aa, Artificial Sequence
  Description of Artificial Sequence: Synthetic peptide
DPNSGWGAPCGGDY

SEQ ID NO: 210 - protein, 13 aa, Artificial Sequence
  Description of Artificial Sequence: Synthetic peptide
DPQTRPGKPCADY

SEQ ID NO: 211 - protein, 13 aa, Artificial Sequence
  Description of Artificial Sequence: Synthetic peptide
DPISTITRICDPY

SEQ ID NO: 212 - protein, 12 aa, Artificial Sequence
  Description of Artificial Sequence: Synthetic peptide
EGTIPVGACPNY

SEQ ID NO: 213 - protein, 13 aa, Artificial Sequence
  Description of Artificial Sequence: Synthetic peptide
DPIATISRRCDSY

SEQ ID NO: 214 - protein, 11 aa, Artificial Sequence
  Description of Artificial Sequence: Synthetic peptide
GASCAVDLFSY

SEQ ID NO: 215 - protein, 12 aa, Artificial Sequence
  Description of Artificial Sequence: Synthetic peptide
EGTIPVGVCPNY

SEQ ID NO: 216 - protein, 12 aa, Artificial Sequence
  Description of Artificial Sequence: Synthetic peptide
EGTVPVGVCPNY

SEQ ID NO: 217 - protein, 15 aa, Artificial Sequence
  Description of Artificial Sequence: Synthetic peptide
AKYGGTSLAPYTYNY

SEQ ID NO: 218 - protein, 18 aa, Artificial Sequence
  Description of Artificial Sequence: Synthetic peptide
ARYGFVPSTWYLPERYNY

SEQ ID NO: 219 - protein, 9 aa, Artificial Sequence
  Description of Artificial Sequence: Synthetic peptide
YTYSSCTMG

SEQ ID NO: 220 - protein, 9 aa, Artificial Sequence
  Description of Artificial Sequence: Synthetic peptide
FTFRLAAMR

SEQ ID NO: 221 - protein, 9 aa, Artificial Sequence
  Description of Artificial Sequence: Synthetic peptide
NTYCEYNMS

SEQ ID NO: 222 - protein, 9 aa, Artificial Sequence
  Description of Artificial Sequence: Synthetic peptide
YTYSNNCMG

SEQ ID NO: 223 - protein, 6 aa, Artificial Sequence
  Description of Artificial Sequence: Synthetic peptide
YTFCMA

SEQ ID NO: 224 - protein, 9 aa, Artificial Sequence
  Description of Artificial Sequence: Synthetic peptide
YTSGNYWMG

SEQ ID NO: 225 - protein, 9 aa, Artificial Sequence
  Description of Artificial Sequence: Synthetic peptide
FTFSRSAMT

SEQ ID NO: 226 - protein, 9 aa, Artificial Sequence
  Description of Artificial Sequence: Synthetic peptide
FTFRLTAMR

SEQ ID NO: 227 - protein, 9 aa, Artificial Sequence
  Description of Artificial Sequence: Synthetic peptide
DTYSSCTMG

SEQ ID NO: 228 - protein, 9 aa, Artificial Sequence
  Description of Artificial Sequence: Synthetic peptide
FTFRTSAMT

SEQ ID NO: 229 - protein, 9 aa, Artificial Sequence
  Description of Artificial Sequence: Synthetic peptide
FTFRLSAMR

SEQ ID NO: 230 - protein, 9 aa, Artificial Sequence
  Description of Artificial Sequence: Synthetic peptide
FTFSSSAMT

SEQ ID NO: 231 - protein, 9 aa, Artificial Sequence
  Description of Artificial Sequence: Synthetic peptide
FNFRLYAMR

SEQ ID NO: 232 - protein, 9 aa, Artificial Sequence
  Description of Artificial Sequence: Synthetic peptide
FTFSTSAMT

SEQ ID NO: 233 - protein, 9 aa, Artificial Sequence
  Description of Artificial Sequence: Synthetic peptide
FTFSSGAMT

SEQ ID NO: 234 - protein, 9 aa, Artificial Sequence
  Description of Artificial Sequence: Synthetic peptide
FTFSNYAMR

SEQ ID NO: 235 - protein, 16 aa, Artificial Sequence
  Description of Artificial Sequence: Synthetic peptide
MLISDGSTFYADSVKG

SEQ ID NO: 236 - protein, 16 aa, Artificial Sequence
  Description of Artificial Sequence: Synthetic peptide
GIDSRGSTIYADSVKG

SEQ ID NO: 237 - protein, 16 aa, Artificial Sequence
  Description of Artificial Sequence: Synthetic peptide
GVDSDGSTRYSESVKG

SEQ ID NO: 238 - protein, 17 aa, Artificial Sequence
  Description of Artificial Sequence: Synthetic peptide
NIYTGGGRTTYADSVKG

SEQ ID NO: 239 - protein, 17 aa, Artificial Sequence
  Description of Artificial Sequence: Synthetic peptide
RFYTRDGYTYYSDSVKG

SEQ ID NO: 240 - protein, 17 aa, Artificial Sequence
  Description of Artificial Sequence: Synthetic peptide
TLWTGGASTFYGDSVKG

SEQ ID NO: 241 - protein, 16 aa, Artificial Sequence
  Description of Artificial Sequence: Synthetic peptide
GIDSGGTTVYADSVKG

SEQ ID NO: 242 - protein, 16 aa, Artificial Sequence
  Description of Artificial Sequence: Synthetic peptide
MVFSDGSTFYADSVKG

SEQ ID NO: 243 - protein, 16 aa, Artificial Sequence
  Description of Artificial Sequence: Synthetic peptide
GIDSAGSTIYADSVKG

SEQ ID NO: 244 - protein, 16 aa, Artificial Sequence
  Description of Artificial Sequence: Synthetic peptide
MLMGDGSTFYADSVKG

SEQ ID NO: 245 - protein, 17 aa, Artificial Sequence
  Description of Artificial Sequence: Synthetic peptide
RFYTRDSYTYYSDSVKG

SEQ ID NO: 246 - protein, 16 aa, Artificial Sequence
  Description of Artificial Sequence: Synthetic peptide
GIDSGGSTIYADSVKG

SEQ ID NO: 247 - protein, 17 aa, Artificial Sequence
  Description of Artificial Sequence: Synthetic peptide
RFYTRDGYTYYSGSVKG

SEQ ID NO: 248 - protein, 16 aa, Artificial Sequence
  Description of Artificial Sequence: Synthetic peptide
GIDSRGSTIYADSVEG

SEQ ID NO: 249 - protein, 8 aa, Artificial Sequence
  Description of Artificial Sequence: Synthetic peptide
ATLGSRTV

SEQ ID NO: 250 - protein, 8 aa, Artificial Sequence
  Description of Artificial Sequence: Synthetic peptide
GVYGDTYS

SEQ ID NO: 251 - protein, 19 aa, Artificial Sequence
  Description of Artificial Sequence: Synthetic peptide
YVCTFCSGNSCYYEYKYYY

SEQ ID NO: 252 - protein, 12 aa, Artificial Sequence
  Description of Artificial Sequence: Synthetic peptide
GSCGSARSEYSY

SEQ ID NO: 253 - protein, 14 aa, Artificial Sequence
  Description of Artificial Sequence: Synthetic peptide
DLARCSSNKNDFRY

SEQ ID NO: 254 - protein, 18 aa, Artificial Sequence
  Description of Artificial Sequence: Synthetic peptide
DPALRLGANILRPAEYKY

SEQ ID NO: 255 - protein, 10 aa, Artificial Sequence
  Description of Artificial Sequence: Synthetic peptide
GLPWGNTWRT

SEQ ID NO: 256 - protein, 8 aa, Artificial Sequence
  Description of Artificial Sequence: Synthetic peptide
ATLGSRTI

SEQ ID NO: 257 - protein, 14 aa, Artificial Sequence
  Description of Artificial Sequence: Synthetic peptide
DLTRCSSNKNDFRY

SEQ ID NO: 258 - protein, 8 aa, Artificial Sequence
  Description of Artificial Sequence: Synthetic peptide
GVYGDTHS

SEQ ID NO: 259 - protein, 10 aa, Artificial Sequence
  Description of Artificial Sequence: Synthetic peptide
GLPWGNIWRT

SEQ ID NO: 260 - protein, 122 aa, Artificial Sequence
  Description of Artificial Sequence: Synthetic polypeptide
QVQLQESGGGSVQAGGSLRLSCAASGYTYCSYDMSWYRQAPGKKREFVSAFNSDGTTSYADSVKGRFTISQDKAKNTVYLQMNSLKPEDTAMYYCKTDPHVQSSGGYCPPYWGQGTQVTVSS

SEQ ID NO: 261 - protein, 122 aa, Artificial Sequence
  Description of Artificial Sequence: Synthetic polypeptide
QVQLQESGGGSVQAGGSLRLSCAASGYTYCSYDMSWYRQAPGKKREFVSSFNSDGSTSYADSVKGRFTISQDNAKNTVYLQMNSLKPEDTAMYYCKTDPHADWGAPCGGDYWGQGTQVTVSS

SEQ ID NO: 262 - protein, 121 aa, Artificial Sequence
  Description of Artificial Sequence: Synthetic polypeptide
QVQLQESGGGSVQAGESLRLSCAASGYTYCTYDMTWYRQAPGKKREFVSGIHSDGTTSYADSVKGRFTISQDNAENTVYLQMNSLKPEDTAMYYCKTDPIATITRRCDSYWGQGTQVTVSS

SEQ ID NO: 263 - protein, 122 aa, Artificial Sequence
  Description of Artificial Sequence: Synthetic polypeptide
QVQLQESGGGSVQAGGSLRLSCAASGSTYCTYDMTWYRQAPGKRREFVSAINSDGSTSYADSVKGRFTISQDNAKNTVYLQMNSLKPEDTAMYYCKTDPNSGWGAPCGGDYWGQGTQVTVSS

SEQ ID NO: 264 - protein, 122 aa, Artificial Sequence
  Description of Artificial Sequence: Synthetic polypeptide
QVQLQESGGGSVQAGGSLRLSCAASGYTYCSYDMSWYRQAPGKKREFVSAIASDGSTSYADSLKGRFTISQDNAKNTVYLQMNSLKPEDTAMYYCKTDPHVQSSGGYCPPYWGQGTQVTVSS

SEQ ID NO: 265 - protein, 121 aa, Artificial Sequence
  Description of Artificial Sequence: Synthetic polypeptide
QVQLQESGGGLVQPGGSLRLSCAASGYTYCSYDMGWYRQAPGKKRKFVSSINSDGTTSYADSVKGRFTISQDNAKNTVYLQMNSLKPEDTAMYYCKTDPQTRPGKPCADYWGQGTQVTVSS

SEQ ID NO: 266 - protein, 121 aa, Artificial Sequence
  Description of Artificial Sequence: Synthetic polypeptide
QVQLQESGGGSVQAGGSLRLSCAASGYTYCNYDIAWYRQAPGKERKFVSAIASDGITSYADSVKGRFTISQDNAKNTVYLQMNSLKPEDTAMYYCKTDPISTITRICDPYWGQGTQVTVSS

SEQ ID NO: 267 - protein, 120 aa, Artificial Sequence
  Description of Artificial Sequence: Synthetic polypeptide
QVQLQESGGDSVQAGGSLRLSCAASGYTYCSYDMKWYRQAPGKEREFVSGIDSDGSISYADSVKGRFTISQDNAKNTVYLQMNSLKPEDTAMYYCKTEGTIPVGACPNYWGQGTQVTVSS

SEQ ID NO: 268 - protein, 121 aa, Artificial Sequence
  Description of Artificial Sequence: Synthetic polypeptide
QVQLQESGGGLVQAGGSLRLSCAASGYTYCSYDMSWYRQAPGKERKFVSSINSDGTTSYADSVKGRFTISQDNAKNTVYLQMNSLKPEDTAMYYCKTDPQTRPGKPCADYWGQGTQVTVSS

SEQ ID NO: 269 - protein, 121 aa, Artificial Sequence
  Description of Artificial Sequence: Synthetic polypeptide
QVQLQESGGGSVQAGGSLKLSCAASGYTYCNYDIAWYRQAPGKERKFVSAIASDGSTSYADSVKGRFTISQDNAKNTVYLQMNSLKPEDTAMYYCKTDPIATMTRRCDPYWGQGTQVTVSS

SEQ ID NO: 270 - protein, 121 aa, Artificial Sequence
  Description of Artificial Sequence: Synthetic polypeptide
QVQLQESGGGSVQAGGSLRLSCAASGYTYCSYDMTWYRQAPGKKREFVSAIDSDGSTSYADSVKGRFTISQDNAKNTVYLQMNSLKPEDTAMYYCKTDPIATISRRCDSYWGQGTQVTVSS

SEQ ID NO: 271 - protein, 120 aa, Artificial Sequence
  Description of Artificial Sequence: Synthetic polypeptide
QVQLQESGGGSVQAGGSLRLSCAASGYTSSSRCMGWFRQAPGKEREGVARIYTPTRTTWYADSVKGRFTISQDNAKNTVYLEMASLKPEDTAKYFCAAGASCAVDLFSYWGQGTQVTVSS

SEQ ID NO: 272 - protein, 120 aa, Artificial Sequence
  Description of Artificial Sequence: Synthetic polypeptide
QVQLQESGGGSVQAGGSLRLSCAASGYTYCSYDMKWYRQAPGKKREFVSAIDSDGSTSYADSVKGRFTISQDNAKNTVYLQMNSLKPEDTAMYYCKTEGTIPVGVCPNYWGQGTQVTVSS

SEQ ID NO: 273 - protein, 120 aa, Artificial Sequence
  Description of Artificial Sequence: Synthetic polypeptide
QVQLQESGGGLVQPGGSLRLSCAASGYTYCSYDMKWYRQAPGKKREFVSAIDSDGSTSYADSVKGRFTISQDNAKNTVYLQMNSLKPEDTAMYYCKTEGTVPVGVCPNYWGQGTQVTVSS

SEQ ID NO: 274 - protein, 124 aa, Artificial Sequence
  Description of Artificial Sequence: Synthetic polypeptide
QVQLQESGGGSVQAGGSLRLSCAASPGTYTSRYMGWFRQAPGKEREGVATIWPAGGNTVYADSVKGRFTISQDGAKKTVYLQMNSLKPEDTAMYYCAAAKYGGTSLAPYTYNYWGQGTQVTVSS

SEQ ID NO: 275 - protein, 121 aa, Artificial Sequence
  Description of Artificial Sequence: Synthetic polypeptide
QVQLQESGGGSVEAGGALTLSCVASGYTYCNYDIAWYRQAPGKERKFVSAIASDGSTSYADSVKGRFTISQDNAKNTVYLQMNSLKPEDTAMYYCKTDPIATMTRRCDPYWGQGTQVTVSS

SEQ ID NO: 276 - protein, 128 aa, Artificial Sequence
  Description of Artificial Sequence: Synthetic polypeptide
QVQLQESGGGSVQAGGSLRLSCTASGYTFSTMKYMGWFRQAPGKEREGVAAIWIAAGNTYYADSVKGRFTISQDNTKNTVYLQMNSLKPEDTALYYCAAARYGFVPSTWYLPERYNYWGQGTQVTVSS

SEQ ID NO: 277 - protein, 121 aa, Artificial Sequence
  Description of Artificial Sequence: Synthetic polypeptide
QVQLQESGGGSVQAGGSLRLACAASGYTYCNYDIAWYRQAPGKERKFVSAIASDGSTSYADSVKGRFTISQDNAKNTVYLQMNSLKPEDTAMYYCKTDPIATMTRRCDPYWGQGTQVTVSS

SEQ ID NO: 278 - protein, 120 aa, Artificial Sequence
  Description of Artificial Sequence: Synthetic polypeptide
QVQLQESGGGSVQAGGSLRLSCAASGYTSCSYDMSWYRQAPGKKREFVSAIHSDGTTSYADSMKGRFTISQDNAKNTVYLQMNSLKPEDTAMYYCKTDPNYSDHVCPPYWGRGTQVTVSS

SEQ ID NO: 279 - DNA, 378 bp, Artificial Sequence
  Description of Artificial Sequence: Synthetic polynucleotide
caggtccagctccaggagtctggcggtggctcagtacaagctgggggctctctgcgtttgtcctgtgtggcgagcgggtacggatactgtgggtacgacatgagttggtacagacaggcccctggcaaggaacgtgaatttgtggccctcatcacttctgatcgctccattagctacgaggattctgtcaaagctcgctttatcatttcccgcgacaacgccgctaacactggttatctggacatgactagactgacccccgatgacacggccatttactattgcaagaccagtgcagcggcccgcgaatcttcctggtgtcgctctcgctaccgcgtggcatcatggggccagggtactcaggtcaccgtgtctagc

SEQ ID NO: 280 - DNA, 381 bp, Artificial Sequence
  Description of Artificial Sequence: Synthetic polynucleotide
caagtccaactccaggagtctggtgggggctctgttcaagctggcgggtccctgcgcctttcctgtaccgccagcggctacacgtactctagcgccttcatggcttggtttcggcaggcccctggaaaagagagagagggagtggcagctatctacactcgtgacggcggaaccgtgtacgctgatagtgtcaagggccgcttcaccatttcccaggataatgccaagaatatcctgtatctccagatgaactcccttaaagccgaagacactgcgatgtactattgcgcagccaaaatcccgcagccaggccgggcttctttgctggatagccaaacctacgactattggggtcaaggcactcaggttaccgtgtcttcc

SEQ ID NO: 281 - DNA, 378 bp, Artificial Sequence
  Description of Artificial Sequence: Synthetic polynucleotide
caggtccagcttcaggagagcggcggaggctccgtgcaggctgggggatctttgagactcagctgcgtggccagtggctactcttactgtgggtacgacatgatgtggtatcgccaagcgccgggcaaggaacgtgagttcgtggcgctcatcacttccgactactcaattcgttacgaggattccgttgagggccgcttcagcatttctcgtgacaacgcgaagaacacaggatacttgctgatgagtaacctcacccccgccgataccgctatttattactgcaagacaagtacagctgccagggagagcagttggtgtcggtctcgctatcgtgtggcctcctggggacagggcacccaagtaaccgtgtcatca

SEQ ID NO: 282 - DNA, 378 bp, Artificial Sequence
  Description of Artificial Sequence: Synthetic polynucleotide
caggtgcagctccaggaatctggtgggggcagtgttcaggctggtggcagcctgagacttagctgcgtggcttctggctatggttactgtgggtacgacatgagctggtatcggcagacccccggaaaggagcgggagttcgtagcgctcatcacaagtgaccgcatcgcctcctatgaagactccgttaagggtcgctttatcattagccgggacaatgccaagaacacaggttacctcgatatgactcgggtcacacctgacgataccgctatctattactgcaagacttctgcggctgcccgtgaaaacagctggtgccgctcaagataccgggtggcctcctggggacagggaactcaggtcaccgtctctagc

SEQ ID NO: 283 - DNA, 378 bp, Artificial Sequence
  Description of Artificial Sequence: Synthetic polynucleotide
caggtgcagttgcaggagagcggaggcggatctgtgcaggccggtggatttctgcggctgtcttgcgtggcgagcggctatggctattgcggatacgacatgagctggtatcgccaggttccgggtaaggagcgtgagttcgtcgctctgattacctctgatcgctctgtgtcctatgaggactccgttaagggtagattctctatctctcgcgataatgctaagaacacagcctacctggagatgaacagactgacccccgacgataccgctgtctattactgtaagacctccacagccgctcgcgagaataactggtgccgctctcgctatagaatcgcctattggggtcagggtacacaagttaccgtatcctcc

SEQ ID NO: 284 - DNA, 381 bp, Artificial Sequence
  Description of Artificial Sequence: Synthetic polynucleotide
caggtgcagttgcaggagagtggcgggggctctgttcaggctggtggatcattgcgtctgagctgtgctgcctcccgctacacctacactaataacttcatggcttggtttagacaagctcctggcaaggaacgcgaaggcgttgccgcgatttataccggagacggttacgcatattacttctattccgtgaagggccgcttcacaatctcccaggataacgacgaaaatatgctctacttgcagatgaactccctcaaacctgaggacacggcaatgtactattgtgcggctatggagcgccgtatcggaactcgccgtatgaccgaaaacgctgagtataagtattggggacaaggaacccaggtgaccgtatcctcc

SEQ ID NO: 285 - DNA, 378 bp, Artificial Sequence
  Description of Artificial Sequence: Synthetic polynucleotide
caggtccagttgcaggagtctggtggcggaagcgtgcaggctgggggcagcctcaggctgtcctgtgctgtgtccgggtacgactactgcggctacgacgtgcgctggtatcgccgtgcccccggcaaggagagggagttcgtctccgggattgattccgatggctctaccagttacgcagattccgtcaagggtcgttttaccattagtcaggataacgctgagaacacaagctatctgcacatgttctcactgaagcctgaggatacggccatgtactattgcaagactgagtcccccgcaggtgaatccgcctggtgtcgtaactttcgcggcatggactactggggaaagggcacccaggtcactgtgtcttct

SEQ ID NO: 286 - DNA, 378 bp, Artificial Sequence
  Description of Artificial Sequence: Synthetic polynucleotide
caggtgcagctccaggaatcaggcggtgggtccgtgcaggcaggagggagtctgcgcctgtcctgtgtggcctccggttacagctactgcggctacgatatgatgtggtataggcaagctccagggaaggagcgtgagttcgtggcccttatcacatctgactattccatccgctacgaggactccgtggagggaagattttcaatctccagagacaacgcaaagaacaccggatacctcctgatgtctaacctgaccccagccgacacggcaatctattactgtaaaacctccacagcagcgagggagtccagctggtgcaggtccagataccgtgttgcctcctggggacagggcactcaggtgacggtgagttct

SEQ ID NO: 287 - DNA, 378 bp, Artificial Sequence
  Description of Artificial Sequence: Synthetic polynucleotide
caggtgcagctccaggagtccggtggcgggagcgtgcaggctggcggatctctgcggctcagttgcgtcgcctcagggtattcctattgtggctacgatatgatgtggtatcgtcaggcccccggcaaggagcgcgagttcgtcgccctgattacaagcgattattcaatccgttatgaagattccgtggaggggcgcttctccatcagtcgcgacaacgccaaaaacactggctaccttctgatgtcaaacctgactcccgctgacaccgcgatctactattgtaaaacctcaacggctgcccgcgagtccggctggtgccggtctaggtatcgtgtggccagctgggggcagggcactcaggtcaccgtgtcatcc

SEQ ID NO: 288 - DNA, 378 bp, Artificial Sequence
  Description of Artificial Sequence: Synthetic polynucleotide
caggtccagctgcaagaatccggtggaggctctgtgcaggcgggtgggtccctgcgcctgtcttgcgccgtgtctggctatgattattgcggatatgacgtgcgctggtatcgccaggctcccggcaaggaacgcgagtttgtctctgggattgactcagacggcagcactagctatgccgactccgtgaaaggtcgcttcaccatttcccaagacaacgccgagaataccagctatctgcacatgttcagcctcaaacctgaagatactgccatgtattactgtaagacggagagtcccgcaggcgaatccgcttggtgtcggaatttcaggggaatggactactggggcaagggtactcaagtgaccgtaagctct

SEQ ID NO: 289 - DNA, 378 bp, Artificial Sequence
  Description of Artificial Sequence: Synthetic polynucleotide
caggtgcagctccaggagagcggcggaggctccgtgcaggcgggcgggagcctgcgtctgtcttgtgccgtatctggctatgactattgcggttacgacgttcgctggtacaggcaggctccgggcaaggagcgtgagtttgtcagcgggattgacagtgacggctccacctcttatgcggattccgtgaagggacgcttcacaatttcccaggataacgcagagaacacctcctacctccacatgttcagcctcaaacccgaagatactgctatgtattactgtaaaacagagagcccagccggggagtctgcttggtgtcgtaactttcgcggcatggactactggggcaagggaacccaggtgaccgtctcttcc

SEQ ID NO: 290 - DNA, 381 bp, Artificial Sequence
  Description of Artificial Sequence: Synthetic polynucleotide
caggtgcaactccaagagagcggaggcgggagtgttcaggccgggggctctctgcggctgtcctgcaccgcctctggttacacctactccagcgccttcatggcctggttccggcaggcacctggcaaggaacgcgaaggcgtagccgctatctatacgcgcgatgggggtacagtttatgctgatagcgttaaaggacgcttcactatctcccaggacaacgccaaaaacaccctgtacttgcagatgaactccctcaaacctgaagatacggcgatgtactattgtgcggcaaagatgcctcagcccggacgcgcaagtctgcttgactctcaaacttatgattactggggccaagggactcaggtgaccgttagctcc

SEQ ID NO: 291 - DNA, 378 bp, Artificial Sequence
  Description of Artificial Sequence: Synthetic polynucleotide
caggtgcagttgcaggaaagcggcggtggctcagtccaggccgggggcttcttgcgcttgagttgcgtggcgagcggatatggctactgtggctacgatatgagctggtatcgtcaggctccgggcaaggaacgtgagttcgtcgcgctcatcactagcgaaagagtcatctcctacgaagactccgttaagggccgcttttccatttctcgcgacaacgccgagaacacgggctaccttgaaatgaatagactgactcccgacgatactgccatctactattgcaagacaagcgccgctgcacgcgagtcctcttggtgcaggtctcgctaccgcgtggcttcttgggggcaggggacccaggtgaccgtatcatcc

SEQ ID NO: 292 - DNA, 378 bp, Artificial Sequence
  Description of Artificial Sequence: Synthetic polynucleotide
caggttcaactccaggagtccgggggcggttccgtgcaggctgggggctcccttagacttagctgtgccgtgtctggatacgattactgtgggtatgacgtgcggtggtacagacgcgctccgggaaaggaacgcgagttcgtgagcggaattgattccgatggcagcacctcctatgcggattctgtgaagggccgcttcactatctctcaagacaacgccgagaacactagctacctgcacatgttcagtctgaaaccggaggataccgcgatgtattactgtaagaccgagtctcctgctggagagagcgcgtggtgcagaaacttccgtggaatggactattggggtaaaggaactcaggtgactgtgtccagt

SEQ ID NO: 293 - DNA, 378 bp, Artificial Sequence
  Description of Artificial Sequence: Synthetic polynucleotide
caagtgcagctccaggaatctggaggcggaagcgtacaggccggtggctcactccggctttcttgcgctgtgtcaggttacgactattgtggatatgatgtccggtggtataggcaagcgccgggaaaggagcgcgagttcgtgagcggtatcaactctgacggctccacctcctacgccgactctgtcaagggccgctttacaatttctcaggacaacgcagagaacacctcttacctgcacatgttcagcttgaagccggaggacaccgcgatgtactattgtaagactgagtcccccgctggagagtctgcatggtgccgtaattttcgcggcatggactattgggggaaaggtactcaggttaccgtaagctca

SEQ ID NO: 294 - DNA, 381 bp, Artificial Sequence
  Description of Artificial Sequence: Synthetic polynucleotide
caggtacagctccaggagagtggaggcgggtcagtgcaggccgggggctcactgcgcttgagctgcaccgcgagcggttacacctacagctccgcattcatggcttggttcaggcaagccccaggcaaggagcgcgagggcgtggctgccatgtatacccgcgacgggggcaccgtgtatgccgattccgtgaagggccgtttcaccatctcccaggataacgctaagaacaccctctacctccagatccacactctcaaagccgaagacacggctatgtactattgcgccgcgaagatccctcaacctggcagggcaagccttctggactcccagacgtatgactattggggccaggggactcaggttacagtgtccagc

SEQ ID NO: 295 - DNA, 378 bp, Artificial Sequence
  Description of Artificial Sequence: Synthetic polynucleotide
caggtgcagctccaggaatccggcggtgggtctgtgcaggcagggggttttctccgcttgagctgtgtggctagtggatacggttattgtggatacgacatgagctggtatcgccaagtaccgggcaaggagcgtgagtttgtggccctcatcacctctgatcgctccgtgtcttatgaggacagcgtgaagggccgcttcagcatcagtcgcgacaacgccaagaacaccgcttatctggaaatgaacagactcaccccggatgacacagctatctactattgcaagacctccacagcggccagagagaataactggtgccggtcccgctaccgcatcgcgtcctggggccagggcacccaggtgactgtctcctct

SEQ ID NO: 296 - DNA, 381 bp, Artificial Sequence
  Description of Artificial Sequence: Synthetic polynucleotide
caggtgcagttgcaggagtctggagggggcagcgtgcaggccggaggctccctccgcctcagctgcgcggcctcccggtacacctacaccaataacttcatggcatggttcaggcaggccccaggaaaggagcgtgagggggtcgccgcaatctataccggagacggctacgcctattactttgactccgttaaagggcgtttcaccatcagtcaagacaacgacaaaaacatgctctacctccagatgaatagcttgaagccggaggataccgcaatgtactattgtgccgcgatggagagacgctccggtcggcgtcgcatgactgaaaatgccgagtacaagtactgggggcaggggactcaggtgaccgtgagcagc

SEQ ID NO: 297 - DNA, 378 bp, Artificial Sequence
  Description of Artificial Sequence: Synthetic polynucleotide
caagttcagctccaggagagtggaggcggttccgtacaggctggcggaagtctgcgcctctcctgcgccgtctccggttacgactattgtgggtacgacgtgcgctggtatagacaggctcctggaaaggagcgtgagtttgtgagtggcatcaactccgacggtagcacctcctatgctgattctgtgaagggtcgctttacaatctcacaggacaacgccgaaaacacttcctatctgcacatgttcagcctcaagcccgaagacaccgcaatgtactattgtaagactgaaggtccagctggcgagagtgcatggtgcaggaattttaggggcatggactactggggcaagggcacccaggtcaccgtgtcttca

SEQ ID NO: 298 - DNA, 381 bp, Artificial Sequence
  Description of Artificial Sequence: Synthetic polynucleotide
caggtgcagttgcaggaatcaggaggcggttctgtgcaggccggaggcagcctgcgtctgagctgcaccgcttctgggtacacctactcaagtgccttcatggcctggtttcggcaagcgcccggcaaggaacgcgagggagttgcggccatctacaccagggacggcagtcccgtgtacgctgactccctgaagggccgtttcaccatcagccaggataacgcaaagaacaccctgcacctccagatgaacagcctgaaacctgaggacacagctatgtattactgcgcggccaaaatccctgagcctggaagaatcagcctccttgactcccagacctacgactactggggtcacggcactcaggtgactgtgtcttct

SEQ ID NO: 299 - DNA, 381 bp, Artificial Sequence
  Description of Artificial Sequence: Synthetic polynucleotide
caggttcaactccaagagtctggaggcgggtccgtgcaggctgggggctccctcagactgtcctgtactgcgtcagggtacacctacagctccgctttcatggcttggttccggcaagctccgggcaaggagcgcgagggcgtggccgcgatgtatacccgcgacggtggcaccgtgtacgccgactctgttaaaggccgcttcaccatctcccaggataacgccaagaacaccctgtacctccagatgaactctttgaagaccgaggataccgctatgtactattgcgccgcaaaaattccccagccgggccgtgcttcccttctggacagccaaacctatgattactggggccagggcacacaggtgaccgtgtcctcc

SEQ ID NO: 300 - DNA, 381 bp, Artificial Sequence
  Description of Artificial Sequence: Synthetic polynucleotide
caggtgcaacttcaggaatctggcggtggcagcgtgcaggctggtggctccctgcgcctgagctgtactgcttccggctacacatactctagtgcgttcatggcctggttcaggcaagctccgggaaaggagcgcgagggtgtggcggccatttatacacgcgacggaggcaccgtgtacgctgactctgtcaagggccgcttcaccatctcacaggacaatgcaaaaaataccctctaccttcagatgaacagcctgaaggcagaggacacagcaatgtattactgtgcagccaagatcccacaacccggacgcgcgtccctcctggattcacagacctacgactactggggccagggcacgcaggttactgtatcaagc

SEQ ID NO: 301 - DNA, 381 bp, Artificial Sequence
  Description of Artificial Sequence: Synthetic polynucleotide
caggtgcagctccaggaaagcgggggaggttccgtccaggccggtggctccctccgcctgtcatgcacagcgagcggttacacgtatagctccgcctttatggcctggtttagacaggccccagggaaagaacgtgagggagtggctgcaatttacacccgcgatggcgggactgtttacgccgatagcgtcaagggtcgctttaccatcagccaggacaacgctaaaaacaccctctatctccagatgaatagcctgaaggccgaggacactgcgatgtattactgcgccgctaagatccctcaacctggccgcgccagcttgctggatagccagacatacgattactggggtcagggaacacaagtgacggtcagcagc

SEQ ID NO: 302 - DNA, 378 bp, Artificial Sequence
  Description of Artificial Sequence: Synthetic polynucleotide
caggtgcagctccaggagagcggcgggggctccgtacaggccggtggatcactccgcctgagctgtgctgtgagcgggtacgactattgcggatacgacgtgcgctggtatcgccaagctccagggaaggaaagggagttcgtgagcggaattgattccgatggctccaccagttatgccgactccgttaaaggaaggtttaccatctcccaagataacgccgagaacacctcctatctgcatatgttttccctgaaacccgaggataccgctatgtattactgtaagacagagagccctgccggagagtccgcctggtgccgcaactttcggggcatggactactggggaaagggcacccaggtgacagtgtctagc

SEQ ID NO: 303 - DNA, 378 bp, Artificial Sequence
  Description of Artificial Sequence: Synthetic polynucleotide
caggtgcagctgcaagaatcaggaggtggatctgtgcaagctgggggctctttgcgcctgtcctgtgtcgcctccggctatagctattgcggctatgacatgatgtggtacaggcaagccccaggtaaggagagggagtttgtggctctcatcacctccgactacagcattcgctatgaagatagtgtcgagggacgcttctccatttctcgcgacaacgcgaagaacactggctatttgctgatgagtaacctcacccccgccgacaccgcgatctactattgcaaaacatctaccgccgctcgggaaagtagctggtgtaggtcacgttatagggtcgcttcctggggtcagggcacgcaggtgaccgtctcatcc

SEQ ID NO: 304 - DNA, 381 bp, Artificial Sequence
  Description of Artificial Sequence: Synthetic polynucleotide
caggtgcagttgcaggagagcggaggcggatctgtgcaggcaggcggaagcctccgcctgtcttgcgccgcttcccggtacacctacacaaataactttatggcatggttccgccaagcgcccggcaaggagcgcgagggtgtcgcggccatttacacaggtgatggctacgcctattacttcgactccgtgaaaggcaggttcacgatctcccaggataacgacaagaatatgttgtatcttcagatgaactctctgaaacctgaggacaccgctatgtactattgtgcagctatggaacgcaggtcaggcaggcgcaggatgaccgagaacgccgagtacaagtactggggccagggcacccaggtgaccgtgtcttca

SEQ ID NO: 305 - DNA, 363 bp, Artificial Sequence
  Description of Artificial Sequence: Synthetic polynucleotide
caggtgcagctccaggagtctggaggcggttccgtccaggccggggaaacgctccggcttagctgcaccgtctccggtttcaccattgatgactccgaaatgggttggtatcgccaagcgcccggccatgagtgcgaactggtggccagcggaagttccgacgatgacacctattacgtggactcagtgaagggtcgctttacgatctctctggataacgccaaaaacatggtgtacctccagatgaactcactcaagccagaggatacagcagtttattactgtgccactggacctacataccctcccaaggatggtgactgcgcacactggggtcaaggcacccaggtcactgtctcctcc

SEQ ID NO: 306 - DNA, 381 bp, Artificial Sequence
  Description of Artificial Sequence: Synthetic polynucleotide
caagtccagctccaggagtctgggggaggctcagtgcaagctggtggatctcttcgcctgtcttgcaccgcttctgggtacacctatagctctgccttcatggcctggtttaggcaagcgcctggcaaggagcgggagggcgtcgccgctatctacacccgcgacggcagtccggtttatgccgactccctgaagggtagatttactatctctcaggataatgcaaagaatacgctgcacttgcagatgaactccctcaaacccgaggacacggccatgtattactgtgctgcaaaaatcccagagcctggtcggatctccctcctggattcacagacctacgactactggggccacggcacccaggtgacagtctcttcc

SEQ ID NO: 307 - DNA, 378 bp, Artificial Sequence
  Description of Artificial Sequence: Synthetic polynucleotide
caggtgcagctccaggagtccggtggcggaagcgtgcaggccggtggctccctgcggttgagttgcgcggtctcaggttacgattattgtggctacgacgtgcgctggtatagacgcgctcctggcaaggagcgtgagttcgtgtctggcatcgactccgatggctctacttcatacgctgattccgtcaaaggccgtttcaccatctctcaggataacgccgagaacacctcctaccttcacatgttctctctgaagcccgaggatactgcaatgtattactgtaagactgagtctcctgccggagaatccgcctggtgtcgtaactttcgtggcatggactactggggtaagggaacccaggtgactgtatcttcc

SEQ ID NO: 308 - DNA, 378 bp, Artificial Sequence
  Description of Artificial Sequence: Synthetic polynucleotide
caggtccagttgcaggagtctggtggaggctccgtccaagctgggggctttcttaggctgtcatgtgtggcatccggctatgggtattgtggctatgatatgtcctggtatagacaagcgcccggcaaggagcgcgagttcgtggcgctgattaccagcgagcgcgttatcagctacgaggactccgtcaaaggcagattctccatctcacgcgacaacgccgagaacacaggctatctggaaatgaatcgtttgacacctgatgacaccgctatctactattgcaagacctctgcggctgcgcgtgagtctagctggtgccgttcccgctatagagtggcttcttggggtcagggaacccaggtgacagtctccagc

SEQ ID NO: 309 - DNA, 378 bp, Artificial Sequence
  Description of Artificial Sequence: Synthetic polynucleotide
caggtacagctccaggagtctggaggcgggagcgtgcaggcaggcggttccctgcgtctgtcctgcgtcgcctctgggtatgggtactgcggctacgatatgtcctggtatcgtcaggctcccggcaaagaaagagagttcgtagccctcatcacatctgaccggagcatttcctacgaagactccgtcaaggcccgcttcattatctcacgggataacgcagccaacaccggatacctggacatgactcgcctgacccccgatgacactgctatctattactgcaagacgagcgcggcagctcgcgagagttcttggtgccggtcccggtacagggtggcgtcctggggccaggggactcaggtcaccgtctcctcc

SEQ ID NO: 310 - DNA, 378 bp, Artificial Sequence
  Description of Artificial Sequence: Synthetic polynucleotide
caggtgcaactccaggagagtggaggtggctcagtacaggccgggggaagcctccgtctgagctgtgccgtgtccggctacgattactgtggttacgacgtgcggtggtatcgccaggcccctggtaaggaaagagagttcgtgtccggcatcgacagcgatggtagcacatcttacgccgactccgtgaagggccgcttcacaatctcccaggacaacgccgaaaacacgtcttacctccatatgttttccctgaaacctgaagacaccgctatgtattactgcaagaccgagtctcccgctggcgagtcagcatggtgtaggaactttcgcggcatggactattggggtaagggcacccaggtgacggtgagttct

SEQ ID NO: 311 - DNA, 378 bp, Artificial Sequence
  Description of Artificial Sequence: Synthetic polynucleotide
caggtgcagctccaggaaagcggcgggggaagcgtgcaggcaggaggctcccttcggttgagctgcgtggccagcggctacagctactgcggctacgacatgatgtggtatcgccaagctccggggaaggagcgcgagttcgtcgccctcatcaccagtgattattctatccgctacgaagactctgtggaaggtaggttctccattagcagagacaacgcaaagaacactggatacctgcttatgagcaacctcacacccgccgacactgccatctactattgtaagacctctaccgccgctcgcgaaagctcctggtgcaggtcccgctatcgcgtggccagttggggtcagggaacccaggtgacggtatctagc

SEQ ID NO: 312 - DNA, 381 bp, Artificial Sequence
  Description of Artificial Sequence: Synthetic polynucleotide
caggttcagttgcaggagtctggaggtggcagtgtgcaagctggaggctccctccgcctgagttgcgctgccagcagatatacctatacgaataactttatggcttggtttagacaggcccccggtaaagagcgggaaggtgtggccgcgatttacaccggcgatggctacgcctattacttttacagcgtgaagggacgtttcaccatttctcaggataacgatgaaaacatgctgtatctccaaatgaactctctgaagcctgaagacaccgctatgtattactgcgcggctatggagcgcaggatcggaacaagacgcatgactgagaacgctgagtataaatattggggacaaggcacacaggtgacagttagctcc

SEQ ID NO: 313 - DNA, 378 bp, Artificial Sequence
  Description of Artificial Sequence: Synthetic polynucleotide
caggtccaactccaggagtccgggggagggtctgtgcaggcgggtggctccctgcgcctgagctgtgtcgcgtctggttactcctactgtggatatgatatgatgtggtatagacaggccccaggtaaggagcgcgagtttgtggccctgattaccagcgactacagtatccgctatgaggattccgtggagggccgcttctctatctcacgcgacaacgccaagaatacaggctacctcctgatgagcaacctgacccctgccgacacagccatttattactgcaagacctccaccgccgcgcgtgaatccggctggtgcaggtcacgctatcgtgtcgccagctggggtcaggggacacaggtgacggtgtcatct

SEQ ID NO: 314 - DNA, 378 bp, Artificial Sequence
  Description of Artificial Sequence: Synthetic polynucleotide
caagtgcagttgcaagaatcaggaggcgggtccgtgcaggcgggcggatctctgcgtctgtcttgtgctgtctccggttatgactactgtggttacgacgtgcgctggtatcgccaggcccctggtaaggaacgtgagttcgtgagcgggatcaatagcgacggctccacctcttatgccgacagtgtgaagggtaggtttaccatcagtcaagacaacgccgagaacacatcctaccttcatatgttctctctcaagcctgaggataccgcaatgtactattgcaagacggagtccccagcaggtgagtccgcttggtgcagaaactttcgcggcatggattattgggggaagggaacccaggtcaccgtgtcttcc

SEQ ID NO: 315 - DNA, 378 bp, Artificial Sequence
  Description of Artificial Sequence: Synthetic polynucleotide
caggtgcaacttcaggaatccggtggcggatctgttcaggctggcggattcctgcgcctgtcttgcgtggccagtggctacggctactgcggctatgatatgtcatggtatcgccaagtgcccggcaaggagcgcgagtttgtagccctcatcacatctgatcgttctgtcagctacgaagacagtgtcaagggccgcttttccatcagccgcgataatgcgaagaacacggcctacctggagatgaacagactgacaccggatgacaccgctgtatattactgtaagacctcaacggctgccagagagaataattggtgccgttctcgctaccgcatcgcttattggggccagggaacacaggtcacagtctcctcc

SEQ ID NO: 316 - DNA, 378 bp, Artificial Sequence
  Description of Artificial Sequence: Synthetic polynucleotide
caggtgcaactccaggagagcgggggaggttccgttcaggccgggggttccctcagattgtcttgtgccgtctccgggtacgattactgtggctatgacgtgcgctggtatcggcaggctcctgggaaggagcgggagttcgtgagtggcattaactcagacgggtctacctcctatgccgacagcgttaagggcaggtttactatcagtcaggacaatgcggagaataccagttacctgcacatgttcagcctcaagcccgaggataccgccatgtattactgcaagacagagggtccagctggcgagtccgcatggtgccgcaacttcaggggtatggactactggggcaagggtactcaggtgactgtgtcctct

SEQ ID NO: 317 - DNA, 381 bp, Artificial Sequence
  Description of Artificial Sequence: Synthetic polynucleotide
caggtgcagttgcaggagtcaggcgggggctctgtccaggctgggggctctctgagactgtcttgtactgcgtctggttacacgtacagttctgcctttatggcctggtttcggcaagcgcccggaaaggagcgcgagggtgttgctgccatgtatacccgtgatggcggaaccgtctacgcagattctgttaagggtcgtttcacaatctcccaggacaatgcgaaaaataccctctatctccagatccacaccttgaaggctgaggacaccgcgatgtattactgtgctgccaagatcccgcagcctggccgcgcttccctgctcgacagccagacatacgactactggggtcagggcacacaggttaccgtgagtagt

SEQ ID NO: 318 - DNA, 381 bp, Artificial Sequence
  Description of Artificial Sequence: Synthetic polynucleotide
caagtccaactccaggaaagcggaggtggcagcgtccaggccgggggctctctgagactgtcttgtaccgcttccggctatacatattcctctgcctttatggcatggttccgccaagcgccaggcaaggagcgcgagggcgtcgccgctatgtataccagagacggaggcaccgtctacgctgacagcgtcaagggacgcttcacaatctcccaggacaacgccaagaatactttgtatctccagatgaatagcctcaagacggaggacaccgcaatgtattactgcgctgcaaaaatccctcagccaggtcgcgcctccctcctggacagtcagacctatgattattggggccaggggacccaggtgactgtctcctcc

SEQ ID NO: 319 - DNA, 378 bp, Artificial Sequence
  Description of Artificial Sequence: Synthetic polynucleotide
caggtacagttgcaggagtccggcggaggcagcgttcaggccggtggcttcctgaggctgtcctgcgtcgccagcggctatggatattgcggctacgatatgtcctggtacagacaggtccctgggaaagaacgcgagttcgtggctcttatcacatccgacaggtccgtgtcctatgaggactctgtcaagggccgtttcagcatcagccgtgacaacgcaaaaaacacggcttacttggagatgaaccggcttacccccgacgataccgcgatttattactgcaagaccagcacagcagccagggaaaataattggtgtcggagccgttatcgtatcgcctcttggggacagggaacccaggtgactgtctcctca

SEQ ID NO: 320 - DNA, 378 bp, Artificial Sequence
  Description of Artificial Sequence: Synthetic polynucleotide
caggtgcagctccaggagtccggcggaggctcagtacaagctggcggttcactcaggttgagttgtgtcgccagtggctacggctattgtggctatgatatgtcttggtatcgccagacccccggcaaggagcgtgagttcgtggcactcatcacgtccgaccggatcgcctcttacgaagactctgtcaagggccgttttattatcagccgcgacaacgcaaaaaacactggttatctcgacatgactcgggtgacccccgatgacactgccatctactattgcaaaacctctgctgcggcccgcgagaactcctggtgccgtagtcgctaccgcgtcgcctcctggggacagggtacacaggtgaccgttagctcc

SEQ ID NO: 321 - DNA, 378 bp, Artificial Sequence
  Description of Artificial Sequence: Synthetic polynucleotide
caggtccaactgcaagagtctggcggtggctccgtgcaggctggcggtagtctgcgcctgtcttgtgcagtcagcgggtacgactactgcggttatgatgtcagatggtatcgccgtgctcccggcaaggaacgcgagttcgtctctggcattgactccgacggctctacctcctatgccgatagcgtaaagggaaggttcaccatcagccaggacaacgctgagaacaccagctacttgcacatgttctcccttaaacctgaggacacagctatgtattactgtaaaactgagagcccggctggcgagagcgcctggtgtcgcaactttcgtggcatggactactggggtaagggcacccaggttactgtctctagt

SEQ ID NO: 322 - DNA, 381 bp, Artificial Sequence
  Description of Artificial Sequence: Synthetic polynucleotide
caggtgcaacttcaggagagcggtggcggttcagtgcaggctgggggaagcctgcgcctgtcttgcaccgcttccggctacacctattccagtgccttcatggcctggttccgccaggcccctggaaaggaacgcgaaggcgtggctgccatttatacacgggatgggggaaccgtctacgcggactccgtcaagggaagattcaccattagccaggataatgctaagaacatcctgtacctccagatgaactccctcaaagccgaggatactgctatgtactattgtgccgctaagattccgcagccaggccgggcatccctcctggacagccagacctatgactactggggacaggggacccaggtgaccgtgtcttcc

SEQ ID NO: 323 - DNA, 381 bp, Artificial Sequence
  Description of Artificial Sequence: Synthetic polynucleotide
caggtgcagctccaggagtccggcggtggcagtgtccaggcaggaggcagtctgcgtctgtcttgcactgcctcaggctacacatactcaagcgcattcatggcctggttcaggcaggcccctgggaaggagcgcgagggtgtggcagctatctacacccgcgatggcggtactgtgtacgccgatagtgtcaaggggcgctttaccatttctcaggacaacgcgaagaacaccctgtacttgcagatgaacagcctgaagccggaggatactgctatgtattactgcgccgcaaaaatgccccagccgggccgcgcgtctttgctggattcccagacatacgactactgggggcagggcacccaggttacggttagctcc

SEQ ID NO: 324 - DNA, 366 bp, Artificial Sequence
  Description of Artificial Sequence: Synthetic polynucleotide
caggttcagctgcaagagagcgggggtgggtctgtgcaggctggtggcagcttgcgccttagttgcgcggcttctggttatacttattgttcctacgatatgtcatggtatcgtcaggctcctggcaagaaacgggagttcgtctctgccttcaactccgatggcaccactagctatgcagattctgtgaaaggcagattcaccatctctcaggacaaggccaagaataccgtgtacctccagatgaacagcctgaagccagaggataccgctatgtactattgcaagacagatcctcacgtgcaatcctctggtggctactgtccgccctactggggccagggcacacaggtaacggttagttcc

SEQ ID NO: 325 - DNA, 366 bp, Artificial Sequence
  Description of Artificial Sequence: Synthetic polynucleotide
caggtgcagctccaggaatctggcggaggttccgtgcaggccggtggcagcctgaggctcagctgcgccgcgtccgggtatacctactgttcctacgatatgtcctggtatcggcaggctccgggtaaaaagagagagtttgtgtccagctttaacagcgacggcagtacatcttacgctgactccgtgaagggtcgcttcaccattagccaggataacgcaaaaaacacagtgtaccttcagatgaacagtctgaagccagaggacaccgccatgtattactgcaagacggacccgcacgctgattggggtgccccttgcgggggcgattattggggccaaggcacccaggtgactgtttcttcc

SEQ ID NO: 326 - DNA, 363 bp, Artificial Sequence
  Description of Artificial Sequence: Synthetic polynucleotide
caggtgcagctccaggaatctgggggcggttctgtgcaagcgggcgagagcctgagactgagctgcgccgcgagcggctacacctactgtacctatgacatgacttggtacagacaggcccccggaaaaaagcgcgagttcgtcagcggtatccatagcgacggtactacctcttacgcagattccgtgaaaggccgcttcacaatttctcaggacaatgccgaaaacaccgtgtacctccagatgaactccctgaagccagaggataccgcgatgtattactgcaagacagaccccatcgccaccatcacccgccggtgcgactcatactgggggcagggcactcaggtcacagtctcatct

SEQ ID NO: 327 - DNA, 366 bp, Artificial Sequence
  Description of Artificial Sequence: Synthetic polynucleotide
caggtgcaacttcaggaatcaggaggcggtagtgtgcaagcgggcggaagtctgcgcctgagctgcgctgcctccgggagcacgtattgtacgtatgatatgacgtggtacaggcaggcccctggcaagcgcagggagttcgtgagtgcaatcaactcagatggcagcacctcttatgctgacagcgtgaaagggagattcactatctcccaggacaacgcaaaaaacaccgtctatctccagatgaactctctgaagcccgaagacaccgcgatgtattactgtaagactgatcctaacagcggatggggcgctccttgcggtggcgattattggggacagggcacccaagtgacagttagcagc

SEQ ID NO: 328 - DNA, 366 bp, Artificial Sequence
  Description of Artificial Sequence: Synthetic polynucleotide
caggtgcagcttcaggaaagcggtgggggctccgtccaggcaggcggttcccttcgcctttcttgtgccgcttctggttatacttactgttcatacgatatgtcttggtatcgccaggctcccggcaaaaagcgtgagttcgtctctgccatcgcctccgatggctcaacgtcctacgcggacagtctcaagggtcgcttcaccatttcccaggataatgcaaagaacaccgtgtatctccagatgaactcactgaagcccgaagacacagccatgtactattgcaagactgacccacacgtacagtcttccggcggatactgcccaccttactggggacagggaacccaggtgacagtgagttct

SEQ ID NO: 329 - DNA, 363 bp, Artificial Sequence
  Description of Artificial Sequence: Synthetic polynucleotide
caggtgcagcttcaggaaagtggcgggggtctggtgcagccgggcgggtccctccggctgtcctgtgctgccagcggctacacctattgcagctatgatatgggctggtatcgccaggcccctggaaaaaagagaaagtttgtgtccagcattaacagcgatgggaccacttcttacgctgacagtgttaaagggcgtttcacgatctcccaggacaacgctaaaaacaccgtgtatctccagatgaatagcctgaagcccgaggacaccgcaatgtattactgtaaaactgacccccagacacgtcccggtaagccatgtgctgattattggggccaggggacccaggtgaccgtcagctcc

SEQ ID NO: 330 - DNA, 363 bp, Artificial Sequence
  Description of Artificial Sequence: Synthetic polynucleotide
caggtgcagctccaggagtccggtggcgggtctgtccaagctggcggttcccttcgcctgtcctgtgcagccagtggatatacgtattgcaactacgacatcgcctggtatagacaggcccctggcaaagagcgcaagttcgtatccgcaatcgccagtgacggtatcacctcttatgctgactctgtgaagggtcggttcactatctcccaggataacgctaagaacacagtctacctccagatgaacagcttgaagccggaggacactgcgatgtactattgcaagactgatccgatttccaccatcacaaggatctgcgacccgtactggggccagggcacccaagtgactgtgtcatca

SEQ ID NO: 331 - DNA, 360 bp, Artificial Sequence
  Description of Artificial Sequence: Synthetic polynucleotide
caagtgcagttgcaggagagcggtggcgattctgtgcaggccggaggctccctccgcctgtcctgtgccgcttcaggctacacgtattgttcttatgatatgaagtggtatcgccaggccccaggtaaggaacgcgagttcgtcagcggtattgattccgacggcagtattagctacgccgactccgtgaaaggccgcttcacaattagccaggacaacgcgaaaaacaccgtgtacctccagatgaactctctgaagccagaggataccgccatgtactattgcaagactgagggcactatccccgtaggtgcatgtcctaactactggggccagggcactcaggtaaccgtcagtagc

SEQ ID NO: 332 - DNA, 363 bp, Artificial Sequence
  Description of Artificial Sequence: Synthetic polynucleotide
caagtccagctccaggagagcggcggtggcttggtgcaggccggtggctccctgagactgagctgtgcagcctccgggtatacatattgcagctacgacatgagttggtatcgccaggcaccgggcaaggagagaaagtttgtgtcctctatcaattcagatggcacaacctcctacgccgactcagtcaagggtcgtttcactatttctcaggacaacgctaagaacaccgtgtacctccagatgaacagcctgaagcctgaggatacggccatgtactattgcaaaactgacccccagactagacctggcaagccgtgcgcggactattggggtcagggcacgcaagtcaccgtgtcctca

SEQ ID NO: 333 - DNA, 363 bp, Artificial Sequence
  Description of Artificial Sequence: Synthetic polynucleotide
caggtgcagttgcaggagagcggcggagggtctgtgcaggctggaggtagcctgaagctgtcctgtgctgccagcggctacacctactgtaactacgatatcgcgtggtatcggcaagcgcccggcaaagagcgtaagttcgtgtccgctatcgcctccgatggctctacttcctatgccgacagcgttaaaggtcgcttcaccatctcccaagacaacgccaaaaatacagtgtatcttcagatgaactctctgaaacccgaggatactgcgatgtattactgcaagactgatccaatcgctaccatgaccaggcgctgcgacccttactggggccagggcacccaggtgacggtatcctca

SEQ ID NO: 334 - DNA, 363 bp, Artificial Sequence
  Description of Artificial Sequence: Synthetic polynucleotide
caagtccagcttcaggagtctggtgggggctctgtccaggccgggggctctctgagactgtcttgtgcagcctccgggtacacctactgttcctatgacatgacttggtatcgtcaagcacctggcaaaaagcgtgagttcgtgtctgccatcgactccgacggctctacctcctacgccgactctgtgaaaggtaggttcacaatctcccaggataacgcaaagaatactgtgtacttgcagatgaactccttgaagcccgaggatactgccatgtactattgcaagacagaccctattgctactatctctcgtaggtgtgatagttactggggacaaggcacccaggttaccgtatccagt

SEQ ID NO: 335 - DNA, 360 bp, Artificial Sequence
  Description of Artificial Sequence: Synthetic polynucleotide
caggtgcagctccaagaatctggtggagggtcagtgcaggccggaggcagcctgcgcctgtcttgcgctgcaagcggttacaccagctcctctcgctgtatgggatggttccggcaagctccgggcaaggaaagggaaggagtcgctcgtatctacaccccaaccagaactacgtggtacgccgatagcgtcaaggggcgcttcaccatcagccaggataacgccaagaataccgtgtacctggagatggccagcctcaagccagaggacacggcgaagtatttttgcgctgccggggcgtcctgcgccgtggatttgttctcttactggggtcagggcactcaggtcaccgtgtcaagc

SEQ ID NO: 336 - DNA, 360 bp, Artificial Sequence
  Description of Artificial Sequence: Synthetic polynucleotide
caggttcagctccaggagtccggcggtggctctgtgcaggccggtggctccctgagactgtcctgcgcggcttcaggatacacgtactgctcctatgatatgaagtggtatcgtcaggctccaggcaaaaagagggagttcgtgagcgcgattgattccgatgggagtacctcctacgcggactctgtgaagggacgctttactatttcccaggacaacgcgaagaatacggtctacctgcaaatgaactccctcaagccggaggataccgctatgtattactgtaaaaccgagggaacaattcctgtcggcgtctgccctaattattgggggcagggcacacaagtgactgtctcctcc

SEQ ID NO: 337 - DNA, 360 bp, Artificial Sequence
  Description of Artificial Sequence: Synthetic polynucleotide
caagtccagctgcaagagtctggtgggggcctggtgcaaccagggggcagcttgagactctcctgcgctgccagcgggtatacatactgtagctatgatatgaagtggtacaggcaagcccctggcaaaaagcgcgagttcgtgtccgccatcgactccgacggttccactagctacgcggattccgtgaagggaaggttcactatttctcaggataacgccaagaacaccgtctacctccagatgaactccctgaagccagaggacaccgccatgtactattgtaagaccgagggcacagtgcctgtgggcgtctgtccaaattattggggtcaaggcacccaggtcacagtatcctct

SEQ ID NO: 338 - DNA, 372 bp, Artificial Sequence
  Description of Artificial Sequence: Synthetic polynucleotide
caagtgcagctgcaagagagcggtggcgggtccgtgcaagcaggtggctccttgcgcctgtcctgcgctgccagccccggcacctacacatcccgttatatgggatggtttcgccaggcacctggaaaggaacgcgagggggttgcgactatctggcccgctggcggtaacaccgtttacgccgatagcgtgaaagggcgcttcaccattagtcaagacggggccaaaaagaccgtgtacctccagatgaactccctgaaacctgaagacactgctatgtactattgtgccgcagctaagtacggcgggactagcctggctccttacacatataactactggggccaaggcacccaggtgacagtctcttct

SEQ ID NO: 339 - DNA, 363 bp, Artificial Sequence
  Description of Artificial Sequence: Synthetic polynucleotide
caggtccaactccaggaatccggtgggggcagcgtcgaggccgggggcgcactcaccctctcctgcgtcgcaagcggctatacgtactgtaactacgacattgcttggtatcgccaggccccaggcaaggagcgcaagttcgtttccgccatcgcctctgatggaagcacaagttacgcagattccgtgaaaggccggttcacaatctcacaggacaacgctaagaacaccgtctacttgcagatgaacagtctgaagcccgaagacaccgccatgtattactgcaagaccgatcccatcgccactatgacacgtcgctgtgacccctactggggccaaggcactcaggtgaccgtgagttcc

SEQ ID NO: 340 - DNA, 384 bp, Artificial Sequence
  Description of Artificial Sequence: Synthetic polynucleotide
caggtacagctccaagaatctggcggtggctccgtgcaggccggtggctccttgcgtctgtcctgcaccgccagtggatatactttctccaccatgaagtacatgggatggttccgccaggctccgggaaaggagagggagggcgttgcggccatttggatcgccgctggcaacacttattacgccgattccgtgaaaggccgctttaccatttcccaggacaacacaaagaacaccgtttacctccagatgaatagcctgaagccagaggataccgccctctactattgcgcggcagccaggtacggctttgtccccagcacttggtatctccccgagcgttacaactattggggccagggaactcaggtgactgtcagttcc

SEQ ID NO: 341 - DNA, 363 bp, Artificial Sequence
  Description of Artificial Sequence: Synthetic polynucleotide
caggtgcagttgcaggagtccggcggtgggtctgtgcaggcgggcgggagcctgcgcttggcctgcgccgcaagcggttatacctactgcaattacgacatcgcgtggtatcggcaggcccccggtaaggagcgtaagttcgtgtccgccatcgcgtctgacggaagcacctcttatgccgatagcgtgaaaggaagatttacaatctcccaggataacgccaaaaacaccgtctatttgcagatgaatagtctgaagccagaggacacggctatgtattactgcaagaccgatccaatcgctaccatgaccaggcgctgcgacccatattggggccagggcacgcaggtcaccgtatcctcc

SEQ ID NO: 342 - DNA, 360 bp, Artificial Sequence
  Description of Artificial Sequence: Synthetic polynucleotide
caggtccagttgcaggaaagcggcggtggatcagtccaggctggtgggagtctgcgtctgagctgtgctgccagcggttatacatcctgctcttacgatatgtcttggtacagacaggcccctggcaagaaaagagagttcgtttccgccattcatagtgatggcacaacctcctacgccgacagcatgaaggggaggttcactatctcccaggataatgctaagaataccgtttatctccagatgaactcactgaaaccggaggacaccgcaatgtactattgcaagacggaccctaactactcagaccacgtgtgcccgccttactggggacgcggtactcaagtgactgtgtcaagc

SEQ ID NO: 343 - DNA, 347 bp, Artificial Sequence
  Description of Artificial Sequence: Synthetic polynucleotide
caagtgcagctgcaagaatctggcggaggcttgtgcaagctggcggttccttgcgtctgagctgcgccgcatctgggtatacctacagtagctgcacaatgggttggtatagacaagcgcccggtaaggagcgggaactggtgtccatgctgatctctgacggcagtactttttacgccgactccgtgaagggcagattcacattctcccaggagtacgccaagaacaccgtctaccttcagatgaacagtctgaagcctgaggacaccgctatgtactattgcggctgcgcaacgctcggctcccgcacggtctggggtcagggcacccaggtcaccgtgtcctct

SEQ ID NO: 344 - DNA, 348 bp, Artificial Sequence
  Description of Artificial Sequence: Synthetic polynucleotide
caggttcagcttcaggagtctggtggcggttccgtacaggccgggggctcccttactctcagctgtaccgcgccgggttttactttccggttggcggccatgcgctgggttcgccaggctccgggaaaaggactggagtgggttagcggcattgattccaggggctccaccatctatgccgactctgtgaaggggaggttcacaattagcaaagataatgccaaaaacactctttaccttcagctcaattctttgaaaactgaggatactgcaatgtattactgcgcccagggcgtatacggcgacacttactccgggagccaaggaacacaggtgactgtatcttct

SEQ ID NO: 345 - DNA, 381 bp, Artificial Sequence
  Description of Artificial Sequence: Synthetic polynucleotide
caggtccagttgcaagagtcagggggtgggtccgtacaggcaggaggttccctccgcctcagctgcactgcctctgtcaacacatactgcgaatacaatatgtcctggtatcggcaggcccctggcaaggagagagagttcgtgtccggcgttgactctgatggttccacccgctacagcgagagcgttaaggggcgcttcaccatctcccaggacaacgccaagaacactatgtacctccagatgaatggtctgaagcccgaggacaccgctatgtactattgtaagacatacgtttgtaccttctgttcaggcaacagctgctactatgaatataagtactattacgagggccagggaacgcaggttaccgtatcctct

SEQ ID NO: 346 - DNA, 363 bp, Artificial Sequence
  Description of Artificial Sequence: Synthetic polynucleotide
caggtgcaactccaggagtccggcgggggtagcgtccaggctgggggctccctccgcctgagttgtgctgccagcggttatacttactctaataattgcatgggctggtttaggcaagctccgggcaaggaccgcgagagaattgccaatatctacacaggaggtggcagaactacctacgcagatagtgtgaagggccggttcaccatttctcaggacagtgcgaagtccactgtgtacctccagatgaactcactgaagccggaggacaccgcgatgtattactgcgccgctggctcctgtgggagcgctcgttccgaatactcatactggggccagggcacccaggtgaccgtgtcctcc

SEQ ID NO: 347 - DNA, 360 bp, Artificial Sequence
  Description of Artificial Sequence: Synthetic polynucleotide
caagtgcagctccaggaatctgggggcgggtctgtccaagctggcgggagcctccgcctgagttgtgctgcctctgggtacaccttttgtatggcttggttccgccaagcgcctgggaaggaacgcgagggtgtcgcacgcttctatacacgtgatggatacacatattactctgacagcgttaagggcagattcactatctctcagaataacgctaagaataccctctacttgcagatgaactctctgaaaagcgaggacaccgctatgtactattgcgcagcggatttggcccgctgttccagcaacaagaatgactttcgttactggggtcaggggacacaggtgacagttagtagc

SEQ ID NO: 348 - DNA, 381 bp, Artificial Sequence
  Description of Artificial Sequence: Synthetic polynucleotide
caggtccagctccaggagtcaggaggtggctccgttcaggctggtggctccctccggctgtcctgtgccgcaagcggatacacgtctggaaactactggatgggatggttccgtcaagcccccggcaaagaacgcgagggcgtggctactctgtggactggtggagcctcaaccttctacggcgactctgttaagggccgtttcaccattagtcgcgataacttcaaaaacacactctaccttcagatgaactccctgaaggtcgaggatacagccatgtattactgcgccgctgaccctgccctgcgtctgggagctaacatcctgcgccctgctgaatacaaatattggggtcaagggacacaggtgactgtcagctca

SEQ ID NO: 349 - DNA, 354 bp, Artificial Sequence
  Description of Artificial Sequence: Synthetic polynucleotide
caggtgcagctccaggagtccggcggtggcctggtacagcccggtggcagcttgcgcctgagctgcgccgcttctggatttacattctcccgcagcgccatgacatgggttcgccaggctccaggcaagggcctcgactgggtgtccggcattgacagtggcggaactaccgtgtacgcagattctgttaagggaagattcaccatctcccgcgactccgccaagaacaccctgtacttgcaaatgaacagcctcaagacagaagacacagccgtgtattactgtgccatcggactgccgtggggcaacacatggcgtaccaggggacagggcacacaggtgacagtctcctca

SEQ ID NO: 350 - DNA, 348 bp, Artificial Sequence
  Description of Artificial Sequence: Synthetic polynucleotide
caggtgcagttgcaggagtccggcggaggcagcgtgcaagccggaggtagcctccgcctgagctgcacagcgagccgctacacctatagcagttgcactatgggttggtatcgtcaggcccccggcaaggagagggaactcgtgtcaatggtgttttctgacggttccaccttctacgccgactccgttaaaggtcggttcaccttctctcaggaaaacgccaaaaacaccgtgtacctccagatgaactccctgaaacccgaggataccgctatgtactattgcggatgcgctacactcggctcaagaactatctggggccaaggcactcaggtgactgtctcctcc

SEQ ID NO: 351 - DNA, 348 bp, Artificial Sequence
  Description of Artificial Sequence: Synthetic polynucleotide
caggtgcagctccaggaaagcggaggtggcctcgtgcagcctggtggctccctgagactgtcttgcgcaacatctggattcaccttcaggctcactgctatgcgttgggtgagacaagccccagggaagggcgtcgaatgggtgtctggaatcgactccgctggctctacgatctacgccgacagcgtgaagggccgcttcactatctccaaagataatgctaagaacactctgtatctgcaaatgaacagcctcaaaaccgaagacacagctatgtactattgtgcgcagggtgtctacggtgatacctacagcggttctcagggcactcaggtgacggtgtccagc

SEQ ID NO: 352 - DNA, 348 bp, Artificial Sequence
  Description of Artificial Sequence: Synthetic polynucleotide
caggtccagctccaggagagcggcgggggcagcgtgcaggctgggggttccctgagactgtcctgtgctgcctccggcgatacctacagttcatgtactatgggctggtatcgccaggctccgggcaaggagcgcgacctcgtgagcatgttgatgggcgatggtagcaccttttacgccgatagtgtgaagggccgtttcacctttagccaggagaacgctaaaaacactgtgtacttgcagatgaacagcttgaagcccgaggacactgcaatgtattactgtggctgcgccacgctgggctcccgcacaatttggggccagggcacccaggtgaccgtttctagc

SEQ ID NO: 353 - DNA, 348 bp, Artificial Sequence
  Description of Artificial Sequence: Synthetic polynucleotide
caggtccagctccaggaaagcggcggtggaagcgtgcaggccggtggcagtcttcgcctgtcttgcgctgcgtctggttacacctactcctcatgcactatgggttggtacaggcaggccccagggaaggagcgcgagctggtatccatgctcatttctgacgggtccaccttctacgccgattctgtcaagggcaggtttaccttttcccaggaaaacgccaagtccactgtctatctgcaaatgaactccttgaagcccgaagacactgccatgtattactgtggctgtgcgacgcttggctcaaggacggtgtggggccagggcacgcaggtaaccgtttccagc

SEQ ID NO: 354 - DNA, 348 bp, Artificial Sequence
  Description of Artificial Sequence: Synthetic polynucleotide
caggtgcagctccaagagtctggcggaggctccgtgcaggctggcgggagcctgcgcttgtcctgcgcagccagcggctatacctactcctcttgtactatgggctggtataggcaagcgcctggcaaggagcgcgaactcgtcagcatgttgatctctgacggaagcaccttctacgctgattctgtgaaggggcgttttaccttcagccaggagaacgctaagaacaccgtgtacctccagatgaactctctgaaacctgaagataccgctatgtactattgcggatgcgctaccctgggttctaggaccgtttggggtcagggaacacaggtgacagtatcttcc

SEQ ID NO: 355 - DNA, 348 bp, Artificial Sequence
  Description of Artificial Sequence: Synthetic polynucleotide
caggtccagctccaggaatctggcgggggcctcgtgcaaccagggggttccctgagattgtcttgcgcaaccagtggttttaccttccgcctcgctgccatgcgttgggtgcggcaagcgcccggcaagggcctggagtgggtgtctgggattgattctcggggctccacaatctacgcggacagcgtcaagggtcgcttcactatctctaaggacaatgctaagaacactctgtaccttcagttgaactctctgaaaaccgaggataccgctatgtattactgtgctcagggagtgtatggcgatacatactccggctcccaggggacgcaagtgactgtgagttct

SEQ ID NO: 356 - DNA, 354 bp, Artificial Sequence
  Description of Artificial Sequence: Synthetic polynucleotide
caggtgcagcttcaggagtccggcgggggcctggttcagcccggtgggtccctgcgcctgtcatgcgcagcttccggcttcacctttaggacatctgccatgacttgggtgcgtcaggctcctggcaagggcctcgactgggtgagcggcatcgacagcgggggaaccacagtgtatgccgactccgtcaagggacgcttcaccattagccgcgactccgccaagaacaccctctaccttcagatgaacagcctgaagacggaagacaccgccgtttattactgcgcaatggggctgccttggggcaacacctggaggactcggggccagggaactcaggtgaccgtgtcttcc

SEQ ID NO: 357 - DNA, 348 bp, Artificial Sequence
  Description of Artificial Sequence: Synthetic polynucleotide
caggtgcagcttcaggagtcaggcgggggcagcgtgcaggccggaggctccttgaggctgagttgcgcggccagcggctacacatattctagctgcacaatggggtggtatcgccaggcacccggaaaggagagggaactcgtgtctatggtgttctctgacggctccacattctacgccgattctgtgaagggccggtttaccttctcacaggagaatgccaaaaacaccgtgtatctccagatgaactctttgaagccagaggacacagccatgtattactgtggatgcgctaccctgggctcccgtaccatctggggtcagggcacccaggtgactgtcagctct

SEQ ID NO: 358 - DNA, 348 bp, Artificial Sequence
  Description of Artificial Sequence: Synthetic polynucleotide
caagtccagctccaggagagcgggggcggttccgtccaggcgggcggaagcctccgcctttcatgtgcagctagtggcgacacgtacagctcctgtactatgggctggtacaggcaggccccaggtaaggagcgcgatctggtgtctatgctgatgggcgacggcagtaccttttacgctgatagcgtcaagggccgtttcaccttttctcaggagaacgccaagaataccgtctatcttcaaatgaataacctcaagccagaagatactgctatgtactattgtggttgtgccaccctggggtccagaacaatctggggacagggcacccaggtcactgtgtcctct

SEQ ID NO: 359 - DNA, 348 bp, Artificial Sequence
  Description of Artificial Sequence: Synthetic polynucleotide
caagtccagcttcaggagtctggcgggggctcagtgcaagcaggaggtagcctgaggctgagctgcgctgccagtggttttactttccgcctcaccgccatgcgctgggtgcgccaggcccccggcaagggcctggagtgggtgagcggaatcgactccaggggcagcactatttatgccgactcagtgaaggggagatttactatctccaaggacaatgcaaaaaacaccctttaccttcaactgaactctttgaagaccgaggacacggccatgtattactgcgcacagggagtctacggggacacctactctggctctcagggcacccaggtcactgtgtctagc

SEQ ID NO: 360 - DNA, 348 bp, Artificial Sequence
  Description of Artificial Sequence: Synthetic polynucleotide
caagtccagctccaggagagcggcgggggcctggtgcagcccggtggctctttgaggctcagctgtgctgcctccggcttcacattccgcctgactgcaatgcgttgggtgaggcaggctcctggcaagggtctggagtgggtctctggtatcgacagtagaggctccaccatctacgcagatagcgtaaagggacgcttcaccatctccaaagataacgctaagaacaccctctacctccagcttaacagcctgaagaccgaggacacagctatgtactattgtgcacaaggcgtctacggcgatacctattccggttcccagggcactcaggtgaccgtctcctcc

SEQ ID NO: 361 - DNA, 348 bp, Artificial Sequence
  Description of Artificial Sequence: Synthetic polynucleotide
caggttcagcttcaggagagcggcggtggcctggtccaacctgggggaagcctccgtctgagctgcgccgcatctggattcacctttaggctgtcagctatgcgctgggtccgtcaggccccagggaagggcctggaatgggttagcgggatcgactctcgcgggtctacgatttatgccgactcagtcaaggggcgcttcacgatctctaaggacaacgctaagaacaccctgtacttgcagctgaacagcctgaagaccgaggatacggctatgtattactgtgcgcagggggtctacggggacacctactcaggatcacagggcacccaagtgaccgtgagttcc

SEQ ID NO: 362 - DNA, 354 bp, Artificial Sequence
  Description of Artificial Sequence: Synthetic polynucleotide
caggtacagctccaggagtccggcggaggccttgtgcagcctggtggctccttgagactgagctgtgccgcttccggttttacattctccagctcagccatgacatgggtgagacaggcccctggaaagggactggactgggtttctggcattgactcagggggcacgaccgtgtatgctgactctgttaagggccgcgccaccatcctcaaggacaacgctaagaacacactctacctccagatgaactccctgaagactgaggacacagctgtctactattgtgctactggtctgccttggggtaacacctggcggaccaggggccagggcactcaggtgactgtctcctca

SEQ ID NO: 363 - DNA, 354 bp, Artificial Sequence
  Description of Artificial Sequence: Synthetic polynucleotide
caggtgcagctccaggagtcaggcggtggactcgttcagccgggtggctccctgcgcctcagttgtgcgacctctggctttaccttctccagctccgctatgacctgggtgaggcaagcacctgggaagggcctcgattgggtctccggcattgattctggaggcaccactgtctacgccgacagcgtgaagggcagattcacaatcagtaaggacaacgctaagaacactctgtacctgcaaatgaacagcctgaagaccgaggacaccgctgtttattactgcgcaacgggactgccttggggtaatacttggaggactaccggccagggaactcaggtgactgtgagttcc

SEQ ID NO: 364 - DNA, 360 bp, Artificial Sequence
  Description of Artificial Sequence: Synthetic polynucleotide
caggtgcagctccaggaatccggtggaggctccgtgcaagcgggcgggtccctgcgcctcagctgtgcagcttctggctataccttctgcatggcctggtttcgccaggcccctgggaaggagagggagggggtggcccgcttttacactagagacagctatacttactatagcgactccgtgaaggggcgctttacgattagccagaataacgccaagaataccttgtacctccagatgaatagtctgaagtccgaggacaccgccatgtattactgtgctgccgaccttacgaggtgcagctccaataagaacgacttccgctactggggccagggtactcaggtcactgtgtccagc

SEQ ID NO: 365 - DNA, 348 bp, Artificial Sequence
  Description of Artificial Sequence: Synthetic polynucleotide
caagtgcagctccaggaaagcggaggcggtctggtccaaccaggagggtccctgcgtctgtcctgcgcggcctccggctttaatttcagactgtatgcgatgcgttgggttcgtcaagcgcccggtaagggcgtggagtgggtgtccggtatcgactcaggaggctctaccatctatgctgactctgtgaagggccgctttaccatcagcaaggacaacgctaaaaataccctgtacttgcagctgaactctctgaaaaccgaggacactgccatgtattactgcgcccagggtgtgtacggcgacacctactctggttcccaaggcacccaggtgacggtctcctcc

SEQ ID NO: 366 - DNA, 360 bp, Artificial Sequence
  Description of Artificial Sequence: Synthetic polynucleotide
caagtgcagctccaggagagcggtggcggttctgtgcaagcgggtgggtccctgcggctgagctgcgctgtgtctggttataccttctgtatggcctggttccgccaggctccgggaaaggagcgcgaaggcgtggctcggttctacaccagagacggttacacatactattccggcagcgtgaagggcaggttcacgatcagccagaataacgctaaaaacaccctgtacctgcaaatgaacagcctgaagagcgaggataccgcgatgtattactgcgcagccgacttgaccagatgctcttccaacaaaaacgacttccgctactggggtcagggcacccaagtcactgtgtcctcc

SEQ ID NO: 367 - DNA, 348 bp, Artificial Sequence
  Description of Artificial Sequence: Synthetic polynucleotide
caagtccagctccaggagagtggaggcggaagcgtgcaggccggtggctccctgagactttcatgcgcagcgtccggctatacatattcttcctgcactatgggctggtacagacaagcgccgggcaaggagcgtgagttggtgagtatgctcatcagcgatggcagtaccttttatgcggactctgtcaagggccgcttcaccttctctcaagagaacgctaaaaacacagtttacctccagatgaactccctgaagcccgaagacactgccatgtatttttgtgggtgtgccactcttggctccaggacggtgtggggccagggcacccaggttaccgtgagcagt

SEQ ID NO: 368 - DNA, 360 bp, Artificial Sequence
  Description of Artificial Sequence: Synthetic polynucleotide
caggtccagctgcaagagtctggagggggcagcgtgcaggctggcggttctctgcgcctgagctgcgctgcgagtgggtacactttctgtatggcatggtttcgccaagctcccggtaaggagcgcgaaggtgtggcccgcttttatactagggacggttacacatattactcagactctgtgaagggccgttttaccatttcccaaaacaatgcaaaaaacaccctgtaccttcagatgaactctctcaaaagcgaggatactgctatgtattactgcgccgcagacctgaccagatgttcatccaataagaatgacttccgctactggggccaagggacccaggtgaccgtgagcagt

SEQ ID NO: 369 - DNA, 348 bp, Artificial Sequence
  Description of Artificial Sequence: Synthetic polynucleotide
caggtccagctgcaagaatctggtgggggttccgtgcaagccggaggcagcctgaggctcagctgcgccgcaagcggatacacatattcctcttgcactatgggttggtatcgccaggccccaggcaaggaacgtgagctggtctctatgctcatcagtgatggcagcaccttttacgctgattctgtgaagggcagatttacctcttcccaggagaacgctaaaaacactgtgtaccttcagatgaacagcctgaagccagaggacaccgcgatgtactattgcggctgcgcaaccctggggagcaggacagtgtgggggcagggcacacaggtgaccgtctcctca

SEQ ID NO: 370 - DNA, 348 bp, Artificial Sequence
  Description of Artificial Sequence: Synthetic polynucleotide
caggtccagctgcaagagagcggaggcgggctcgtgcagccgggtggcagcctgcgcctttcctgcgctgcgtctggcttcaccttcaggctcaccgctatgagatgggttagacaagctcccggcaagggtctggaatgggtgagcggcatcgacagcagaggtagcacgatctacgctgattccgtcaagggacggttcacaatttccagagacaacgccaagaacacactgtaccttcagttgaactccctgaagaccgaagacgccgctatgtactattgtgcgcagggcgtgtacggcgatacctactcaggctcccagggcacccaggtaacggtgagttcc

SEQ ID NO: 371 - DNA, 354 bp, Artificial Sequence
  Description of Artificial Sequence: Synthetic polynucleotide
caggtacagctgcaagagagtggcggaggcctcgttcaacccggagggagtctgcgcctgtcttgtgctgcctccggcttcaccttttccacttccgctatgacctgggtcaggcaggcccccggcaagggactggactgggtaagtggcatcgactccggtggcactaccgtgtacgcggactccgtgaaaggccgcttcactattagcaaggacaacgctaaaaacacactctacctccagatgaacagcctcaagacagaggacactgccgtgtactattgcgcgaccggcctgccttggggcaacacctggcgcacaagaggtcaagggacacaggtcactgtgagcagc

SEQ ID NO: 372 - DNA, 348 bp, Artificial Sequence
  Description of Artificial Sequence: Synthetic polynucleotide
caggtgcagctgcaagaaagcggaggcggacttgttcagcctgggggctccttgcggctgtcctgtgctgcctcaggctttacttttcgtctgacagccatgcggtgggtgcggcaggcccctggcaagggtctcgaatgggtttccggtattgactctcgcggctctactatctacgccgactctgtgaagggccgtttcaccatctccaaggataatgccaaaaacacgctgtacttgcagcttaatagcttgaagaccgaggatacggccatgtactattgtgctcagggcgtttacggcgacacttattctggctcccttggcacgcaggtcacggtttctagc

SEQ ID NO: 373 - DNA, 348 bp, Artificial Sequence
  Description of Artificial Sequence: Synthetic polynucleotide
caggtgcagctccaggagagcggaggcggactggtgcagccaggtggcagcctgaggctctcctgtgcggcctcaggttttacctttcgcctgacagccatgcggtgggtcagacaagcgcctgggaaaggtctggagtgggtgtctggtatcgactctcgcggttccaccatctacgccgattctgtgaaggggcgctttacaattagccgcgacaacgccaagaacaccctgtacctccagctcaattccctgaagaccgaggacaccgcgatgtactattgtgcgcaaggcgtctatggggatacctatagcggttctcagggaacccaggtgactgtttccagc

SEQ ID NO: 374 - DNA, 354 bp, Artificial Sequence
  Description of Artificial Sequence: Synthetic polynucleotide
caggtgcagctccaggaatctgggggaggcctggttcgccctgggggtagcctgagactgagctgtgcagcctctggattcactttctcccgttccgcaatgacctgggtccgccaggccccaggcaaggggttggattgggtgtctggcattgattccgggggcaccactgtgtacgcggactccgtgaagggccgcttcaccatcagccgcgatagcgccaaaaacacgctgtatctccagatgaacagcctgaagaccgaggacactgccgtctactattgtgctatcggcctgccctggggcaacacatggcgtacacgcggtcagggcacgcaggtgaccgtgtcttct

SEQ ID NO: 375 - DNA, 354 bp, Artificial Sequence
  Description of Artificial Sequence: Synthetic polynucleotide
caggtccagcttcaggaaagcggaggcggactggtgcagcccggaggcagtctgcgtctcagctgtacgaccagcgggtttactttctctagtagcgcaatgacttgggtgaggcaggctccgggcaagggtctggactgggtcagcggtatcgacagcggcgggacgactgtgtatgccgattcagtgaaaggacggttcactatctcaaaggacaacgccaaaaacacactgtaccttcagatgaactccctgaagaccgaagacacagcggtgtattactgcgccacagggttgccttggggcaacacttggcgcaccactggacaagggacgcaggtgaccgtttcctct

SEQ ID NO: 376 - DNA, 348 bp, Artificial Sequence
  Description of Artificial Sequence: Synthetic polynucleotide
caggtgcagctccaagagagtggcggaggcctggtgcagcccggtggctctctgaggttgtcttgtgctgcctctggctttaccttcagactgacagccatgcgctgggtccgccaggctcctggtaagggactggagtgggtaagcggtatcgactccagagggagcaccatctatgctgattccgttaagggacggttcaccatctctaaggataatgccaagaacaccctgtatctccagttgaactccctgaaaaccgaggacaccgcgatgtactattgcgcacagggcgtgtatggcgacactcacagcggctctcaaggcacccaggtgaccgtgtcttcc

SEQ ID NO: 377 - DNA, 354 bp, Artificial Sequence
  Description of Artificial Sequence: Synthetic polynucleotide
caggtccagctccaagaatccggcggagggctggtacagccaggaggcagtcttaggctggcttgctctgcgtccggcttcacattttccagctctgccatgacctgggtgcgccaggcacccggaaagggcctggactgggtgagcgggattgatagcggaggcaccacggtgtatgctgacagtgtaaaaggacgcgccactatcctgaaggacaatgccaagaacaccctctatttgcagatgaacagcctgaagactgaagatactgctgtgtattactgtgcaacgggcctgccttggggaaacacttggcggacgcggggccagggcacgcaggtgaccgtgtcttcc

SEQ ID NO: 378 - DNA, 348 bp, Artificial Sequence
  Description of Artificial Sequence: Synthetic polynucleotide
caggttcagctgcaagaatctggtggcggaagcgtgcaagcgggtggctctcttcgtctctcttgtgctgcatccggcgacacctacagctcctgcacaatggggtggtatcgtcaggcccctggcaaggagcgggatctggtcagcatggtcttctctgacggcagcacattctacgctgactccgtcaagggacgtttcaccttctctcaggagaacgcgaagaatactgtgtatcttcagatgaacagcctgaagccggaggatacagcaatgtattactgcggttgcgcgaccctgggtagcaggaccatctggggtcaaggcacccaggtgacagtgtcctcc

SEQ ID NO: 379 - DNA, 354 bp, Artificial Sequence
  Description of Artificial Sequence: Synthetic polynucleotide
caggtccagcttcaggaatcaggaggcgggcttgtgcagccgggaggcagcctgcgcctgtcctgcgcaacctccggctttaccttctccagcggagccatgacctgggtgcggcaggcccccggtaagggcctggattgggtgtctggcatcgactccggcggaaccactgtgtacgctgattctgtgaagggtcgcttcacaattagtaaggacaacgctaagaacaccctgtacctccagatgaactcattgaagacagaggataccgccgtgtactattgtgcaaccggcctcccctgggggaacacctggcgcaccactggtcagggaacacaagtaaccgtgagcagc

SEQ ID NO: 380 - DNA, 354 bp, Artificial Sequence
  Description of Artificial Sequence: Synthetic polynucleotide
caggtccagctccaggagagtggcgggggactcgtgcagcctggtggctctttgcgcctgagctgcgcggcaagcggatttacattttccaccagtgctatgacctgggtgcgccaggctcccggcaagggactggactgggtaagcggtattgattccggcggaacgactgtgtacgctgatagcgtaaagggccgctttaccatcagcaaagacaacgccaaaaataccctttacctgcaaatgaactctttgaagacggaggacaccgctgtgtattactgcgccactggcctcccttggggcaacatctggagaacccgtggtcagggcacccaggttaccgtgtcctcc

SEQ ID NO: 381 - DNA, 354 bp, Artificial Sequence
  Description of Artificial Sequence: Synthetic polynucleotide
caggtccaactccaggagtccggcggaggcttggtgcagcctggaggctctctgcggctgtcctgcgccgcatcaggttttacgttttctcggtctgccatgacctgggtcagacaggcaccaggcaagggcctggattgggtgtccggtattgactctggtggcactaccgtgtatgccgactccgttaagggccgtttcaccatctccagggactctgccaagaacacattgtatttgcaaatgaacggcctcaaaactgaggacaccgcagtctactattgtgcaatcgggcttccgtggggcaacacgtggagaaccaggggccaggggactcaggtcaccgtgtcatcc

SEQ ID NO: 382 - DNA, 348 bp, Artificial Sequence
  Description of Artificial Sequence: Synthetic polynucleotide
caggtccagctccaggagtcaggtggaggcctggtgcaacccggaggctccctccgcctgtcctgtgcagcctccggctttacctttcgcctgaccgcgatgaggtgggttcggcaggcccctggcaaagggctggagtgggttagcggcatcgactccaggggctccaccatttacgccgactctgtcaaggggcgtttcaccatttctaaggacaacgctaagaataccctctacctccagctcaactccctgaagagtgaagacaccgccatgtattactgtgcccagggcgtctacggagacacttacagcgggtcccagggtactcaggtgaccgtgtcttcc

SEQ ID NO: 383 - DNA, 354 bp, Artificial Sequence
  Description of Artificial Sequence: Synthetic polynucleotide
caagtccagttgcaggagtcaggaggtggcctggtgcaacccggtggctccctccgtctgacctgcgctgcgtctggtttcactttctcaacttcagctatgacatgggtccgccaggcaccggggaagggcctcgactgggtatctgggatcgacagcggaggcaccactgtctatgccgattccgtgaaaggacgcttcactattagcaaggacaacgctaaaaacaccctgtatttgcagatgaatagcctcaaaactgaagatactgccgtttactattgcgccactggcctcccctggggcaacacctggcgcacaaggggtcagggtactcaggtaaccgtgtcctct

SEQ ID NO: 384 - DNA, 348 bp, Artificial Sequence
  Description of Artificial Sequence: Synthetic polynucleotide
caggtgcagctgcaagagtccggtggcggtctggtgcagcccggaggcagtctgaggctctcctgcgctgcctctggattcaccttcagcaactacgctatgcgctgggtgcggcaggcccccggcaagggcctggagtgggtcagtggcatcgacagtcgcggaagtactatttatgccgactccgtgaagggaaggttcactatttccaaggacaacgccaaaaacactctgtacttgcagctgaactccttgaagactgaggacactgccatgtactattgcgcccagggagtctatggggacacctattccgggagccagggcactcaggtgaccgtgtcaagt

SEQ ID NO: 385 - DNA, 360 bp, Artificial Sequence
  Description of Artificial Sequence: Synthetic polynucleotide
caggttcagctccaggaatccggtggagggtccgtgcaggccgggggcagcctgagactgtcctgtgctgcctctggttatacgttttgcatggcgtggttccggcaggctcctggaaaagagcgcgagggcgtggcaagattctacactagagatggttacacctattactccgactctgtcaaggggaggtttaccatctctcaggacaacgccaagaacactttgtacctccagatgaactccctgaagtctgaggacaccgccatgtattactgtgcagccgatctgacccggtgcagttccaacaagaacgatttccgctattggggccagggcacacaggtcacagtctcctcc

SEQ ID NO: 386 - DNA, 348 bp, Artificial Sequence
  Description of Artificial Sequence: Synthetic polynucleotide
caagtacagctccaagagtctgggggaggtcttgtgcagcccggaggctctttgcgtctgtcatgtgcggccagcggattcacattcaggctgtctgcaatgcgttgggtgcgccaagcgcctggcaaggggtttgaatgggtgtctggaattgattcccgtggctctaccatctatgccgattctgttaaaggccgctttaccatctccaaggataacgcaaaaaacacactgtacttgcagctgaatagcctgaaaactgaggacaccgctatgtattactgcgctcagggagtgtatggcgacacttattccggcagccagggcactcaggtgacagttagctcc

SEQ ID NO: 387 - DNA, 348 bp, Artificial Sequence
  Description of Artificial Sequence: Synthetic polynucleotide
caggtgcagctccaggagtccggcggtggactcgtgcaacccggcggttcccttagattgtcttgcgccgcttcaggttttacctttcgcttgtccgctatgcggtgggttcgccaagcgccaggaaaaggcctggagtgggtctccggtattgattccagaggctccaccatctacgccgactctgtcgagggcaggttcaccatcagcaaggacaacgcaaagaacaccctgtatcttcagcttaatagtctgaagaccgaggacactgcgatgtattactgcgctcagggagtgtacggtgatacctactccggctcccagggaactcaggtgaccgtctccagc

SEQ ID NO: 388 - DNA, 348 bp, Artificial Sequence
  Description of Artificial Sequence: Synthetic polynucleotide
caagttcagttgcaggagagcggagggggcctggttcagccgggaggctccctgaggctgtcctgcgctgcgagtggcttcacttttaggttgtccgctatgcgctgggtgcgccaggctcctgggaagggtctggagtgggtgtctgggattgactccagaggtagtaccatttacgccgactccgtcaagggacgcttcaccatctccaaggacgatgccaagaacaccctgtatctccagctgaactcactcaagaccgaagacacggcaatgtattactgtgcccagggtgtgtatggtgacacttactctggctctcagggcactcaagtgaccgtttcttcc

SEQ ID NO: 389 - protein, 6 aa, Artificial Sequence
  Description of Artificial Sequence: Synthetic His tag
  SITE (1)..(6): This sequence may encompass 3-6 residues
HHHHHH

SEQ ID NO: 390 - protein, 8 aa, Artificial Sequence
  Description of Artificial Sequence: Synthetic 8xHis tag
HHHHHHHH